(19) United States
(12) Patent Application Publication
(10) Pub. No.: US 2022/0307001 A1
(43) Pub. Date: Sep. 29, 2022

(54) EVOLVED CAS9 VARIANTS AND USES THEREOF

(71) Applicant: President and Fellows of Harvard College, Cambridge, MA (US)

(72) Inventors: David R. Liu, Cambridge, MA (US); Johnny Hao Hu, Cambridge, MA (US)

(73) Assignee: President and Fellows of Harvard College, Cambridge, MA (US)

(21) Appl. No.: 16/976,047

(22) PCT Filed: Feb. 27, 2019
(86) PCT No.: PCT/US2019/019794

§ 371 Date: Aug. 26, 2020

Related U.S. Application Data
(60) Provisional application No. 62/660,666, filed on Apr. 20, 2018.
(60) Provisional application No. 62/636,154, filed on Feb. 27, 2018.

(51) Int. Cl. C12N 9/22; C07K 14/47; C12N 9/78; C12N 15/113

(52) U.S. Cl. CPC C12N 9/22; C07K 14/4702; C12N 9/78; A61K 38/00; C07K 2319/60; C07K 2319/61; C12N 15/113

(57) ABSTRACT

Some aspects of this disclosure provide strategies, systems, reagents, methods, and kits that are useful for engineering Cas9 and Cas9 variants that have increased activity on target sequences that do not contain the canonical PAM sequence (e.g., NGG). In some embodiments, fusion proteins comprising such Cas9 variants and nucleic acid editing domains, e.g., deaminase domains, are provided.

### RELATED APPLICATIONS

This application claims priority under 35 U.S.C. § 119(e) to U.S. provisional patent applications, U.S. Ser. No. 62/636,154 filed Feb. 27, 2018, and U.S. Ser. No. 62/660,666 filed Apr. 20, 2018; each of which is incorporated herein by reference.

### BACKGROUND OF THE INVENTION

Targeted editing of nucleic acid sequences, for example, the targeted cleavage or the targeted introduction of a specific modification into genomic DNA, is a highly promising approach for the study of gene function and also has the potential to provide new therapies for human genetic diseases.⁴⁰ An ideal nucleic acid editing technology possesses three characteristics: (1) high efficiency of installing the desired modification; (2) minimal off-target activity; and (3) the ability to be programmed to edit precisely any site in a given nucleic acid, e.g., any site within the human genome.⁴¹ Current genome engineering tools, including engineered zinc finger nucleases (ZFNs),⁴² transcription activator like effector nucleases (TALENs),⁴³ and most recently, the RNA-guided DNA endonuclease Cas9,⁴⁴ effect sequence-specific DNA cleavage in a genome. This programmable cleavage can result in mutation of the DNA at the cleavage site via non-homologous end joining (NHEJ) or replacement of the DNA surrounding the cleavage site via homology-directed repair (HDR).^{45,46}

One drawback of the current technologies is that both NHEJ and HDR are stochastic processes that typically result in modest gene editing efficiencies as well as unwanted gene alterations that can compete with the desired alteration.⁴⁷ Since many genetic diseases in principle can be treated by effecting a specific nucleotide change at a specific location in the genome (for example, a C to T change in a specific codon of a gene associated with a disease),⁴⁸ the development of a programmable way to achieve such precise gene editing would represent both a powerful new research tool, as well as a potential new approach to gene editing-based human therapeutics.

Another drawback of current genome engineering tools is that they are limited with respect to the DNA sequences that can be targeted. When using ZNFs or TALENS, a new protein must be generated for each individual target sequence. While Cas9 can be targeted to virtually any target sequence by providing a suitable guide RNA, Cas9 technology is still limited with respect to the sequences that can be targeted by a strict requirement for a protospacer-adjacent motif (PAM), typically of the nucleotide sequence 5′-NGG-3′, that must be present immediately adjacent to the 3′-end of the targeted nucleic acid sequence in order for the Cas9 to bind and act upon the target sequence. The PAM requirement thus limits the sequences that can be efficiently targeted by Cas9.

Significantly, 80-90% of protein mutations responsible for human disease arise from the substitution, deletion, or insertion of only a single nucleotide.⁴⁵ Most current strategies for single-base gene correction include engineered nucleases (which rely on the creation of double-strand breaks, DSBs, followed by stochastic, inefficient homology-directed repair, HDR), and DNA-RNA chimeric oligonucleotides.⁶¹ The latter strategy involves the design of an RNA/DNA sequence to base pair with a specific target sequence in genomic DNA except at the nucleotide to be edited. The resulting mismatch is recognized by the cell's endogenous repair system and fixed, leading to a change in the sequence of either the chimera or the genome. Both of these strategies suffer from low gene editing efficiencies and unwanted gene alterations, as they are subject to both the stochasticity of HDR and the competition between HDR and non-homologous end-joining, NHEJ.⁶²-6⁴ HDR efficiencies vary according to the location of the target gene within the genome,⁶⁵ the state of the cell cycle,⁶⁶ and the type of cell/tissue.⁶⁷ The development of a direct, programmable way to install a specific type of base modification at a precise location in genomic DNA with enzyme-like efficiency and no stochasticity therefore represents a powerful new approach to gene editing-based research tools and human therapeutics.

### SUMMARY OF THE INVENTION

The clustered regularly interspaced short palindromic repeat (CRISPR) system is a recently discovered prokaryotic adaptive immune system⁴⁹ that has been modified to enable robust and general genome engineering in a variety of organisms and cell lines.⁵⁰ CRISPR-Cas (CRISPR-associated) systems are protein-RNA complexes that use an RNA molecule (sgRNA) as a guide to localize the complex to a target nucleic acid sequence via base-pairing.⁵¹ In the natural systems, a Cas protein then acts as an endonuclease to cleave the targeted DNA sequence.⁵² The target nucleic acid sequence must be both complementary to the sgRNA and also contain a “protospacer-adjacent motif” (PAM) at the 3′-end of the complementary region in order for the system to function.⁵³ The requirement for a PAM sequence limits the use of Cas9 technology, especially for applications that require precise Cas9 positioning, such as base editing, which requires a PAM approximately 13-17 nucleotides from the target base^{51,52}, and some forms of homology-directed repair⁵⁴, which are most efficient when DNA cleavage occurs ˜10-20 base pairs away from a desired alteration⁵⁵⁻⁵⁷ To address this limitation, researchers have harnessed natural CRISPR nucleases with different PAM requirements and engineered existing systems to accept variants of naturally recognized PAMs. Other natural CRISPR nucleases shown to function efficiently in mammalian cells include Staphylococcus aureus Cas9 (SaCas9)⁵⁸, Acidaminococcus sp. Cpf1 (AsCpf1), Lachnospiraceae bacterium Cpf1⁵⁹, Campylobacter jejuni Cas9⁶⁰, Streptococcus thermophilus Cas9⁶¹, and Neisseria meningitides Cas9^{62,63}. None of these mammalian cell-compatible CRISPR nucleases, however, offers a PAM that occurs as frequently as that of SpCas9.

Provided herein are novel Cas9 variants that exhibit activity on target sequences that do not include the canonical PAM sequence (5′-NGG-3′, where N is any nucleotide) at the 3′-end. These Cas9 domains are also referred to herein as “xCas9” domains. Such Cas9 variants are not restricted to target sequences that include the canonical PAM sequence 5′-NGG-3′ at the 3′-end. The Cas9 domains provided herein comprise one or more mutations and are capable of recognizing a target nucleic acid sequence that does not comprise the canonical 5′-NGG-3′ PAM at the 3′-end. For example, any of the Cas9 domains provided herein may recognize a target nucleic acid sequence that comprises a 5′-NGT-3′, 5′-NGA-3′, 5′-NGC-3′, or 5′-NNG-3′ PAM at the 3′-end, wherein N is an A, G, T, or C. In some embodiments, the 3′ end of the target sequence is directly adjacent to a AAA, AAC, AAG, AAT, CAA, CAC, CAG, CAT, GAA, GAC, GAG, GAT, TAA, TAC, TAG, TAT, ACA, ACC, ACG, ACT, CCA, CCC, CCG, CCT, GCA, GCC, GCG, GCT, TCA, TCC, TCG, TCT, AGA, AGC, AGT, CGA, CGC, CGT, GGA, GGC, GGT, TGA, TGC, TGT, ATA, ATC, ATG, ATT, CTA, CTC, CTG, CTT, GTA, GTC, GTG, GTT, TTA, TTC, TTG, or TTT PAM sequence. In some embodiments, the 3′ end of the target sequence is directly adjacent to a PAM sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA.

The potential of the Cas9 system for genome engineering is immense. Its unique ability to bring proteins to specific sites in a genome programmed by the sgRNA can be developed into a variety of site-specific genome engineering tools beyond nucleases that introduce double-strand breaks to initiate cellular repair processes that often result in the random insertions or deletions at a target site specified by the guide RNA (gRNA). Cas9 domains that have been evolved to recognize non-NGG PAM sequences greatly expand the breadth of targets available for site-sensitive genome editing applications, such as single-nucleotide base editing, which enables direct and irreversible conversion of one target DNA base into another in a programmable manner, and homology-directed repair (HDR).

Among the known Cas proteins, Streptococcus pyogenes Cas9 has been mostly widely used as a tool for genome engineering.⁵⁴ This Cas9 domain is a large, multi-domain protein containing two distinct nuclease domains. Point mutations can be introduced into Cas9 to abolish nuclease activity, resulting in a dead Cas9 (dCas9) that still retains its ability to bind DNA in a sgRNA-programmed manner.⁵⁵ In principle, such Cas9 variants, when fused to another protein or domain, can target that protein to virtually any DNA sequence simply by co-expression with an appropriate sgRNA. Thus, this disclosure also comtemplates fusion proteins comprising such Cas9 variants and a nucleic acid editing domain (e.g., a deaminase, a nuclease, a nickase, a recombinase, a methyltransferase, a methylase, an acetylase, an acetyltransferase, a transcriptional activator, or a transcriptional repressor domain), as well as the use of such fusion proteins in correcting mutations in a genome (e.g., the genome of a human subject) that are associated with disease, generating mutations in a genome to prevent or treat a disease, or generating mutations in a genome (e.g., the human genome) to decrease or prevent expression of a gene.

Some aspects of this disclosure provide strategies, systems, proteins, nucleic acids, compositions, cells, reagents, methods, and kits that are useful for the targeted binding, editing, and/or cleaving of nucleic acids, including editing a single site within a subject's genome, e.g., a human subject's genome. In some embodiments, Cas9 domains are provided that comprise at least one, at least two, at least three, at least four, at least five, at least six, at least seven, at least eight, at least nine, at least ten, or more mutations as compared to a naturally occurring Cas9 domain, and that exhibit activity on target sequences that do not include the canonical PAM (5′-NGG-3′, where N is any nucleotide) at the 3′-end. In addition, in some embodiments, the Cas9 domains exhibit greater DNA specificity and/or lower off-target acvitity than a Streptococcus pyogenes Cas9 domain (SpCas9) on target sequences that include the canonical 5′-NGG-3′ PAM at the 3′-end. Additionally, in some embodiments, the Cas9 domains have minimal off-target activity when targeting a target sequence that does not comprise the canonical 5′-NGG-3′ PAM at the 3′-end. Examples of such Cas9 mutations are provided in FIG. 1F and FIG. 15. These Cas9 domains establish that there is no necessary trade-off between Cas9 editing efficiency, PAM compatability, and nucleic acid (e.g., DNA) specificity. In some embodiments, fusion proteins of Cas9 and nucleic acid editing domains, e.g., deaminase domains, are provided. In some embodiments, methods for targeted nucleic acid binding, modifying, editing, and/or cleaving are provided. In some embodiments, reagents (e.g., vectors) and kits for the generation of targeted nucleic acid binding, modifying, editing, and/or cleaving proteins, e.g., fusion proteins of Cas9 variants and nucleic acid editing domains, are provided.

Some aspects of this disclosure provide Cas9 domains comprising an amino acid sequence that is at least 80% identical to the amino acid sequence of Cas9 as provided by any of the sequences set forth in SEQ ID NOs: 9-262, wherein the amino acid sequence of the Cas9 domain comprises at least one mutation in an amino acid residue selected from the group consisting of amino acid residues 51, 86, 115, 261, 274, 331, 319, 341, 388, 405, 435, 461, 510, 522, 548, 593, 653, 712, 715, 772, 777, 798, 811, 839, 847, 955, 967, 991, 1139, 1199, 1227, 1229, 1296, and 1318 of S. pyogenes Cas9 having the amino acid sequence provided in SEQ ID NO: 9, or in a corresponding amino acid residue in any of the amino acid sequences provided in SEQ ID NOs: 10-262. In some embodiments, the Cas9 domain comprises a RuvC and an HNH domain. In some embodiments, the amino acid sequence of the Cas9 domain is not identical to the amino acid sequence of a naturally occurring Cas9 domain. In some embodiments, the Cas9 domain is a nuclease-inactive Cas9 domain. In some embodiments, the Cas9 domain is a Cas9 nickase. In some embodiments, the amino acid sequence of the Cas9 domain comprises at least one mutation selected from the group consisting of X51I, X86L, X115H, X261G, X274E, X331Y, X319T, X341H, X388K, X405Y, X435N, X461I, X510E, X522D, X548V, X593A, X653S, X712K, X715V, X772R, X777N, X798K, X811I, X839G, X847F, X955I, X967K, X991V, X1139A, X1199T, X1227S, X1229S, X1296N, and X1318S of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 domain comprises at least one mutation selected from the group consisting of L51I, F86L, R115H, D261G, D274E, D331Y, A319T, Q341H, E388K, F405Y, D435N, R461I, K510E, N522D, I548V, T593A, R653S, Q712K, G715V, S777N, K772R, E798K, L811I, D839G, L847F, V955I, R967K, A991V, V1139A, P1199T, A1227S, P1229S, K1296N, and L1318S of the amino acid sequence provided in SEQ ID NO: 9, or a corresponding mutation in any of the amino acid sequences provided in SEQ ID NOs: 10-262.

In some embodiments, the Cas9 domain further comprises at least one mutation in an amino acid residue selected from the group consisting of amino acid residues 108, 141, 175, 217, 230, 257, 262, 267, 284, 294, 324, 405, 409, 466, 480, 543, 673, 694, 711, 1063, 1207, 1219, 1224, 1256, 1264, 1356, and 1362 of S. pyogenes Cas9 having the amino acid sequence provided in SEQ ID NO: 9, or in a corresponding amino acid residue in any of the amino acid sequences provided in SEQ ID NOs: 10-262. In some embodiments, the amino acid sequence of the Cas9 domain comprises at least one mutation selected from the group consisting of X108G, X141Q, X175T, X217A, X230F, X230S, X257N, X262T, X267G, X284N, X294R, X324L, X405I, X409I, X466A, X480K, X543D, X673E, X694I, X711E, X1063V, X1207G, X1219V, X1224N, X1256K, X1264Y, X1356I, and X1362P of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 domain comprises at least one mutation selected from the group consisting of E108G, K141Q, N175T, S217A, P230F, P230S, D257N, A262T, S267G, D284N, K294R, R324L, F405I, S409I, T466A, E480K, E543D, K673E, M694I, A711E, I1063V, E1207G, E1219V, K1224N, Q1256K, H1264Y, L1356I, and L1362P of the amino acid sequence provided in SEQ ID NO: 9, or a corresponding mutation in any of the amino acid sequences provided in SEQ ID NOs: 10-262.

In some embodiments, the Cas9 domain further comprises at least one mutation in an amino acid residue selected from the group consisting of amino acid residues 23, 122, 137, 182, 394, 474, 554, 654, 660, 727, 763, 845, 847, 1100, 1135, 1218, 1224, 1333, 1335, and 1337 of S. pyogenes Cas9 having the amino acid sequence provided in SEQ ID NO: 9, or in a corresponding amino acid residue in any of the amino acid sequences provided in SEQ ID NOs: 10-262. In some embodiments, the amino acid sequence of the Cas9 domain comprises at least one mutation selected from the group consisting of X23N, X122, X137, X182, X394H, X474I, X554R, X654L, X660, X727P, X763I, X845, X847, X1100I, X1135, X1218, X1224N, X1333, X1335, and X1337 of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 domain comprises at least one mutation selected from the group consisting of D23N, Q394H, T474I, K554R, R654L, L727P, M763I, V1100I, and K1224N of the amino acid sequence provided in SEQ ID NO: 9, or a corresponding mutation in any of the amino acid sequences provided in SEQ ID NOs: 10-262.

Some aspects of this disclosure provide Cas9 domains comprising an amino acid sequence that is at least 80% identical to the amino acid sequence of Cas9 as provided by any of the sequences set forth in SEQ ID NOs: 9-262, wherein the amino acid sequence of the Cas9 domain comprises at least one mutation in an amino acid residue selected from the group consisting of amino acid residues 108, 175, 217, 230, 257, 262, 267, 294, 324, 409, 461, 466, 480, 543, 673, 694, 711, 777, 1063, 1207, 1219, 1256, 1264, and 1356 ofS. pyogenes Cas9 having the amino acid sequence provided in SEQ ID NO: 9, or in a corresponding amino acid residue in any of the amino acid sequences provided in SEQ ID NOs: 10-262. In some embodiments, the amino acid sequence of the Cas9 domain comprises at least one mutation selected from the group consisting of X108G, X175T, X217A, X230F, X257N, X262T, A267G, X294R, X324L, X409I, X461I, X466A, X480K, X543D, X673E, X694I, X711E, X777N, X1063V, X1207G, X1219V, X1256K, X1264Y, and X1356I of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 domain comprises at least one mutation selected from the group consisting of E108G, N175T, S217A, P230F, D257N, A262T, S267G, K294R, R324L, S409I, R461I, T466A, E480K, E543D, K673E, M694I, A711E, S777N, I1063V, E1207G, E1219V, Q1256K, H1264Y, and L1356I of the amino acid sequence provided in SEQ ID NO: 9, or a corresponding mutation in any of the amino acid sequences provided in SEQ ID NOs: 10-262.

Other aspects of this disclosure provide Cas9 domains comprising an amino acid sequence that is at least 80% identical to the amino acid sequence of Cas9 as provided by any of the sequences set forth in SEQ ID NOs: 10-262, wherein the amino acid sequence of the Cas9 domain comprises at least one mutation in an amino acid residue selected from the group consisting of amino acid residues 108, 217, 262, 409, 480, 543, 694, 1219, and 1356 of the amino acid sequence provided in SEQ ID NO: 9, or in a corresponding amino acid residue in any of the amino acid sequences provided in SEQ ID NOs: 10-262. In some embodiments, the amino acid sequence of the Cas9 domain comprises at least one mutation selected from the group consisting of X108G, X217A, X262T, X409I, X480K, X543D, X694I, X1219V, and X1356I of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 domain comprises at least one mutation selected from the group consisting of E108G, S217A, A262T, S409I, E480K, E543D, M694I, E1219V, and L1356I of the amino acid sequence provided in SEQ ID NO: 9, or a corresponding mutation in any of the amino acid sequences provided in in SEQ ID NOs: 10-262.

Other aspects of this disclosure provide Cas9 domains comprising an amino acid sequence that is at least 80% identical to the amino acid sequence of Cas9 as provided by any of the sequences set forth in SEQ ID NOs: 10-262, wherein the amino acid sequence of the Cas9 domain comprises at least one mutation in an amino acid residue selected from the group consisting of amino acid residues 262, 324, 409, 480, 543, 694, and 1219 of the amino acid sequence provided in SEQ ID NO: 9, or in a corresponding amino acid residue in any of the amino acid sequences provided in SEQ ID NOs: 10-262. In some embodiments, the amino acid sequence of the Cas9 domain comprises at least one mutation selected from the group consisting of X262T, X324L, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 domain comprises at least one mutation selected from the group consisting of A262T, R324L, S409I, E480K, E543D, M694I, and E1219V of the amino acid sequence provided in SEQ ID NO: 9, or a corresponding mutation in any of the amino acid sequences provided in in SEQ ID NOs: 10-262.

In some embodiments, the Cas9 domain comprises a HNH nuclease domain. The HNH nuclease domain of Cas9 functions to cleave the DNA strand complementary to the guide RNA (gRNA). Its active site consists of a Ppa-metal fold, and its histidine 840 activates a water molecule to attack the scissile phosphate, which is more electrophilic due to coordination with a magnesium ion, resulting in cleavage of the the 3′-5′ phosphate bond. In some embodiments, the amino acid sequence of the HNH domain is at least 80%, at least 85%, at least 90%, at least 92%, at least 95%, at least 96%, at least 97%, at least 98%, at least 99%, or at least 99.5% identical to the amino acid sequence of the HNH domain of any of SEQ ID NOs: 9-262. In some embodiments, the amino acid sequence of the HNH domain is identical to the amino acid sequence of the HNH domain of any of SEQ ID NOs: 9-262.

In some embodiments, the Cas9 domain comprises a RuvC domain. The RuvC domain of Cas9 cleaves the non-target DNA strand. It is encoded by sequentially disparate sites which interact in the tertiary structure to form the RuvC cleaveage domain and consists of an RNase H fold structure. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 9-262. In some embodiments, the amino acid sequence of the RuvC domain is identical to the amino acid sequence of the RuvC domain of any of SEQ ID NOs: 9-262.

In some embodiments, the Cas9 domain comprises one or more mutations that affects (e.g., inhibits) the ability of Cas9 to cleave one or both strands of a nucleic acid (e.g. DNA) duplex. In some embodiments, the Cas9 domain comprises a D10A and/or a H840A mutation of the amino acid sequence provided in SEQ ID NO: 9, or a corresponding mutation in any of the amino acid sequences provided in SEQ ID NOs: 10-262. In some embodiments, the Cas9 domain comprises a D10X₁ and/or a H840X₂ mutation of the amino acid sequence provided in SEQ ID NO: 9, or a corresponding mutation in any of the amino acid sequences provided in SEQ ID NOs: 10-262, wherein X₁ is any amino acid except for D, and X₂ is any amino acid except for H. In some embodiments, the Cas9 domain comprises an D10A mutation of the amino acid sequence provided in SEQ ID NO: 9, or a corresponding mutation in any of the amino acid sequences provided in SEQ ID NOs: 10-262. In some embodiments, the Cas9 domain comprises an H at amino acid residue 840 of the amino acid sequence provided in SEQ ID NO: 9, or a corresponding residue in any of the amino acid sequences provided in SEQ ID NOs: 10-262. In some embodiments, the Cas9 domain comprises an H840A mutation of the amino acid sequence provided in SEQ ID NO: 9, or a corresponding mutation in any of the amino acid sequences provided in SEQ ID NOs: 10-262. In some embodiments, the Cas9 domain comprises a D at amino acid residue 10 of the amino acid sequence provided in SEQ ID NO: 9, or a corresponding residue in any of the amino acid sequences provided in SEQ ID NOs: 10-262.

In some embodiments, the Cas9 domain of the present disclosure exhibits increased binding (e.g., increased DNA binding specificity) activity and/or lower off-target activity, on a target sequence that does not include the canonical PAM sequence (5′-NGG-3′) at its 3′-end as compared to Streptococcus pyogenes Cas9 as provided by SEQ ID NO: 9.

In some embodiments, the amino acid sequence of the Cas9 domain comprises any of the mutations provided herein. For example, in some embodiments, the amino acid sequence of the Cas9 domain comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, or at least twenty-four mutations selected from the group consisting of X108G, X175T, X217A, X230F, X257N, X262T, A267G, X294R, X324L, X409I, X461I, X466A, X480K, X543D, X673E, X694I, X711E, X777N, X1063V, X1207G, X1219V, X1256K, X1264Y, and X1356I of the amino acid sequence provided in SEQ ID NO: 9, or a corresponding mutation in any of the amino acid sequences provided in SEQ ID NOs: 10-262, wherein X represents any amino acid. In some embodiments, the mutations may be E108G, N175T, S217A, P230F, D257N, A262T, S267G, K294R, R324L, S409I, R461I, T466A, E480K, E543D, K673E, M694I, A711E, S777N, I1063V, E1207G, E1219V, Q1256K, H1264Y, and L1356I of the amino acid sequence provided in SEQ ID NO: 9, or a corresponding mutation in any of the amino acid sequences provided in SEQ ID NOs: 10-262. In some embodiments, the amino acid sequence of the Cas9 domain comprises at least one, at least two, at least three, at least four, at least five, at least six, at least seven, at least eight, or at least nine mutations selected from the group consisting of X108G, X217A, X262T, X409I, X480K, X543D, X694I, X1219V, and X1356I of the amino acid sequence provided in SEQ ID NO: 9, or a corresponding mutation in any of the amino acid sequences provided in SEQ ID NOs: 10-262, wherein X represents any amino acid. In some embodiments, mutations may be E108G, S217A, A262T, S409I, E480K, E543D, M694I, E1219V, and L1356I of the amino acid sequence provided in SEQ ID NO: 9, or a corresponding mutation in any of the amino acid sequences provided in SEQ ID NOs: 10-262. In some embodiments, the amino acid sequence of the Cas9 domain comprises at least one, at least two, at least three, at least four, at least five, at least six, or at least seven mutations selected from the group consisting of X262T, X324L, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid. In some embodiments, the mutations may be A262T, R324L, S409I, E480K, E543D, M694I, and E1219V of the amino acid sequence provided in SEQ ID NO: 9, or a corresponding mutation in any of the amino acid sequences provided in SEQ ID NOs: 10-262.

As one example, the Cas9 domain may exhibit increased binding to a target sequence, may exhibit increased binding activity at the target sequence, or may exhibit an increase in other activities (e.g., deamination of a nucleobase within the target sequence), depending on whether the Cas 9 protein is fused to an additional domain, such as an enzyme that has enzymatic activity or a transcription factor that modulates expression of one or more genes. In some embodiments, the enzymatic activity modifies a target nucleic acid. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the enzymatic activity is nuclease activity. In certain embodiments, the enzymatic activity is deaminase activity.

In some embodiments, any of the Cas9 domains provided herein may be fused to a second protein (e.g., a fusion protein). In some embodiments, the second protein is a protein that has an activity. In some embodiments, the activity is an enzymatic activity. In some embodiments, the second protein is an effector protein. In some embodiments, the effector protein is capable of modulating expression of a gene. In some embodiments, the effector domain is a nucleic acid editing domain.

In some embodiments, any of the Cas9 domains provided herein may be fused to a protein that has an enzymatic activity. In some embodiments, the enzymatic activity modifies a target nucleic acid. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some embodiments, the enzymatic activity is nuclease activity. In some embodiments, the nuclease activity introduces a double strand break in the target DNA. In some embodiments, the nuclease activity does not introduce a double strand break in the target DNA. In some embodiments, the nuclease activity introduces a nick in one strand in a double-stranded target DNA. In some embodiments, the enzymatic activity is deamination activity. In some embodiments, the deamination activity is cytidine (C) deamination activity. In some embodiments, the deamination activity is adenosine (A) deamination activity.

In some embodiments, any of the Cas9 domains provided herein may be fused to a nucleic acid editing domain. In some embodiments, the nucleic acid editing domain comprises a deaminase domain. In some embodiments, the deaminase domain catalyzes the removal of an amine group from a molecule. In some embodiments, the deaminase domain is a cytidine deaminase domain. In some embodiments, the cytidine deaminase domain deaminates cytidine (C) to yield uracil (U). In some embodiments, the deaminase domain is an adenosine deaminase domain. In some embodiments, the adenosine deaminase domain deaminates adenosine (A) to yield inosine (I).

In some embodiments, the any of the Cas9 domains provided herein may be fused to a transcriptional activator or transcriptional repressor domain. Transcriptional activator domains are regions of a transcription factor which may activate transcription of a gene from a promoter through an interaction or multiple interactions with a DNA binding domain, general transcription factors, and RNA polymerase. In some embodiments, the transcriptional activator domain is a VPR transcriptional activator domain. Transcriptional repressor domains are regions of a transcription factor which may repress transcription of a gene from a protomer through an interaction or multiple interactions with a DNA binding domain, general transcription factors, and RNA polymerase.

In some embodiments, the Cas9 domain exhibits activity on a target sequence having a 3′ end that is not directly adjacent to the canonical PAM sequence (5′-NGG-3′), or a target sequence that does not comprise the canonical PAM sequence (5′-NGG-3′), wherein N is A, C, G, or T. In some embodiments, the activity is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes Cas9 as provided by SEQ ID NO: 9 on the same target sequence.

In some embodiments, the 3′-end of the target sequence is directly adjacent to an NGT, NGA, NGC, or NNG sequence, wherein N is an A, G, T, or C. In some embodiments, the 3′ end of the target sequence is directly adjacent to a AAA, AAC, AAG, AAT, CAA, CAC, CAG, CAT, GAA, GAC, GAG, GAT, TAA, TAC, TAG, TAT, ACA, ACC, ACG, ACT, CCA, CCC, CCG, CCT, GCA, GCC, GCG, GCT, TCA, TCC, TCG, TCT, AGA, AGC, AGT, CGA, CGC, CGT, GGA, GGC, GGT, TGA, TGC, TGT, ATA, ATC, ATG, ATT, CTA, CTC, CTG, CTT, GTA, GTC, GTG, GTT, TTA, TTC, TTG, or TTT PAM sequence. In some embodiments, the 3′-end of the target sequence is directly adjacent to an CGG, AGT, TGG, AGT, CGT, GGG, CGT, TGT, GGT, AGC, CGC, TGC, GGC, AGA, CGA, TGA, GGA, GAA, GAT, or CAA sequence.

In some embodiments, the Cas9 domain activity is measured by a nuclease assay or a nucleic acid binding assay, which are known in the art and would be apparent to the skilled artisan. As provided herein, the Cas9 domain may be fused to one or more domains that confer an activity to the protein, such as a nucleic acid editing activity (e.g., deaminase activity or transcriptional activation activity), which may be measured (e.g., by a deaminase assay or transcriptional activation assay). In some embodiments, the Cas9 domain is fused to a deaminase domain and its activity may be measured using a deaminase assay. In some embodiments, the Cas9 domain is fused to a deaminase domain and its activity may be measured using PCR. In some embodiments, the Cas9 domain is fused to a deaminase domain and its activity may be measured by sequencing the target site. In some embodiments, the Cas9 domain is fused to a deaminase domain and its activity may be measured using high throughput sequencing. In some embodiments, the Cas9 domain is fused to a transcriptional activation domain, and its activity may be measured using a transcriptional activation assay, for example, reporter activation assay where the reporter, e.g., GFP or luciferase, among others, is expressed in response to Cas9 binding to a target sequence.

The fusion proteins of the present disclosure may comprise one or more additional features. For example, in some embodiments, the fusion protein comprises a nuclear localization signal (NLS). In some embodiments, the NLS of the fusion protein is located between the nucleic acid editing domain and the Cas9 domain. In some embodiments, the NLS of the fusion protein is located C-terminal to the Cas9 domain. In some embodiments, the NLS is located N-terminal to the Cas9 domain. In some embodiments, the NLS comprises the amino acid sequence PKKKRKV (SEQ ID NO: 520), MDSLLMNRRKFLYQFKNVRWAKGRRETYLC (SEQ ID NO: 521), or SPKKKRKVEAS (SEQ ID NO: 522). In some embodiments, a NLS may be combined with any of the linkers listed above. As another example, in some embodiments, the fusion protein may further comprise one or more uracil glycosylase inhibitor (UGI) domains. In some embodiments, one UGI domain is located C-terminal to the Cas9 domain. In some embodiments, two UGI domains are located C-terminal to the Cas9 domain. As yet another exmaple, in some embodiments, the fusion protein further comprises a Gam protein. In some embodiments, the Gam protein is located N-terminal to the Cas9 domain.

In some embodiments, the nucleic acid editing domain comprises an enzymatic domain. In some embodiments, the nucleic acid editing domain comprises a nuclease, a nickase, a recombinase, a deaminase, a methyltransferase, a methylase, an acetylase, an acetyltransferase, which may have nuclease activity, nickase activity, recombinase activity, deaminase activity, methyltransferase activity, methylase activity, acetylase activity, or acetyltransferase activity, respectively.

In some embodiments, the nucleic acid editing domain is a deaminase domain. In some embodiments, the deaminase is a cytosine deaminase or a cytidine deaminase. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the effector domain is at least 80%, at least 85%, at least 90%, at least 92%, at least 95%, at least 96%, at least 97%, at least 98%, at least 99%, or at least 99.5% identical to the cytidine deaminase domain of any one of SEQ ID NOs: 400-409.

Some aspects of this disclosure provide fusion proteins comprising a Cas9 domain fused to a nucleic acid editing domain, e.g., a deaminase, and a uracil glycosylase inhibitor (UGI). In some embodiments, the fusion protein comprises a Cas9 domain, a cytidine deaminase, and a UGI domain. In some embodiments, the fusion protein comprises a Cas9 domain and one or more adenosine deaminase domains. Some aspects of this disclosure are based on the recognition that such fusion proteins may exhibit an increased nucleic acid editing efficiency as compared to fusion proteins not that do not comprise a UGI domain. Domains such as the deaminase domains and UGI domains have been described and are within the scope of this disclosure. For example, domains such as deaminase domains and UGI domains have been described in U.S. patent application U.S. Ser. No. 15/331,852, filed Oct. 22, 2016, and International Patent Application No. PCT/US2016/058,344, filed Oct. 22, 2016; the entire contents of each is incorporated herein by reference. It would be appreciated by one of skill in the art that the deaminase domains and UGI domains described in the foregoing references are within the scope of this disclosure and may be fused with any of the Cas9 proteisn provided herein. In some embodiments, the UGI domain comprises the amino acid sequence of SEQ ID NO: 500. In some embodiments, the effector domain is a deaminase domain. In some embodiments, the deaminase is an adenosine deaminase. In some embodiments, the adenosine deaminase is the adenosine deaminase acting on tRNA (ADAT) from Escherichia coli (TadA, for tRNA adenosine deaminase A). It should be appreciated that E. coli TadA (ecTadA) deaminases also include truncations of ecTadA. In some embodiments, the adenosine deaminase comprises the amino acid sequence of SEQ ID NO: 400. In some embodiments, the adenosine deaminase domain comprises the amino acid sequence of SEQ ID NO: 458. In some embodiments, the effector domain is at least 80%, at least 85%, at least 90%, at least 92%, at least 95%, at least 96%, at least 97%, at least 98%, at least 99%, or at least 99.5% identical to the deaminase domain of any one of SEQ ID NOs: 400-458. In some embodiments, the adenosine deaminases provided herein are capable of deaminating an adenosine in a DNA molecule. Other aspects of the disclosure provide fusion proteins comprising a Cas9 domain described herein and an adenosine deaminase domain, for example, an engineered adenosine deaminase domain comprising one or more mutations in the amino acid sequence of SEQ ID NO: 400 capable of deaminating adenosine in DNA. In some embodiments, the fusion protein comprises one or more of a nuclear localization sequence (NLS), an inhibitor of inosine base excision repair (e.g., dISN), and/or a linker. Engineered adenosine deaminase domains have been previsouly described, for example, in International Patent Application No. PCT/US2017/045381, filed Aug. 3, 2017, and U.S. patent application U.S. Ser. No. 15/791,085, filed Oct. 23, 2017; the entire contents of each of which is incorporated herein by reference. It should be appreciated that the adenosine deaminase domains described in the foregoing references are within the scope of this disclosure and may be fused with any of the Cas9 domains provided herein.

In some embodiments, the fusion protein comprising a Cas9 domain provided herein exhibits increased activity on a target sequence that does not comprise the canonical PAM (5′-NGG-3′) at its 3′ end as compared to the activity of a fusion protein comprising a Streptococcus pyogenes Cas9 domain as provided by SEQ ID NO: 9. In some embodiments, the 3′-end of the target sequence is directly adjacent to an NGT, NGA, NGC, or NNG sequence, wherein N is A, G, T, or C. In some embodiments, the 3′-end of the target sequence is directly adjacent to an CGG, AGT, TGG, AGT, CGT, GGG, CGT, TGT, GGT, AGC, CGC, TGC, GGC, AGA, CGA, TGA, GGA, GAA, GAT, or CAA sequence.

Some aspects of this disclosure provide complexes comprising a Cas9 domain provided herein, and a guide RNA (gRNA) bound to the Cas9 domain. Some aspects of this disclosure provide complexes comprising a fusion protein comprising a Cas9 domain as provided herein, and a guide RNA (gRNA) bound to the Cas9 domain. In some embodiments, the guide RNA binds to a target nucleic acid sequence. In some embodiments, the target sequence is a DNA sequence. In some embodiments, the target sequence is a sequence in the genome of a mammal. In some embodiments, the target sequence is a sequence in the genome of a human. In some embodiments, the target sequence is a sequence in the genome of a plant. In some embodiments, the target sequence is a sequence in the genome of a microorganism. In some embodiments, the 3′-end of the target sequence is not immediately adjacent to the canonical PAM sequence (5′-NGG-3′).

Some aspects of this disclosure provide methods of using the Cas9 domains, fusion proteins, or complexes provided herein. For example, the disclosure provides methods comprising contacting a nucleic acid molecule (a) with a Cas9 domain or a fusion protein as provided herein and a guide RNA, wherein the guide RNA is about 15-100 nucleotides long and comprises a sequence of at least about 10 contiguous nucleotides that is complementary to a target sequence; or (b) with a Cas9 domain, a fusion protein comprising a Cas9 domain, or a Cas9 domain or fusion protein complex with a gRNA as provided herein. In some embodiments, the 3′-end of the target sequence is not immediately adjacent to the canonical PAM sequence (5′-NGG-3′). In some embodiments, the 3′-end of the target sequence is directly adjacent to an NGT, NGA, NGC, or NNG sequence, wherein N is A, G, T, or C. In some embodiments, the 3′-end of the target sequence is directly adjacent to an CGG, AGT, TGG, AGT, CGT, GGG, CGT, TGT, GGT, AGC, CGC, TGC, GGC, AGA, CGA, TGA, GGA, GAA, GAT, or CAA sequence. In some embodiments, the target DNA sequence comprises a sequence associated with a disease or disorder. In some embodiments, the target DNA sequence comprises a point mutation associated with a disease or disorder. In some embodiments, the activity of the Cas9 domain, the fusion protein comprising a Cas9 domain, or the complex results in correction of the point mutation. In some embodiments, the step of contacting is performed in vitro in a cell. In some embodiments, the step of contacting is performed in vivo in a subject.

Some aspects of this disclosure provide kits comprising a nucleic acid construct, comprising (a) a nucleotide sequence encoding a Cas9 domain or a fusion protein comprising a Cas9 domain as provided herein; and (b) a heterologous promoter that drives expression of the sequence of (a). In some embodiments, the kit further comprises an expression construct encoding a guide RNA backbone, wherein the construct comprises a cloning site positioned to allow the cloning of a nucleic acid sequence identical or complementary to a target sequence into the guide RNA backbone.

Some aspects of this disclosure provide polynucleotides encoding any of the Cas9 domains, fusion proteins, or guide RNA bound to the Cas9 domain or fusion protein comprising a Cas9 domain provided herein. Some aspects of this disclosure provide vectors comprising such polynucleotides. In some embodiments, the vector comprises a heterologous promoter driving expression of the polynucleotide.

Some aspects of this disclosure provide cells comprising any of the Cas9 domains/proteins, fusion proteins, nucleic acid molecules, complexes, and/or vectors as provided herein.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIGS. 1A-1E show phage-assisted continuous evolution (PACE) of Cas9 variants with broadened PAM compatibility. FIG. 1A, PACE takes place in a fixed-volume “lagoon” that is continuously diluted with fresh host E. coli cells. Upon infection, each selection phage (SP) that encodes a Cas9 variant capable of binding the target PAM and protospacer on the accessory plasmid (AP) induces expression of gene III, resulting in infectious progeny phage that propagate the active Cas9 variant in subsequent host cells.

FIG. 1B, Anatomy of a phage-infected host cell during PACE. The host cell carries the AP, which links Cas9 target DNA binding to phage propagation, and the mutagenesis plasmid (MP), which elevates mutagenesis during PACE. FIGS. 1C-1D, The crystal structure of SpCas9 with the location of xCas9 mutations shown. FIG. 1E, Genotypes of some evolved xCas9 variants, shaded by evolution stage. See FIG. 16 for 95 xCas9 variant genotypes.

FIGS. 2A-2C show transcriptional activation and genomic DNA cleavage by evolved xCas9 3.7 in human cells. FIG. 2A, Transcriptional activation by dSpCas9-VPR and dxCas9(3.7)-VPR targeting GFP reporter plasmids containing the same protospacer but different PAM sites in HEK293T cells. FIG. 2B, Genomic DNA cleavage in HEK293-GFP cells containing a genomically integrated GFP reporter gene by SpCas9 or xCas9 3.7. After 5 days, the cells were analyzed for loss of GFP fluorescence by flow cytometry. FIG. 2C, DNA cleavage of endogenous genomic DNA sites with NGG and non-NGG PAMs by SpCas9 and xCas9 3.7 in HEK293T cells. Indel rates were measured by high throughput sequencing (HTS) 5 days after plasmid transfection. Values and error bars reflect the mean and standard deviation of n=3 biologically independent samples. Target sites are shown in Tables 8 (R1 site), 11, and 12.

FIGS. 3A-3D show cytidine and adenine base editing by xCas9. FIG. 3A, The C⋅G to T⋅A conversion frequencies for 20 endogenous genomic loci in HEK293T cells at the most efficiently edited base 3 days after plasmid transfection are shown. FIG. 3B, The A⋅T to G⋅C conversion frequencies for seven endogenous genomic loci in HEK293T cells at the most efficiently edited base 5 days after plasmid transfection are shown. Values and error bars in FIG. 3A and FIG. 3B reflect the mean and standard deviation of n=3 biologically independent samples. See FIG. 17 for complete HTS results across the protospacer. FIG. 3C, Fraction of T⋅A to C⋅G pathogenic SNPs in ClinVar⁵² that, in principle, can be corrected by SpCas9-BE3 (left), xCas9(3.7)-BE3 (middle), or xCas9(3.7)-BE3+all BE3 variants reported to date (right). FIG. 3D, Fraction of G⋅C to A⋅T pathogenic SNPs in ClinVar that, in principle, can be corrected by SpCas9-ABE (left) or xCas9(3.7)-ABE (right).

FIGS. 4A-4F show off-target editing analysis of xCas9. FIG. 4A, GUIDE-seq32 was performed on SpCas9 and xCas9 3.7 nucleases. Six endogenous genomic sites with NGG PAMs were tested in HEK293T or U20S cells. The percentage of off-target sequencing reads relative to total reads are shown. FIGS. 4B-4D, All GUIDE-seq on-target reads (▪) and off-target reads for three sites in HEK293T cells are shown for SpCas9 and xCas9 3.7. See FIGS. 14A-14E for additional GUIDE-seq results. FIGS. 4E-4F, GUIDE-seq results for two endogenous genomic non-NGG PAM sites in HEK293T cells. No on-target GUIDE-seq reads (▪) were detected for SpCas9 at either of these non-NGG sites. See FIGS. 14A-14E for GUIDE-seq analysis of xCas9 3.6 and FIGS. 15A-15C for HTS validation of GUIDE-seq results. Target sequences are shown in Table 15.

FIGS. 5A-5D show optimization of Cas9 PACE. Luciferase expression in E. coli was used as a proxy of gene III expression during efforts to link Cas9 binding to gene expression for PACE. FIGS. 5A-5B, Seven guide RNAs targeting the luciferase reporter (G1-G7′, see Table 1), as well as a scrambled guide RNA negative control (G0) were tested without dCas9 (white bars) and with ω-dCas9 (FIG. 5A) or dCas9-ω (FIG. 5B) fusions (grey bars). FIG. 5C, Tests of seven different linkers between ω and dCas9. See Table 2 for linker sequences. FIG. 5D, Evolution of w-dCas9 on an NGG PAM site in PACE yielded variants (PACE1, PACE2, and PACE3) that were tested in comparison with canonical (“wt”) ω-dCas9, ω tethered to PACE1 dCas9, and the I12N ω mutant tethered to canonical dCas9. Values and error bars reflect the mean and standard deviation of n=3 biologically independent samples.

FIGS. 6A-6C show PAM profiling of xCas9 variants. FIG. 6A, A plasmid library containing a protospacer with all possible NNN PAM sequences and a spectinomycin resistance gene was electroporated into E. coli along with a plasmid expressing SpCas9 or the xCas9 variant shown in separate experiments. PAMs that are cleaved are depleted from the library when plated on media containing spectinomycin. HTS of the library before versus after selection enables quantification of the change in library composition, resulting in a sequence logo³⁹ for the PAM preference of SpCas9 (left) and xCas9 3.7 (right). FIGS. 6B-6C, PAM depletion scores of Cas9 variants from spectinomycin selection in E. coli, calculated as described previously³⁵, with 1.0 representing complete cleavage of that PAM sequence. Scores for NGN, NNG, GAA, GAT, and CAA are shown in FIG. 6B, while the rest of the PAM sequences are shown in FIG. 6C.

FIGS. 7A-7C show transcriptional activation of reporter site PAM libraries with xCas9. Transcriptional activation by dSpCas9-VPR and dxCas9-VPR variants, transfected as plasmids, on GFP reporter plasmids containing different PAM sites in HEK293T cells. FIG. 7A, earlier generations of xCas9 variants were tested on the R1 site with the NGG, NNN, or NNNNN PAM libraries. Values and error bars reflect the mean and standard deviation of n=3 biologically independent samples. FIGS. 7B-7C, Transcriptional activators dxCas9(3.6)-VPR and dxCas9(3.7)-VPR were tested on two different protospacer reporters, R1 in FIG. 7B and R2 in FIG. 7C, containing adjacent NGG, NNN, or NNNNN PAM libraries. Values and error bars reflect the mean and standard deviation of n=3 biologically independent samples.

FIGS. 8A-8D show transcriptional activation with xCas9 2.0. FIGS. 8A-8D, The transcriptional activator dxCas9(2.0)-VPR was tested on the R1 protospacer (FIGS. 7A-7C and Table 8) with each of the 64 possible NNN PAMs (NAN, NCN, NGN, and NTN) in HEK293T cells. Values and error bars reflect the mean and standard deviation of n=3 biologically independent samples.

FIGS. 9A-9E show transcriptional activation with xCas9 3.7 on all 64 NNN PAM sites and endogenous gene activation in human cells. FIGS. 9A-9D, The transcriptional activator dxCas9(3.7)-VPR was tested on the R1 protospacer (FIGS. 7A-7C and Table 8) with each of the 64 possible NNN PAMs (NAN, NCN, NGN, and NTN) in HEK293T cells. FIG. 9E, Endogenous gene activation was tested using both dSpCas9-VPR and dxCas9(3.7)-VPR to activate expression of the NEUROD1, ASCL1, MIAT, or RHOXF2 at six total sites. RNA expression as measured by RT-qPCR was compared to background expression levels for each gene (measured in the control with no sgRNA) and was normalized to ACTB expression. Values and error bars reflect the mean and standard deviation of n=3 biologically independent samples. Target sites are in Table 9.

FIGS. 10A-10D show transcriptional activation with xCas9 3.6 on all 64 NNN PAM sites. FIGS. 10A-10D, The transcriptional activator dxCas9(3.6)-VPR was tested on the R1 protospacer (FIGS. 7A-7C and Table 8) with each of the 64 possible NNN PAMs (NAN, NCN, NGN, and NTN) in HEK293T cells. Values and error bars reflect the mean and standard deviation of n=3 biologically independent samples.

FIGS. 11A-11D show genomic DNA cleavage and base editing by evolved xCas9 3.6. FIG. 11A, Genomic DNA cleavage in HEK293-GFP cells containing a genomically integrated GFP gene by SpCas9 or xCas9 3.6, transfected as plasmids. After 5 days, the cells were analyzed for loss of GFP fluorescence by flow cytometry. Sequences for all target sites are listed in Table 11. FIG. 11B, DNA cleavage of endogenous genomic DNA sites with a variety of NGG and non-NGG PAMs by SpCas9 and xCas9 3.6 in HEK293T cells. Indel rates were measured by HTS 5 days after plasmid transfection. Sequences for all target sites are listed in Table 12. FIG. 11C, 20 sites containing NG, GAA, GAT, or CAA PAM sites were tested for C⋅G to T⋅A base editing in HEK293T cells by SpCas9-BE3 or xCas9(3.6)-BE3. The C⋅G to T⋅A conversion frequency by HTS at the most efficiently edited base 3 days after plasmid transfection is shown. FIG. 11D, Of the 20 sites in FIG. 11C, seven contained an A in the canonical window for ABE editing⁵ and were tested for A⋅T to G⋅C base editing by SpCas9-ABE and xCas9(3.6)-ABE. The A⋅T to G⋅C conversion frequency by HTS at the most efficiently edited base 5 days after plasmid transfection is shown. Values and error bars reflect the mean and standard deviation of n=3 biologically independent samples. Complete HTS results across the protospacer are provided in FIG. 17.

FIGS. 12A-12C show negative controls lacking guide RNA for nuclease and base editing experiments. To verify genomic DNA cleavage and base editing results, the same sites were sequenced after treatment with SpCas9 nuclease, SpCas9-BE3, or SpCas9-ABE but without any sgRNA. FIG. 12A, Indel rates at endogenous target sites 5 days after treatment of HEK293T cells with SpCas9. FIG. 12B, Target C⋅G to T⋅A conversion 3 days after treatment of HEK293T cells with SpCas9-BE3. FIG. 12C, Target A⋅T to G⋅C base conversion 5 days after treatment of HEK293T cells with SpCas9-ABE. Values and error bars reflect the mean and standard deviation of n=3 biologically independent samples. Complete HTS results across the protospacer are provided in FIG. 17.

FIGS. 13A-13F show Cytidine base editing at 15 additional genomic sites and xCas9 base editing with the BE4 architecture. FIG. 13A, Base editing by SpCas9-BE3 and xCas9(3.7)-BE3 at 15 sites within the FANCF gene in HEK293T cells. The C⋅G to T⋅A conversion frequency at the most efficiently edited base 3 days after plasmid transfection is shown. FIG. 13B, Test of xCas9 3.7 in the BE4 architecture³⁴ on the same sites tested in FIGS. 3A-3D. The C⋅G to T⋅A conversion frequency in HEK293T cells at the most efficiently edited base 3 days after plasmid transfection is shown. FIGS. 13C-13D, Indel frequency following treatment with BE3 or BE4 variants targeting sites with NGG PAMs (FIG. 13C) and non-NGG PAMs (FIG. 13D). FIGS. 13E-13F, Product distribution among edited DNA sequence reads (reads in which the target C is mutated) following treatment with BE3 or BE4 variants targeting sites with NGG PAMs (FIG. 13E) and non-NGG PAMs (FIG. 13F). Since SpCas9 has minimal activity on non-NGG PAM sites, only xCas9(3.7)-BE3 and xCas9(3.7)-BE4 data is compared on non-NGG PAM sites. Values and error bars reflect the mean and standard deviation of n=3 biologically independent samples. Target sites are in Table 12.

FIGS. 14A-14E show additional characterization of xCas9 3.7 and xCas9 3.6 by GUIDE-seq. In addition to the GUIDE-seq data shown in FIGS. 4A-4F, two additional sites in HEK293T cells (FIGS. 14A-14B) and two sites in U2OS cells (FIGS. 14C-14D) were analyzed after treatment with SpCas9 and xCas9 3.7. FIG. 14E, All six GUIDE-seq sites with an NGG PAM that were tested for SpCas9 and xCas9 3.7 in HEK293T and U2OS cells were also tested with xCas9 3.6. On-target reads (▪) and off-target reads for all sites are shown. Target sequences are listed in Table 15.

FIGS. 15A-15D show validation by high-throughput sequencing of GUIDE-seq results. The most frequent off-target sites identified by GUIDE-seq were verified by HTS of genomic DNA following treatment of HEK293T cells with SpCas9 or xCas9 3.7. (FIGS. 15A-15B), or following treatment with SpCas9 or xCas9 3.6 (FIGS. 15C-15D). New sites with non-NGG PAMs that were identified as xCas9 off-target sites were also analyzed in (FIGS. 15A-15D). Values and error bars reflect the mean and standard deviation of n=3 biologically independent samples. Target sequences are in Table 16.

FIG. 16 shows the genotypes of evolved Cas9 variants.

FIG. 17 shows HTS sequencing results at 35 genomic sites treated with BE3⁵ and ABE⁷ variants. HEK293T cells and U20S were treated with SpCas9-BE3, xCas9(3.6)-BE3, xCas9(3.7)-BE3, SpCas9-ABE, xCas9(3.6)-ABE, or xCas9(3.7)-ABE and an sgRNA that targets each of the 35 sites below. One arbitrarily chosen replicate is shown; the data for all replicates is available from the NCBI sequencing read archive. Sites 1-10 correspond to SEQ ID NOs: 606-625. FANCF sites 1-15 correspond to SEQ ID NOs: 626-640.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

The term “base editor (BE),” or “nucleobase editor (NBE),” as used herein, refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid. In some embodiments, the base editor is capable of deaminating a base within a DNA molecule. In some embodiments, the base editor is capable of deaminating a cytosine (C) in DNA. In some embodiments, the base editor is a fusion protein comprising a nucleic acid programmable DNA binding protein fused to a nucleic acid editing domain. In some embodiments, the base editor is a fusion protein comprising a nucleic acid programmable DNA binding protein (napDNAbp) fused to a cytidine deaminase domain. In some embodiments, the base editor comprises a Cas9 domain (e.g., an evolved Cas9 domain), or an evolved version of a CasX, CasY, Cpf1, C2c1, C2c2, C2c3, or Argonaute protein that comprises one or more mutations homologous to the mutations provided herein fused to a cytidine deaminase. In some embodiments, the base editor comprises a Cas9 nickase (Cas9n) fused to an cytidine deaminase domain. In some embodiments, the base editor comprises a nuclease-inactive Cas9 (dCas9) fused to a cytidine deaminase domain. In some embodiments, the base editor includes an inhibitor of base excision repair, for example, a UGI domain or a dISN domain.

In some embodiments, the base editor is capable of deaminating an adenosine (A) in DNA. In some embodiments, the base editor is a fusion protein comprising a nucleic acid programmable DNA binding protein fused to a nucleic acid editing domain. In some embodiments, the base editor is a fusion protein comprising a nucleic acid programmable DNA binding protein (napDNAbp) fused to an adenosine deaminase domain. In some embodiments, the base editor is a fusion protein comprising a nucleic acid programmable DNA binding protein (napDNAbp) fused to one or more adenosine deaminase domains. In some embodiments, the base editor is a fusion protein comprising a nucleic acid programmable DNA binding protein (napDNAbp) fused to two adenosine deaminase domains. In some embodiments, the base editor comprises a Cas9 (e.g., an evolvedCas9 domain), or an evolved version of a CasX, CasY, Cpf1, C2c1, C2c2, C2c3, or Argonaute protein that comprises one or more mutations homologous to the mutations provided herein fused to an adenosine deaminase domain. In some embodiments, the base editor comprises a Cas9 nickase (Cas9n) fused to an adenosine deaminase domain. In some embodiments, the base editor comprises a Cas9 nickase (Cas9n) fused to two adenosine deaminase domains. In some embodiments, the base editor comprises a nuclease-inactive Cas9 (dCas9) fused to an adenosine deaminase domain. In some embodiments, the base editor comprises a nuclease-inactive Cas9 (dCas9) fused to two adenosine deaminase domains. In some embodiments, the base editor is fused to an inhibitor of base excision repair, for example, a UGI domain, or a dISN domain.

The term “nucleic acid programmable DNA binding protein” or “napDNAbp” refers to a protein that associates with a nucleic acid (e.g., DNA or RNA), such as a guide nucleic acid (e.g., gRNA), that guides the napDNAbp to a specific nucleic acid sequence, for example, by hybridizing to the target nucleic acid sequence. For example, a Cas9 domain can associate with a guide RNA that guides the Cas9 domain to a specific DNA sequence that has complementary to the guide RNA. In some embodiments, the napDNAbp is a class 2 microbial CRISPR-Cas effector. In some embodiments, the napDNAbp is a Cas9 domain, for example, a nuclease active Cas9, a Cas9 nickase (Cas9n), or a nuclease inactive Cas9 (dCas9). Examples of nucleic acid programmable DNA binding proteins include, without limitation, Cas9 (e.g., an evolved Cas9 domain), or an evolved version of a CasX, CasY, Cpf1, C2c1, C2c2, C2c3, or Argonaute protein that comprises one or more mutations homologous to the mutations provided herein. It should be appreciated, however, that nucleic acid programmable DNA binding proteins also include nucleic acid programmable proteins that bind RNA. For example, the napDNAbp may be associated with a nucleic acid that guides the napDNAbp to an RNA. Other nucleic acid programmable DNA binding proteins are also within the scope of this disclosure, though they may not be specifically described in this Application.

In some embodiments, the napDNAbp is an “RNA-programmable nuclease” or “RNA-guided nuclease.” The terms are used interchangeably herein and refer to a nuclease that forms a complex with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). Guide RNAs can exist as a complex of two or more RNAs, or as a single RNA molecule. Guide RNAs that exist as a single RNA molecule may be referred to as single-guide RNAs (sgRNAs), though “gRNA” is also used to refer to guide RNAs that exist as either single molecules or as a complex of two or more molecules. Typically, gRNAs that exist as a single RNA species comprise two domains: (1) a domain that shares homology to a target nucleic acid (i.e., directs binding of a Cas9 complex to the target); and (2) a domain that binds a Cas9 domain. In some embodiments, domain (2) corresponds to a sequence known as a tracrRNA and comprises a stem-loop structure. In some embodiments, domain (2) is identical or homologous to a tracrRNA as provided in Jinek et al., Science 337:816-821 (2012), the entire contents of which is incorporated herein by reference. Other examples of gRNAs (e.g., those including domain 2) can be found in International Patent Application PCT/US2014/054252, filed Sep. 5, 2014, entitled “Switchable Cas9 Nucleases And Uses Thereof,” and International Patent Application PCT/US2014/054247, filed Sep. 5, 2014, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will bind two or more Cas9 domains and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (also known as Csn1) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference).

Because RNA-programmable nucleases (e.g., Cas9) use RNA:DNA hybridization to target DNA cleavage sites, these proteins are able to target, in principle, any sequence specified by the guide RNA. Methods of using RNA-programmable nucleases, such as Cas9, for site-specific cleavage (e.g., to modify a genome) are known in the art (see e.g., Cong, L. et al., Multiplex genome engineering using CRISPR/Cas systems. Science 339, 819-823 (2013); Mali, P. et al., RNA-guided human genome engineering via Cas9. Science 339, 823-826 (2013); Hwang, W. Y. et al., Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acids Research (2013); Jiang, W. et al., RNA-guided editing of bacterial genomes using CRISPR-Cas systems. Nature Biotechnology 31, 233-239 (2013); the entire contents of each of which are incorporated herein by reference).

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, proteins comprising fragments of Cas9 are provided. In some embodiments, the fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of wild type Cas9. In some embodiments, wild type Cas9 corresponds to Cas9 from Streptococcus pyogenes (NCBI Reference Sequence: NC_017053.1, SEQ ID NO:1 (nucleotide); SEQ ID NO:2 (amino acid)). ATGGATAAGAAATACTCAATAGGCTTAGATATCGGCACAAATAGCGTCGGATGGGCGGTGATCACTGATGATTAT AAGGTTCCGTCTAAAAAGTTCAAGGTTCTGGGAAATACAGACCGCCACAGTATCAAAAAAAATCTTATAGGGGCT CTTTTATTTGGCAGTGGAGAGACAGCGGAAGCGACTCGTCTCAAACGGACAGCTCGTAGAAGGTATACACGTCGG AAGAATCGTATTTGTTATCTACAGGAGATTTTTTCAAATGAGATGGCGAAAGTAGATGATAGTTTCTTTCATCGA CTTGAAGAGTCTTTTTTGGTGGAAGAAGACAAGAAGCATGAACGTCATCCTATTTTTGGAAATATAGTAGATGAA GTTGCTTATCATGAGAAATATCCAACTATCTATCATCTGCGAAAAAAATTGGCAGATTCTACTGATAAAGCGGAT TTGCGCTTAATCTATTTGGCCTTAGCGCATATGATTAAGTTTCGTGGTCATTTTTTGATTGAGGGAGATTTAAAT CCTGATAATAGTGATGTGGACAAACTATTTATCCAGTTGGTACAAATCTACAATCAATTATTTGAAGAAAACCCT ATTAACGCAAGTAGAGTAGATGCTAAAGCGATTCTTTCTGCACGATTGAGTAAATCAAGACGATTAGAAAATCTC ATTGCTCAGCTCCCCGGTGAGAAGAGAAATGGCTTGTTTGGGAATCTCATTGCTTTGTCATTGGGATTGACCCCT

(SEQ ID NO: 1)
ATGGATAAGAAATACTCAATAGGCTTAGATATCGGCACAAATAGCGTCGGATGGGCGGTGATCACTGATGATTAT
AAGGTTCCGTCTAAAAAGTTCAAGGTTCTGGGAAATACAGACCGCCACAGTATCAAAAAAAATCTTATAGGGGCT
CTTTTATTTGGCAGTGGAGAGACAGCGGAAGCGACTCGTCTCAAACGGACAGCTCGTAGAAGGTATACACGTCGG
AAGAATCGTATTTGTTATCTACAGGAGATTTTTTCAAATGAGATGGCGAAAGTAGATGATAGTTTCTTTCATCGA
CTTGAAGAGTCTTTTTTGGTGGAAGAAGACAAGAAGCATGAACGTCATCCTATTTTTGGAAATATAGTAGATGAA
GTTGCTTATCATGAGAAATATCCAACTATCTATCATCTGCGAAAAAAATTGGCAGATTCTACTGATAAAGCGGAT
TTGCGCTTAATCTATTTGGCCTTAGCGCATATGATTAAGTTTCGTGGTCATTTTTTGATTGAGGGAGATTTAAAT
CCTGATAATAGTGATGTGGACAAACTATTTATCCAGTTGGTACAAATCTACAATCAATTATTTGAAGAAAACCCT
ATTAACGCAAGTAGAGTAGATGCTAAAGCGATTCTTTCTGCACGATTGAGTAAATCAAGACGATTAGAAAATCTC
ATTGCTCAGCTCCCCGGTGAGAAGAGAAATGGCTTGTTTGGGAATCTCATTGCTTTGTCATTGGGATTGACCCCT
AATTTTAAATCAAATTTTGATTTGGCAGAAGATGCTAAATTACAGCTTTCAAAAGATACTTACGATGATGATTTA
GATAATTTATTGGCGCAAATTGGAGATCAATATGCTGATTTGTTTTTGGCAGCTAAGAATTTATCAGATGCTATT
TTACTTTCAGATATCCTAAGAGTAAATAGTGAAATAACTAAGGCTCCCCTATCAGCTTCAATGATTAAGCGCTAC
GATGAACATCATCAAGACTTGACTCTTTTAAAAGCTTTAGTTCGACAACAACTTCCAGAAAAGTATAAAGAAATC
TTTTTTGATCAATCAAAAAACGGATATGCAGGTTATATTGATGGGGGAGCTAGCCAAGAAGAATTTTATAAATTT
ATCAAACCAATTTTAGAAAAAATGGATGGTACTGAGGAATTATTGGTGAAACTAAATCGTGAAGATTTGCTGCGC
AAGCAACGGACCTTTGACAACGGCTCTATTCCCCATCAAATTCACTTGGGTGAGCTGCATGCTATTTTGAGAAGA
CAAGAAGACTTTTATCCATTTTTAAAAGACAATCGTGAGAAGATTGAAAAAATCTTGACTTTTCGAATTCCTTAT
TATGTTGGTCCATTGGCGCGTGGCAATAGTCGTTTTGCATGGATGACTCGGAAGTCTGAAGAAACAATTACCCCA
TGGAATTTTGAAGAAGTTGTCGATAAAGGTGCTTCAGCTCAATCATTTATTGAACGCATGACAAACTTTGATAAA
AATCTTCCAAATGAAAAAGTACTACCAAAACATAGTTTGCTTTATGAGTATTTTACGGTTTATAACGAATTGACA
AAGGTCAAATATGTTACTGAGGGAATGCGAAAACCAGCATTTCTTTCAGGTGAACAGAAGAAAGCCATTGTTGAT
TTACTCTTCAAAACAAATCGAAAAGTAACCGTTAAGCAATTAAAAGAAGATTATTTCAAAAAAATAGAATGTTTT
GATAGTGTTGAAATTTCAGGAGTTGAAGATAGATTTAATGCTTCATTAGGCGCCTACCATGATTTGCTAAAAATT
ATTAAAGATAAAGATTTTTTGGATAATGAAGAAAATGAAGATATCTTAGAGGATATTGTTTTAACATTGACCTTA
TTTGAAGATAGGGGGATGATTGAGGAAAGACTTAAAACATATGCTCACCTCTTTGATGATAAGGTGATGAAACAG
CTTAAACGTCGCCGTTATACTGGTTGGGGACGTTTGTCTCGAAAATTGATTAATGGTATTAGGGATAAGCAATCT
GGCAAAACAATATTAGATTTTTTGAAATCAGATGGTTTTGCCAATCGCAATTTTATGCAGCTGATCCATGATGAT
AGTTTGACATTTAAAGAAGATATTCAAAAAGCACAGGTGTCTGGACAAGGCCATAGTTTACATGAACAGATTGCT
AACTTAGCTGGCAGTCCTGCTATTAAAAAAGGTATTTTACAGACTGTAAAAATTGTTGATGAACTGGTCAAAGTA
ATGGGGCATAAGCCAGAAAATATCGTTATTGAAATGGCACGTGAAAATCAGACAACTCAAAAGGGCCAGAAAAAT
TCGCGAGAGCGTATGAAACGAATCGAAGAAGGTATCAAAGAATTAGGAAGTCAGATTCTTAAAGAGCATCCTGTT
GAAAATACTCAATTGCAAAATGAAAAGCTCTATCTCTATTATCTACAAAATGGAAGAGACATGTATGTGGACCAA
GAATTAGATATTAATCGTTTAAGTGATTATGATGTCGATCACATTGTTCCACAAAGTTTCATTAAAGACGATTCA
ATAGACAATAAGGTACTAACGCGTTCTGATAAAAATCGTGGTAAATCGGATAACGTTCCAAGTGAAGAAGTAGTC
AAAAAGATGAAAAACTATTGGAGACAACTTCTAAACGCCAAGTTAATCACTCAACGTAAGTTTGATAATTTAACG
AAAGCTGAACGTGGAGGTTTGAGTGAACTTGATAAAGCTGGTTTTATCAAACGCCAATTGGTTGAAACTCGCCAA
ATCACTAAGCATGTGGCACAAATTTTGGATAGTCGCATGAATACTAAATACGATGAAAATGATAAACTTATTCGA
GAGGTTAAAGTGATTACCTTAAAATCTAAATTAGTTTCTGACTTCCGAAAAGATTTCCAATTCTATAAAGTACGT
GAGATTAACAATTACCATCATGCCCATGATGCGTATCTAAATGCCGTCGTTGGAACTGCTTTGATTAAGAAATAT
CCAAAACTTGAATCGGAGTTTGTCTATGGTGATTATAAAGTTTATGATGTTCGTAAAATGATTGCTAAGTCTGAG
CAAGAAATAGGCAAAGCAACCGCAAAATATTTCTTTTACTCTAATATCATGAACTTCTTCAAAACAGAAATTACA
CTTGCAAATGGAGAGATTCGCAAACGCCCTCTAATCGAAACTAATGGGGAAACTGGAGAAATTGTCTGGGATAAA
GGGCGAGATTTTGCCACAGTGCGCAAAGTATTGTCCATGCCCCAAGTCAATATTGTCAAGAAAACAGAAGTACAG
ACAGGCGGATTCTCCAAGGAGTCAATTTTACCAAAAAGAAATTCGGACAAGCTTATTGCTCGTAAAAAAGACTGG
GATCCAAAAAAATATGGTGGTTTTGATAGTCCAACGGTAGCTTATTCAGTCCTAGTGGTTGCTAAGGTGGAAAAA
GGGAAATCGAAGAAGTTAAAATCCGTTAAAGAGTTACTAGGGATCACAATTATGGAAAGAAGTTCCTTTGAAAAA
AATCCGATTGACTTTTTAGAAGCTAAAGGATATAAGGAAGTTAAAAAAGACTTAATCATTAAACTACCTAAATAT
AGTCTTTTTGAGTTAGAAAACGGTCGTAAACGGATGCTGGCTAGTGCCGGAGAATTACAAAAAGGAAATGAGCTG
GCTCTGCCAAGCAAATATGTGAATTTTTTATATTTAGCTAGTCATTATGAAAAGTTGAAGGGTAGTCCAGAAGAT
AACGAACAAAAACAATTGTTTGTGGAGCAGCATAAGCATTATTTAGATGAGATTATTGAGCAAATCAGTGAATTT
TCTAAGCGTGTTATTTTAGCAGATGCCAATTTAGATAAAGTTCTTAGTGCATATAACAAACATAGAGACAAACCA
ATACGTGAACAAGCAGAAAATATTATTCATTTATTTACGTTGACGAATCTTGGAGCTCCCGCTGCTTTTAAATAT
TTTGATACAACAATTGATCGTAAACGATATACGTCTACAAAAGAAGTTTTAGATGCCACTCTTATCCATCAATCC
ATCACTGGTCTTTATGAAACACGCATTGATTTGAGTCAGCTAGGAGGTGACTGA
(SEQ ID NO: 2)
MDKKYSIGLDIGTNSVGWAVITDDYKVPSKKFKVLGNTDRHSIKKNLIGALLFGSGETAEATRLKRTARRRYTRR
KNRICYLQEIFSNEMAKVDDSFFHRLEESFLVEEDKKHERHPIFGNIVDEVAYHEKYPTIYHLRKKLADSTDKAD
LRLIYLALAHMIKFRGHFLIEGDLNPDNSDVDKLFIQLVQIYNQLFEENPINASRVDAKAILSARLSKSRRLENL
IAQLPGEKRNGLFGNLIALSLGLTPNFKSNFDLAEDAKLQLSKDTYDDDLDNLLAQIGDQYADLFLAAKNLSDAI
LLSDILRVNSEITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISGVEDRFNASLGAYHDLLKI
IKDKDFLDNEENEDILEDIVLTLTLFEDRGMIEERLKTYAHLFDDKVMKQLKRRRYTGWGRLSRKLINGIRDKQS
GKTILDFLKSDGFANRNFMQLIHDDSLTFKEDIQKAQVSGQGHSLHEQIANLAGSPAIKKGILQTVKIVDELVKV
MGHKPENIVIEMARENQTTQKGQKNSRERMKRIEEGIKELGSQILKEHPVENTQLQNEKLYLYYLQNGRDMYVDQ
ELDINRLSDYDVDHIVPQSFIKDDSIDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQHKHYLDEIIEQISEF
SKRVILADANLDKVLSAYNKHRDKPIREQAENIIHLFTLTNLGAPAAFKYFDTTIDRKRYTSTKEVLDATLIHQS
ITGLYETRIDLSQLGGD
(single underline: HNH domain; double underline: RuvC domain)

In some embodiments, wild type Cas9 corresponds to, or comprises SEQ ID NO:3 (nucleotide) and/or SEQ ID NO: 4 (amino acid):

(SEQ ID NO: 3)
ATGGATAAAAAGTATTCTATTGGTTTAGACATCGGCACTAATTCCGTTGGATGGGCTGTCATAACCGATGAATAC
AAAGTACCTTCAAAGAAATTTAAGGTGTTGGGGAACACAGACCGTCATTCGATTAAAAAGAATCTTATCGGTGCC
CTCCTATTCGATAGTGGCGAAACGGCAGAGGCGACTCGCCTGAAACGAACCGCTCGGAGAAGGTATACACGTCGC
AAGAACCGAATATGTTACTTACAAGAAATTTTTAGCAATGAGATGGCCAAAGTTGACGATTCTTTCTTTCACCGT
TTGGAAGAGTCCTTCCTTGTCGAAGAGGACAAGAAACATGAACGGCACCCCATCTTTGGAAACATAGTAGATGAG
GTGGCATATCATGAAAAGTACCCAACGATTTATCACCTCAGAAAAAAGCTAGTTGACTCAACTGATAAAGCGGAC
CTGAGGTTAATCTACTTGGCTCTTGCCCATATGATAAAGTTCCGTGGGCACTTTCTCATTGAGGGTGATCTAAAT
CCGGACAACTCGGATGTCGACAAACTGTTCATCCAGTTAGTACAAACCTATAATCAGTTGTTTGAAGAGAACCCT
ATAAATGCAAGTGGCGTGGATGCGAAGGCTATTCTTAGCGCCCGCCTCTCTAAATCCCGACGGCTAGAAAACCTG
ATCGCACAATTACCCGGAGAGAAGAAAAATGGGTTGTTCGGTAACCTTATAGCGCTCTCACTAGGCCTGACACCA
AATTTTAAGTCGAACTTCGACTTAGCTGAAGATGCCAAATTGCAGCTTAGTAAGGACACGTACGATGACGATCTC
GACAATCTACTGGCACAAATTGGAGATCAGTATGCGGACTTATTTTTGGCTGCCAAAAACCTTAGCGATGCAATC
CTCCTATCTGACATACTGAGAGTTAATACTGAGATTACCAAGGCGCCGTTATCCGCTTCAATGATCAAAAGGTAC
GATGAACATCACCAAGACTTGACACTTCTCAAGGCCCTAGTCCGTCAGCAACTGCCTGAGAAATATAAGGAAATA
TTCTTTGATCAGTCGAAAAACGGGTACGCAGGTTATATTGACGGCGGAGCGAGTCAAGAGGAATTCTACAAGTTT
ATCAAACCCATATTAGAGAAGATGGATGGGACGGAAGAGTTGCTTGTAAAACTCAATCGCGAAGATCTACTGCGA
AAGCAGCGGACTTTCGACAACGGTAGCATTCCACATCAAATCCACTTAGGCGAATTGCATGCTATACTTAGAAGG
CAGGAGGATTTTTATCCGTTCCTCAAAGACAATCGTGAAAAGATTGAGAAAATCCTAACCTTTCGCATACCTTAC
TATGTGGGACCCCTGGCCCGAGGGAACTCTCGGTTCGCATGGATGACAAGAAAGTCCGAAGAAACGATTACTCCA
TGGAATTTTGAGGAAGTTGTCGATAAAGGTGCGTCAGCTCAATCGTTCATCGAGAGGATGACCAACTTTGACAAG
AATTTACCGAACGAAAAAGTATTGCCTAAGCACAGTTTACTTTACGAGTATTTCACAGTGTACAATGAACTCACG
AAAGTTAAGTATGTCACTGAGGGCATGCGTAAACCCGCCTTTCTAAGCGGAGAACAGAAGAAAGCAATAGTAGAT
CTGTTATTCAAGACCAACCGCAAAGTGACAGTTAAGCAATTGAAAGAGGACTACTTTAAGAAAATTGAATGCTTC
GATTCTGTCGAGATCTCCGGGGTAGAAGATCGATTTAATGCGTCACTTGGTACGTATCATGACCTCCTAAAGATA
ATTAAAGATAAGGACTTCCTGGATAACGAAGAGAATGAAGATATCTTAGAAGATATAGTGTTGACTCTTACCCTC
TTTGAAGATCGGGAAATGATTGAGGAAAGACTAAAAACATACGCTCACCTGTTCGACGATAAGGTTATGAAACAG
TTAAAGAGGCGTCGCTATACGGGCTGGGGACGATTGTCGCGGAAACTTATCAACGGGATAAGAGACAAGCAAAGT
GGTAAAACTATTCTCGATTTTCTAAAGAGCGACGGCTTCGCCAATAGGAACTTTATGCAGCTGATCCATGATGAC
TCTTTAACCTTCAAAGAGGATATACAAAAGGCACAGGTTTCCGGACAAGGGGACTCATTGCACGAACATATTGCG
AATCTTGCTGGTTCGCCAGCCATCAAAAAGGGCATACTCCAGACAGTCAAAGTAGTGGATGAGCTAGTTAAGGTC
ATGGGACGTCACAAACCGGAAAACATTGTAATCGAGATGGCACGCGAAAATCAAACGACTCAGAAGGGGCAAAAA
AACAGTCGAGAGCGGATGAAGAGAATAGAAGAGGGTATTAAAGAACTGGGCAGCCAGATCTTAAAGGAGCATCCT
GTGGAAAATACCCAATTGCAGAACGAGAAACTTTACCTCTATTACCTACAAAATGGAAGGGACATGTATGTTGAT
CAGGAACTGGACATAAACCGTTTATCTGATTACGACGTCGATCACATTGTACCCCAATCCTTTTTGAAGGACGAT
TCAATCGACAATAAAGTGCTTACACGCTCGGATAAGAACCGAGGGAAAAGTGACAATGTTCCAAGCGAGGAAGTC
GTAAAGAAAATGAAGAACTATTGGCGGCAGCTCCTAAATGCGAAACTGATAACGCAAAGAAAGTTCGATAACTTA
ACTAAAGCTGAGAGGGGTGGCTTGTCTGAACTTGACAAGGCCGGATTTATTAAACGTCAGCTCGTGGAAACCCGC
CAAATCACAAAGCATGTTGCACAGATACTAGATTCCCGAATGAATACGAAATACGACGAGAACGATAAGCTGATT
CGGGAAGTCAAAGTAATCACTTTAAAGTCAAAATTGGTGTCGGACTTCAGAAAGGATTTTCAATTCTATAAAGTT
AGGGAGATAAATAACTACCACCATGCGCACGACGCTTATCTTAATGCCGTCGTAGGGACCGCACTCATTAAGAAA
TACCCGAAGCTAGAAAGTGAGTTTGTGTATGGTGATTACAAAGTTTATGACGTCCGTAAGATGATCGCGAAAAGC
GAACAGGAGATAGGCAAGGCTACAGCCAAATACTTCTTTTATTCTAACATTATGAATTTCTTTAAGACGGAAATC
ACTCTGGCAAACGGAGAGATACGCAAACGACCTTTAATTGAAACCAATGGGGAGACAGGTGAAATCGTATGGGAT
AAGGGCCGGGACTTCGCGACGGTGAGAAAAGTTTTGTCCATGCCCCAAGTCAACATAGTAAAGAAAACTGAGGTG
CAGACCGGAGGGTTTTCAAAGGAATCGATTCTTCCAAAAAGGAATAGTGATAAGCTCATCGCTCGTAAAAAGGAC
TGGGACCCGAAAAAGTACGGTGGCTTCGATAGCCCTACAGTTGCCTATTCTGTCCTAGTAGTGGCAAAAGTTGAG
AAGGGAAAATCCAAGAAACTGAAGTCAGTCAAAGAATTATTGGGGATAACGATTATGGAGCGCTCGTCTTTTGAA
AAGAACCCCATCGACTTCCTTGAGGCGAAAGGTTACAAGGAAGTAAAAAAGGATCTCATAATTAAACTACCAAAG
TATAGTCTGTTTGAGTTAGAAAATGGCCGAAAACGGATGTTGGCTAGCGCCGGAGAGCTTCAAAAGGGGAACGAA
CTCGCACTACCGTCTAAATACGTGAATTTCCTGTATTTAGCGTCCCATTACGAGAAGTTGAAAGGTTCACCTGAA
GATAACGAACAGAAGCAACTTTTTGTTGAGCAGCACAAACATTATCTCGACGAAATCATAGAGCAAATTTCGGAA
TTCAGTAAGAGAGTCATCCTAGCTGATGCCAATCTGGACAAAGTATTAAGCGCATACAACAAGCACAGGGATAAA
CCCATACGTGAGCAGGCGGAAAATATTATCCATTTGTTTACTCTTACCAACCTCGGCGCTCCAGCCGCATTCAAG
TATTTTGACACAACGATAGATCGCAAACGATACACTTCTACCAAGGAGGTGCTAGACGCGACACTGATTCACCAA
TCCATCACGGGATTATATGAAACTCGGATAGATTTGTCACAGCTTGGGGGTGACGGATCCCCCAAGAAGAAGAGG
AAAGTCTCGAGCGACTACAAAGACCATGACGGTGATTATAAAGATCATGACATCGATTACAAGGATGACGATGAC
AAGGCTGCAGGA
(SEQ ID NO: 4)
MDKKYSIGLAIGTNSVGWAVITDEYKVPSKKFKVLGNTDRHSIKKNLIGALLFDSGETAEATRLKRTARRRYTRR
KNRICYLQEIFSNEMAKVDDSFFHRLEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKYFDTTIDRKRYTSTKEVLDATLIHQ
SITGLYETRIDLSQLGGD
(single underline: HNH domain; double underline: RuvC domain)

In some embodiments, wild type Cas9 corresponds to Cas9 from Streptococcus pyogenes (NCBI Reference Sequence: NC_002737.2, SEQ ID NO: 5 (nucleotide); and Uniport Reference Sequence: Q99ZW2, SEQ ID NO: 9 (amino acid).

(SEQ ID NO: 5)
ATGGATAAGAAATACTCAATAGGCTTAGATATCGGCACAAATAGCGTCGGATGGGCGGTGATCACTGATGAATAT
AAGGTTCCGTCTAAAAAGTTCAAGGTTCTGGGAAATACAGACCGCCACAGTATCAAAAAAAATCTTATAGGGGCT
CTTTTATTTGACAGTGGAGAGACAGCGGAAGCGACTCGTCTCAAACGGACAGCTCGTAGAAGGTATACACGTCGG
AAGAATCGTATTTGTTATCTACAGGAGATTTTTTCAAATGAGATGGCGAAAGTAGATGATAGTTTCTTTCATCGA
CTTGAAGAGTCTTTTTTGGTGGAAGAAGACAAGAAGCATGAACGTCATCCTATTTTTGGAAATATAGTAGATGAA
GTTGCTTATCATGAGAAATATCCAACTATCTATCATCTGCGAAAAAAATTGGTAGATTCTACTGATAAAGCGGAT
TTGCGCTTAATCTATTTGGCCTTAGCGCATATGATTAAGTTTCGTGGTCATTTTTTGATTGAGGGAGATTTAAAT
CCTGATAATAGTGATGTGGACAAACTATTTATCCAGTTGGTACAAACCTACAATCAATTATTTGAAGAAAACCCT
ATTAACGCAAGTGGAGTAGATGCTAAAGCGATTCTTTCTGCACGATTGAGTAAATCAAGACGATTAGAAAATCTC
ATTGCTCAGCTCCCCGGTGAGAAGAAAAATGGCTTATTTGGGAATCTCATTGCTTTGTCATTGGGTTTGACCCCT
AATTTTAAATCAAATTTTGATTTGGCAGAAGATGCTAAATTACAGCTTTCAAAAGATACTTACGATGATGATTTA
GATAATTTATTGGCGCAAATTGGAGATCAATATGCTGATTTGTTTTTGGCAGCTAAGAATTTATCAGATGCTATT
TTACTTTCAGATATCCTAAGAGTAAATACTGAAATAACTAAGGCTCCCCTATCAGCTTCAATGATTAAACGCTAC
GATGAACATCATCAAGACTTGACTCTTTTAAAAGCTTTAGTTCGACAACAACTTCCAGAAAAGTATAAAGAAATC
TTTTTTGATCAATCAAAAAACGGATATGCAGGTTATATTGATGGGGGAGCTAGCCAAGAAGAATTTTATAAATTT
ATCAAACCAATTTTAGAAAAAATGGATGGTACTGAGGAATTATTGGTGAAACTAAATCGTGAAGATTTGCTGCGC
AAGCAACGGACCTTTGACAACGGCTCTATTCCCCATCAAATTCACTTGGGTGAGCTGCATGCTATTTTGAGAAGA
CAAGAAGACTTTTATCCATTTTTAAAAGACAATCGTGAGAAGATTGAAAAAATCTTGACTTTTCGAATTCCTTAT
TATGTTGGTCCATTGGCGCGTGGCAATAGTCGTTTTGCATGGATGACTCGGAAGTCTGAAGAAACAATTACCCCA
TGGAATTTTGAAGAAGTTGTCGATAAAGGTGCTTCAGCTCAATCATTTATTGAACGCATGACAAACTTTGATAAA
AATCTTCCAAATGAAAAAGTACTACCAAAACATAGTTTGCTTTATGAGTATTTTACGGTTTATAACGAATTGACA
AAGGTCAAATATGTTACTGAAGGAATGCGAAAACCAGCATTTCTTTCAGGTGAACAGAAGAAAGCCATTGTTGAT
TTACTCTTCAAAACAAATCGAAAAGTAACCGTTAAGCAATTAAAAGAAGATTATTTCAAAAAAATAGAATGTTTT
GATAGTGTTGAAATTTCAGGAGTTGAAGATAGATTTAATGCTTCATTAGGTACCTACCATGATTTGCTAAAAATT
ATTAAAGATAAAGATTTTTTGGATAATGAAGAAAATGAAGATATCTTAGAGGATATTGTTTTAACATTGACCTTA
TTTGAAGATAGGGAGATGATTGAGGAAAGACTTAAAACATATGCTCACCTCTTTGATGATAAGGTGATGAAACAG
CTTAAACGTCGCCGTTATACTGGTTGGGGACGTTTGTCTCGAAAATTGATTAATGGTATTAGGGATAAGCAATCT
GGCAAAACAATATTAGATTTTTTGAAATCAGATGGTTTTGCCAATCGCAATTTTATGCAGCTGATCCATGATGAT
AGTTTGACATTTAAAGAAGACATTCAAAAAGCACAAGTGTCTGGACAAGGCGATAGTTTACATGAACATATTGCA
AATTTAGCTGGTAGCCCTGCTATTAAAAAAGGTATTTTACAGACTGTAAAAGTTGTTGATGAATTGGTCAAAGTA
ATGGGGCGGCATAAGCCAGAAAATATCGTTATTGAAATGGCACGTGAAAATCAGACAACTCAAAAGGGCCAGAAA
AATTCGCGAGAGCGTATGAAACGAATCGAAGAAGGTATCAAAGAATTAGGAAGTCAGATTCTTAAAGAGCATCCT
GTTGAAAATACTCAATTGCAAAATGAAAAGCTCTATCTCTATTATCTCCAAAATGGAAGAGACATGTATGTGGAC
CAAGAATTAGATATTAATCGTTTAAGTGATTATGATGTCGATCACATTGTTCCACAAAGTTTCCTTAAAGACGAT
TCAATAGACAATAAGGTCTTAACGCGTTCTGATAAAAATCGTGGTAAATCGGATAACGTTCCAAGTGAAGAAGTA
GTCAAAAAGATGAAAAACTATTGGAGACAACTTCTAAACGCCAAGTTAATCACTCAACGTAAGTTTGATAATTTA
ACGAAAGCTGAACGTGGAGGTTTGAGTGAACTTGATAAAGCTGGTTTTATCAAACGCCAATTGGTTGAAACTCGC
CAAATCACTAAGCATGTGGCACAAATTTTGGATAGTCGCATGAATACTAAATACGATGAAAATGATAAACTTATT
CGAGAGGTTAAAGTGATTACCTTAAAATCTAAATTAGTTTCTGACTTCCGAAAAGATTTCCAATTCTATAAAGTA
CGTGAGATTAACAATTACCATCATGCCCATGATGCGTATCTAAATGCCGTCGTTGGAACTGCTTTGATTAAGAAA
TATCCAAAACTTGAATCGGAGTTTGTCTATGGTGATTATAAAGTTTATGATGTTCGTAAAATGATTGCTAAGTCT
GAGCAAGAAATAGGCAAAGCAACCGCAAAATATTTCTTTTACTCTAATATCATGAACTTCTTCAAAACAGAAATT
ACACTTGCAAATGGAGAGATTCGCAAACGCCCTCTAATCGAAACTAATGGGGAAACTGGAGAAATTGTCTGGGAT
AAAGGGCGAGATTTTGCCACAGTGCGCAAAGTATTGTCCATGCCCCAAGTCAATATTGTCAAGAAAACAGAAGTA
CAGACAGGCGGATTCTCCAAGGAGTCAATTTTACCAAAAAGAAATTCGGACAAGCTTATTGCTCGTAAAAAAGAC
TGGGATCCAAAAAAATATGGTGGTTTTGATAGTCCAACGGTAGCTTATTCAGTCCTAGTGGTTGCTAAGGTGGAA
AAAGGGAAATCGAAGAAGTTAAAATCCGTTAAAGAGTTACTAGGGATCACAATTATGGAAAGAAGTTCCTTTGAA
AAAAATCCGATTGACTTTTTAGAAGCTAAAGGATATAAGGAAGTTAAAAAAGACTTAATCATTAAACTACCTAAA
TATAGTCTTTTTGAGTTAGAAAACGGTCGTAAACGGATGCTGGCTAGTGCCGGAGAATTACAAAAAGGAAATGAG
CTGGCTCTGCCAAGCAAATATGTGAATTTTTTATATTTAGCTAGTCATTATGAAAAGTTGAAGGGTAGTCCAGAA
GATAACGAACAAAAACAATTGTTTGTGGAGCAGCATAAGCATTATTTAGATGAGATTATTGAGCAAATCAGTGAA
TTTTCTAAGCGTGTTATTTTAGCAGATGCCAATTTAGATAAAGTTCTTAGTGCATATAACAAACATAGAGACAAA
CCAATACGTGAACAAGCAGAAAATATTATTCATTTATTTACGTTGACGAATCTTGGAGCTCCCGCTGCTTTTAAA
TATTTTGATACAACAATTGATCGTAAACGATATACGTCTACAAAAGAAGTTTTAGATGCCACTCTTATCCATCAA
TCCATCACTGGTCTTTATGAAACACGCATTGATTTGAGTCAGCTAGGAGGTGACTGA
(SEQ ID NO: 9)
MDKKYSIGLDIGTNSVGWAVITDEYKVPSKKFKVLGNTDRHSIKKNLIGALLFDSGETAEATRLKRTARRRYTRR
KNRICYLQEIFSNEMAKVDDSFFHRLEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKYFDTTIDRKRYTSTKEVLDATLIHQ
SITGLYETRIDLSQLGGD
(single underline: HNH domain; double underline: RuvC domain)

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis I (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1); Geobacillus stearothermophilus (NCBI Ref: NZ_CP008934.1); or Neisseria. meningitidis (NCBI Ref: YP_002342100.1) or to a Cas9 from any other organism (e.g., a Cas9 from an organism listed in Example 1).

In some embodiments, a Cas9 domain comprising one or more mutations provided herein is at least about 70% identical, at least about 80% identical, at least about 90% identical, at least about 92%, at least about 95% identical, at least about 98% identical, at least about 99% identical, at least about 99.5% identical, or at least about 99.9% identical to SEQ ID NO: 9. In some embodiments, variants of a Cas9 domain comprising one or more mutations provided herein (e.g., variants of SEQ ID NO: 9) are provided having amino acid sequences which are shorter, or longer than SEQ ID NO: 9, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more.

In some embodiments, dCas9 corresponds to, or comprises in part or in whole, a Cas9 amino acid sequence having one or more mutations that inactivate the Cas9 nuclease activity. For example, in some embodiments, a dCas9 domain comprises D10A and/or H840A mutation. An exemplary dCas9 domain comprises the amino acid sequence of SEQ ID NO: 6. dCas9 (D10A and H840A):

(SEQ ID NO: 6)
MDKKYSIGLAIGTNSVGWAVITDEYKVPSKKFKVLGNTDRHSIKKNLIGALLFDSGETAEATRLKRTARRRYTRR
KNRICYLQEIFSNEMAKVDDSFFHRLEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLYLYYLQNGRDMYVD
QELDINRLSDYDVDAIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKYFDTTIDRKRYTSTKEVLDATLIHQ
SITGLYETRIDLSQLGGD
(D10A and H840A mutations shown in bold; see, e.g., Qi et al.,
Repurposing CRISPR as an RNA-guided platform for sequence-specific control
of gene expression. Cell. 2013; 152(5):1173-83, the entire contents of
which are incorporated herein by reference).

In some embodiments, the Cas9 domain comprises a D10A mutation, while the residue at position 840 remains a histidine in the amino acid sequence as provided in SEQ ID NO: 9, or at corresponding positions in any of the amino acid sequences provided in SEQ ID NOs: 10-262. Without wishing to be bound by any particular theory, the presence of the catalytic residue H840 restores the activity of the Cas9 to cleave the non-edited (e.g., non-deaminated) strand containing a G opposite the targeted C. Restoration of H840 (e.g., from A840) does not result in the cleavage of the target strand containing the C. Such Cas9 variants are able to generate a single-strand DNA break (nick) at a specific location based on the gRNA-defined target sequence, leading to repair of the non-edited strand, ultimately resulting in a base change (e.g., a G to A change) on the non-edited strand. Briefly, the C of a C-G base pair can be deaminated to a U by a deaminase, e.g., an APOBEC deaminase. Nicking the non-edited strand, the strand having the G, facilitates removal of the G via mismatch repair mechanisms. Uracil-DNA glycosylase inhibitor protein (UGI) inhibits Uracil-DNA glycosylase (UDG), which prevents removal of the U.

In other embodiments, dCas9 variants having mutations other than D10A and H840A are provided, which, e.g., result in nuclease inactivated Cas9 (dCas9). Such mutations, by way of example, include other amino acid substitutions at D10 and H820, or other substitutions within the nuclease domains of Cas9 (e.g., substitutions in the HNH nuclease subdomain and/or the RuvC1 subdomain).

The term “Cas9 nickase” or “Cas9n,” as used herein, refers to a Cas9 domain that is capable of cleaving one strand of the duplexed nucleic acid molecule (e.g., a duplexed DNA molecule). In some embodiments, a Cas9 nickase comprises a D10A mutation and has a histidine at position H840 of SEQ ID NO: 9, or a corresponding mutation in any of SEQ ID NOs: 10-262. For example, in some embodiments, a Cas9 nickase comprises the amino acid sequence as set forth in SEQ ID NO: 7. Such a Cas9 nickase (Cas9n) has an active HNH nuclease domain and is able to cleave the non-targeted strand of DNA, i.e., the strand bound by the gRNA. Further, such a Cas9 nickase has an inactive RuvC nuclease domain and is not able to cleave the targeted strand of the DNA, i.e., the strand where base editing is desired. In some embodiments, any of the Cas9 domains provided herein comprises a H840A mutation (SEQ ID NO: 7). In some embodiments, any of the Cas9 domains provided herein comprises a H840A mutation (SEQ ID NO: 8). Exemplary Cas9 nickases are shown below. However, it should be appreciated that additional Cas9 nickases that generate a single-stranded DNA break of a DNA duplex would be apparent to the skilled artisan and are within the scope of this disclosure.

Cas9n (D10A):

(SEQ ID NO: 7)
MDKKYSIGLAIGTNSVGWAVITDEYKVPSKKFKVLGNTDRHSIKKNLIGALLFDSGETAEATRLKRTARRRYTRR
KNRICYLQEIFSNEMAKVDDSFFHRLEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKYFDTTIDRKRYTSTKEVLDATLIHQ
SITGLYETRIDLSQLGGD
(single underline: HNH domain; double underline:
RuvC domain; D10A mutation shown in bold)

### Cas9n (H840A):

(SEQ ID NO: 8)
MDKKYSIGLDIGTNSVGWAVITDEYKVPSKKFKVLGNTDRHSIKKNLIGALLFDSGETAEATRLKRTARRRYTRR
KNRICYLQEIFSNEMAKVDDSFFHRLEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLYLYYLQNGRDMYVD
QELDINRLSDYDVDAIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKYFDTTIDRKRYTSTKEVLDATLIHQ
SITGLYETRIDLSQLGGD
(single underline: HNH domain; double underline:
RuvC domain; H840A mutation shown in bold)

In some embodiments, Cas9 fusion proteins as provided herein comprise the full-length amino acid sequence of a Cas9 domain, e.g., one of the sequences provided above. In other embodiments, however, fusion proteins as provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or a sgRNA, but does not comprise a functional nuclease domain, e.g., it comprises only a truncated version of a nuclease domain or no nuclease domain at all. Exemplary amino acid sequences of suitable Cas9 domains and Cas9 fragments are provided herein, and additional suitable sequences of Cas9 domains and Cas9 fragments will be apparent to those of skill in the art. In some embodiments, a Cas9 fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 domain. In some embodiments, a Cas9 fragment comprises at least at least 100 amino acids in length. In some embodiments, the Cas9 fragment is at least 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, or at least 1600 amino acids of a corresponding wild type Cas9 domain. In some embodiments, the Cas9 fragment comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues of a corresponding wild type Cas9 domain. In some embodiments, the wild-type protein is S. pyogenes Cas9 (spCas9) of SEQ ID NO: 9.

In some embodiments, Cas9 fusion proteins as provided herein comprise the full-length amino acid sequence of a Cas9 domain, e.g., one of the Cas9 sequences provided herein. In other embodiments, however, fusion proteins as provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or sgRNA, but does not comprise a functional nuclease domain, e.g., in that it comprises only a truncated version of a nuclease domain or no nuclease domain at all. Exemplary amino acid sequences of suitable Cas9 domains and Cas9 fragments are provided herein, and additional suitable sequences of Cas9 domains and fragments will be apparent to those of ordinary skill in the art. In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis I (NCBI Ref: NC_018721.1); Streptococcus thermophilus (NCBI Ref: YP_820832.1); Geobacillus stearothermophilus (NCBI Ref: NZ_CP008934.1); Listeria innocua (NCBI Ref: NP_472073.1); Campylobacter jejuni (NCBI Ref: YP_002344900.1); or Neisseria. meningitidis (NCBI Ref: YP_002342100.1).

The term “deaminase” or “deaminase domain,” as used herein, refers to a protein or enzyme that catalyzes a deamination reaction. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism, that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytidine deaminase domain, catalyzing the hydrolytic deamination of cytosine to uracil. In some embodiments, the cytidine deaminase catalyzes the hydrolytic deamination of cytidine or cytosine in deoxyribonucleic acid (DNA). In some embodiments, the cytidine deaminase domain comprises the amino acid sequence of any one of SEQ ID NO: 350-389. In some embodiments, the cytidine deaminase or cytidine deaminase domain is a naturally-occurring cytidine deaminase from an organism, such as a human, chimpanzee, gorilla, monkey, cow, dog, rat, or mouse. In some embodiments, the cytidine deaminase or cytidine deaminase domain is a variant of a naturally-occurring cytidine deaminase from an organism, that does not occur in nature. For example, in some embodiments, the cytidine deaminase or cytidine deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring cytidine deaminase from an organism, such as a human, chimpanzee, gorilla, monkey, cow, dog, rat, or mouse.

In some embodiments, the deaminase or deaminase domain is an adenosine deaminase, which catalyzes the hydrolytic deamination of adenine or adenosine. In some embodiments, the deaminase or deaminase domain is an adenosine deaminase, catalyzing the hydrolytic deamination of adenosine or deoxyadenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. In some embodiments, the adenosine deaminase comprises the amino acid sequence of any one of SEQ ID NOs: 400-408. In some embodiments, the adenosine deaminase comprises the amino acid sequence of any one of SEQ ID NOs: 409-458.

In some embodiments, the TadA deaminase is an N-terminal truncated TadA. In certain embodiments, the adenosine deaminase comprises the amino acid sequence:

(SEQ ID NO: 400)
MSEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRV
VFGARDAKTGAAGSLMDVLHHPGMNHRVEITEGILADECAALLSDFFRMRR
QEIKAQKKAQSSTD.

In some embodiments the TadA deaminase is a full-length E. coli TadA deaminase. For example, in certain embodiments, the adenosine deaminase comprises the amino acid sequence:

(SEQ ID NO: 401)
MRRAFITGVFFLSEVEFSHEYWMRHALTLAKRAWDEREVPVGAVLVHNNRV
IGEGWNRPIGRHDPTAHAEIMALRQGGLVMQNYRLIDATLYVTLEPCVMCA
GAMIHSRIGRVVFGARDAKTGAAGSLMDVLHHPGMNHRVEITEGILADECA
ALLSDFFRMRRQEIKAQKKAQSSTD

It should be appreciated, however, that additional adenosine deaminases useful in the present application would be apparent to the skilled artisan and are within the scope of this disclosure. For example, the adenosine deaminase may be a homolog of an ADAT. Exemplary ADAT homologs include, without limitation:

Staphylococcus aureus TadA:

(SEQ ID NO: 402)
MGSHMTNDIYFMTLAIEEAKKAAQLGEVPIGAIITKDDEVIARAHNLRETL
QQPTAHAEHIAIERAAKVLGSWRLEGCTLYVTLEPCVMCAGTIVMSRIPRV
VYGADDPKGGCSGSLMNLLQQSNFNHRAIVDKGVLKEACSTLLTTFFKNLR
ANKKSTN

Bacillus subtilis TadA:

(SEQ ID NO: 403)
MTQDELYMKEAIKEAKKAEEKGEVPIGAVLVINGEIIARAHNLRETEQRSI
AHAEMLVIDEACKALGTWRLEGATLYVTLEPCPMCAGAVVLSRVEKVVFGA
FDPKGGCSGTLMNLLQEERFNHQAEVVSGVLEEECGGMLSAFFRELRKKKK
AARKNLSE

Salmonella typhimurium TadA:

(SEQ ID NO: 404)
MPPAFITGVTSLSDVELDHEYWMRHALTLAKRAWDEREVPVGAVLVHNHRV
IGEGWNRPIGRHDPTAHAEIMALRQGGLVLQNYRLLDTTLYVTLEPCVMCA
GAMVHSRIGRVVFGARDAKTGAAGSLIDVLHHPGMNHRVEIIEGVLRDECA
TLLSDFFRMRRQEIKALKKADRAEGAGPAV

Shewanella putrefaciens TadA:

(SEQ ID NO: 405)
MDEYWMQVAMQMAEKAEAAGEVPVGAVLVKDGQQIATGYNLSISQHDPTAH
AEILCLRSAGKKLENYRLLDATLYITLEPCAMCAGAMVHSRIARVVYGARD
EKTGAAGTVVNLLQHPAFNHQVEVTSGVLAEACSAQLSRFFKRRRDEKKAL
KLAQRAQQGIE

Haemophilus influenzae F3031 (H. influenzae) TadA:

(SEQ ID NO: 406)
MDAAKVRSEFDEKMMRYALELADKAEALGEIPVGAVLVDDARNIIGEGWNL
SIVQSDPTAHAEIIALRNGAKNIQNYRLLNSTLYVTLEPCTMCAGAILHSR
IKRLVFGASDYKTGAIGSRFHFFDDYKMNHTLEITSGVLAEECSQKLSTFF
QKRREEKKIEKALLKSLSDK

Caulobacter crescentus TadA:

(SEQ ID NO: 407)
MRTDESEDQDHRMMRLALDAARAAAEAGETPVGAVILDPSTGEVIATAGNG
PIAAHDPTAHAEIAAMRAAAAKLGNYRLTDLTLVVTLEPCAMCAGAISHAR
IGRVVFGADDPKGGAVVHGPKFFAQPTCHWRPEVTGGVLADESADLLRGFF
RARRKAKI

Geobacter sulfurreducens TadA:

(SEQ ID NO: 408)
MSSLKKTPIRDDAYWMGKAIREAAKAAARDEVPIGAVIVRDGAVIGRGHNL
REGSNDPSAHAEMIAIRQAARRSANWRLTGATLYVTLEPCLMCMGAIILAR
LERVVFGCYDPKGGAAGSLYDLSADPRLNHQVRLSPGVCQEECGTMLSDFF
RDLRRRKKAKATPALFIDERKVPPEP

The term “effective amount,” as used herein, refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. In some embodiments, an effective amount of a fusion protein provided herein, e.g., of a fusion protein comprising a Cas9 domain and a nucleic acid editing domain (e.g., a deaminase domain) may refer to the amount of the fusion protein that is sufficient to induce editing of a target site specifically bound and edited by the fusion protein. As will be appreciated by the skilled artisan, the effective amount of an agent, e.g., a fusion protein, a nuclease, a deaminase, a recombinase, a hybrid protein, a protein dimer, a complex of a protein (or protein dimer) and a polynucleotide, or a polynucleotide, may vary depending on various factors such as, for example, on the desired biological response, e.g., on the specific allele, genome, or target site to be edited; on the cell or tissue being targeted; and on the agent (e.g., Cas9 domain, fusion protein, vector, cell, etc.) being used.

The term “immediately adjacent” as used in the context of two nucleic acid sequences refers to two sequences that directly abut each other as part of the same nucleic acid molecule and are not separated by one or more nucleotides. Accordingly, sequences are immediately adjacent, when the nucleotide at the 3′-end of one of the sequences is directly connected to nucleotide at the 5′-end of the other sequence via a phosphodiester bond.

The term “linker,” as used herein, refers to a chemical group or a molecule linking two molecules or moieties, e.g., two domains of a fusion protein, such as, for example, a nuclease-inactive Cas9 domain and a nucleic acid editing domain (e.g., a deaminase domain). A linker may be, for example, an amino acid sequence, a peptide, or a polymer of any length and composition. In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. In some embodiments, a linker joins a Cas9n and a nucleic-acid editing protein. In some embodiments, a linker joins an RNA-programmable nuclease domain and a UGI domain. In some embodiments, a linker joins a dCas9 and a UGI domain. In some embodiments, a linker joins a Cas9n and a UGI domain. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker comprises the amino acid sequence of any one of SEQ ID NOs: 300-318. In some embodiments, the linker is 1-100 amino acids in length, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, a linker comprises the amino acid sequence SGSETPGTSESATPES (SEQ ID NO: 306), which may also be referred to as the XTEN linker. In some embodiments, a linker comprises the amino acid sequence SGGS (SEQ ID NO: 309). In some embodiments, a linker comprises the amino acid sequence (SGGS)₂-SGSETPGTSESATPES-(SGGS)₂ (SEQ ID NO: 308), which may also be referred to as (SGGS)₂-XTEN-(SGGS)₂. In some embodiments, a linker comprises (SGGS)ₙ(SEQ ID NO: 305), (GGGS)ₙ (SEQ ID NO: 300), (GGGGS)ₙ(SEQ ID NO: 301), (G)ₙ(SEQ ID NO: 302), (EAAAK)ₙ (SEQ ID NO: 303), (GGS)ₙ(SEQ ID NO: 304), SGGS(GGS)ₙ (SEQ ID NO: 307), (SGGS)ₙ-SGSETPGTSESATPES-(SGGS)ₙ(SEQ ID NO: 310), or (XP)ₙ motif, or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15. In some embodiments, n is 1, 3, or 7. In some embodiments, the linker comprises the amino acid sequence

(SEQ ID NO: 314)
GGSGGSPGSPAGSPTSTEEGTSESATPESGPGTSTEPSEGSAPGSPAGSPT
STEEGTSTEPSEGSAPGTSTEPSEGSAPGTSESATPESGPGSEPATSGGSG
GS,
(SEQ ID NO: 315)
SGGSSGGSSGSETPGTSESATPES,
(SEQ ID NO: 316)
SGGSSGGSSGSETPGTSESATPESSGGSSGGSSGGSSGGS,
(SEQ ID NO: 317)
SGGSSGGSSGSETPGTSESATPESSGGSSGGSSGGSSGGSSGSETPGTSES
ATPESSGGSSGGS,
or
(SEQ ID NO: 318)
PGSPAGSPTSTEEGTSESATPESGPGTSTEPSEGSAPGSPAGSPTSTEEGT
STEPSEGSAPGTSTEPSEGSAPGTSESATPESGPGSEPATS.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, an RNA is an RNA associated with the Cas9 system. For example, the RNA may be a CRISPR RNA (crRNA), a trans-encoded small RNA (tracrRNA), a single guide RNA (sgRNA), or a guide RNA (gRNA).

The term “nucleic acid editing domain,” as used herein refers to a protein or enzyme capable of making one or more modifications (e.g., deamination of a cytidine residue) to a nucleic acid (e.g., DNA or RNA). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain. In some embodiments, the nucleic acid editing domain is a deaminase, a nuclease, a nickase, a recombinase, a methyltransferase, a methylase, an acetylase, an acetyltransferase, a transcriptional activator, or a transcriptional repressor domain. In some embodiments, the nucleic acid editing domain is a deaminase domain (e.g., a cytidine deaminase, such as an APOBEC or an AID deaminase, or an adenosine deaminase, such as ecTadA). In some embodiments, the nucleic acid editing domain is a cytidine deaminase domain (e.g., an APOBEC or an AID deaminase). In some embodiments, the nucleic acid editing domain is an adenosine deaminase domain (e.g., an ecTadA).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences. In some embodiments, a NLS comprises the amino acid sequence

(SEQ ID NO: 520)
PKKKRKV
or
(SEQ ID NO: 521)
MDSLLMNRRKFLYQFKNVRWAKGRRETYLC.

The term “proliferative disease,” as used herein, refers to any disease in which cell or tissue homeostasis is disturbed in that a cell or cell population exhibits an abnormally elevated proliferation rate. Proliferative diseases include hyperproliferative diseases, such as pre-neoplastic hyperplastic conditions and neoplastic diseases. Neoplastic diseases are characterized by an abnormal proliferation of cells and include both benign and malignant neoplasias. Malignant neoplasia is also referred to as cancer.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, or synthetic, or any combination thereof. The term “fusion protein” as used herein refers to a hybrid polypeptide which comprises protein domains from at least two different proteins, or at least two identical protein domains (i.e., a homodimer). One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic acid editing protein. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via protein expression and purification, which is especially suited for fusion proteins comprising a peptide linker. Methods for protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a plant or a fungus. In some embodiments, the subject is a research animal (e.g., a rat, a mouse, or a non-human primate). In some embodiments, the subject is genetically engineered, e.g., a genetically engineered non-human subject. The subject may be of either sex, of any age, and at any stage of development.

The term “target site” refers to a nucleic acid sequence or a nucleotide within a nucleic acid that is targeted or modified by an effector domain that is fused to a napDNAbp. In some embodiments, a “target site” is a sequence within a nucleic acid molecule that is deaminated by a deaminase or a fusion protein comprising a deaminase, (e.g., a dCas9-deaminase fusion protein or a Cas9n-deaminase fusion protein provided herein). In some embodiments, the target site refers to a sequence within a nucleic acid molecule that is cleaved by a napDNAbp (e.g., a nuclease active Cas9 domain) provided herein. The target site is contained within a target sequence (e.g., a target sequence comprising a reporter gene, or a target sequence comprising a gene located in a safe harbor locus).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “pharmaceutical composition,” as used herein, refers to a composition that can be administrated to a subject in the context of treatment of a disease or disorder. In some embodiments, a pharmaceutical composition comprises an active ingredient, e.g., a nuclease or a nucleic acid encoding a nuclease, and a pharmaceutically acceptable excipient.

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 500. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 500. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 500. In some embodiments, a UGI comprises an amino acid sequence homologous to the amino acid sequence set forth in SEQ ID NO: 500, or an amino acid sequence homologous to a fragment of the amino acid sequence set forth in SEQ ID NO: 500. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 500. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 500. In some embodiments, the UGI comprises the amino acid sequence of SEQ ID NO: 500, as set forth below. Exemplary Uracil-DNA glycosylase inhibitor (UGI; >sp|P14739|UNGI_BPPB2) MTNLSDIIEKETGKQLVIQESILMLPEEVEEVIGNKPESDILVHTAYDESTDENVMLLTSDAPEYKPWALVIQDS NGENKIKML (SEQ ID NO: 500).

The term “catalytically inactive inosine-specific nuclease,” or “dead inosine-specific nuclease (dISN),” as used herein, refers to a protein that is capable of inhibiting an inosine-specific nuclease. Without wishing to be bound by any particular theory, catalytically inactive inosine glycosylases (e.g., alkyl adenine glycosylase [AAG]) will bind inosine, but will not create an abasic site or remove the inosine, thereby sterically blocking the newly-formed inosine moiety from DNA damage/repair mechanisms. In some embodiments, the catalytically inactive inosine-specific nuclease may be capable of binding an inosine in a nucleic acid but does not cleave the nucleic acid. Exemplary catalytically inactive inosine-specific nucleases include, without limitation, catalytically inactive alkyl adenosine glycosylase (AAG nuclease), for example, from a human, and catalytically inactive endonuclease V (EndoV nuclease), for example, from E. coli. In some embodiments, the catalytically inactive AAG nuclease comprises an E125Q mutation as shown in SEQ ID NO: 510, or a corresponding mutation in another AAG nuclease. In some embodiments, the catalytically inactive AAG nuclease comprises the amino acid sequence set forth in SEQ ID NO: 510. In some embodiments, the catalytically inactive EndoV nuclease comprises an D35A mutation as shown in SEQ ID NO: 51I, or a corresponding mutation in another EndoV nuclease. In some embodiments, the catalytically inactive EndoV nuclease comprises the amino acid sequence set forth in SEQ ID NO: 51I. It should be appreciated that other catalytically inactive inosine-specific nucleases (dISNs) would be apparent to the skilled artisan and are within the scope of this disclosure.

Truncated AAG (H. sapiens) nuclease (E125Q); mutated residue shown in bold.

(SEQ ID NO: 510)
KGHLTRLGLEFFDQPAVPLARAFLGQVLVRRLPNGTELRGRIVETQAYLGP
EDEAAHSRGGRQTPRNRGMFMKPGTLYVYIIYGMYFCMNISSQGDGACVLL
RALEPLEGLETMRQLRSTLRKGTASRVLKDRELCSGPSKLCQALAINKSFD
QRDLAQDEAVWLERGPLEPSEPAVVAAARVGVGHAGEWARKPLRFYVRGSP
WVSVVDRVAEQDTQA

EndoV nuclease (D35A); mutated residue shown in bold.

(SEQ ID NO: 511)
DLASLRAQQIELASSVIREDRLDKDPPDLIAGAAVGFEQGGEVTRAAMVLL
KYPSLELVEYKVARIATTMPYIPGFLSFREYPALLAAWEMLSQKPDLVFVD
GHGISHPRRLGVASHFGLLVDVPTIGVAKKRLCGKFEPLSSEPGALAPLMD
KGEQLAWVWRSKARCNPLFIATGHRVSVDSALAWVQRCMKGYRLPEPTRWA
DAVASERPAFVRYTANQP

### DETAILED DESCRIPTION OF CERTAIN EMBODIMENTS OF THE INVENTION

Some aspects of this disclosure provide Cas9 domains that efficiently target nucleic acid sequences that do not include the canonical PAM sequence (5′-NGG-3′, where N is any nucleotide, for example A, T, G, or C) at their 3′-ends. In some embodiments, the Cas9 domains provided herein comprise one or more mutations identified in directed evolution experiments using a target sequence library comprising randomized PAM sequences. The non-PAM restricted Cas9 domains provided herein are useful for targeting DNA sequences that do not comprise the canonical PAM sequence at their 3′-end and thus greatly extend the applicability and usefulness of Cas9 technology for gene editing. The evolution of Cas9 domains that are not restricted to the canonical 5′-NGG-3′ PAM sequence has been previously described, for example, in International Patent Application No., PCT/US2016/058345, filed Oct. 22, 2016, and published as Publication No. WO 2017/070633, published Apr. 27, 2017, entitled “Evolved Cas9 Proteins for Gene Editing” which is herein incorporated by reference in its entirety. In addition to the Cas9 mutations identified and proteins listed in Patent Publication No. WO 2017/070633, provided herein are novel additional mutations and Cas9 domains that have activity on target sequences comprising non-canonical PAM sequences. It should be understood that any of the mutations listed in Patent Publication No. WO 2017/070633 may be combined with or used in lieu of any of the mutations or Cas9 domains disclosed herein, unless explicity stated otherwise.

Some aspects of this disclosure provide fusion proteins that comprise a Cas9 domain and an effector domain, for example, a nucleic acid editing domain, such as, e.g., a deaminase domain. The deamination of a nucleobase by a deaminase can lead to a point mutation at the specific residue, which is referred to herein as nucleic acid editing. Fusion proteins comprising a Cas9 domain or variant thereof and a nucleic acid editing domain can thus be used for the targeted editing of nucleic acid sequences. Such fusion proteins are useful for targeted editing of DNA in vitro, e.g., for the generation of mutant cells or animals; for the introduction of targeted mutations, e.g., for the correction of genetic defects in cells ex vivo, e.g., in cells obtained from a subject that are subsequently re-introduced into the same or another subject; and for the introduction of targeted mutations, e.g., the correction of genetic defects or the introduction of deactivating mutations in disease-associated genes in a subject in vivo. Typically, the Cas9 domain of the fusion proteins described herein is a Cas9 domain comprising one or more mutations provided herein (e.g., an “xCas9” domain) that has impaired nuclease activity (e.g., a nuclease-inactive xCas9 domain). For example, in some embodiments, the Cas9 domain comprises a D10A and/or a H840A mutation in the amino acid sequence provided in SEQ ID NO: 9. Methods for the use of fusion proteins comprising Cas9 as described herein are also provided.

Additional suitable nuclease-inactive Cas9 domains will be apparent to those of skill in the art based on this disclosure. Such additional exemplary suitable nuclease-inactive Cas9 domains include, but are not limited to, D10A, D839A, H840A, N863A, D10A/D839A, D10A/H840A, D10A/N863A, D839A/H840A, D839A/N863A, D10A/D839A/H840A, and D10A/D839A/H840A/N863A mutant proteins (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference). In some embodiments, the Cas9 domain comprises a D10A mutation of the amino acid sequence provided in SEQ ID NO: 9, or a corresponding mutation in any of the amino acid sequences provided in SEQ ID NOs: 10-262. An exemplary Cas9 domain comprising a D10A mutation is shown in SEQ ID NO: 7.

Cas9 Domains with Activity on Non-Canonical PAMs

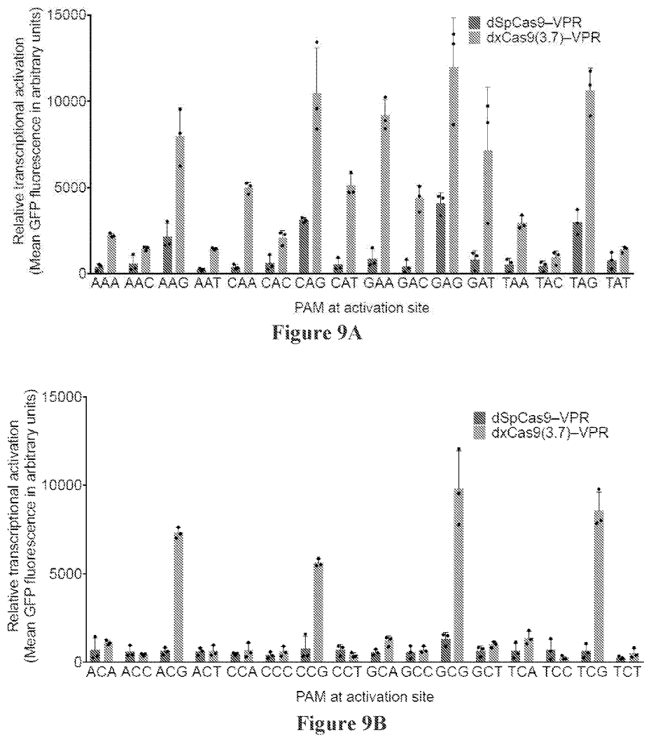

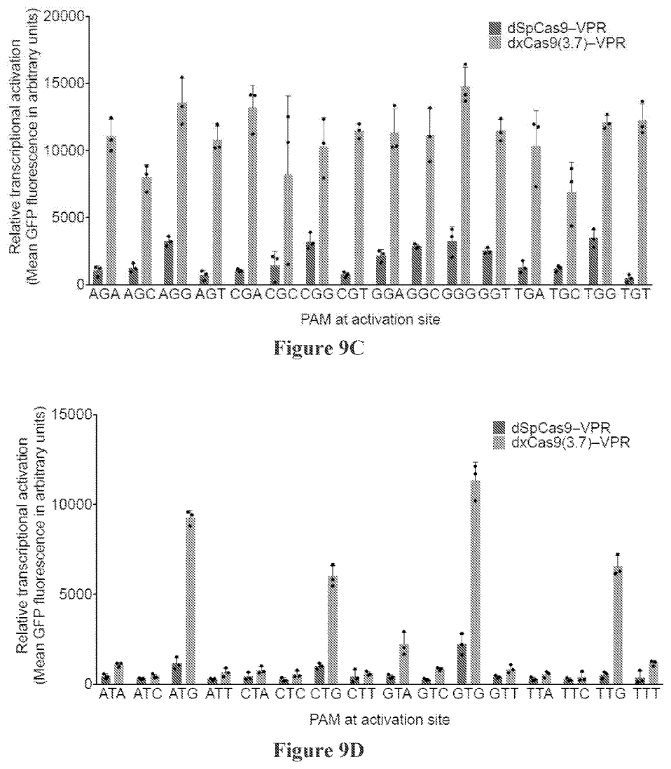

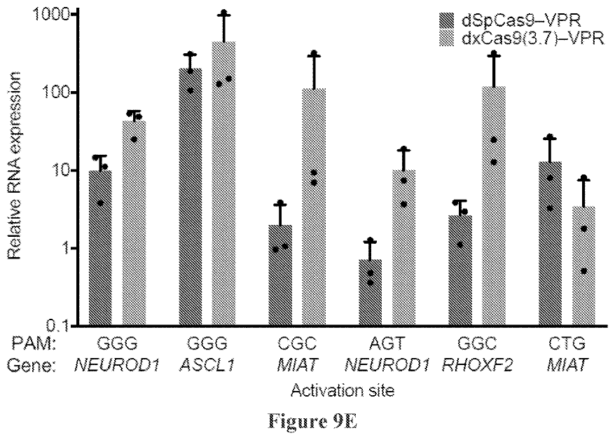

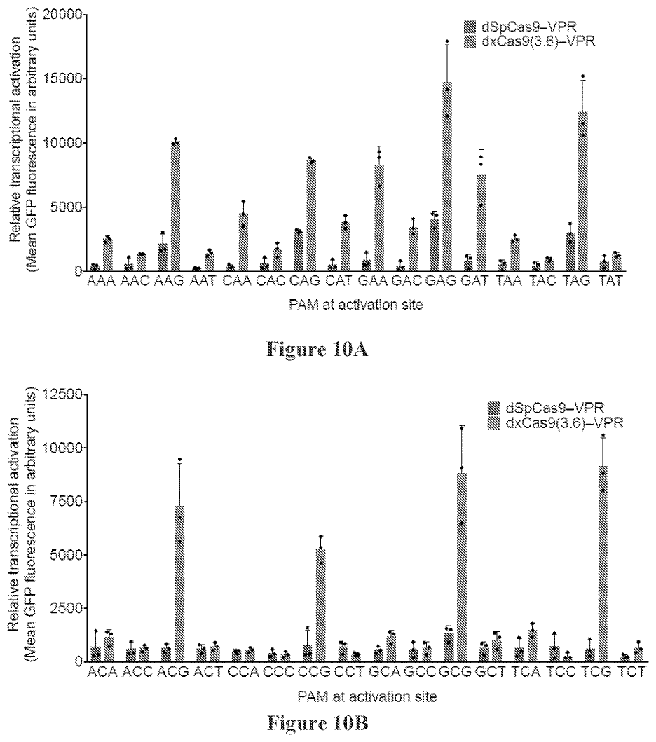

Some aspects of this disclosure provide Cas9 domains that exhibit activity on a target sequence that does not comprise the canonical PAM (5′-NGG-3′, where N is A, C, G, or T) at its 3′-end. In some embodiments, the Cas9 domain exhibits activity on a target sequence comprising a 5′-NGG-3′ PAM sequence at its 3′-end. In some embodiments, the Cas9 domain exhibits activity on a target sequence comprising a 5′-NNG-3′ PAM sequence at its 3′-end. In some embodiments, the Cas9 domain exhibits activity on a target sequence comprising a 5′-NNA-3′ PAM sequence at its 3′-end. In some embodiments, the Cas9 domain exhibits activity on a target sequence comprising a 5′-NNC-3′ PAM sequence at its 3′-end. In some embodiments, the Cas9 domain exhibits activity on a target sequence comprising a 5′-NNT-3′ PAM sequence at its 3′-end. In some embodiments, the Cas9 domain exhibits activity on a target sequence comprising a 5′-NGT-3′ PAM sequence at its 3′-end. In some embodiments, the Cas9 domain exhibits activity on a target sequence comprising a 5′-NGA-3′ PAM sequence at its 3′-end. In some embodiments, the Cas9 domain exhibits activity on a target sequence comprising a 5′-NGC-3′ PAM sequence at its 3′-end. In some embodiments, the Cas9 domain exhibits activity on a target sequence comprising a 5′-GAA-3′ PAM sequence at its 3′-end. In some embodiments, the Cas9 domain exhibits activity on a target sequence comprising a 5′-GAT-3′ PAM sequence at its 3′-end. Additional non-limiting examples of non-canonical PAM sequences that may be present in a target sequence of a Cas9 domain are shown in FIG. 9.

It should be appreciated that any of the amino acid mutations described herein, (e.g., A262T) from a first amino acid residue (e.g., A) to a second amino acid residue (e.g., T) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., a A262T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine. The skilled artisan would recognize that such conservative amino acid substitutions will likely have minor effects on protein structure and are likely to be well tolerated without compromising function. In some embodiments, any amino of the amino acid mutations provided herein from one amino acid to a threonine may be an amino acid mutation to a serine. In some embodiments, any amino of the amino acid mutations provided herein from one amino acid to an arginine may be an amino acid mutation to a lysine. In some embodiments, any amino of the amino acid mutations provided herein from one amino acid to an isoleucine, may be an amino acid mutation to an alanine, valine, methionine, or leucine. In some embodiments, any amino of the amino acid mutations provided herein from one amino acid to a lysine may be an amino acid mutation to an arginine. In some embodiments, any amino of the amino acid mutations provided herein from one amino acid to an aspartic acid may be an amino acid mutation to a glutamic acid or asparigine. In some embodiments, any amino of the amino acid mutations provided herein from one amino acid to a valine may be an amino acid mutation to an alanine, isoleucine, methionine, or leucine. In some embodiments, any amino of the amino acid mutations provided herein from one amino acid to a glycine may be an amino acid mutation to an alanine. It should be appreciated, however, that additional conserved amino acid residues would be recognized by the skilled artisan and any of the amino acid mutations to other conserved amino acid residues are also within the scope of this disclosure.

Some aspects of this disclosure provide Cas9 domains comprising an amino acid sequence that is at least 80% identical to the amino acid sequence of Cas9 as provided by any of the sequences set forth in SEQ ID NOs: 9-262, wherein the amino acid sequence of the Cas9 domain comprises at least one mutation in an amino acid residue selected from the group consisting of amino acid residues 51, 86, 115, 261, 274, 331, 319, 341, 388, 405, 435, 461, 510, 522, 548, 593, 653, 712, 715, 772, 777, 798, 811, 839, 847, 955, 967, 991, 1139, 1199, 1227, 1229, 1296, and 1318 of S. pyogenes Cas9 having the amino acid sequence provided in SEQ ID NO: 9, or in a corresponding amino acid residue in any of the amino acid sequences provided in SEQ ID NOs: 10-262. In some embodiments, the Cas9 domain comprises a RuvC and an HNH domain. In some embodiments, the amino acid sequence of the Cas9 domain is not identical to the amino acid sequence of a naturally occurring Cas9 domain. In some embodiments, the Cas9 domain is a nuclease-inactive Cas9 domain. In some embodiments, the Cas9 domain is a Cas9 nickase. In some embodiments, the amino acid sequence of the Cas9 domain comprises at least one mutation selected from the group consisting of X51I, X86L, X115H, X261G, X274E, X331Y, X319T, X341H, X388K, X405Y, X435N, X461I, X510E, X522D, X548V, X593A, X653S, X712K, X715V, X772R, X777N, X798K, X811I, X839G, X847F, X955I, X967K, X991V, X1139A, X1199T, X1227S, X1229S, X1296N, and X1318S of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 domain comprises at least one mutation selected from the group consisting of L51I, F86L, R115H, D261G, D274E, D331Y, A319T, Q341H, E388K, F405Y, D435N, R461I, K510E, N522D, I548V, T593A, R653S, Q712K, G715V, S777N, K772R, E798K, L811I, D839G, L847F, V955I, R967K, A991V, V1139A, P1199T, A1227S, P1229S, K1296N, and L1318S of the amino acid sequence provided in SEQ ID NO: 9, or a corresponding mutation in any of the amino acid sequences provided in SEQ ID NOs: 10-262.

In some embodiments, the Cas9 domain further comprises at least one mutation in an amino acid residue selected from the group consisting of amino acid residues 108, 141, 175, 217, 230, 257, 262, 267, 284, 294, 324, 405, 409, 466, 480, 543, 673, 694, 711, 1063, 1207, 1219, 1224, 1256, 1264, 1356, and 1362 of S. pyogenes Cas9 having the amino acid sequence provided in SEQ ID NO: 9, or in a corresponding amino acid residue in any of the amino acid sequences provided in SEQ ID NOs: 10-262. In some embodiments, the amino acid sequence of the Cas9 domain comprises at least one mutation selected from the group consisting of X108G, X141Q, X175T, X217A, X230F, X230S, X257N, X262T, X267G, X284N, X294R, X324L, X405I, X409I, X466A, X480K, X543D, X673E, X694I, X711E, X1063V, X1207G, X1219V, X1224N, X1256K, X1264Y, X1356I, and X1362P of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 domain comprises at least one mutation selected from the group consisting of E108G, K141Q, N175T, S217A, P230F, P230S, D257N, A262T, S267G, D284N, K294R, R324L, F405I, S409I, T466A, E480K, E543D, K673E, M694I, A711E, I1063V, E1207G, E1219V, K1224N, Q1256K, H1264Y, L1356I, and L1362P of the amino acid sequence provided in SEQ ID NO: 9, or a corresponding mutation in any of the amino acid sequences provided in SEQ ID NOs: 10-262.

In some embodiments, the Cas9 domain further comprises at least one mutation in an amino acid residue selected from the group consisting of amino acid residues 23, 122, 137, 182, 394, 474, 554, 654, 660, 727, 763, 845, 847, 1100, 1135, 1218, 1224, 1333, 1335, and 1337 of S. pyogenes Cas9 having the amino acid sequence provided in SEQ ID NO: 9, or in a corresponding amino acid residue in any of the amino acid sequences provided in SEQ ID NOs: 10-262. In some embodiments, the amino acid sequence of the Cas9 domain comprises at least one mutation selected from the group consisting of X23N, X122, X137, X182, X394H, X474I, X554R, X654L, X660, X727P, X763I, X845, X847, X1100I, X1135, X1218, X1224N, X1333, X1335, and X1337 of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 domain comprises at least one mutation selected from the group consisting of D23N, Q394H, T474I, K554R, R654L, L727P, M763I, V1100I, and K1224N of the amino acid sequence provided in SEQ ID NO: 9, or a corresponding mutation in any of the amino acid sequences provided in SEQ ID NOs: 10-262.

In another aspect, this disclosure provides Cas9 domains comprising an amino acid sequence that is at least 80% identical to the amino acid sequence of Streptococcus pyogenes Cas9 as provided by SEQ ID NO: 9, comprising the RuvC and HNH domains of SEQ ID NO: 9, wherein the amino acid sequence of the Cas9 domain comprises at least one, at least two, at least three, at least four, at least five, at least six, at least seven, at least eight, at least nine, at least ten, or more mutations in an amino acid residue selected from the group consisting of amino acid residues 108, 175, 217, 230, 257, 262, 267, 294, 324, 409, 461, 466, 480, 543, 673, 694, 711, 777, 1063, 1207, 1219, 1256, 1264, and 1356 of S. pyogenes Cas9 having the amino acid sequence provided in SEQ ID NO: 9, or in a corresponding amino acid residue in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits activity (e.g., increased activity, increased binding) on a target sequence that does not comprise the canonical PAM (5′-NGG-3′) at its 3′-end as compared to Streptococcus pyogenes Cas9 as provided by SEQ ID NO: 9. In some embodiments, the Cas9 domain exhibits activity on a target sequence that comprises the canonical PAM (5′-NGG-3′) at its 3′-end that is similar, substantially similar, or increased compared to the activity of Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, or at least twenty-four mutations selected from the group consisting of X108G, X175T, X217A, X230F, X257N, X262T, A267G, X294R, X324L, X409I, X461I, X466A, X480K, X543D, X673E, X694I, X711E, X777N, X1063V, X1207G, X1219V, X1256K, X1264Y, and X1356I of the amino acid sequence provided in SEQ ID NO: 9, or a corresponding mutation in any of the amino acid sequences provided in SEQ ID NOs: 10-262, wherein X represents any amino acid at the corresponding position. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, or at least twenty-four mutations selected from the group consisting of E108G, N175T, S217A, P230F, D257N, A262T, S267G, K294R, R324L, S409I, R461I, T466A, E480K, E543D, K673E, M694I, A711E, S777N, I1063V, E1207G, E1219V, Q1256K, H1264Y, and L1356I of the amino acid sequence provided in SEQ ID NO: 9, or a corresponding mutation in any of the amino acid sequences provided in SEQ ID NOs: 10-262. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In another aspect, this disclosure provides Cas9 domains comprising an amino acid sequence that is at least 90% identical to the amino acid sequence of Streptococcus pyogenes Cas9 as provided by SEQ ID NO: 9, wherein the amino acid sequence of the Cas9 domain comprises at least one, at least two, at least three, at least four, at least five, or at least six mutations in an amino acid residue selected from the group consisting of amino acid residues 267, 294, 480, 543, 1219, and 1256 of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, or at least six mutations selected from the group consisting of X267G, X294R, X480K, X543D, X1219V, and X1256K of the amino acid sequence provided in SEQ ID NO: 9, or a corresponding mutation in any of the amino acid sequences provided in SEQ ID NOs: 10-262, wherein X represents any amino acid at the corresponding position, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, or at least six mutations selected from the group consisting of S267G, K294R, E480K, E543D, E1219V, and Q1256K of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In another aspect, provided herein are Cas9 domains comprising an amino acid sequence that is at least 90% identical to the amino acid sequence of Streptococcus pyogenes Cas9 as provided by SEQ ID NO: 9, wherein the amino acid sequence of the Cas9 domain comprises at least one, at least two, at least three, at least four, at least five, or at least six mutations in an amino acid residue selected from the group consisting of amino acid residues 294, 480, 543, 711, 1219, and 1356 of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, or at least six mutations selected from the group consisting of X294R, X480K, X543D, X711E, X1219V, and X1256K of the amino acid sequence provided in SEQ ID NO: 9, or a corresponding mutation in any of the amino acid sequences provided in SEQ ID NOs: 10-262, wherein X represents any amino acid at the corresponding position, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, or at least six mutations selected from the group consisting of K294R, E480K, E543D, A711E, E1219V, and Q1256K of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain comprises a K294R, E480K, E543D, A711E, E1219V, and Q1256K mutation in the amino acid sequence provided by SEQ ID NO: 9, or the corresponding mutations in any one of the amino acid sequences provided by SEQ ID NOs: 10-262. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In yet another aspect, provided herein are Cas9 domains comprising an amino acid sequence that is at least 90% identical to the amino acid sequence of Streptococcus pyogenes Cas9 as provided by SEQ ID NO: 9, wherein the amino acid sequence of the Cas9 domain comprises at least one, at least two, at least three, at least four, at least five, or at least six mutations in an amino acid residue selected from the group consisting of amino acid residues 262, 409, 480, 543, 694, and 1219 of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, or at least six mutations selected from the group consisting of X262T, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid at the corresponding position, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, or at least six mutations selected from the group consisting of A262T, S409I, E480K, E543D, M694I, and E1219V of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In another aspect, provided herein are Cas9 domains comprising an amino acid sequence that is at least 90% identical to the amino acid sequence of Streptococcus pyogenes Cas9 as provided by SEQ ID NO: 9, wherein the amino acid sequence of the Cas9 domain comprises at least one, at least two, at least three, at least four, at least five, at least six, at least seven, at least eight, or at least nine mutations in an amino acid residue selected from the group consisting of amino acid residues 108, 217, 262, 409, 480, 543, 694, 1219, and 1356 of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, at least six, at least seven, at least eight, or at least nine mutations selected from the group consisting of X108G, X217A, X262T, X409I, X480K, X543D, X694I, X1219V, and X1356I of the amino acid sequence provided in SEQ ID NO: 9, or a corresponding mutation in any of the amino acid sequences provided in SEQ ID NOs: 10-262, wherein X represents any amino acid at the corresponding position, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, at least six, at least seven, at least eight, or at least nine mutations selected from the group consisting of E108G, S217A, A262T, S409I, E480K, E543D, M694I, E1219V, and L1356I of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain comprises a E108G, S217A, A262T, S409I, E480K, E543D, M694I, E1219V, and L1356I mutation of the amino acid sequence provided in SEQ ID NO: 9, or a corresponding mutation in any of the amino acid sequences provided in SEQ ID NOs: 10-262. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In another aspect, provided herein are Cas9 domains comprising an amino acid sequence that is at least 90% identical to the amino acid sequence of Streptococcus pyogenes Cas9 as provided by SEQ ID NO: 9, wherein the amino acid sequence of the Cas9 domain comprises at least one, at least two, at least three, at least four, at least five, at least six, or at least seven mutations in an amino acid residue selected from the group consisting of amino acid residues 262, 324, 409, 480, 543, 694, and 1219 of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, at least six, or at least seven mutations selected from the group consisting of X262T, X324L, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid at the corresponding position, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, at least six, or at least seven mutations selected from the group consisting of A262T, R324L, S409I, E480K, E543D, M694I, and E1219V of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain comprises a A262T, R324L, S409I, E480K, E543D, M694I, and E1219V mutation of the amino acid sequence provided in SEQ ID NO: 9, or a corresponding mutation in any of the amino acid sequences provided in SEQ ID NOs: 10-262. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In another aspect, provided herein are Cas9 domains comprising an amino acid sequence that is at least 90% identical to the amino acid sequence of Streptococcus pyogenes Cas9 as provided by SEQ ID NO: 9, wherein the amino acid sequence of the Cas9 domain comprises at least one, at least two, at least three, at least four, at least five, at least six, at least seven, at least eight, or at least nine mutations in an amino acid residue selected from the group consisting of amino acid residues 108, 262, 409, 461, 480, 543, 673, 694, and 1219 of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, at least six, at least seven, at least eight, or at least nine mutations selected from the group consisting of X108G, X262T, X409I, X461I, X480K, X543D, X673E, X694I, and X1219V of the amino acid sequence provided in SEQ ID NO: 9, or a corresponding mutation in any of the amino acid sequences provided in SEQ ID NOs: 10-262, wherein X represents any amino acid at the corresponding position, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, at least six, at least seven, at least eight, or at least nine mutations selected from the group consisting of E108G, A262T, S409I, R461I, E480K, E543D, K673E, M694I, and E1219V of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain comprises a E108G, A262T, S409I, R461I, E480K, E543D, K673E, M694I, and E1219V mutation of the amino acid sequence provided in SEQ ID NO: 9, or a corresponding mutation in any of the amino acid sequences provided in SEQ ID NOs: 10-262. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In another aspect, provided herein are Cas9 domains comprising an amino acid sequence that is at least 90% identical to the amino acid sequence of Streptococcus pyogenes Cas9 as provided by SEQ ID NO: 9, wherein the amino acid sequence of the Cas9 domain comprises at least one, at least two, at least three, at least four, at least five, at least six, at least seven, at least eight, or at least nine mutations in an amino acid residue selected from the group consisting of amino acid residues 108, 262, 409, 480, 543, 694, 777, 1219, and 1356 of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, at least six, at least seven, at least eight, or at least nine mutations selected from the group consisting of X108G, X262T, X409I, X480K, X543D, X694I, X777N, X1219V, and X1356I of the amino acid sequence provided in SEQ ID NO: 9, or a corresponding mutation in any of the amino acid sequences provided in SEQ ID NOs: 10-262, wherein X represents any amino acid at the corresponding position, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises at least one, at least two, at least three, at least four, at least five, at least six, at least seven, at least eight, or at least nine mutations selected from the group consisting of E108G, A262T, S409I, E480K, E543D, M694I, S777N, E1219V, and L1356I of the amino acid sequence provided in SEQ ID NO: 9, or a corresponding mutation in any of the amino acid sequences provided in SEQ ID NOs: 10-262, wherein the amino acid sequence of the Cas9 domain is not identical to the amino acid sequence of a naturally occurring Cas9 domain, and wherein the Cas9 domain exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the Cas9 domain comprises a E108G, A262T, S409I, E480K, E543D, M694I, S777N, E1219V, and L1356I mutation of the amino acid sequence provided in SEQ ID NO: 9, or a corresponding mutation in any of the amino acid sequences provided in SEQ ID NOs: 10-262. In some embodiments, the Cas9 domain further comprises a D10A and/or a H840A mutation of the amino acid sequence provided in SEQ ID NO: 9, or a corresponding mutation in any of the amino acid sequences provided in SEQ ID NOs: 10-262.

In some embodiments, the amino acid sequence of the Cas9 domain comprises an X108G mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X108A.

In some embodiments, the amino acid sequence of the Cas9 domain comprises an E108G mutation in the amino acid sequence provided in SEQ ID NO: 9, or a corresponding mutation in any of the amino acid sequences provided in SEQ ID NOs: 10-262. In some embodiments, the mutation is E108A.

In some embodiments, the amino acid sequence of the Cas9 domain comprises an X175T mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X175A, X175V, or X175S.

In some embodiments, the amino acid sequence of the Cas9 domain comprises an N175T mutation in the amino acid sequence provided in SEQ ID NO: 9, or a corresponding mutation in any of the amino acid sequences provided in SEQ ID NOs: 10-262. In some embodiments, the mutation is N175A, N175V, or N175S.

In some embodiments, the amino acid sequence of the Cas9 domain comprises an X217A mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X217G, X217V, X217L, or X217I.

In some embodiments, the amino acid sequence of the Cas9 domain comprises an S217A mutation in the amino acid sequence provided in SEQ ID NO: 9, or a corresponding mutation in any of the amino acid sequences provided in SEQ ID NOs: 10-262. In some embodiments, the mutation is S217G, S217V, S217L, or S217I.

In some embodiments, the amino acid sequence of the Cas9 domain comprises an X230F mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X230Y.

In some embodiments, the amino acid sequence of the Cas9 domain comprises an P230F mutation in the amino acid sequence provided in SEQ ID NO: 9, or a corresponding mutation in any of the amino acid sequences provided in SEQ ID NOs: 10-262. In some embodiments, the mutation is P230Y.

In some embodiments, the amino acid sequence of the Cas9 domain comprises an X257N mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X257Q.

In some embodiments, the amino acid sequence of the Cas9 domain comprises an D257N mutation in the amino acid sequence provided in SEQ ID NO: 9, or a corresponding mutation in any of the amino acid sequences provided in SEQ ID NOs: 10-262. In some embodiments, the mutation is D257Q.

In some embodiments, the amino acid sequence of the Cas9 domain comprises an X262T mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X262S or X262V.

In some embodiments, the amino acid sequence of the Cas9 domain comprises an A262T mutation in the amino acid sequence provided in SEQ ID NO: 9, or a corresponding mutation in any of the amino acid sequences provided in SEQ ID NOs: 10-262. In some embodiments, the mutation is A262S or A262V.

In some embodiments, the amino acid sequence of the Cas9 domain comprises an X267G mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X267A.

In some embodiments, the amino acid sequence of the Cas9 domain comprises an S267G mutation in the amino acid sequence provided in SEQ ID NO: 9, or a corresponding mutation in any of the amino acid sequences provided in SEQ ID NOs: 10-262. In some embodiments, the mutation is S267A.

In some embodiments, the amino acid sequence of the Cas9 domain comprises an X294R mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X294A.

In some embodiments, the amino acid sequence of the Cas9 domain comprises an K294R mutation in the amino acid sequence provided in SEQ ID NO: 9, or a corresponding mutation in any of the amino acid sequences provided in SEQ ID NOs: 10-262. In some embodiments, the mutation is K294A.

In some embodiments, the amino acid sequence of the Cas9 domain comprises an X324L mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X294V, X294A, or X294I.

In some embodiments, the amino acid sequence of the Cas9 domain comprises an R324L mutation in the amino acid sequence provided in SEQ ID NO: 9, or a corresponding mutation in any of the amino acid sequences provided in SEQ ID NOs: 10-262. In some embodiments, the mutation is R294V, R294A, or R294I.

In some embodiments, the amino acid sequence of the Cas9 domain comprises an X409I mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X409A, X409L, X409M, or X409V.

In some embodiments, the amino acid sequence of the Cas9 domain comprises an S409I mutation in the amino acid sequence provided in SEQ ID NO: 9, or a corresponding mutation in any of the amino acid sequences provided in SEQ ID NOs: 10-262. In some embodiments, the mutation is S409A, S409L, S409M, or S409V.

In some embodiments, the amino acid sequence of the Cas9 domain comprises an X461I mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X461A, X461L, X461M, or X461V.

In some embodiments, the amino acid sequence of the Cas9 domain comprises an R461I mutation in the amino acid sequence provided in SEQ ID NO: 9, or a corresponding mutation in any of the amino acid sequences provided in SEQ ID NOs: 10-262. In some embodiments, the mutation is R461A, R461L, R461M, or R461V.

In some embodiments, the amino acid sequence of the Cas9 domain comprises an X466A mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X466G.

In some embodiments, the amino acid sequence of the Cas9 domain comprises an T466A mutation in the amino acid sequence provided in SEQ ID NO: 9, or a corresponding mutation in any of the amino acid sequences provided in SEQ ID NOs: 10-262. In some embodiments, the mutation is T466G.

In some embodiments, the amino acid sequence of the Cas9 domain comprises an X480K mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X480R.

In some embodiments, the amino acid sequence of the Cas9 domain comprises an E480K mutation of the amino acid sequence provided in SEQ ID NO: 9, or a corresponding mutation in any of the amino acid sequences provided in SEQ ID NOs: 10-262. In some embodiments, the mutation is E480R.

In some embodiments, the amino acid sequence of the Cas9 domain comprises an X543D mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X543N.

In some embodiments, the amino acid sequence of the Cas9 domain comprises an E543D mutation of the amino acid sequence provided in SEQ ID NO: 9, or a corresponding mutation in any of the amino acid sequences provided in SEQ ID NOs: 10-262. In some embodiments, the mutation is E543N.

In some embodiments, the amino acid sequence of the Cas9 domain comprises an X673E mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X673D.

In some embodiments, the amino acid sequence of the Cas9 domain comprises an K673E mutation of the amino acid sequence provided in SEQ ID NO: 9, or a corresponding mutation in any of the amino acid sequences provided in SEQ ID NOs: 10-262. In some embodiments, the mutation is K673D.

In some embodiments, the amino acid sequence of the Cas9 domain comprises an X694I mutation in the amino acid sequence provided in SEQ ID NO: 9, or a corresponding mutation in any of the amino acid sequences provided in SEQ ID NOs: 10-262, wherein X represents any amino acid. In some embodiments, the mutation is X694A, X694L, X694S, or X694V.

In some embodiments, the amino acid sequence of the Cas9 domain comprises an M694I mutation in the amino acid sequence provided in SEQ ID NO: 9, or a corresponding mutation in any of the amino acid sequences provided in SEQ ID NOs: 10-262. In some embodiments, the mutation is M694A, M694L, M694S, or M694V.

In some embodiments, the amino acid sequence of the Cas9 domain comprises an X711E mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X711D.

In some embodiments, the amino acid sequence of the Cas9 domain comprises an A711E mutation of the amino acid sequence provided in SEQ ID NO: 9, or a corresponding mutation in any of the amino acid sequences provided in SEQ ID NOs: 10-262. In some embodiments, the mutation is A711D.

In some embodiments, the amino acid sequence of the Cas9 domain comprises an X777N mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X777Q.

In some embodiments, the amino acid sequence of the Cas9 domain comprises an S777N mutation of the amino acid sequence provided in SEQ ID NO: 9, or a corresponding mutation in any of the amino acid sequences provided in SEQ ID NOs: 10-262. In some embodiments, the mutation is S777Q.

In some embodiments, the amino acid sequence of the Cas9 domain comprises an X1063V mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X1063A, X1063M, or X1063L.

In some embodiments, the amino acid sequence of the Cas9 domain comprises an I1063V mutation of the amino acid sequence provided in SEQ ID NO: 9, or a corresponding mutation in any of the amino acid sequences provided in SEQ ID NOs: 10-262. In some embodiments, the mutation is I1063A, I1063M, or I1063L.

In some embodiments, the amino acid sequence of the Cas9 domain comprises an X1207G mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X1207A.

In some embodiments, the amino acid sequence of the Cas9 domain comprises an E1207G mutation of the amino acid sequence provided in SEQ ID NO: 9, or a corresponding mutation in any of the amino acid sequences provided in SEQ ID NOs: 10-262, wherein X represents any amino acid. In some embodiments, the mutation is E1207A.

In some embodiments, the amino acid sequence of the Cas9 domain comprises an X1219V mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X1219A, X1219I, X1219M, or X1219L.

In some embodiments, the amino acid sequence of the Cas9 domain comprises an E1219V mutation of the amino acid sequence provided in SEQ ID NO: 9, or a corresponding mutation in any of the amino acid sequences provided in SEQ ID NOs: 10-262. In some embodiments, the mutation is E1219A, E1219I, E1219M or E1219L.

In some embodiments, the amino acid sequence of the Cas9 domain comprises an X1256K mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X1256R.

In some embodiments, the amino acid sequence of the Cas9 domain comprises an Q1256K mutation of the amino acid sequence provided in SEQ ID NO: 9, or a corresponding mutation in any of the amino acid sequences provided in SEQ ID NOs: 10-262. In some embodiments, the mutation is Q1256R.

In some embodiments, the amino acid sequence of the Cas9 domain comprises an X1264Y mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X1264F.

In some embodiments, the amino acid sequence of the Cas9 domain comprises an H1264Y mutation of the amino acid sequence provided in SEQ ID NO: 9, or a corresponding mutation in any of the amino acid sequences provided in SEQ ID NOs: 10-262. In some embodiments, the mutation is H1264F.

In some embodiments, the amino acid sequence of the Cas9 domain comprises an X1365I mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X1356A or X1356V.

In some embodiments, the amino acid sequence of the Cas9 domain comprises an L1365I mutation of the amino acid sequence provided in SEQ ID NO: 9, or a corresponding mutation in any of the amino acid sequences provided in SEQ ID NOs: 10-262. In some embodiments, the mutation is L1356A or L1356V.

In some embodiments, the amino acid sequence of the Cas9 domain comprises one or more mutations selected from the group consisting of X51I, X86L, X115H, X141Q, X230S, X261G, X274E, X284N, X331Y, X319T, X341H, X388K, X405Y, X405I, X435N, X510E, X522D, X548V, X593A, X653S, X712K, X715V, X772R, X798K, X811I, X839G, X847F, X955I, X967K, X991V, X1139A, X1199T, X1224N, X1227S, X1229S, X1296N, X1318S, and X1362P of the amino acid sequence provided in SEQ ID NO: 9, or a corresponding mutation in any of the amino acid sequences provided in SEQ ID NOs: 10-262, wherein X represents any amino acid at the corresponding position.

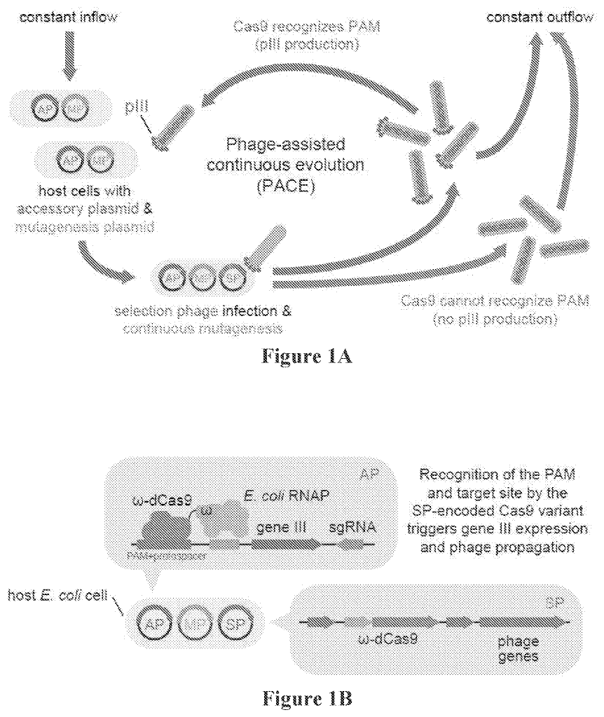

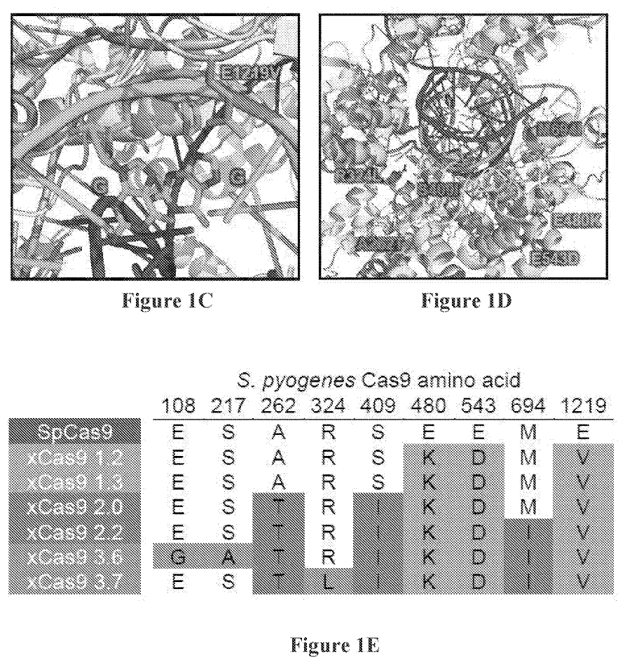

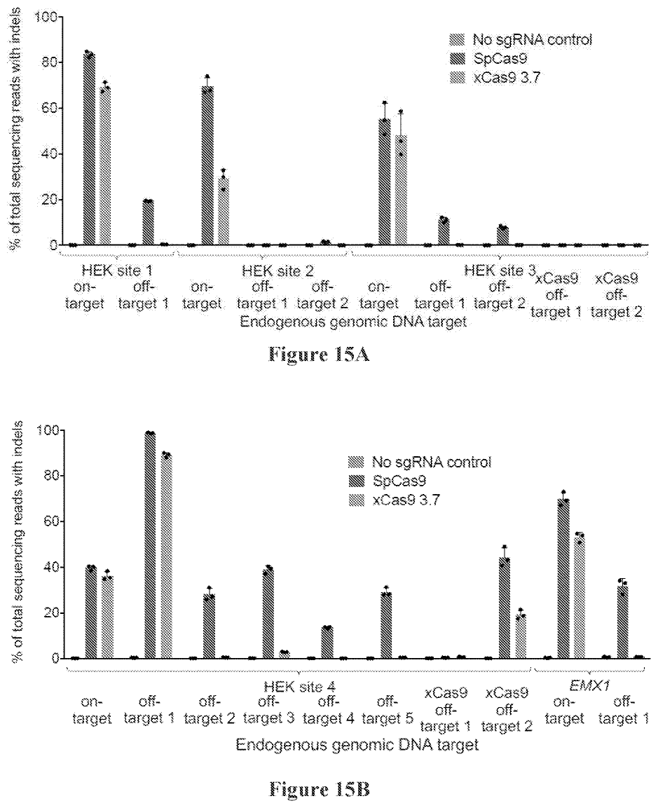

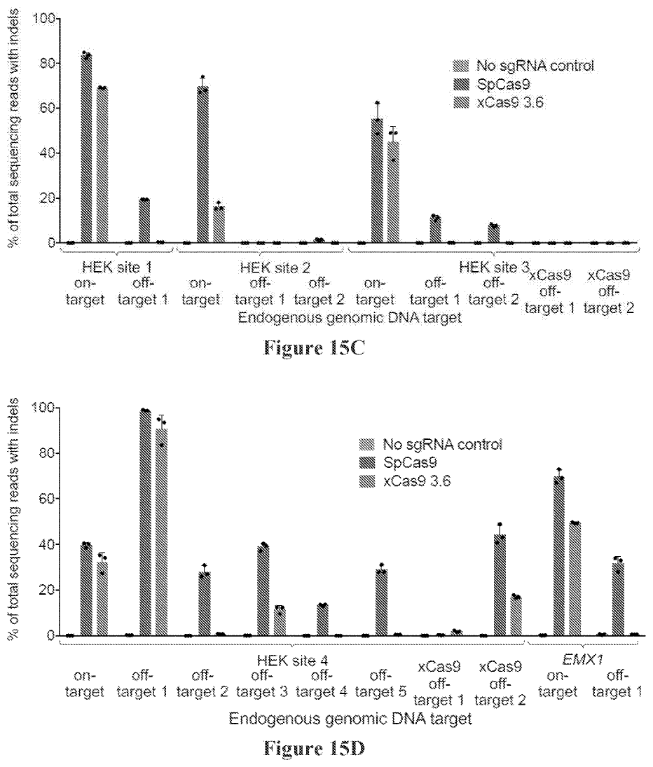

In some embodiments, the amino acid sequence of the Cas9 domain comprises one or more mutations selected from the group consisting of L51I, F86L, R115H, K141Q, P230S, D261G, D274E, D284N, D331Y, A319T, Q341H, E388K, F405Y, F405I, D435N, K510E, N522D, I548V, T593A, R653S, Q712K, G715V, K772R, E798K, L811I, D839G, L847F, V955I, R967K, A991V, V1139A, P1199T, K1224N, A1227S, P1229S, K1296N, L1318S, and L1362P of the amino acid sequence provided in SEQ ID NO: 9, or a corresponding mutation in any of the amino acid sequences provided in SEQ ID NOs: 10-262. In some embodiments, one or more of the Cas9 mutations is selected from the mutations listed in FIG. 1F and/or FIG. 15.

In one aspect, the amino acid sequence of the Cas9 domain comprises the combination of mutations selected from the group consisting of (X480K, X543D, and X1219V); (X294R, X480K, X543D, X1219V, and X1256K); (X294R, X480K, X543D, X711E, X1219V, and X1256K); (X175T, X267G, X294R, X480K, X543D, X1219V, and X1256K); (X267G, X294R, X480K, X543D, X1219V, and X1256K); (X230F, X267G, X294R, X480K, X543D, X1219V, and X1256K); (X294R, X480K, X543D, X711E, X1207G, X1219V, and X1256K); (X257N, X267G, X294R, X466A, X480K, X543D, X1063V, X1219V, and X1256K); (X262T, X409I, X480K, X543D, X694I, and X1219V); (X262T, X409I, X480K, X543D, X694I, X1264Y, and X1219V); (X262T, X409I, X480K, X543D, X694I, X1219V, and X1356I); (X108G, X262T, X409I, X461I, X480K, X543D, X673E, X694I, and X1219V); (X108G, X262T, X409I, X480K, X543D, X694I, X777N, X1219V, and X1356I); and (X108G, X262T, X409I, X480K, X543D, X673E, X694I, X1219V, and X1356I) of the amino acid sequence provided in SEQ ID NO: 9, or a corresponding mutation in any of the amino acid sequences provided in SEQ ID NOs: 10-262, wherein X is any amino acid at the corresponding position. In some embodiments, the amino acid sequence of the Cas9 domain comprises the combination of mutations selected from the group consisting of (E480K, E543D, and E1219V); (K294R, E480K, E543D, E1219V, and Q1256K); (K294R, E480K, E543D, A711E, E1219V, and Q1256K); (N175T, S267G, K294R, E480K, E543D, E1219V, and Q1256K); (S267G, K294R, E480K, E543D, E1219V, and Q1256K); (P230F, S267G, K294R, E480K, E543D, E1219V, and Q1256K); (K294R, E480K, E543D, A711E, E1207G, E1219V, and Q1256K); (D257N, S267G, K294R, T466A, E480K, E543D, I1063V, E1219V, and Q1256K); (A262T, S409I, E480K, E543D, M694I, and E1219V); (A262T, S409I, E480K, E543D, M694I, H1264Y, and E1219V); (A262T, S409I, E480K, E543D, M694I, E1219V, and L1356I); (E108G, A262T, S409I, R461I, E480K, E543D, K673E, M694I, and E1219V); (E108G, A262T, S409I, E480K, E543D, M694I, S777N, E1219V, and L1356I); and (E108G, A262T, S409I, E480K, E543D, K673E, M694I, E1219V, and L1356I) of the amino acid sequence provided in SEQ ID NO: 9, or a corresponding mutation in any of the amino acid sequences provided in SEQ ID NOs: 10-262.

In some embodiments, the Cas9 domain exhibits activity on a target sequence having a 3′-end that is not directly adjacent to the canonical PAM sequence (5′-NGG-3′), or on a target sequence that does not comprise the canonical PAM sequence (5′-NGG-3′), that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes Cas9 as provided by SEQ ID NO: 9 on the same target sequence. In some embodiments, the Cas9 domain exhibits activity on a target sequence having a 3′-end that is not directly adjacent to the canonical PAM sequence (5′-NGG-3′), or on a target sequence that does not comprise the canonical PAM sequence (5′-NGG-3′), that is at least 10%, at least 15%, at least 20%, at least 30%, at least 40%, at least 50%, at least 60%, at least 70%, at least 80%, or at least 90% greater than the activity of Streptococcus pyogenes Cas9 as provided by SEQ ID NO: 9 on the same target sequence. In some embodiments, the 3′-end of the target sequence is directly adjacent to an NGT, NGA, NGC, and NNG sequence, wherein N is A, G, T, or C. In some embodiments, the 3′-end of the target sequence is directly adjacent to an AAA, AAC, AAG, AAT, CAA, CAC, CAG, CAT, GAA, GAC, GAG, GAT, TAA, TAC, TAG, TAT, ACA, ACC, ACG, ACT, CCA, CCC, CCG, CCT, GCA, GCC, GCG, GCT, TCA, TCC, TCG, TCT, AGA, AGC, AGT, CGA, CGC, CGT, GGA, GGC, GGT, TGA, TGC, TGT, ATA, ATC, ATG, ATT, CTA, CTC, CTG, CTT, GTA, GTC, GTG, GTT, TTA, TTC, TTG, or TTT PAM sequence. In some embodiments, the 3′-end of the target sequence is directly adjacent to an CGG, AGT, TGG, AGT, CGT, GGG, CGT, TGT, GGT, AGC, CGC, TGC, GGC, AGA, CGA, TGA, GGA, GAA, GAT, or CAA sequence. In some embodiments, the Cas9 domain activity is measured by a nuclease assay, a deamination assay, a transcriptional activation assay, a binding assay, or by PCR or sequencing. In some embodiments, the transcriptional activation assay is a reporter activation assay, such as a GFP activation assay. Exemplary methods for measuring binding activity (e.g., of Cas9) using transcriptional activation assays are known in the art and would be apparent to the skilled artisan. For example, methods for measuring Cas9 activity using the tripartite activator VPR have been described in Chavez A., et al., “Highly efficient Cas9-mediated transcriptional programming.” Nature Methods 12, 326-328 (2015), the entire contents of which are incorporated by reference herein.

In some embodiments, the amino acid sequence of the HNH domain is at least 80%, at least 85%, at least 90%, at least 92%, at least 95%, at least 96%, at least 97%, at least 98%, at least 99%, or at least 99.5% identical to the amino acid sequence of the HNH domain of any of SEQ ID NOs: 2, 4, or 9. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 10-262. In some embodiments, the Cas9 domain comprises the RuvC and HNH domains of SEQ ID NO: 9. In some embodiments, the Cas9 domain comprises a D10A and/or a H840A mutation of the amino acid sequence provided in SEQ ID NO: 9, or a corresponding mutation in any of the amino acid sequences provided in SEQ ID NOs: 10-262.

### Cas9 Fusion Proteins

Any of the Cas9 domains (e.g., Cas9 domains that recognize a non-canonical PAM sequence) disclosed herein may be fused to a second protein, thus providing fusion proteins that comprise a Cas9 domain as provided herein and a second protein, or a “fusion partner.” In some embodiments, the second protein is an effector domain. As used herein, an “effector domain” refers to a molecule (e.g., a protein) that regulates a biological activity and/or is capable of modifying a biological molecule (e.g., a protein, or a nucleic acid such as DNA or RNA). In some embodiments, the effector domain is a protein. In some embodiments, the effector domain is capable of modifying a protein (e.g., a histone). In some embodiments, the effector domain is capable of modifying DNA (e.g., genomic DNA). In some embodiments, the effector domain is capable of modifying RNA (e.g., mRNA). In some embodiments, the effector molecule is a nucleic acid editing domain. In some embodiments, the effector molecule is capable of regulating an activity of a nucleic acid (e.g., transcription, and/or translation). Exemplary effector domains include, without limitation, a deaminase, a nuclease, a nickase, a recombinase, a methyltransferase, a methylase, an acetylase, an acetyltransferase, a transcriptional activator, or a transcriptional repressor domain. In some embodiments, the effector domain is a nucleic acid editing domain. Some aspects of the disclosure provide fusion proteins comprising a Cas9 domain and a nucleic acid editing domain.

In some embodiments, the fusion proteins provided herein exhibit increased activity on a target sequence that does not comprise the canonical PAM (5′-NGG-3′) at its 3′ end as compared to a fusion protein comprising Streptococcus pyogenes Cas9 as provided by SEQ ID NO: 9. In some embodiments, the fusion protein exhibits an activity on a target sequence having a 3′ end that is not directly adjacent to the canonical PAM sequence (5′-NGG-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of a fusion protein comprising Streptococcus pyogenes Cas9 as provided by SEQ ID NO: 9 on the same target sequence. In some embodiments, the 3′ end of the target sequence is directly adjacent to a sequence selected from the group consisting of NGT, NGA, NGC, and NNG, wherein N is an A, G, T, or C. In some embodiments, the 3′ end of the target sequence is directly adjacent to a sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA. In some embodiments, the fusion protein activity is measured by a nuclease assay, a deamination assay, a transcriptional activation assay, a binding assay, PCR, or sequencing. In some embodiments, the transcriptional activation assay is a GFP activation assay. In some embodiments, sequencing is used to measure indel formation. In some embodiments, the increased activity is increased binding. In some embodiments, the increased activity is increased deamination of a nucleobase in the target sequence.

Some aspects of the disclosure provide a fusion protein comprising a Cas9 domain fused to a nucleic acid editing domain, wherein the nucleic acid editing domain is fused to the N-terminus of the Cas9 domain. In some embodiments, the nucleic acid editing domain is fused to the C-terminus of the Cas9 domain. In some embodiments, the Cas9 domain and the nucleic acid editing-editing domain are fused via a linker. In some embodiments, the linker comprises a (GGGS)ₙ (SEQ ID NO: 300), a (GGGGS)ₙ (SEQ ID NO: 301), a (G)ₙ(SEQ ID NO: 302), an (EAAAK)ₙ (SEQ ID NO: 303), a (GGS)ₙ(SEQ ID NO: 304), (SGGS)ₙ(SEQ ID NO: 305), an SGSETPGTSESATPES (SEQ ID NO: 306) motif (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), a SGGS(GGS)ₙ (SEQ ID NO: 307), (SGGS)ₙ-SGSETPGTSESATPES-(SGGS)ₙ(SEQ ID NO: 310), or an (XP)ₙ motif, or a combination of any of these, wherein n is independently an integer between 1 and 30. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. In some embodiments, the linker comprises a (GGS)ₙ motif, wherein n is 1, 2, 3, 4, 5, 6, 7, 8, 9, 10, 11, 12, 13, 14 or 15. In some embodiments, the linker comprises a (GGS)ₙ motif, wherein n is 1, 3, or 7. In some embodiments, the linker comprises the amino acid sequence SGSETPGTSESATPES (SEQ ID NO: 306). In some embodiments, the linker comprises the amino acid sequence (SGGS)₂-SGSETPGTSESATPES-(SGGS)₂ (SEQ ID NO: 308). Additional suitable linker motifs and linker configurations will be apparent to those of ordinary skill in the art. In some embodiments, suitable linker motifs and configurations include those described in Chen et al., Fusion protein linkers: property, design and functionality. Adv. Drug Deliv. Rev. 2013; 65(10):1357-69, the entire contents of which are incorporated herein by reference. Additional suitable linker sequences will be apparent to those of ordinary skill in the art based on the instant disclosure. In some embodiments, the general architecture of exemplary Cas9 fusion proteins provided herein comprises the structure:
[NH₂]-[nucleic acid editing domain]-[Cas9 domain]-[COOH],
[NH₂]-[nucleic acid editing domain]-[linker]-[Cas9 domain]-[COOH],
[NH2]-[Cas9 domain]-[nucleic acid editing domain]-[COOH], or
[NH2]-[Cas9 domain]-[linker]-[nucleic acid editing domain]-[COOH]
   wherein NH₂ is the N-terminus of the fusion protein, and COOH is the C-terminus of the fusion protein. In some embodiments, the “]-[” used in the general architecture above indicates the presence of an optional linker sequence.

The fusion proteins of the present disclosure may comprise one or more additional features. For example, in some embodiments, the fusion protein comprises a nuclear localization sequence (NLS). In some embodiments, the NLS of the fusion protein is localized between the nucleic acid editing domain and the Cas9 domain. In some embodiments, the NLS of the fusion protein is localized C-terminal to the Cas9 domain. In some embodiments, the NLS of the fusion protein is localized N-terminal to the Cas9 domain. In some embodiments, the NLS comprises the amino acid sequence of SEQ ID NO: 520. In some embodiments, the NLS comprises the amino acid sequence of SEQ ID NO: 521.

Other exemplary features that may be present are localization sequences, such as cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of ordinary skill in the art. In some embodiments, the fusion protein comprises one or more His tags.

In some embodiments, the nucleic acid editing domain is a deaminase. In some embodiments, the deaminase is a cytidine deaminase. For example, in some embodiments, the general architecture of exemplary Cas9 fusion proteins with a cytidine deaminase domain comprises the structure:
[NH₂]-[NLS]-[cytidine deaminase]-[Cas9]-[COOH],
[NH₂]-[Cas9]-[cytidine deaminase]-[COOH],
[NH₂]-[cytidine deaminase]-[Cas9]-[COOH], or
[NH₂]-[cytidine deaminase]-[Cas9]-[NLS]-[COOH],
   wherein NLS is a nuclear localization sequence, NH₂ is the N-terminus of the fusion protein, and COOH is the C-terminus of the fusion protein. Nuclear localization sequences are known in the art and would be apparent to the skilled artisan. For example, NLS sequences are described in Plank et al., International PCT Application, PCT/EP2000/011690, the contents of which are incorporated herein by reference for their disclosure of exemplary nuclear localization sequences. In some embodiments, a NLS comprises the amino acid sequence PKKKRKV (SEQ ID NO: 520) or MDSLLMNRRKFLYQFKNVRWAKGRRETYLC (SEQ ID NO: 521). In some embodiments, a linker is inserted between the Cas9 and the cytidine deaminase. In some embodiments, the NLS is located C-terminal of the Cas9 domain. In some embodiments, the NLS is located N-terminal of the Cas9 domain. In some embodiments, the NLS is located between the cytidine deaminase and the Cas9 domain. In some embodiments, the NLS is located N-terminal of the cytidine deaminase domain. In some embodiments, the NLS is located C-terminal of the cytidine deaminase domain. In some embodiments, the “]-[” used in the general architecture above indicates the presence of an optional linker sequence.

In some embodiments, the fusion protein comprises any one of nucleic acid editing domains provided herein. In some embodiments, the nucleic acid editing domain is a cytidine deaminase domain provided herein. In some embodiments, the nucleic acid editing domain is a cytidine deaminase domain comprising the amino acid sequence set forth in any one of SEQ ID NOs: 350-389.

In some embodiments, the cytidine deaminase domain and the Cas9 domain are fused to each other via a linker. Various linker lengths and flexibilities between the deaminase domain (e.g., AID, APOBEC family deaminase) and the Cas9 domain can be employed, for example, ranging from very flexible linkers of the form (GGGS)ₙ (SEQ ID NO: 300), (GGGGS)ₙ (SEQ ID NO: 301), (GGS)ₙ(SEQ ID NO: 304), and (G). (SEQ ID NO: 302), to more rigid linkers of the form (EAAAK)ₙ (SEQ ID NO: 303), (SGGS)ₙ(SEQ ID NO: 305), SGGS(GGS)ₙ (SEQ ID NO: 307), SGSETPGTSESATPES (SEQ ID NO: 306) (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), (SGGS)ₙ-SGSETPGTSESATPES-(SGGS)ₙ(SEQ ID NO: 310), and (XP)ₙ, wherein n is an integer between 1 and 30, inclusive, in order to achieve the optimal length for deaminase activity for the specific application. In some embodiments, the linker comprises a (GGS)ₙ motif, wherein n is 1, 3, or 7. In some embodiments, the linker comprises a SGSETPGTSESATPES (SEQ ID NO: 306) motif. In some embodiments, the linker comprises a (SGGS)₂-SGSETPGTSESATPES-(SGGS)₂ (SEQ ID NO: 308) motif.

In some embodiments, the fusion protein comprises a Cas9 domain (e.g., a Cas9 domain comprising one or more mutations that recognizes a non-canonical PAM sequence) fused to a cytidine deaminase domain, wherein the fusion protein comprises the amino acid sequence of SEQ ID NO: 533 or 537. In some embodiments, the fusion protein comprises the amino acid sequence of SEQ ID NO: 533. In some embodiments, the fusion protein consists of the amino acid sequence of SEQ ID NO: 533. In some embodiments, the fusion protein comprises the amino acid sequence of SEQ ID NO: 537. In some embodiments, the fusion protein is of the amino acid sequence of SEQ ID NO: 537.

xCas9 3.7 (xCas9 3.7-Linker(4aa)-NLS):

(SEQ ID NO: 533)
MDKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSN
FDLAED [symbol] KLQLSKDTYDDDLDNLLAQIGDQYADLFLAAKNLSDAILLSDIL
RVNTEITKAPLSASMIK [symbol] YDEHHQDLTLLKALVRQQLPEKYKEIFFDQSKN
GYAGYIDGGASQEEFYKFIKPILEKMDGTEELLVKLNREDLLRKQRTFDNG [symbol]
IPHQIHLGELHAILRRQEDFYPFLKDNREKIEKILTFRIPYYVGPLARGN
SRFAWMTRKSEETITPWNFE [symbol] VVDKGASAQSFIERMTNFDKNLPNEKVLPK
HSLLYEYFTVYNELTKVKYVTEGMRKPAFLSG [symbol] QKKAIVDLLFKTNRKVTV
KQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLS
RKLINGIRDKQSGKTILDFLKSDGFANRNF [symbol] QLIHDDSLTFKEDIQKAQVS
GQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKD
FQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSS
FEKNPIDFLEAKGYKEVKKDLIIKLPKYSLFELENGRKRMLASAG [symbol] LQKGN
ELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKY
FDTTIDRKRYTSTKEVLDATLIHQSITGLYETRIDLSQLGGD [symbol] PKKKR
KV

xCas9 3.6 (xCas9 3.6-Linker(4aa)-NLS):

(SEQ ID NO: 537)
MDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLE
ESFLV [symbol] EDKKHERHPIFGNIVDEVAYHEKYPTIYHLRKKLVDSTDKADLRL
IYLALAHMIKFRGHFLIEGDLNPDNSDVDKLFIQLVQTYNQLFEENPINAS
GVDAKAILSARL [symbol] KSRRLENLIAQLPGEKKNGLFGNLIALSLGLTPNFKSN
FDLAED [symbol] KLQLSKDTYDDDLDNLLAQIGDQYADLFLAAKNLSDAILLSDIL
RVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNG [symbol]
IPHQIHLGELHAILRRQEDFYPFLKDNREKIEKILTFRIPYYVGPLARGN
SRFAWMTRKSEETITPWNFE [symbol] VVDKGASAQSFIERMTNFDKNLPNEKVLPK
HSLLYEYFTVYNELTKVKYVTEGMRKPAFLSG [symbol] QKKAIVDLLFKTNRKVTV
KQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLS
RKLINGIRDKQSGKTILDFLKSDGFANRNF [symbol] QLIHDDSLTFKEDIQKAQVS
GQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKD
FQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSS
FEKNPIDFLEAKGYKEVKKDLIIKLPKYSLFELENGRKRMLASAG [symbol] LQKGN
ELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKY
FDTTIDRKRYTSTKEVLDATLIHQSITGLYETRID [symbol] SQLGGD [symbol] PKKKR
KV

Some aspects of the disclosure relate to fusion proteins that comprise a uracil glycosylase inhibitor (UGI) domain. In some embodiments, any of the fusion proteins provided herein that comprise a Cas9 domain (e.g., a Cas9 domain comprising one or more mutations that recognizes a non-canonical PAM sequence) may be further fused to a UGI domain either directly or via a linker. Some aspects of this disclosure provide deaminase-dCas9 fusion proteins, deaminase-nuclease active Cas9 fusion proteins and deaminase-Cas9 nickase fusion proteins with increased nucleobase editing efficiency. Without wishing to be bound by any particular theory, cellular DNA-repair response to the presence of U:G heteroduplex DNA may be responsible for the decrease in nucleobase editing efficiency in cells. For example, uracil DNA glycosylase (UDG) catalyzes removal of U from DNA in cells, which may initiate base excision repair, with reversion of the U:G pair to a C:G pair as the most common outcome. A Uracil DNA Glycosylase Inhibitor (UGI) may inhibit human UDG activity. Thus, this disclosure contemplates a fusion protein comprising a Cas9 domain and a nucleic acid editing domain (e.g., a deaminase) further fused to a UGI domain. In some embodiments, the fusion protein comprising a Cas9 nickase-nucleic acid editing domain further fused to a UGI domain. In some embodiments, the fusion protein comprising a dCas9-nucleic acid editing domain further fused to a UGI domain. It should be understood that the use of a UGI domain may increase the editing efficiency of a nucleic acid editing domain that is capable of catalyzing, for example, a C to U change. For example, fusion proteins comprising a UGI domain may be more efficient in deaminating C residues.

In some embodiments, the fusion protein comprises the structure:
[nucleic acid editing domain]-[optional linker sequence]-[Cas9]-[optional linker sequence]-[UGI];
[nucleic acid editing domain]-[optional linker sequence]-[UGI]-[optional linker sequence]-[Cas9];
[UGI]-[optional linker sequence]-[nucleic acid editing domain]-[optional linker sequence]-[Cas9];
[UGI]-[optional linker sequence]-[Cas9]-[optional linker sequence]-[nucleic acid editing domain];
[Cas9]-[optional linker sequence]-[nucleic acid editing domain]-[optional linker sequence]-[UGI]; or
[Cas9]-[optional linker sequence]-[UGI]-[optional linker sequence]-[nucleic acid editing domain].

In some embodiments, the fusion protein comprises the structure:
[deaminase]-[optional linker sequence]-[Cas9]-[optional linker sequence]-[UGI];
[deaminase]-[optional linker sequence]-[UGI]-[optional linker sequence]-[Cas9];
[UGI]-[optional linker sequence]-[deaminase]-[optional linker sequence]-[Cas9];
[UGI]-[optional linker sequence]-[Cas9]-[optional linker sequence]-[deaminase];
[Cas9]-[optional linker sequence]-[deaminase]-[optional linker sequence]-[UGI]; or
[Cas9]-[optional linker sequence]-[UGI]-[optional linker sequence]-[deaminase].

In some embodiments, the fusion protein comprises the structure:
[cytidine deaminase]-[optional linker sequence]-[Cas9]-[optional linker sequence]-[UGI];
[cytidine deaminase]-[optional linker sequence]-[UGI]-[optional linker sequence]-[Cas9];
[UGI]-[optional linker sequence]-[cytidine deaminase]-[optional linker sequence]-[Cas9];
[UGI]-[optional linker sequence]-[Cas9]-[optional linker sequence]-[cytidine deaminase];
[Cas9]-[optional linker sequence]-[cytidine deaminase]-[optional linker sequence]-[UGI]; or
[Cas9]-[optional linker sequence]-[UGI]-[optional linker sequence]-[cytidine deaminase].

In some embodiments, the fusion proteins provided herein do not comprise a linker sequence. In some embodiments, one or both of the optional linker sequences are present.

In some embodiments, the “-” used in the general architecture above indicates the presence of an optional linker sequence. In some embodiments, the fusion proteins comprising a UGI domain further comprise a nuclear targeting sequence, for example, a nuclear localization sequence. In some embodiments, fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the UGI protein. In some embodiments, the NLS is fused to the C-terminus of the UGI protein. In some embodiments, the NLS is fused to the N-terminus of the Cas9 domain. In some embodiments, the NLS is fused to the C-terminus of the Cas9 domain. In some embodiments, the NLS is fused to the N-terminus of the deaminase. In some embodiments, the NLS is fused to the C-terminus of the deaminase. In some embodiments, the NLS is fused to the N-terminus of the second Cas9. In some embodiments, the NLS is fused to the C-terminus of the second Cas9. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. In some embodiments, the NLS comprises an amino acid sequence as set forth in SEQ ID NO: 520 or SEQ ID NO: 521.

In some embodiments, a UGI domain comprises a wild-type UGI or a UGI as set forth in SEQ ID NO: 500. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 500. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 500. In some embodiments, a UGI comprises an amino acid sequence homologous to the amino acid sequence set forth in SEQ ID NO: 500 or an amino acid sequence homologous to a fragment of the amino acid sequence set forth in SEQ ID NO: 500. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 500. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 500. In some embodiments, the UGI comprises the following amino acid sequence:

Uracil-DNA Glycosylase Inhibitor (>sp|P14739|UNGI_BPPB2)

(SEQ ID NO: 500)
MTNLSDIIEKETGKQLVIQESILMLPEEVEEVIGNKPESDILVHTAYDEST
DENVMLLTSDAPEYKPWALVIQDSNGENKIKML

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), the entire contents of each of which are incorporated herein by reference.

It should be appreciated that additional proteins may be uracil glycosylase inhibitors. For example, other proteins that are capable of inhibiting (e.g., sterically blocking) a uracil-DNA glycosylase base-excision repair enzyme are within the scope of this disclosure. Additionally, any proteins that block or inhibit base-excision repair as also within the scope of this disclosure. In some embodiments, a protein that binds DNA is used. In another embodiment, a substitute for UGI is used. In some embodiments, a uracil glycosylase inhibitor is a protein that binds single-stranded DNA. For example, a uracil glycosylase inhibitor may be a Erwinia tasmaniensis single-stranded binding protein. In some embodiments, the single-stranded binding protein comprises the amino acid sequence (SEQ ID NO: 501). In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. For example, a uracil glycosylase inhibitor is a UdgX. In some embodiments, the UdgX comprises the amino acid sequence (SEQ ID NO: 502). As another example, a uracil glycosylase inhibitor is a catalytically inactive UDG. In some embodiments, a catalytically inactive UDG comprises the amino acid sequence (SEQ ID NO: 503). It should be appreciated that other uracil glycosylase inhibitors would be apparent to the skilled artisan and are within the scope of this disclosure. In some embodiments, a uracil glycosylase inhibitor is a protein that is homologous to any one of SEQ ID NOs: 501-503. In some embodiments, a uracil glycosylase inhibitor is a protein that is at least 50% identical, at least 55% identical, at least 60% identical, at least 65% identical, at least 70% identical, at least 75% identical, at least 80% identical at least 85% identical, at least 90% identical, at least 95% identical, at least 96% identical, at least 98% identical, at least 99% identical, or at least 99.5% identical to any one of SEQ ID NOs: 501-503.

Erwinia tasmaniensis SSB (themostable single-stranded DNA binding protein)

(SEQ ID NO: 501)
MASRGVNKVILVGNLGQDPEVRYMPNGGAVANITLATSESWRDKQTGETKE
KTEWHRVVLFGKLAEVAGEYLRKGSQVYIEGALQTRKWTDQAGVEKYTTEV
VVNVGGTMQMLGGRSQGGGASAGGQNGGSNNGWGQPQQPQGGNQFSGGAQQ
QARPQQQPQQNNAPANNEPPIDFDDDIP

UdgX (binds to Uracil in DNA but does not excise)

(SEQ ID NO: 502)
MAGAQDFVPHTADLAELAAAAGECRGCGLYRDATQAVFGAGGRSARIMMIG
EQPGDKEDLAGLPFVGPAGRLLDRALEAADIDRDALYVTNAVKHFKFTRAA
GGKRRIHKTPSRTEVVACRPWLIAEMTSVEPDVVVLLGATAAKALLGNDFR
VTQHRGEVLHVDDVPGDPALVATVHPSSLLRGPKEERESAFAGLVDDLRVA
ADVRP

UDG (catalytically inactive human UDG, binds to Uracil in DNA but does not excise)

(SEQ ID NO: 503)
MIGQKTLYSFFSPSPARKRHAPSPEPAVQGTGVAG
VPEESGDAAAIPAKKAPAGQEEPGTPPSSPLSAEQ
LDRIQRNKAAALLRLAARNVPVGFGESWKKHLSGE
FGKPYFIKLMGFVAEERKHYTVYPPPHQVFTWTQM
CDIKDVKVVILGQEPYHGPNQAHGLCFSVQRPVPP
PPSLENIYKELSTDIEDFVHPGHGDLSGWAKQGVL
LLNAVLTVRAHQANSHKERGWEQFTDAVVSWLNQN
SNGLVFLLWGSYAQKKGSAIDRKRHHVLQTAHPSP
LSVYRGFFGCRHFSKTNELLQKSGKKPIDWKEL

In some embodiments, the fusion protein comprises a Cas9 domain (e.g., a Cas9 domain comprising one or more mutations that recognizes a non-canonical PAM sequence) fused to a cytidine deaminase domain, wherein the fusion protein comprises or consists of the amino acid sequence of SEQ ID NO: 534 or 538. In some embodiments, the fusion protein comprises the amino acid sequence of SEQ ID NO: 534. In some embodiments, the fusion protein consists of the amino acid sequence of SEQ ID NO: 534. In some embodiments, the fusion protein comprises the amino acid sequence of SEQ ID NO: 538. In some embodiments, the fusion protein consists of the amino acid sequence of SEQ ID NO: 538. In some embodiments, the fusion protein comprises the amino acid sequence of SEQ ID NO: 536. In some embodiments, the fusion protein consists of the amino acid sequence of SEQ ID NO: 536. In some embodiments, the fusion protein comprises an amino acid sequence that is at least 70%, at least 75%, at least 80%, at least 85%, at least 90%, at least 95%, at least 96%, at least 97%, at least 98%, at least 99%, or at least 99.5% identical to the amino acid sequence as set forth in SEQ ID NOs: 534 or 538. In some embodiments, the Cas9 domain of SEQ ID NO: 536 is replaced with any of the Cas9 domains comprising one or more mutations provided herein.

xCas9(3.7)-BE3 ( [symbol] -linker(16aa)- [symbol] -linker(4aa)-UGI-linker(4aa)-NLS):

(SEQ ID NO: 534)

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

DKKYSIGLAIGTNSVGWAVITDEYKVPSKKFKVLG
NTDRHSIKKNLIGALLFDSGETAEATRLKRTARRR
YTRRKNRICYLQEIFSNEMAKVDDSFFHRLEESFL
VEEDKKHERHPIFGNIVDEVAYHEKYPTIYHLRKK
LVDSTDKADLRLIYLALAHMIKFRGHFLIEGDLNP
DNSDVDKLFIQLVQTYNQLFEENPINASGVDAKAI
LSARLSKSRRLENLIAQLPGEKKNGLFGNLIALSL
GLTPNFKSNFDLAED [symbol] KLQLSKDTYDDDLDNLLAQ
IGDQYADLFLAAKNLSDAILLSDILRVNTEITKAP
LSASMIK [symbol] YDEHHQDLTLLKALVRQQLPEKYKEIF
FDQSKNGYAGYIDGGASQEEFYKFIKPILEKMDGT
EELLVKLNREDLLRKQRTFDNG [symbol] IPHQIHLGELHA
ILRRQEDFYPFLKDNREKIEKILTFRIPYYVGPLA
RGNSRFAWMTRKSEETITPWNFE [symbol] VVDKGASAQSF
IERMTNFDKNLPNEKVLPKHSLLYEYFTVYNELTK
VKYVTEGMRKPAFLSG [symbol] QKKAIVDLLFKTNRKVTV
KQLKEDYFKKIECFDSVEISGVEDRFNASLGTYHD
LLKIIKDKDFLDNEENEDILEDIVLTLTLFEDREM
IEERLKTYAHLFDDKVMKQLKRRRYTGWGRLSRKL
INGIRDKQSGKTILDFLKSDGFANRNF [symbol] QLIHDDS
LTFKEDIQKAQVSGQGDSLHEHIANLAGSPAIKKG
ILQTVKVVDELVKVMGRHKPENIVIEMARENQTTQ
KGQKNSRERMKRIEEGIKELGSQILKEHPVENTQL
QNEKLYLYYLQNGRDMYVDQELDINRLSDYDVDHI
VPQSFLKDDSIDNKVLTRSDKNRGKSDNVPSEEVV
KKMKNYWRQLLNAKLITQRKFDNLTKAERGGLSEL
DKAGFIKRQLVETRQITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEIGKATAKYFFYSNIMNFFKTEIT
LANGEIRKRPLIETNGETGEIVWDKGRDFATVRKV
LSMPQVNIVKKTEVQTGGFSKESILPKRNSDKLIA
RKKDWDPKKYGGFDSPTVAYSVLVVAKVEKGKSKK
LKSVKELLGITIMERSSFEKNPIDFLEAKGYKEVK
KDLIIKLPKYSLFELENGRKRMLASAG [symbol] LQKGNEL
ALPSKYVNFLYLASHYEKLKGSPEDNEQKQLFVEQ
HKHYLDEIIEQISEFSKRVILADANLDKVLSAYNK
HRDKPIREQAENIIHLFTLTNLGAPAAFKYFDTTI
DRKRYTSTKEVLDATLIHQSITGLYETRIDLSQLG
GD [symbol] TNLSDIIEKETGKQLVIQESILMLPEEVE
EVIGNKPESDILVHTAYDESTDENVMLLTSDAPEY
KPWALVIQDSNGENKIKML [symbol] PKKKRKV

xCas9(3.6)-BE3 ( [symbol] -linker(16aa)- [symbol] -linker(4aa)-UGI-linker(4aa)-NLS):

(SEQ ID NO: 538)

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

D
KKYSIGLAIGTNSVGWAVITDEYKVPSKKFKVLGN
TDRHSIKKNLIGALLFDSGETAEATRLKRTARRRY
TRRKNRICYLQEIFSNEMAKVDDSFFHRLEESFLV [symbol]
EDKKHERHPIFGNIVDEVAYHEKYPTIYHLRKKL
VDSTDKADLRLIYLALAHMIKFRGHFLIEGDLNPD
NSDVDKLFIQLVQTYNQLFEENPINASGVDAKAIL
SARL [symbol] KSRRLENLIAQLPGEKKNGLFGNLIALSLG
LTPNFKSNFDLAED [symbol] KLQLSKDTYDDDLDNLLAQI
GDQYADLFLAAKNLSDAILLSDILRVNTEITKAPL
SASMIKRYDEHHQDLTLLKALVRQQLPEKYKEIFF
DQSKNGYAGYIDGGASQEEFYKFIKPILEKMDGTE
ELLVKLNREDLLRKQRTFDNG [symbol] IPHQIHLGELHAI
LRRQEDFYPFLKDNREKIEKILTFRIPYYVGPLAR
GNSRFAWMTRKSEETITPWNFE [symbol] VVDKGASAQSFI
ERMTNFDKNLPNEKVLPKHSLLYEYFTVYNELTKV
KYVTEGMRKPAFLSG [symbol] QKKAIVDLLFKTNRKVTVK
QLKEDYFKKIECFDSVEISGVEDRFNASLGTYHDL
LKIIKDKDFLDNEENEDILEDIVLTLTLFEDREMI
EERLKTYAHLFDDKVMKQLKRRRYTGWGRLSRKLI
NGIRDKQSGKTILDFLKSDGFANRNF [symbol] QLIHDDSL
TFKEDIQKAQVSGQGDSLHEHIANLAGSPAIKKGI
LQTVKVVDELVKVMGRHKPENIVIEMARENQTTQK
GQKNSRERMKRIEEGIKELGSQILKEHPVENTQLQ
NEKLYLYYLQNGRDMYVDQELDINRLSDYDVDHIV
PQSFLKDDSIDNKVLTRSDKNRGKSDNVPSEEVVK
KMKNYWRQLLNAKLITQRKFDNLTKAERGGLSELD
KAGFIKRQLVETRQITKHVAQILDSRMNTKYDEND
KLIREVKVITLKSKLVSDFRKDFQFYKVREINNYH
HAHDAYLNAVVGTALIKKYPKLESEFVYGDYKVYD
VRKMIAKSEQEIGKATAKYFFYSNIMNFFKTEITL
ANGEIRKRPLIETNGETGEIVWDKGRDFATVRKVL
SMPQVNIVKKTEVQTGGFSKESILPKRNSDKLIAR
KKDWDPKKYGGFDSPTVAYSVLVVAKVEKGKSKKL
KSVKELLGITIMERSSFEKNPIDFLEAKGYKEVKK
DLIIKLPKYSLFELENGRKRMLASAG [symbol] LQKGNELA
LPSKYVNFLYLASHYEKLKGSPEDNEQKQLFVEQH
KHYLDEIIEQISEFSKRVILADANLDKVLSAYNKH
RDKPIREQAENIIHLFTLTNLGAPAAFKYFDTTID
RKRYTSTKEVLDATLIHQSITGLYETRID [symbol] SQLGG
D [symbol] TNLSDIIEKETGKQLVIQESILMLPEEVEE
VIGNKPESDILVHTAYDESTDENVMLLTSDAPEYK
PWALVIQDSNGENKIKML [symbol] PKKKRKV

BE3 (rAPOBEC1-XTEN-Cas9n-UGI-NLS)

(SEQ ID NO: 536)
MSSETGPVAVDPTLRRRIEPHEFEVFFDPRELRKE
TCLLYEINWGGRHSIWRHTSQNTNKHVEVNFIEKF
TTERYFCRNTRCSITWFLSWSPCGECSRAITEFLS
RYPHVTLFIYIARLYHHADPRNRQGLRDLISSGVT
IQIMTEQESGYCWRNFNYSPSNEAHWPRYPHLWVR
LYVLELYCIILGLPPCLNILRRKQPQLTFFTIALQ
SCHYQRLPPHILWATGLKSGSETPGTSESATPESD
KKYSIGLAIGTNSVGWAVITDEYKVPSKKFKVLGN
TDRHSIKKNLIGALLFDSGETAEATRLKRTARRRY
TRRKNRICYLQEIFSNEMAKVDDSFFHRLEESFLV
EEDKKHERHPIFGNIVDEVAYHEKYPTIYHLRKKL
VDSTDKADLRIYLALAHMIKFRGHFLIEGDLNPDN
SDVDKLFIQLVQTYNQLFEENPINASGVDAKAILS
ARLSKSRRLENLIAQLPGEKKNGLFGNLIALSLGL
TPNFKSNFDLAEDAKLQLSKDTYDDDLDNLLAQIG
DQYADLFLAAKNLSDAILLSDILRVNTEITKAPLS
ASMIKRYDEHHQDLTLLKALVRQQLPEKYKEIFFD
QSKNGYAGYIDGGASQEEFYKFIKPILEKMDGTEE
LLVKLNREDLLRKQRTFDNGSIPHQIHLGELHAIL
RRQEDFYPFLKDNREKIEKILTFRIPYYVGPLARG
NSRFAWMTRKSEETITPWNFEEVVDKGASAQSFIE
RMTNFDKNLPNEKVLPKHSLLYEYFTVYNELTKVK
YVTEGMRKPAFLSGEQKKAIVDLLFKTNRKVTFVK
QLKEDYFKKIECFDSVEISGVEDRFNASLGTYHDL
LKIIKDKDFLDNEENEDILEDIVLTLTLFEDREMI
EERLKTYAHLFDDKVMKQLKRRRYTGWGRLSRKLI
NGIRDKQSGKTILDFLKSDGFANRNFMQLIHDDSL
TFKEDIQKAQVSGQGDSLHEHIANLAGSPAIKKGI
LQTRIKVVDELVKVMGRHKPENIVIEMARENQTTQ
KGQKNSRERMKRIEEGIKELGSQILKEHPVENTQL
QNEKLYLYYLQNGRDMYVDQELDINRLSDYDVDHI
VPQSFLKDDSIDNKVLTRSDKNRGKSDNVPSEEVV
KKMKNYWRQLLNAKLITQRKFDNLTKAERGGLSEL
DKAGFIKRQLVETRQITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEIGKATAKYFFYSNIMNFFKTEIT
LANGEIRKRPLIETNGETGEIVAIDKGRDFATVRK
VLSMPQVNIVKKTEVQTGGFSKESILPKRNSDKLI
ARKKDVVDPKKYGGFDSPTVAYSVLVVAKVEKGKS
KKLKSVKELLGMMERSSFEKNPIDFLEAKGYKEVK
KDLIIKLPKYSLFELENGRKRMLASAGELQKGNEL
ALPSKYVNFLYLASHYEKLKGSPEDNEQKQLFVEQ
HKHYLDEHECASEFSKRVILADANLDKVLSAYNKH
RDKPIREQAENIIHLFTLTNLGAPAAFKYFDTTID
RKRYTSTKEVLDATLIHQSITGLYETRIDLSQLGG
DSGGSTNLSDIIEKETGKQLVIQESILMLPEEVEE
VIGNKPESDILVHTAYDESTDENVMLLTSDAPEYK
PWALVIQDSNGENKIKMLSGGSPKKKRKV

In some embodiments, any of the fusion proteins provided herein comprise a second UGI domain. In some embodiments, the second UGI domain comprises a wild-type UGI or a UGI as set forth in SEQ ID NO: 500. In some embodiments, the UGI proteins provided herein include fragments of UGI and proteins homologous to a UGI or a UGI fragment. For example, in some embodiments, the second UGI domain comprises a fragment of the amino acid sequence set forth in SEQ ID NO: 500. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 500. In some embodiments, the second UGI domain comprises an amino acid sequence homologous to the amino acid sequence set forth in SEQ ID NO: 500 or an amino acid sequence homologous to a fragment of the amino acid sequence set forth in SEQ ID NO: 500. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 500. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 500.

In some embodiments, the fusion protein comprises the amino acid sequence of any one of SEQ ID NOs: 540-542. In some embodiments, the fusion protein comprises the amino acid sequence of SEQ ID NO: 540. In some embodiments, the fusion protein consists of the amino acid sequence of SEQ ID NO: 540. In some embodiments, the fusion protein comprises the amino acid sequence of SEQ ID NO: 541. In some embodiments, the fusion protein consists of the amino acid sequence of SEQ ID NO: 541. In some embodiments, the fusion protein comprises the amino acid sequence of SEQ ID NO: 542. In some embodiments, the fusion protein consists of the amino acid sequence of SEQ ID NO: 542. In some embodiments, the fusion protein comprises an amino acid sequence that is at least 70%, at least 75%, at least 80%, at least 85%, at least 90%, at least 95%, at least 96%, at least 97%, at least 98%, at least 99%, or at least 99.5% identical to the amino acid sequence as set forth in SEQ ID NOs: 540 or 541. In some embodiments, the Cas9 domain of SEQ ID NO: 542 is replaced with any of the Cas9 domains comprising one or more mutations provided herein.

xCas9 3.6-BE4 (APOBEC1-Linker(32aa)-xCas9(3.6)n-linker(9aa)-UGI-Linker(9aa)-UGI):

(SEQ ID NO: 540)
MSSETGPVAVDPTLRRRIEPHEFEVFFDPRELRKET
CLLYEINWGGRHSIWRHTSQNTNKHVEVNFIEKFT
TERYFCPNTRCSITWFLSWSPCGECSRAITEFLSR
YPHVTLFIYIARLYHHADPRNRQGLRDLISSGVTI
QIMTEQESGYCWRNFVNYSPSNEAHWPRYPHLWVR
LYVLELYCIILGLPPCLNILRRKQPQLTFFTIALQ
SCHYQRLPPHILWATGLKSGGSSGGSSGSETPGTS
ESATPESSGGSSGGSDKKYSIGLAIGTNSVGWAVI
TDEYKVPSKKFKVLGNTDRHSIKKNLIGALLFDSG
ETAEATRLKRTARRRYTRRKNRICYLQEIFSNEMA
KVDDSFFHRLEESFLV [symbol] EDKKHERHPIFGNIVDEV
AYHEKYPTIYHLRKKLVDSTDKADLRLIYLALAHM
IKFRGHFLIEGDLNPDNSDVDKLFIQLVQTYNQLF
EENPINASGVDAKAILSARL [symbol] KSRRLENLIAQLPG
EKKNGLFGNLIALSLGLTPNFKSNFDLAED [symbol] KLQL
SKDTYDDDLDNLLAQIGDQYADLFLAAKNLSDAIL
LSDILRVNTEITKAPLSASMIKRYDEHHQDLTLLK
ALVRQQLPEKYKEIFFDQSKNGYAGYIDGGASQEE
FYKFIKPILEKMDGTEELLVKLNREDLLRKQRTFD
NG [symbol] IPHQIHLGELHAILRRQEDFYPFLKDNREKIE
KILTFRIPYYVGPLARGNSRFAWMTRKSEETITPW
NFE [symbol] VVDKGASAQSFIERMTNFDKNLPNEKVLPKH
SLLYEYFTVYNELTKVKYVTEGMRKPAFLSG [symbol] QKK
AIVDLLFKTNRKVTVKQLKEDYFKKIECFDSVEIS
GVEDRFNASLGTYHDLLKIIKDKDFLDNEENEDIL
EDIVLTLTLFEDREMIEERLKTYAHLFDDKVMKQL
KRRRYTGWGRLSRKLINGIRDKQSGKTILDFLKSD
GFANRNF [symbol] QLIHDDSLTFKEDIQKAQVSGQGDSLH
EHIANLAGSPAIKKGILQTVKVVDELVKVMGRHKP
ENIVIEMARENQTTQKGQKNSRERMKRIEEGIKEL
GSQILKEHPVENTQLQNEKLYLYYLQNGRDMYVDQ
ELDINRLSDYDVDHIVPQSFLKDDSIDNKVLTRSD
KNRGKSDNVPSEEVVKKMKNYWRQLLNAKLITQRK
FDNLTKAERGGLSELDKAGFIKRQLVETRQITKHV
AQILDSRMNTKYDENDKLIREVKVITLKSKLVSDF
RKDFQFYKVREINNYHHAHDAYLNAVVGTALIKKY
PKLESEFVYGDYKVYDVRKMIAKSEQEIGKATAKY
FFYSNIMNFFKTEITLANGEIRKRPLIETNGETGE
IVWDKGRDFATVRKVLSMPQVNIVKKTEVQTGGFS
KESILPKRNSDKLIARKKDWDPKKYGGFDSPTVAY
SVLVVAKVEKGKSKKLKSVKELLGITIMERSSFEK
NPIDFLEAKGYKEVKKDLIIKLPKYSLFELENGRK
RMLASAG [symbol] LQKGNELALPSKYVNFLYLASHYEKLK
GSPEDNEQKQLFVEQHKHYLDEIIEQISEFSKRVI
LADANLDKVLSAYNKHRDKPIREQAENIIHLFTLT
NLGAPAAFKYFDTTIDRKRYTSTKEVLDATLIHQS
ITGLYETRID [symbol] SQLGGDSGGSGGSGGSTNLSDIIE
KETGKQLVIQESILMLPEEVEEVIGNKPESDILVH
TAYDESTDENVMLLTSDAPEYKPWALVIQDSNGEN
KIKMLSGGSGGSGGSTNLSDIIEKETGKQLVIQES
ILMLPEEVEEVIGNKPESDILVHTAYDESTDENVM
LLTSDAPEYKPWALVIQDSNGENKIKMLSGGSPKK
KRK

xCas9 3.7-BE4 (APOBEC1-Linker(32aa)-xCas9(3.7)n-Linker(9aa)-UGI-Linker(9aa)-UGI):

(SEQ ID NO: 541)
MSSETGPVAVDPTLRRRIEPHEFEVFFDPRELRKET
CLLYEINWGGRHSIWRHTSQNTNKHVEVNFIEKFT
TERYFCPNTRCSITWFLSWSPCGECSRAITEFLSR
YPHVTLFIYIARLYHHADPRNRQGLRDLISSGVTI
QIMTEQESGYCWRNFVNYSPSNEAHWPRYPHLWVR
LYVLELYCIILGLPPCLNILRRKQPQLTFFTIALQ
SCHYQRLPPHILWATGLKSGGSSGGSSGSETPGTS
ESATPESSGGSSGGSDKKYSIGLAIGTNSVGWAVI
TDEYKVPSKKFKVLGNTDRHSIKKNLIGALLFDSG
ETAEATRLKRTARRRYTRRKNRICYLQEIFSNEMA
KVDDSFFHRLEESFLVEEDKKHERHPIFGNIVDEV
AYHEKYPTIYHLRKKLVDSTDKADLRLIYLALAHM
IKFRGHFLIEGDLNPDNSDVDKLFIQLVQTYNQLF
EENPINASGVDAKAILSARLSKSRRLENLIAQLPG
EKKNGLFGNLIALSLGLTPNFKSNFDLAE [symbol] TKLQL
SKDTYDDDLDNLLAQIGDQYADLFLAAKNLSDAIL
LSDILRVNTEITKAPLSASMIK [symbol] YDEHHQDLTLLK
ALVRQQLPEKYKEIFFDQSKNGYAGYIDGGASQEE
FYKFIKPILEKMDGTEELLVKLNREDLLRKQRTFD
NG [symbol] IPHQIHLGELHAILRRQEDFYPFLKDNREKIE
KILTFRIPYYVGPLARGNSRFAWMTRKSEETITPW
NFE [symbol] VVDKGASAQSFIERMTNFDKNLPNEKVLPKH
SLLYEYFTVYNELTKVKYVTEGMRKPAFLSG [symbol] QKK
AIVDLLFKTNRKVTVKQLKEDYFKKIECFDSVEIS
GVEDRFNASLGTYHDLLKIIKDKDFLDNEENEDIL
EDIVLTLTLFEDREMIEERLKTYAHLFDDKVMKQL
KRRRYTGWGRLSRKLINGIRDKQSGKTILDFLKSD
GFANRNF [symbol] QLIHDDSLTFKEDIQKAQVSGQGDSLH
EHIANLAGSPAIKKGILQTVKVVDELVKVMGRHKP
ENIVIEMARENQTTQKGQKNSRERMKRIEEGIKEL
GSQILKEHPVENTQLQNEKLYLYYLQNGRDMYVDQ
ELDINRLSDYDVDHIVPQSFLKDDSIDNKVLTRSD
KNRGKSDNVPSEEVVKKMKNYWRQLLNAKLITQRK
FDNLTKAERGGLSELDKAGFIKRQLVETRQITKHV
AQILDSRMNTKYDENDKLIREVKVITLKSKLVSDF
RKDFQFYKVREINNYHHAHDAYLNAVVGTALIKKY
PKLESEFVYGDYKVYDVRKMIAKSEQEIGKATAKY
FFYSNIMNFFKTEITLANGEIRKRPLIETNGETGE
IVWDKGRDFATVRKVLSMPQVNIVKKTEVQTGGFS
KESILPKRNSDKLIARKKDWDPKKYGGFDSPTVAY
SVLVVAKVEKGKSKKLKSVKELLGITIMERSSFEK
NPIDFLEAKGYKEVKKDLIIKLPKYSLFELENGRK
RMLASAG [symbol] LQKGNELALPSKYVNFLYLASHYEKLK
GSPEDNEQKQLFVEQHKHYLDEIIEQISEFSKRVI
LADANLDKVLSAYNKHRDKPIREQAENIIHLFTLT
NLGAPAAFKYFDTTIDRKRYTSTKEVLDATLIHQS
ITGLYETRIDLSQLGGDSGGSGGSGGSTNLSDIIE
KETGKQLVIQESILMLPEEVEEVIGNKPESDILVH
TAYDESTDENVMLLTSDAPEYKPWALVIQDSNGEN
KIKMLSGGSGGSGGSTNLSDIIEKETGKQLVIQES
ILMLPEEVEEVIGNKPESDILVHTAYDESTDENVM
LLTSDAPEYKPWALVIQDSNGENKIKMLSGGSPKK
KRK

BE4:

(SEQ ID NO: 542)
MSSETGPVAVDPTLRRRIEPHEFEVFFDPRELRKET
CLLYEINWGGRHSIWRHTSQNTNKHVEVNFIEKFT
TERYFCPNTRCSITWFLSWSPCGECSRAITEFLSR
YPHVTLFIYIARLYHHADPRNRQGLRDLISSGVTI
QIMTEQESGYCWRNFVNYSPSNEAHWPRYPHLWVR
LYVLELYCIILGLPPCLNILRRKQPQLTFFTIALQ
SCHYQRLPPHILWATGLKSGGSSGGSSGSETPGTS
ESATPESSGGSSGGSDKKYSIGLAIGTNSVGWAVI
TDEYKVPSKKFKVLGNTDRHSIKKNLIGALLFDSG
ETAEATRLKRTARRRYTRRKNRICYLQEIFSNEMA
KVDDSFFHRLEESFLVEEDKKHERHPIFGNIVDEV
AYHEKYPTIYHLRKKLVDSTDKADLRLIYLALAHM
IKFRGHFLIEGDLNPDNSDVDKLFIQLVQTYNQLF
EENPINASGVDAKAILSARLSKSRRLENLIAQLPG
EKKNGLFGNLIALSLGLTPNFKSNFDLAEDAKLQL
SKDTYDDDLDNLLAQIGDQYADLFLAAKNLSDAIL
LSDILRVNTEITKAPLSASMIKRYDEHHQDLTLLK
ALVRQQLPEKYKEIFFDQSKNGYAGYIDGGASQEE
FYKFIKPILEKMDGTEELLVKLNREDLLRKQRTFD
NGSIPHQIHLGELHAILRRQEDFYPFLKDNREKIE
KILTFRIPYYVGPLARGNSRFAWMTRKSEETITPW
NFEEVVDKGASAQSFIERMTNFDKNLPNEKVLPKH
SLLYEYFTVYNELTKVKYVTEGMRKPAFLSGEQKK
AIVDLLFKTNRKVTVKQLKEDYFKKIECFDSVEIS
GVEDRFNASLGTYHDLLKIIKDKDFLDNEENEDIL
EDIVLTLTLFEDREMIEERLKTYAHLFDDKVMKQL
KRRRYTGWGRLSRKLINGIRDKQSGKTILDFLKSD
GFANRNFMQLIHDDSLTFKEDIQKAQVSGQGDSLH
EHIANLAGSPAIKKGILQTVKVVDELVKVMGRHKP
ENIVIEMARENQTTQKGQKNSRERMKRIEEGIKEL
GSQILKEHPVENTQLQNEKLYLYYLQNGRDMYVDQ
ELDINRLSDYDVDHIVPQSFLKDDSIDNKVLTRSD
KNRGKSDNVPSEEVVKKMKNYWRQLLNAKLITQRK
FDNLTKAERGGLSELDKAGFIKRQLVETRQITKHV
AQILDSRMNTKYDENDKLIREVKVITLKSKLVSDF
RKDFQFYKVREINNYHHAHDAYLNAVVGTALIKKY
PKLESEFVYGDYKVYDVRKMIAKSEQEIGKATAKY
FFYSNIMNFFKTEITLANGEIRKRPLIETNGETGE
IVWDKGRDFATVRKVLSMPQVNIVKKTEVQTGGFS
KESILPKRNSDKLIARKKDWDPKKYGGFDSPTVAY
SVLVVAKVEKGKSKKLKSVKELLGITIMERSSFEK
NPIDFLEAKGYKEVKKDLIIKLPKYSLFELENGRK
RMLASAGELQKGNELALPSKYVNFLYLASHYEKLK
GSPEDNEQKQLFVEQHKHYLDEIIEQISEFSKRVI
LADANLDKVLSAYNKHRDKPIREQAENIIHLFTLT
NLGAPAAFKYFDTTIDRKRYTSTKEVLDATLIHQS
ITGLYETRIDLSQLGGDSGGSGGSGGSTNLSDIIE
KETGKQLVIQESILMLPEEVEEVIGNKPESDILVH
TAYDESTDENVMLLTSDAPEYKPWALVIQDSNGEN
KIKMLSGGSGGSGGSTNLSDIIEKETGKQLVIQES
ILMLPEEVEEVIGNKPESDILVHTAYDESTDENVM
LLTSDAPEYKPWALVIQDSNGENKIKMLSGGSPKK
KRK

In some embodiments, any of the fusion proteins provided herein may further comprise a Gam protein. The term “Gam protein,” as used herein, refers generally to proteins capable of binding to one or more ends of a double strand break of a double stranded nucleic acid (e.g., double stranded DNA). In some embodiments, the Gam protein prevents or inhibits degradation of one or more strands of a nucleic acid at the site of the double strand break. In some embodiments, a Gam protein is a naturally-occurring Gam protein from bacteriophage Mu, or a non-naturally occurring variant thereof. Fusion proteins comprising Gam proteins are described in Komor et al. (2017) Improved Base Excision Repair Inhibition and Bateriophage Mu Gam Protein Yields C:G-to-T:A base editors with higher efficiency and product purity. Sci Adv, 3: eaao4774; the entire contents of which is incorporated by reference herein. In some embodiments, the Gam protein comprises an amino acid sequence that is at least 70%, at least 75%, at least 80%, at least 85%, at least 90%, at least 92%, at least 95%, at least 96%, at least 97%, at least 98%, at least 99%, or at least 99.5% identical to the amino acid sequence provided by SEQ ID NO: 515. In some embodiments, the Gam protein comprises the amino acid sequence of SEQ ID NO: 515. In some embodiments, the fusion protein (e.g., BE4-Gam of SEQ ID NO: 543) comprises a Gam protein, wherein the Cas9 domain of BE4 is replaced with any of the Cas9 domains provided herein.

Gam from Bacteriophage Mu:

(SEQ ID NO: 515)
AKPAKRIKSAAAAYVPQNRDAVITDIKRIGDLQREA
SRLETEMNDAIAEITEKFAARIAPIKTDIETLSKGV
QGWCEANRDELTNGGKVKTANLVTGDVSWRVRPPSV
SIRGMDAVMETLERLGLQRFIRTKQEINKEAILLEP
KAVAGVAGITVKSGIEDFSIIPFEQEAGI

BE4-Gam:

(SEQ ID NO: 543)
MAKPAKRIKSAAAAYVPQNRDAVITDIKRIGDLQRE
ASRLETEMNDAIAEITEKFAARIAPIKTDIETLSK
GVQGWCEANRDELTNGGKVKTANLVTGDVSWRVRP
PSVSIRGMDAVMETLERLGLQRFIRTKQEINKEAI
LLEPKAVAGVAGITVKSGIEDFSIIPFEQEAGISG
SETPGTSESATPESSSETGPVAVDPTLRRRIEPHE
FEVFFDPRELRKETCLLYEINWGGRHSIWRHTSQN
TNKHVEVNFIEKFTTERYFCPNTRCSITWFLSWSP
CGECSRAITEFLSRYPHVTLFIYIARLYHHADPRN
RQGLRDLISSGVTIQIMTEQESGYCWRNFVNYSPS
NEAHWPRYPHLWVRLYVLELYCIILGLPPCLNILR
RKQPQLTFFTIALQSCHYQRLPPHILWATGLKSGG
SSGGSSGSETPGTSESATPESSGGSSGGSDKKYSI
GLAIGTNSVGWAVITDEYKVPSKKFKVLGNTDRHS
IKKNLIGALLFDSGETAEATRLKRTARRRYTRRKN
RICYLQEIFSNEMAKVDDSFFHRLEESFLVEEDKK
HERHPIFGNIVDEVAYHEKYPTIYHLRKKLVDSTD
KADLRLIYLALAHMIKFRGHFLIEGDLNPDNSDVD
KLFIQLVQTYNQLFEENPINASGVDAKAILSARLS
KSRRLENLIAQLPGEKKNGLFGNLIALSLGLTPNF
KSNFDLAEDAKLQLSKDTYDDDLDNLLAQIGDQYA
DLFLAAKNLSDAILLSDILRVNTEITKAPLSASMI
KRYDEHHQDLTLLKALVRQQLPEKYKEIFFDQSKN
GYAGYIDGGASQEEFYKFIKPILEKMDGTEELLVK
LNREDLLRKQRTFDNGSIPHQIHLGELHAILRRQE
DFYPFLKDNREKIEKILTFRIPYYVGPLARGNSRF
AWMTRKSEETITPWNFEEVVDKGASAQSFIERMTN
FDKNLPNEKVLPKHSLLYEYFTVYNELTKVKYVTE
GMRKPAFLSGEQKKAIVDLLFKTNRKVTVKQLKED
YFKKIECFDSVEISGVEDRFNASLGTYHDLLKIIK
DKDFLDNEENEDILEDIVLTLTLFEDREMIEERLK
TYAHLFDDKVMKQLKRRRYTGWGRLSRKLINGIRD
KQSGKTILDFLKSDGFANRNFMQLIHDDSLTFKED
IQKAQVSGQGDSLHEHIANLAGSPAIKKGILQTVK
VVDELVKVMGRHKPENIVIEMARENQTTQKGQKNS
RERMKRIEEGIKELGSQILKEHPVENTQLQNEKLY
LYYLQNGRDMYVDQELDINRLSDYDVDHIVPQSFL
KDDSIDNKVLTRSDKNRGKSDNVPSEEVVKKMKNY
WRQLLNAKLITQRKFDNLTKAERGGLSELDKAGFI
KRQLVETRQITKHVAQILDSRMNTKYDENDKLIRE
VKVITLKSKLVSDFRKDFQFYKVREINNYHHAHDA
YLNAVVGTALIKKYPKLESEFVYGDYKVYDVRKMI
AKSEQEIGKATAKYFFYSNIMNFFKTEITLANGEI
RKRPLIETNGETGEIVWDKGRDFATVRKVLSMPQV
NIVKKTEVQTGGFSKESILPKRNSDKLIARKKDWD
PKKYGGFDSPTVAYSVLVVAKVEKGKSKKLKSVKE
LLGITIMERSSFEKNPIDFLEAKGYKEVKKDLIIK
LPKYSLFELENGRKRMLASAGELQKGNELALPSKY
VNFLYLASHYEKLKGSPEDNEQKQLFVEQHKHYLD
EIIEQISEFSKRVILADANLDKVLSAYNKHRDKPI
REQAENIIHLFTLTNLGAPAAFKYFDTTIDRKRYT
STKEVLDATLIHQSITGLYETRIDLSQLGGDSGGS
GGSGGSTNLSDIIEKETGKQLVIQESILMLPEEVE
EVIGNKPESDILVHTAYDESTDENVMLLTSDAPEY
KPWALVIQDSNGENKIKMLSGGSGGSGGSTNLSDI
IEKETGKQLVIQESILMLPEEVEEVIGNKPESDIL
VHTAYDESTDENVMLLTSDAPEYKPWALVIQDSNG
ENKIKMLSGGSPKKKRK

Some aspects of the disclosure provide fusion proteins comprising a nucleic acid Cas9 domain (e.g.,) and an adenosine deaminase. In some embodiments, any of the fusion proteins provided herein are base editors. Some aspects of the disclosure provide fusion proteins comprising a Cas9 domain (e.g.) and an adenosine deaminase. The Cas9 domain may be any of the Cas9 domains (e.g., a Cas9 domain) provided herein. In some embodiments, any of the Cas9 domains (e.g., a Cas9 domain) provided herein may be fused with any of the adenosine deaminases provided herein. In some embodiments, the fusion protein comprises the structure:
[NH₂]-[adenosine deaminase]-[Cas9]-[COOH]; or
[NH₂]-[Cas9]-[adenosine deaminase]-[COOH].

In some embodiments, the fusion proteins comprising an adenosine deaminase and a Cas9 domain do not include a linker sequence. In some embodiments, a linker is present between the adenosine deaminase domain and the Cas9 domain. In some embodiments, the “-” used in the general architecture above indicates the presence of an optional linker. In some embodiments, the adenosine deaminase and the Cas9 domain are fused via any of the linkers provided herein. For example, in some embodiments the adenosine deaminase and the Cas9 domain are fused via any of the linkers provided below. In some embodiments, the linker comprises the amino acid sequence of any one of SEQ ID NOs: 300-318. In some embodiments, the adenosine deaminase and the Cas9 domain are fused via a linker that comprises between 1 and 200 amino acids. In some embodiments, the adenosine deaminase and the Cas9 domain are fused via a linker that comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. In some embodiments, the adenosine deaminase and the Cas9 domain are fused via a linker that comprises 3, 4, 16, 24, 32, 64, 100, or 104 amino acids in length. In some embodiments, the adenosine deaminase and the Cas9 domain are fused via a linker that comprises the amino acid sequence SGSETPGTSESATPES (SEQ ID NO: 306), SGGSSGGSSGSETPGTSESATPESSGGSSGGS (SEQ ID NO: 310), or GGSGGSPGSPAGSPTSTEEGTSESATPESGPGTSTEPSEGSAPGSPAGSPTSTEEGTST EPSEGSAPGTSTEPSEGSAPGTSESATPESGPGSEPATSGGSGGS (SEQ ID NO: 314). In some embodiments, the adenosine deaminase and the Cas9 domain are fused via a linker comprising the amino acid sequence SGSETPGTSESATPES (SEQ ID NO: 306), which may also be referred to as the XTEN linker. In some embodiments, the linker is 24 amino acids in length. In some embodiments, the linker comprises the amino acid sequence SGGSSGGSSGSETPGTSESATPES (SEQ ID NO: 315). In some embodiments, the linker is 32 amino acids in length. In some embodiments, the linker comprises the amino acid sequence (SGGS)₂-SGSETPGTSESATPES-(SGGS)₂ (SEQ ID NO: 308), which may also be referred to as (SGGS)₂-XTEN-(SGGS)₂. In some embodiments, the linker comprises the amino acid sequence (SGGS)ₙ-SGSETPGTSESATPES-(SGGS)ₙ(SEQ ID NO: 310), wherein n is 0, 1, 2, 3, 4, 5, 6, 7, 8, 9, or 10. In some embodiments, the linker is 40 amino acids in length. In some embodiments, the linker comprises the amino acid sequence SGGSSGGSSGSETPGTSESATPESSGGSSGGSSGGSSGGS (SEQ ID NO: 316). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGG SSGGS (SEQ ID NO: 317). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSA PGTSTEPSEGSAPGTSESATPESGPGSEPATS (SEQ ID NO: 318). In some embodiments, the adenosine deaminase comprises the amino acid sequence of any of one SEQ ID NOs: 400-458. In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 400-458, or to any of the adenosine deaminases provided herein. In some embodiments, the adenosine deaminase comprises the amino acid sequence of SEQ ID NO: 400. In some embodiments, the adenosine deaminase comprises the amino acid sequence of SEQ ID NO: 458.

In some embodiments, the fusion proteins comprising an adenosine deaminase provided herein further comprise one or more nuclear targeting sequences, for example, a nuclear localization sequence (NLS). In some embodiments, a NLS comprises an amino acid sequence that facilitates the importation of a protein, that comprises an NLS, into the cell nucleus (e.g., by nuclear transport). In some embodiments, any of the fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the IBR (e.g., dISN). In some embodiments, the NLS is fused to the C-terminus of the IBR (e.g., dISN). In some embodiments, the NLS is fused to the N-terminus of the Cas9 domain. In some embodiments, the NLS is fused to the C-terminus of the Cas9 domain. In some embodiments, the NLS is fused to the N-terminus of the adenosine deaminase. In some embodiments, the NLS is fused to the C-terminus of the adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. In some embodiments, the NLS comprises an amino acid sequence as set forth in SEQ ID NO: 520 or SEQ ID NO: 521. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, a NLS comprises the amino acid sequence PKKKRKV (SEQ ID NO: 520). In some embodiments, a NLS comprises the amino acid sequence MDSLLMNRRKFLYQFKNVRWAKGRRETYLC (SEQ ID NO: 521).

In some embodiments, the general architecture of exemplary fusion proteins with an adenosine deaminase and a Cas9 domain comprises any one of the following structures, where NLS is a nuclear localization sequence (e.g., any NLS provided herein), NH₂ is the N-terminus of the fusion protein, and COOH is the C-terminus of the fusion protein. Fusion proteins comprising an adenosine deaminase, a napDNAbp, and a NLS:
NH₂-[NLS]-[adenosine deaminase]-[Cas9]-COOH;
NH₂-[adenosine deaminase]-[NLS]-[Cas9]-COOH;
NH₂-[adenosine deaminase]-[Cas9]-[NLS]-COOH;
NH₂-[NLS]-[Cas9]-[adenosine deaminase]-COOH;
NH2-[Cas9]-[NLS]-[adenosine deaminase]-COOH; and
NH₂-[Cas9]-[adenosine deaminase]-[NLS]-COOH.

In some embodiments, the fusion proteins comprising an adenosine deaminase domain provided herein do not comprise a linker. In some embodiments, a linker is present between one or more of the domains or proteins (e.g., adenosine deaminase, Cas9 domain, and/or NLS). In some embodiments, the “-” used in the general architecture above indicates the presence of an optional linker.

Some aspects of the disclosure provide fusion proteins that comprise a Cas9 domain (e.g. a Cas9 domain) and at least two adenosine deaminase domains. Without wishing to be bound by any particular theory, dimerization of adenosine deaminases (e.g., in cis or in trans) may improve the ability (e.g., efficiency) of the fusion protein to modify a nucleic acid base, for example to deaminate adenine. In some embodiments, any of the fusion proteins may comprise 2, 3, 4 or 5 adenosine deaminase domains. In some embodiments, any of the fusion proteins provided herein comprise two adenosine deaminases. In some embodiments, any of the fusion proteins provided herein contain only two adenosine deaminases. In some embodiments, the adenosine deaminases are the same. In some embodiments, the adenosine deaminases are any of the adenosine deaminases provided herein. In some embodiments, the adenosine deaminases are different. In some embodiments, the first adenosine deaminase is any of the adenosine deaminases provided herein, and the second adenosine is any of the adenosine deaminases provided herein, but is not identical to the first adenosine deaminase. As one example, the fusion protein may comprise a first adenosine deaminase and a second adenosine deaminase that both comprise the amino acid sequence of SEQ ID NO: 417, which contains a A106V, D108N, D147Y, and E155V mutation from ecTadA (SEQ ID NO: 400). In some embodiments, the fusion protein may comprise a first adenosine deaminase that comprises the amino acid sequence of SEQ ID NO: 452, which contains a H36L, P48S, R51L, L84F, A106V, D108N, H123Y, S146C, D147Y, E155V, I156F, and K157N mutation from SEQ ID NO: 400, and a second adenosine deaminase domain that comprises the amino amino acid sequence of wild-type ecTadA (SEQ ID NO: 400). In some embodiments, the fusion protein may comprise a first adenosine deaminase that comprises the amino acid sequence of SEQ ID NO: 455, which contains a H36L, P48S, R51L, L84F, A106V, D108N, H123Y, A142N, S146C, D147Y, E155V, I156F, and K157N mutation from SEQ ID NO: 400, and a second adenosine deaminase domain that comprises the amino amino acid sequence of wild-type ecTadA (SEQ ID NO: 400). In some embodiments, the fusion protein may comprise a first adenosine deaminase that comprises the amino acid sequence of SEQ ID NO: 456, which contains a W23L, H36L, P48A, R51L, L84F, A106V, D108N, H123Y, A142N, S146C, D147Y, E155V, I156F, and K157N mutation from SEQ ID NO: 400, and a second adenosine deaminase domain that comprises the amino amino acid sequence of wild-type ecTadA (SEQ ID NO: 400). In some embodiments, the fusion protein may comprise a first adenosine deaminase that comprises the amino acid sequence of SEQ ID NO: 457, which contains a W23L, H36L, P48A, R51L, L84F, A106V, D108N, H123Y, A142N, S146C, D147Y, R152P, E155V, I156F, and K157N mutation from SEQ ID NO: 400, and a second adenosine deaminase domain that comprises the amino amino acid sequence of wild-type ecTadA (SEQ ID NO: 400). In some embodiments, the fusion protein may comprise a first adenosine deaminase that comprises the amino acid sequence of SEQ ID NO: 458, which contains a W23R, H36L, P48A, R51L, L84F, A106V, D108N, H123Y, S146C, D147Y, R152P, E155V, I156F, and K157N mutation from SEQ ID NO: 400, and a second adenosine deaminase domain that comprises the amino amino acid sequence of wild-type ecTadA (SEQ ID NO: 400). Additional fusion protein constructs comprising two adenosine deaminase domains suitable for use herein are illustrated in Gaudelli et al. (2017) Programmable base editing of A⋅T to G⋅C in genomic DNA without DNA cleavage, Nature, 551(23); 464-471; the entire contents of which is incorporated herein by reference.

In some embodiments, the fusion protein comprises two adenosine deaminases (e.g., a first adenosine deaminase and a second adenosine deaminase). In some embodiments, the fusion protein comprises a first adenosine deaminase and a second adenosine deaminase. In some embodiments, the first adenosine deaminase is N-terminal to the second adenosine deaminase in the fusion protein. In some embodiments, the first adenosine deaminase is C-terminal to the second adenosine deaminase in the fusion protein. In some embodiments, the first adenosine deaminase and the second deaminase are fused directly or via a linker. In some embodiments, the linker is any of the linkers provided herein. In some embodiments, the linker comprises the amino acid sequence of any one of SEQ ID NOs: 300-318. In some embodiments, the linker is 32 amino acids in length. In some embodiments, the linker comprises the amino acid sequence (SGGS)₂-SGSETPGTSESATPES-(SGGS)₂ (SEQ ID NO: 308), which may also be referred to as (SGGS)₂-XTEN-(SGGS)₂. In some embodiments, the linker comprises the amino acid sequence (SGGS)ₙ-SGSETPGTSESATPES-(SGGS)ₙ (SEQ ID NO: 310), wherein n is 0, 1, 2, 3, 4, 5, 6, 7, 8, 9, or 10. In some embodiments, the first adenosine deaminase is the same as the second adenosine deaminase. In some embodiments, the first adenosine deaminase and the second adenosine deaminase are any of the adenosine deaminases described herein. In some embodiments, the first adenosine deaminase and the second adenosine deaminase are different. In some embodiments, the first adenosine deaminase is any of the adenosine deaminases provided herein. In some embodiments, the second adenosine deaminase is any of the adenosine deaminases provided herein but is not identical to the first adenosine deaminase. In some embodiments, the first adenosine deaminase is an ecTadA adenosine deaminase. In some embodiments, the first adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 400-458, or to any of the adenosine deaminases provided herein. In some embodiments, the first adenosine deaminase comprises the amino acid sequence of SEQ ID NO: 400. In some embodiments, the second adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 400-458, or to any of the adenosine deaminases provided herein. In some embodiments, the second adenosine deaminase comprises the amino acid sequence of SEQ ID NO: 400. In some embodiments, the first adenosine deaminase and the second adenosine deaminase of the fusion protein comprise the mutations in ecTadA (SEQ ID NO: 400), or corresponding mutations in another adenosine deaminase, such as the amino acid sequences of any one of SEQ ID NOs: 402-408. In some embodiments, the fusion protein comprises the two adenosine deaminases (e.g., a first adenosine deaminase and a second adenosine deaminase) of any one of SEQ ID NOs: 400-458.

In some embodiments, the general architecture of exemplary fusion proteins with a first adenosine deaminase, a second adenosine deaminase, and a Cas9 domain (e.g.) comprises any one of the following structures, where NLS is a nuclear localization sequence (e.g., any NLS provided herein), NH₂ is the N-terminus of the fusion protein, and COOH is the C-terminus of the fusion protein:
NH₂-[first adenosine deaminase]-[second adenosine deaminase]-[Cas9]-COOH;
NH₂-[first adenosine deaminase]-[Cas9]-[second adenosine deaminase]-COOH;
NH₂-[Cas9]-[first adenosine deaminase]-[second adenosine deaminase]-COOH;
NH₂-[second adenosine deaminase]-[first adenosine deaminase]-[Cas9]-COOH;
NH₂-[second adenosine deaminase]-[Cas9]-[first adenosine deaminase]-COOH;
NH₂-[Cas9]-[second adenosine deaminase]-[first adenosine deaminase]-COOH;

In some embodiments, the fusion proteins provided herein do not comprise a linker. In some embodiments, a linker is present between one or more of the domains or proteins (e.g., first adenosine deaminase, second adenosine deaminase, and/or napDNAbp). In some embodiments, the “-” used in the general architecture above indicates the presence of an optional linker.

In some embodiments, a fusion protein comprising a first adenosine deaminase, a second adenosine deaminase, and a Cas9 domain further comprise a NLS. Exemplary fusion proteins comprising a first adenosine deaminase, a second adenosine deaminase, a napDNAbp, and an NLS are shown as follows:
NH₂-[NLS]-[first adenosine deaminase]-[second adenosine deaminase]-[Cas9]-COOH;
NH₂-[first adenosine deaminase]-[NLS]-[second adenosine deaminase]-[Cas9]-COOH;
NH₂-[first adenosine deaminase]-[second adenosine deaminase]-[NLS]-[Cas9]-COOH;
NH₂-[first adenosine deaminase]-[second adenosine deaminase]-[Cas9]-[NLS]-COOH;
NH₂-[NLS]-[first adenosine deaminase]-[Cas9]-[second adenosine deaminase]-COOH;
NH₂-[first adenosine deaminase]-[NLS]-[Cas9]-[second adenosine deaminase]-COOH;
NH₂-[first adenosine deaminase]-[Cas9]-[NLS]-[second adenosine deaminase]-COOH;
NH₂-[first adenosine deaminase]-[Cas9]-[second adenosine deaminase]-[NLS]-COOH;
NH₂-[NLS]-[Cas9]-[first adenosine deaminase]-[second adenosine deaminase]-COOH;
NH₂-[Cas9]-[NLS]-[first adenosine deaminase]-[second adenosine deaminase]-COOH;
NH₂-[Cas9]-[first adenosine deaminase]-[NLS]-[second adenosine deaminase]-COOH;
NH₂-[Cas9]-[first adenosine deaminase]-[second adenosine deaminase]-[NLS]-COOH;
NH₂-[NLS]-[second adenosine deaminase]-[first adenosine deaminase]-[Cas9]-COOH;
NH₂-[second adenosine deaminase]-[NLS]-[first adenosine deaminase]-[Cas9]-COOH;
NH₂-[second adenosine deaminase]-[first adenosine deaminase]-[NLS]-[Cas9]-COOH;
NH₂-[second adenosine deaminase]-[first adenosine deaminase]-[Cas9]-[NLS]-COOH;
NH₂-[NLS]-[second adenosine deaminase]-[Cas9]-[first adenosine deaminase]-COOH;
NH₂-[second adenosine deaminase]-[NLS]-[Cas9]-[first adenosine deaminase]-COOH;
NH₂-[second adenosine deaminase]-[Cas9]-[NLS]-[first adenosine deaminase]-COOH;
NH₂-[second adenosine deaminase]-[Cas9]-[first adenosine deaminase]-[NLS]-COOH;
NH₂-[NLS]-[Cas9]-[second adenosine deaminase]-[first adenosine deaminase]-COOH;
NH₂-[Cas9]-[NLS]-[second adenosine deaminase]-[first adenosine deaminase]-COOH;
NH₂-[Cas9]-[second adenosine deaminase]-[NLS]-[first adenosine deaminase]-COOH;
NH₂-[Cas9]-[second adenosine deaminase]-[first adenosine deaminase]-[NLS]-COOH;

In some embodiments, the fusion proteins provided herein do not comprise a linker. In some embodiments, a linker is present between one or more of the domains or proteins (e.g., first adenosine deaminase, second adenosine deaminase, Cas9 domain, and/or NLS). In some embodiments, the “-” used in the general architecture above indicates the presence of an optional linker.

In some embodiments, the fusion protein comprises a Cas9 domain fused to one or more adenosine deaminase domains (e.g., a first adenosine deaminase and a second adenosine deaminase), wherein the fusion protein comprises or consists of the amino acid sequence of SEQ ID NO: 535 or 539. In some embodiments, the fusion protein comprises the amino acid sequence of SEQ ID NO: 535. In some embodiments, the fusion protein is the amino acid sequence of SEQ ID NO: 535. In some embodiments, the fusion protein comprises the amino acid sequence of SEQ ID NO: 539. In some embodiments, the fusion protein is the amino acid sequence of SEQ ID NO: 539. In some embodiments, the Cas9 domain of SEQ ID NO: 544 is replaced with any of the Cas9 domains provided herein.

xCas9(3.7)-ABE: ( [symbol] -linker(32 aa)-ecTadA*(7.10)-linker(32 aa)- [symbol] -NLS):

(SEQ ID NO: 535)

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

SEVEFSHEYWMR
HALTLAKRARDEREVPVGAVLVLNNRVIGEGWNRA
IGLHDPTAHAEIMALRQGGLVMQNYRLIDATLYVT
FEPCVMCAGAMIHSRIGRVVFGVRNAKTGAAGSLM
DVLHYPGMNHRVEITEGILADECAALLCYFFRMPR
QVFNAQKKAQSSTD [symbol] [symbol] [symbol]
DKKYSIGLAIGTNSVGWAVITDEY
KVPSKKFKVLGNTDRHSIKKNLIGALLFDSGETAE
ATRLKRTARRRYTRRKNRICYLQEIFSNEMAKVDD
SFFHRLEESFLVEEDKKHERHPIFGNIVDEVAYHE
KYPTIYHLRKKLVDSTDKADLRLIYLALAHMIKFR
GHFLIEGDLNPDNSDVDKLFIQLVQTYNQLFEENP
INASGVDAKAILSARLSKSRRLENLIAQLPGEKKN
GLFGNLIALSLGLTPNFKSNFDLAED [symbol] KLQLSKDT
YDDDLDNLLAQIGDQYADLFLAAKNLSDAILLSDI
LRVNTEITKAPLSASMI [symbol] LYDEHHQDLTLLKALVR
QQLPEKYKEIFFDQSKNGYAGYIDGGASQEEFYKF
IKPILEKMDGTEELLVKLNREDLLRKQRTFDNG [symbol] I
PHQIHLGELHAILRRQEDFYPFLKDNREKIEKILT
FRIPYYVGPLARGNSRFAWMTRKSEETITPWNFE [symbol]
VVDKGASAQSFIERMTNFDKNLPNEKVLPKHSLLY
EYFTVYNELTKVKYVTEGMRKPAFLSG [symbol] QKKAIVD
LLFKTNRKVTVKQLKEDYFKKIECFDSVEISGVED
RFNASLGTYHDLLKIIKDKDFLDNEENEDILEDIV
LTLTLFEDREMIEERLKTYAHLFDDKVMKQLKRRR
YTGWGRLSRKLINGIRDKQSGKTILDFLKSDGFAN
RNF [symbol] QLIHDDSLTFKEDIQKAQVSGQGDSLHEHIA
NLAGSPAIKKGILQTVKVVDELVKVMGRHKPENIV
IEMARENQTTQKGQKNSRERMKRIEEGIKELGSQI
LKEHPVENTQLQNEKLYLYYLQNGRDMYVDQELDI
NRLSDYDVDHIVPQSFLKDDSIDNKVLTRSDKNRG
KSDNVPSEEVVKKMKNYWRQLLNAKLITQRKFDNL
TKAERGGLSELDKAGFIKRQLVETRQITKHVAQIL
DSRMNTKYDENDKLIREVKVITLKSKLVSDFRKDF
QFYKVREINNYHHAHDAYLNAVVGTALIKKYPKLE
SEFVYGDYKVYDVRKMIAKSEQEIGKATAKYFFYS
NIMNFFKTEITLANGEIRKRPLIETNGETGEIVWD
KGRDFATVRKVLSMPQVNIVKKTEVQTGGFSKESI
LPKRNSDKLIARKKDWDPKKYGGFDSPTVAYSVLV
VAKVEKGKSKKLKSVKELLGITIMERSSFEKNPID
FLEAKGYKEVKKDLIIKLPKYSLFELENGRKRMLA
SAG [symbol] LQKGNELALPSKYVNFLYLASHYEKLKGSPE
DNEQKQLFVEQHKHYLDEIIEQISEFSKRVILADA
NLDKVLSAYNKHRDKPIREQAENIIHLFTLTNLGA
PAAFKYFDTTIDRKRYTSTKEVLDATLIHQSITGL
YETRIDLSQLGGD [symbol] PKKKRKV

xCas9(3.6)-ABE: ( [symbol] -linker(32 aa)-ecTadA*(7.10)-linker(32 aa)- [symbol] -NLS):

(SEQ ID NO: 539)

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

SEVEFSHEYWMRHALTLAKRARDEREVPVGAVLVL
NNRVIGEGWNRAIGLHDPTAHAEIMALRQGGLVMQ
NYRLIDATLYVTFEPCVMCAGAMIHSRIGRVVFGV
RNAKTGAAGSLMDVLHYPGMNHRVEITEGILADEC
AALLCYFFRMPRQVFNAQKKAQSSTD

[symbol]

[symbol]

[symbol]

DKKYSIGLAIGTNSVGWAVITDEY
KVPSKKFKVLGNTDRHSIKKNLIGALLFDSGETAE
ATRLKRTARRRYTRRKNRICYLQEIFSNEMAKVDD
SFFHRLEESFLV [symbol] EDKKHERHPIFGNIVDEVAYHE
KYPTIYHLRKKLVDSTDKADLRLIYLALAHMIKFR
GHFLIEGDLNPDNSDVDKLFIQLVQTYNQLFEENP
INASGVDAKAILSARL [symbol] KSRRLENLIAQLPGEKKN
GLFGNLIALSLGLTPNFKSNFDLAED [symbol] KLQLSKDT
YDDDLDNLLAQIGDQYADLFLAAKNLSDAILLSDI
LRVNTEITKAPLSASMIKRYDEHHQDLTLLKALVR
QQLPEKYKEIFFDQSKNGYAGYIDGGASQEEFYKF
IKPILEKMDGTEELLVKLNREDLLRKQRTFDNG [symbol] I
PHQIHLGELHAILRRQEDFYPFLKDNREKIEKILT
FRIPYYVGPLARGNSRFAWMTRKSEETITPWNFE [symbol]
VVDKGASAQSFIERMTNFDKNLPNEKVLPKHSLLY
EYFTVYNELTKVKYVTEGMRKPAFLSG [symbol] QKKAIVD
LLFKTNRKVTVKQLKEDYFKKIECFDSVEISGVED
RFNASLGTYHDLLKIIKDKDFLDNEENEDILEDIV
LTLTLFEDREMIEERLKTYAHLFDDKVMKQLKRRR
YTGWGRLSRKLINGIRDKQSGKTILDFLKSDGFAN
RNF [symbol] QLIHDDSLTFKEDIQKAQVSGQGDSLHEHIA
NLAGSPAIKKGILQTVKVVDELVKVMGRHKPENIV
IEMARENQTTQKGQKNSRERMKRIEEGIKELGSQI
LKEHPVENTQLQNEKLYLYYLQNGRDMYVDQELDI
NRLSDYDVDHIVPQSFLKDDSIDNKVLTRSDKNRG
KSDNVPSEEVVKKMKNYWRQLLNAKLITQRKFDNL
TKAERGGLSELDKAGFIKRQLVETRQITKHVAQIL
DSRMNTKYDENDKLIREVKVITLKSKLVSDFRKDF
QFYKVREINNYHHAHDAYLNAVVGTALIKKYPKLE
SEFVYGDYKVYDVRKMIAKSEQEIGKATAKYFFYS
NIMNFFKTEITLANGEIRKRPLIETNGETGEIVWD
KGRDFATVRKVLSMPQVNIVKKTEVQTGGFSKESI
LPKRNSDKLIARKKDWDPKKYGGFDSPTVAYSVLV
VAKVEKGKSKKLKSVKELLGITIMERSSFEKNPID
FLEAKGYKEVKKDLIIKLPKYSLFELENGRKRMLA
SAG [symbol] LQKGNELALPSKYVNFLYLASHYEKLKGSPE
DNEQKQLFVEQHKHYLDEIIEQISEFSKRVILADA
NLDKVLSAYNKHRDKPIREQAENIIHLFTLTNLGA
PAAFKYFDTTIDRKRYTSTKEVLDATLIHQSITG [symbol]
YETRIDLSQLGGD [symbol] [symbol] PKKKRKV

ABE7.10: ecTadA_{(wild-type)}-(SGGS)₂-XTEN-(SGGS)₂-ecTadA_{(w23R_H36L_P48A_R51L_L84F_A106V_D108N_H123Y_S146C_D147Y_R152P_E155V_I156F_K157N)}-(SGGS)₂-XTEN-(SGGS)₂ nCas9 SGGS NLS

(SEQ ID NO: 544)
MSEVEFSHEYWMRHALTLAKRAWDEREVPVGAVLV
HNNRVIGEGWNRPIGRHDPTAHAEIMALRQGGLVM
QNYRLIDATLYVTLEPCVMCAGAMIHSRIGRVVFG
ARDAKTGAAGSLMDVLHHPGMNHRVEITEGILADE
CAALLSDFFRMRRQEIKAQKKAQSSTDSGGSSGGS
SGSETPGTSESATPESSGGSSGGSSEVEFSHEYWM
RHALTLAKRARDEREVPVGAVLVLNNRVIGEGWNR
AIGLHDPTAHAEIMALRQGGLVMQNYRLIDATLYV
TFEPCVMCAGAMIHSRIGRVVFGVRNAKTGAAGSL
MDVLHYPGMNHRVEITEGILADECAALLCYFFRMP
RQVFNAQKKAQSSTDSGGSSGGSSGSETPGTSESA
TPESSGGSSGGSDKKYSIGLAIGTNSVGWAVITDE
YKVPSKKFKVLGNTDRHSIKKNLIGALLFDSGETA
EATRLKRTARRRYTRRKNRICYLQEIFSNEMAKVD
DSFFHRLEESFLVEEDKKHERHPIFGNIVDEVAYH
EKYPTIYHLRKKLVDSTDKADLRLIYLALAHMIKF
RGHFLIEGDLNPDNSDVDKLFIQLVQTYNQLFEEN
PINASGVDAKAILSARLSKSRRLENLIAQLPGEKK
NGLFGNLIALSLGLTPNFKSNFDLAEDAKLQLSKD
TYDDDLDNLLAQIGDQYADLFLAAKNLSDAILLSD
ILRVNTEITKAPLSASMIKRYDEHHQDLTLLKALV
RQQLPEKYKEIFFDQSKNGYAGYIDGGASQEEFYK
FIKPILEKMDGTEELLVKLNREDLLRKQRTFDNGS
IPHQIHLGELHAILRRQEDFYPFLKDNREKIEKIL
TFRIPYYVGPLARGNSRFAWMTRKSEETITPWNFE
EVVDKGASAQSFIERMTNFDKNLPNEKVLPKHSLL
YEYFTVYNELTKVKYVTEGMRKPAFLSGEQKKAIV
DLLFKTNRKVTVKQLKEDYFKKIECFDSVEISGVE
DRFNASLGTYHDLLKIIKDKDFLDNEENEDILEDI
VLTLTLFEDREMIEERLKTYAHLFDDKVMKQLKRR
RYTGWGRLSRKLINGIRDKQSGKTILDFLKSDGFA
NRNFMQLIHDDSLTFKEDIQKAQVSGQGDSLHEHI
ANLAGSPAIKKGILQTVKVVDELVKVMGRHKPENI
VIEMARENQTTQKGQKNSRERMKRIEEGIKELGSQ
ILKEHPVENTQLQNEKLYLYYLQNGRDMYVDQELD
INRLSDYDVDHIVPQSFLKDDSIDNKVLTRSDKNR
GKSDNVPSEEVVKKMKNYWRQLLNAKLITQRKFDN
LTKAERGGLSELDKAGFIKRQLVETRQITKHVAQI
LDSRMNTKYDENDKLIREVKVITLKSKLVSDFRKD
FQFYKVREINNYHHAHDAYLNAVVGTALIKKYPKL
ESEFVYGDYKVYDVRKMIAKSEQEIGKATAKYFFY
SNIMNFFKTEITLANGEIRKRPLIETNGETGEIVW
DKGRDFATVRKVLSMPQVNIVKKTEVQTGGFSKES
ILPKRNSDKLIARKKDWDPKKYGGFDSPTVAYSVL
VVAKVEKGKSKKLKSVKELLGITIMERSSFEKNPI
DFLEAKGYKEVKKDLIIKLPKYSLFELENGRKRML
ASAGELQKGNELALPSKYVNFLYLASHYEKLKGSP
EDNEQKQLFVEQHKHYLDEIIEQISEFSKRVILAD
ANLDKVLSAYNKHRDKPIREQAENIIHLFTLTNLG
APAAFKYFDTTIDRKRYTSTKEVLDATLIHQSITG
LYETRIDLSQLGGDSGGSPKKKRKV

In some embodiments, the fusion proteins provided herein comprising one or more adenosine deaminase domains and a Cas9 domain exhibit an increased activity on a target sequence that does not comprise the canonical PAM (5′-NGG-3′) at its 3′ end as compared to a fusion protein comprising Streptococcus pyogenes Cas9 as provided by SEQ ID NO: 9. In some embodiments, the fusion protein exhibits an activity on a target sequence having a 3′ end that is not directly adjacent to the canonical PAM sequence (5′-NGG-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of a fusion protein comprising Streptococcus pyogenes Cas9 as provided by SEQ ID NO: 9 on the same target sequence. In some embodiments, the 3′ end of the target sequence is directly adjacent to a sequence selected from the group consisting of NGT, NGA, NGC, and NNG, wherein N is an A, G, T, or C. In some embodiments, the 3′ end of the target sequence is directly adjacent to a sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA. In some embodiments, the fusion protein activity is measured by a nuclease assay, a deamination assay, a transcriptional activation assay, or high-throughput sequencing. In some embodiments, the transcriptional activation assay is a GFP activation assay. In some embodiments, high-throughput sequencing is used to measure indel formation.

It should be appreciated that the fusion proteins of the present disclosure may comprise one or more additional features. For example, in some embodiments, the fusion protein may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of ordinary skill in the art. In some embodiments, the fusion protein comprises one or more His tags.

Additional suitable strategies for generating fusion proteins comprising a napDNAbp (e.g., a Cas9 domain) and a nucleic acid editing domain (e.g., a deaminase domain) will be apparent to those of ordinary skill in the art based on this disclosure in combination with the general knowledge in the art. Suitable strategies for generating fusion proteins according to aspects of this disclosure using linkers or without the use of linkers will also be apparent to those of ordinary skill in the art in view of the instant disclosure and the knowledge in the art. For example, Gilbert et al., CRISPR-mediated modular RNA-guided regulation of transcription in eukaryotes. Cell. 2013; 154(2):442-51, showed that C-terminal fusions of Cas9 with VP64 using 2 NLS's as a linker (SPKKKRKVEAS, SEQ ID NO: 522), can be employed for transcriptional activation. Mali et al. (CAS9 transcriptional activators for target specificity screening and paired nickases for cooperative genome engineering. Nat Biotechnol. 2013; 31(9):833-8), reported that C-terminal fusions with VP64 without linker can be employed for transcriptional activation. And Maeder et al. (CRISPR RNA-guided activation of endogenous human genes. Nat Methods. 2013; 10: 977-979), reported that C-terminal fusions with VP64 using a Gly₄Ser (SEQ ID NO: 313) linker can be used as transcriptional activators. Recently, dCas9-FokI nuclease fusions have successfully been generated and exhibit improved enzymatic specificity as compared to the parental Cas9 enzyme (In Guilinger J P, Thompson D B, Liu D R. Fusion of catalytically inactive Cas9 to FokI nuclease improves the specificity of genome modification. Nat. Biotechnol. 2014; 32(6): 577-82; and in Tsai S Q, et al. Dimeric CRISPR RNA-guided FokI nucleases for highly specific genome editing. Nat Biotechnol. 2014; 32(6):569-76. PMID: 24770325 a SGSETPGTSESATPES (SEQ ID NO: 306) or a GGGGS (SEQ ID NO: 313) linker was used in FokI-dCas9 fusion proteins, respectively).

In some embodiments, the Cas9 fusion protein comprises: (i) Cas9 domain; and (ii) a transcriptional activator domain. In some embodiments, the transcriptional activator domain comprises a VPR. VPR is a VP64-SV40-P65-RTA tripartite activator. In some embodiments, VPR comprises a VP64 amino acid sequence encoded by the nucleic acid sequence of SEQ ID NO: 292:

(SEQ ID NO: 292)
GAGGCCAGCGGTTCCGGACGGGCTGACGCATTGGA
CGATTTTGATCTGGATATGCTGGGAAGTGACGCCC
TCGATGATTTTGACCTTGACATGCTTGGTTCGGAT
GCCCTTGATGACTTTGACCTCGACATGCTCGGCAG
TGACGCCCTTGATGATTTCGACCTGGACATGCTGA
TTAACTCTAGATAG

In some embodiments, VPR comprises a VP64 amino acid sequence as set forth in SEQ ID NO: 293:

(SEQ ID NO: 293)
EASGSGRADALDDFDLDMLGSDALDDFDLDMLGSD
ALDDFDLDMLGSDALDDFDLDMLINSR

In some embodiments, VPR comprises a VP64-SV40-P65-RTA amino acid sequence encoded by the nucleic acid sequence of SEQ ID NO: 294:

(SEQ ID NO: 294)
TCGCCAGGGATCCGTCGACTTGACGCGTTGATATC
AACAAGTTTGTACAAAAAAGCAGGCTACAAAGAGG
CCAGCGGTTCCGGACGGGCTGACGCATTGGACGAT
TTTGATCTGGATATGCTGGGAAGTGACGCCCTCGA
TGATTTTGACCTTGACATGCTTGGTTCGGATGCCC
TTGATGACTTTGACCTCGACATGCTCGGCAGTGAC
GCCCTTGATGATTTCGACCTGGACATGCTGATTAA
CTCTAGAAGTTCCGGATCTCCGAAAAAGAAACGCA
AAGTTGGTAGCCAGTACCTGCCCGACACCGACGAC
CGGCACCGGATCGAGGAAAAGCGGAAGCGGACCTA
CGAGACATTCAAGAGCATCATGAAGAAGTCCCCCT
TCAGCGGCCCCACCGACCCTAGACCTCCACCTAGA
AGAATCGCCGTGCCCAGCAGATCCAGCGCCAGCGT
GCCAAAACCTGCCCCCCAGCCTTACCCCTTCACCA
GCAGCCTGAGCACCATCAACTACGACGAGTTCCCT
ACCATGGTGTTCCCCAGCGGCCAGATCTCTCAGGC
CTCTGCTCTGGCTCCAGCCCCTCCTCAGGTGCTGC
CTCAGGCTCCTGCTCCTGCACCAGCTCCAGCCATG
GTGTCTGCACTGGCTCAGGCACCAGCACCCGTGCC
TGTGCTGGCTCCTGGACCTCCACAGGCTGTGGCTC
CACCAGCCCCTAAACCTACACAGGCCGGCGAGGGC
ACACTGTCTGAAGCTCTGCTGCAGCTGCAGTTCGA
CGACGAGGATCTGGGAGCCCTGCTGGGAAACAGCA
CCGATCCTGCCGTGTTCACCGACCTGGCCAGCGTG
GACAACAGCGAGTTCCAGCAGCTGCTGAACCAGGG
CATCCCTGTGGCCCCTCACACCACCGAGCCCATGC
TGATGGAATACCCCGAGGCCATCACCCGGCTCGTG
ACAGGCGCTCAGAGGCCTCCTGATCCAGCTCCTGC
CCCTCTGGGAGCACCAGGCCTGCCTAATGGACTGC
TGTCTGGCGACGAGGACTTCAGCTCTATCGCCGAT
ATGGATTTCTCAGCCTTGCTGGGCTCTGGCAGCGG
CAGCCGGGATTCCAGGGAAGGGATGTTTTTGCCGA
AGCCTGAGGCCGGCTCCGCTATTAGTGACGTGTTT
GAGGGCCGCGAGGTGTGCCAGCCAAAACGAATCCG
GCCATTTCATCCTCCAGGAAGTCCATGGGCCAACC
GCCCACTCCCCGCCAGCCTCGCACCAACACCAACC
GGTCCAGTACATGAGCCAGTCGGGTCACTGACCCC
GGCACCAGTCCCTCAGCCACTGGATCCAGCGCCCG
CAGTGACTCCCGAGGCCAGTCACCTGTTGGAGGAT
CCCGATGAAGAGACGAGCCAGGCTGTCAAAGCCCT
TCGGGAGATGGCCGATACTGTGATTCCCCAGAAGG
AAGAGGCTGCAATCTGTGGCCAAATGGACCTTTCC
CATCCGCCCCCAAGGGGCCATCTGGATGAGCTGAC
AACCACACTTGAGTCCATGACCGAGGATCTGAACC
TGGACTCACCCCTGACCCCGGAATTGAACGAGATT
CTGGATACCTTCCTGAACGACGAGTGCCTCTTGCA
TGCCATGCATATCAGCACAGGACTGTCCATCTTCG
ACACATCTCTGTTTTGA

In some embodiments, VPR comprises a VP64-SV40-P65-RTA amino acid sequence as set forth in SEQ ID NO: 295:

(SEQ ID NO: 295)
SPGIRRLDALISTSLYKKAGYKEASGSGRADALDDFDLDMLGSDALDDFDL
DMLGSDALDDFDLDMLGSDALDDFDLDMLINSRSSGSPKKKRKVGSQYLPD
TDDRHRIEEKRKRTYETFKSIMKKSPFSGPTDPRPPPRRIAVPSRSSASVP
KPAPQPYPFTSSLSTINYDEFPTMVFPSGQISQASALAPAPPQVLPQAPAP
APAPAMVSALAQAPAPVPVLAPGPPQAVAPPAPKPTQAGEGTLSEALLQLQ
FDDEDLGALLGNSTDPAVFTDLASVDNSEFQQLLNQGIPVAPHTTEPMLME
YPEAITRLVTGAQRPPDPAPAPLGAPGLPNGLLSGDEDFSSIADMDFSALL
GSGSGSRDSREGMFLPKPEAGSAISDVFEGREVCQPKRIRPFHPPGSPWAN
RPLPASLAPTPTGPVHEPVGSLTPAPVPQPLDPAPAVTPEASHLLEDPDEE
TSQAVKALREMADTVIPQKEEAAICGQMDLSHPPPRGHLDELTTTLESMTE
DLNLDSPLTPELNEILDTFLNDECLLHAMHISTGLSIFDTSLF

Some aspects of this disclosure provide fusion proteins comprising a transcription activator. In some embodiments, the transcriptional activator is VPR. In some embodiments, the VPR comprises a wild type VPR or a VPR as set forth in SEQ ID NO: 293. In some embodiments, the VPR proteins provided herein include fragments of VPR and proteins homologous to a VPR or a VPR fragment. For example, in some embodiments, a VPR comprises a fragment of the amino acid sequence set forth in SEQ ID NO: 293. In some embodiments, a VPR comprises an amino acid sequence homologous to the amino acid sequence set forth in SEQ ID NO: 293 or an amino acid sequence homologous to a fragment of the amino acid sequence set forth in SEQ ID NO: 293. In some embodiments, proteins comprising VPR or fragments of VPR or homologs of VPR or VPR fragments are referred to as “VPR variants.” A VPR variant shares homology to VPR, or a fragment thereof. For example a VPR variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild type VPR or a VPR as set forth in SEQ ID NO: 293. In some embodiments, the VPR variant comprises a fragment of VPR, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of wild type VPR or a VPR as set forth in SEQ ID NO: 293. In some embodiments, the VPR comprises the amino acid sequence set forth in SEQ ID NO: 293. In some embodiments, the VPR comprises an amino acid sequence encoded by the nucleotide sequence set forth in SEQ ID NO: 292.

In some embodiments, a VPR is a VP64-SV40-P65-RTA triple activator. In some embodiments, the VP64-SV40-P65-RTA comprises a VP64-SV40-P65-RTA as set forth in SEQ ID NO: 295. In some embodiments, the VP64-SV40-P65-RTA proteins provided herein include fragments of VP64-SV40-P65-RTA and proteins homologous to a VP64-SV40-P65-RTA or a VP64-SV40-P65-RTA fragment. For example, in some embodiments, a VP64-SV40-P65-RTA comprises a fragment of the amino acid sequence set forth in SEQ ID NO: 295. In some embodiments, a VP64-SV40-P65-RTA comprises an amino acid sequence homologous to the amino acid sequence set forth in SEQ ID NO: 295 or an amino acid sequence homologous to a fragment of the amino acid sequence set forth in SEQ ID NO: 295. In some embodiments, proteins comprising VP64-SV40-P65-RTA or fragments of VP64-SV40-P65-RTA or homologs of VP64-SV40-P65-RTA or VP64-SV40-P65-RTA fragments are referred to as “VP64-SV40-P65-RTA variants.” A VP64-SV40-P65-RTA variant shares homology to VP64-SV40-P65-RTA, or a fragment thereof. For example a VP64-SV40-P65-RTA variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a VP64-SV40-P65-RTA as set forth in SEQ ID NO: 295. In some embodiments, the VP64-SV40-P65-RTA variant comprises a fragment of VP64-SV40-P65-RTA, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of a VP64-SV40-P65-RTA as set forth in SEQ ID NO: 295. In some embodiments, the VP64-SV40-P65-RTA comprises the amino acid sequence set forth in SEQ ID NO: 295. In some embodiments, the VP64-SV40-P65-RTA comprises an amino acid sequence encoded by the nucleotide sequence set forth in SEQ ID NO: 294.

In some embodiments, the fusion protein comprises the nucleic acid sequence of SEQ ID NO: 532.

dCas9-VPR (dCas9(3.7)-NLS-Linker(22aa)-VP64-Linker(4aa)-NLS-p65AD-Linker(6aa)-RtaAD)

(SEQ ID NO: 532)
ATGGACAAGAAGTACTCCATTGGGCTCGATATCGGCACAAACAGCGTCGGCTGGGCCGTCATTACGGACGAGTAC
AAGGTGCCGAGCAAAAAATTCAAAGTTCTGGGCAATACCGATCGCCACAGCATAAAGAAGAACCTCATTGGCGCC
CTCCTGTTCGACTCCGGGGAGACGGCCGAAGCCACGCGGCTCAAAAGAACAGCACGGCGCAGATATACCCGCAGA
AAGAATCGGATCTGCTACCTGCAGGAGATCTTTAGTAATGAGATGGCTAAGGTGGATGACTCTTTCTTCCATAGG
CTGGAGGAGTCCTTTTTGGTGGAGGAGGATAAAAAGCACGAGCGCCACCCAATCTTTGGCAATATCGTGGACGAG
GTGGCGTACCATGAAAAGTACCCAACCATATATCATCTGAGGAAGAAGCTTGTAGACAGTACTGATAAGGCTGAC
TTGCGGTTGATCTATCTCGCGCTGGCGCATATGATCAAATTTCGGGGACACTTCCTCATCGAGGGGGACCTGAAC
CCAGACAACAGCGATGTCGACAAACTCTTTATCCAACTGGTTCAGACTTACAATCAGCTTTTCGAAGAGAACCCG
ATCAACGCATCCGGAGTTGACGCCAAAGCAATCCTGAGCGCTAGGCTGTCCAAATCCCGGCGGCTCGAAAACCTC
ATCGCACAGCTCCCTGGGGAGAAGAAGAACGGCCTGTTTGGTAATCTTATCGCCCTGTCCCTCGGGCTGACCCCC
AACTTTAAATCTAACTTCGACCTGGCCGAAGATACCAAGCTTCAACTGAGCAAAGACACCTACGATGATGATCTC
GACAATCTGCTGGCCCAGATCGGCGACCAGTACGCAGACCTTTTTTTGGCGGCAAAGAACCTGTCAGACGCCATT
CTGCTGAGTGATATTCTGCGAGTGAACACGGAGATCACCAAAGCTCCGCTGAGCGCTAGTATGATCAAGCTCTAT
GATGAGCACCACCAAGACTTGACTTTGCTGAAGGCCCTTGTCAGACAGCAACTGCCTGAGAAGTACAAGGAAATT
TTCTTCGATCAGTCTAAAAATGGCTACGCCGGATACATTGACGGCGGAGCAAGCCAGGAGGAATTTTACAAATTT
ATTAAGCCCATCTTGGAAAAAATGGACGGCACCGAGGAGCTGCTGGTAAAGCTTAACAGAGAAGATCTGTTGCGC
AAACAGCGCACTTTCGACAATGGAATCATCCCCCACCAGATTCACCTGGGCGAACTGCACGCTATCCTCAGGCGG
CAAGAGGATTTCTACCCCTTTTTGAAAGATAACAGGGAAAAGATTGAGAAAATCCTCACATTTCGGATACCCTAC
TATGTAGGCCCCCTCGCCCGGGGAAATTCCAGATTCGCGTGGATGACTCGCAAATCAGAAGAGACCATCACTCCC
TGGAACTTCGAGAAAGTCGTGGATAAGGGGGCCTCTGCCCAGTCCTTCATCGAAAGGATGACTAACTTTGATAAA
AATCTGCCTAACGAAAAGGTGCTTCCTAAACACTCTCTGCTGTACGAGTACTTCACAGTTTATAACGAGCTCACC
AAGGTCAAATACGTCACAGAAGGGATGAGAAAGCCAGCATTCCTGTCTGGAGATCAGAAGAAAGCTATTGTGGAC
CTCCTCTTCAAGACGAACCGGAAAGTTACCGTGAAACAGCTCAAAGAAGACTATTTCAAAAAGATTGAATGTTTC
GACTCTGTTGAAATCAGCGGAGTGGAGGATCGCTTCAACGCATCCCTGGGAACGTATCACGATCTCCTGAAAATC
ATTAAAGACAAGGACTTCCTGGACAATGAGGAGAACGAGGACATTCTTGAGGACATTGTCCTCACCCTTACGTTG
TTTGAAGATAGGGAGATGATTGAAGAACGCTTGAAAACTTACGCTCATCTCTTCGACGACAAAGTCATGAAGCAG
CTCAAGAGGCGCCGATATACAGGATGGGGGCGGCTGTCAAGAAAACTGATCAATGGGATCCGAGACAAGCAGAGT
GGAAAGACAATCCTGGATTTTCTTAAGTCCGATGGATTTGCCAACCGGAACTTCATTCAGTTGATCCATGATGAC
TCTCTCACCTTTAAGGAGGACATCCAGAAAGCACAAGTTTCTGGCCAGGGGGACAGTCTTCACGAGCACATCGCT
AATCTTGCAGGTAGCCCAGCTATCAAAAAGGGAATACTGCAGACCGTTAAGGTCGTGGATGAACTCGTCAAAGTA
ATGGGAAGGCATAAGCCCGAGAATATCGTTATCGAGATGGCCCGAGAGAACCAAACCACCCAGAAGGGACAGAAG
AACAGTAGGGAAAGGATGAAGAGGATTGAAGAGGGTATAAAAGAACTGGGGTCCCAAATCCTTAAGGAACACCCA
GTTGAAAACACCCAGCTTCAGAATGAGAAGCTCTACCTGTACTACCTGCAGAACGGCAGGGACATGTACGTGGAT
CAGGAACTGGACATCAATCGGCTCTCCGACTACGACGTGGATCATATCGTGCCCCAGTCTTTTCTCAAAGATGAT
TCTATTGATAATAAAGTGTTGACAAGATCCGATAAAAATAGAGGGAAGAGTGATAACGTCCCCTCAGAAGAAGTT
GTCAAGAAAATGAAAAATTATTGGCGGCAGCTGCTGAACGCCAAACTGATCACACAACGGAAGTTCGATAATCTG
ACTAAGGCTGAACGAGGTGGCCTGTCTGAGTTGGATAAAGCCGGTTTCATCAAAAGGCAGCTTGTTGAGACACGC
CAGATCACCAAGCACGTGGCCCAAATTCTCGATTCACGCATGAACACCAAGTACGATGAAAATGACAAACTGATT
CGAGAGGTGAAAGTTATTACTCTGAAGTCTAAGCTGGTCTCAGATTTCAGAAAGGACTTTCAGTTTTATAAGGTG
AGAGAGATCAACAATTACCACCATGCGCATGATGCCTACCTGAATGCAGTGGTAGGCACTGCACTTATCAAAAAA
TATCCCAAGCTTGAATCTGAATTTGTTTACGGAGACTATAAAGTGTACGATGTTAGGAAAATGATCGCAAAGTCT
GAGCAGGAAATAGGCAAGGCCACCGCTAAGTACTTCTTTTACAGCAATATTATGAATTTTTTCAAGACCGAGATT
ACACTGGCCAATGGAGAGATTCGGAAGCGACCACTTATCGAAACAAACGGAGAAACAGGAGAAATCGTGTGGGAC
AAGGGTAGGGATTTCGCGACAGTCCGGAAGGTCCTGTCCATGCCGCAGGTGAACATCGTTAAAAAGACCGAAGTA
CAGACCGGAGGCTTCTCCAAGGAAAGTATCCTCCCGAAAAGGAACAGCGACAAGCTGATCGCACGCAAAAAAGAT
TGGGACCCCAAGAAATACGGCGGATTCGATTCTCCTACAGTCGCTTACAGTGTACTGGTTGTGGCCAAAGTGGAG
AAAGGGAAGTCTAAAAAACTCAAAAGCGTCAAGGAACTGCTGGGCATCACAATCATGGAGCGATCAAGCTTCGAA
AAAAACCCCATCGACTTTCTCGAGGCGAAAGGATATAAAGAGGTCAAAAAAGACCTCATCATTAAGCTTCCCAAG
TACTCTCTCTTTGAGCTTGAAAACGGCCGGAAACGAATGCTCGCTAGTGCGGGCGTGCTGCAGAAAGGTAACGAG
CTGGCACTGCCCTCTAAATACGTTAATTTCTTGTATCTGGCCAGCCACTATGAAAAGCTCAAAGGGTCTCCCGAA
GATAATGAGCAGAAGCAGCTGTTCGTGGAACAACACAAACACTACCTTGATGAGATCATCGAGCAAATAAGCGAA
TTCTCCAAAAGAGTGATCCTCGCCGACGCTAACCTCGATAAGGTGCTTTCTGCTTACAATAAGCACAGGGATAAG
CCCATCAGGGAGCAGGCAGAAAACATTATCCACTTGTTTACTCTGACCAACTTGGGCGCGCCTGCAGCCTTCAAG
TACTTCGACACCACCATAGACAGAAAGCGGTACACCTCTACAAAGGAGGTCCTGGACGCCACACTGATTCATCAG
TCAATTACGGGGCTCTATGAAACAAGAATCGACCTCTCTCAGCTCGGTGGAGACCCCCAAGAAGAAGAGGAAGGT
GTCGCCAGGGATCCGTCGACTTGACGCGTTGATATCAACAAGTTTGTACAAAAAAGCAGGCTACAAAGAGGCCAG
CGGTTCCGGACGGGCTGACGCATTGGACGATTTTGATCTGGATATGCTGGGAAGTGACGCCCTCGATGATTTTGA
CCTTGACATGCTTGGTTCGGATGCCCTTGATGACTTTGACCTCGACATGCTCGGCAGTGACGCCCTTGATGATTT
CGACCTGGACATGCTGATTAACTCTAGAAGTTCCGGATCTCCGAAAAAGAAACGCAAAGTTGGTAGCCAGTACCT
GCCCGACACCGACGACCGGCACCGGATCGAGGAAAAGCGGAAGCGGACCTACGAGACATTCAAGAGCATCATGAA
GAAGTCCCCCTTCAGCGGCCCCACCGACCCTAGACCTCCACCTAGAAGAATCGCCGTGCCCAGCAGATCCAGCGC
CAGCGTGCCAAAACCTGCCCCCCAGCCTTACCCCTTCACCAGCAGCCTGAGCACCATCAACTACGACGAGTTCCC
TACCATGGTGTTCCCCAGCGGCCAGATCTCTCAGGCCTCTGCTCTGGCTCCAGCCCCTCCTCAGGTGCTGCCTCA
GGCTCCTGCTCCTGCACCAGCTCCAGCCATGGTGTCTGCACTGGCTCAGGCACCAGCACCCGTGCCTGTGCTGGC
TCCTGGACCTCCACAGGCTGTGGCTCCACCAGCCCCTAAACCTACACAGGCCGGCGAGGGCACACTGTCTGAAGC
TCTGCTGCAGCTGCAGTTCGACGACGAGGATCTGGGAGCCCTGCTGGGAAACAGCACCGATCCTGCCGTGTTCAC
CGACCTGGCCAGCGTGGACAACAGCGAGTTCCAGCAGCTGCTGAACCAGGGCATCCCTGTGGCCCCTCACACCAC
CGAGCCCATGCTGATGGAATACCCCGAGGCCATCACCCGGCTCGTGACAGGCGCTCAGAGGCCTCCTGATCCAGC
TCCTGCCCCTCTGGGAGCACCAGGCCTGCCTAATGGACTGCTGTCTGGCGACGAGGACTTCAGCTCTATCGCCGA
TATGGATTTCTCAGCCTTGCTGGGCTCTGGCAGCGGCAGCCGGGATTCCAGGGAAGGGATGTTTTTGCCGAAGCC
TGAGGCCGGCTCCGCTATTAGTGACGTGTTTGAGGGCCGCGAGGTGTGCCAGCCAAAACGAATCCGGCCATTTCA
TCCTCCAGGAAGTCCATGGGCCAACCGCCCACTCCCCGCCAGCCTCGCACCAACACCAACCGGTCCAGTACATGA
GCCAGTCGGGTCACTGACCCCGGCACCAGTCCCTCAGCCACTGGATCCAGCGCCCGCAGTGACTCCCGAGGCCAG
TCACCTGTTGGAGGATCCCGATGAAGAGACGAGCCAGGCTGTCAAAGCCCTTCGGGAGATGGCCGATACTGTGAT
TCCCCAGAAGGAAGAGGCTGCAATCTGTGGCCAAATGGACCTTTCCCATCCGCCCCCAAGGGGCCATCTGGATGA
GCTGACAACCACACTTGAGTCCATGACCGAGGATCTGAACCTGGACTCACCCCTGACCCCGGAATTGAACGAGAT
TCTGGATACCTTCCTGAACGACGAGTGCCTCTTGCATGCCATGCATATCAGCACAGGACTGTCCATCTTCGACAC
ATCTCTGTTT

### Complexes Comprising Fusion Proteins

Further provided herein are complexes comprising (i) any of the fusion proteins provided herein, and (ii) a guide RNA bound to the Cas9 domain of the fusion protein. Without wishing to be bound by any particular theory, these fusion proteins can be directed by designing a suitable guide RNA to specify and efficiently target single point mutations in a genome without introducing double-stranded DNA breaks or requiring homology directed repair (HDR). However, the suitability of a target site for base editing (e.g., a point mutation in the genome) is dependent on the presence of a suitably positioned PAM. The broaden PAM compatibility of the Cas9 domains provided herein has the potential to expand the targeting scope of base editors to those target sites that do not lie within approximately 15 nucleotides of a canonical 5′-NGG-3′ PAM sequence. A person of ordinary skill in the art will be able to design a suitable guide RNA (gRNA) sequence to target a desired point mutation based on this disclosure and knowledge in the field. In addition, these fusion proteins comprising a Cas9 domain generate fewer insertions and deletions (indels) and exhibit reduced off-target activity compared to fusion proteins (e.g., base editors) comprising a Cas9 domain that can only recognize the canonical 5′-NGG-3′ PAM sequence.

In some embodiments, the guide RNA is about 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is in the genome of an organism. In some embodiments, the organism is a prokaryote. In some embodiments, the prokaryote is a bacterium. In some embodiments, the bacterium is E. coli. In some embodiments, the organism is a eukaryote. In some embodiments, the organism is a plant or fungus. In some embodiments, the organism is a vertebrate. In some embodiments, the vertebrate is a mammal. In some embodiments, the mammal is a human. In some embodiments, the organism is a cell. In some embodiments, the cell is a human cell. In some embodiments, the cell is a HEK293T or U2OS cell.

In some embodiments, the target sequence comprises a sequence associated with a disease or disorder. In some embodiments, the target sequence comprises a point mutation associated with a disease or disorder. In some embodiments, the target sequence comprises a T→C point mutation. In some embodiments, the complex deaminates the target C point mutation, wherein the deamination results in a sequence that is not associated with a disease or disorder. In some embodiments, the target C point mutation is present in the DNA strand that is not complementary to the guide RNA. In some embodiments, the target sequence comprises a T→A point mutation. In some embodiments, the complex deaminates the target A point mutation, and wherein the deamination results in a sequence that is not associated with a disease or disorder. In some embodiments, the target A point mutation is present in the DNA strand that is not complementary to the guide RNA.

In some embodiments, the complex edits a point mutation in the target sequence. In some embodiments, the point mutation is located between about 10 to about 20 nucleotides upstream of the PAM in the target sequence. In some embodiments, the point mutation is located between about 13 to about 17 nucleotides upstream of the PAM in the target sequence. In some embodiments, the point mutation is about 13 nucleotides upstream of the PAM. In some embodiments, the point mutation is about 14 nucleotides upstream of the PAM. In some embodiments, the point mutation is about 15 nucleotides upstream of the PAM. In some embodiments, the point mutation is about 16 nucleotides upstream of the PAM. In some embodiments, the point mutation is about 17 nucleotides upstream of the PAM.

In some embodiments, the complex exhibits increased deamination efficiency of a point mutation in a target sequence that does not comprise the canonical PAM (5′-NGG-3′) at its 3′ end as compared to the deamination efficiency of a complex comprising Streptococcus pyogenes Cas9 as provided by SEQ ID NO: 9. In some embodiments, the complex exhibits increased deamination efficiency of a point mutation in a target sequence having a 3′ end that is not directly adjacent to the canonical PAM sequence (5′-NGG-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the deamination efficiency of complex comprising the Streptococcus pyogenes Cas9 as provided by SEQ ID NO: 9 on the same target sequence. In some embodiments, the 3′ end of the target sequence is directly adjacent to a sequence selected from the group consisting of NGT, NGA, NGC, and NNG, wherein N is an A, G, T, or C. In some embodiments, the 3′ end of the target sequence is directly adjacent to a sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA. In some embodiments, deamination activity is measured using high-throughput sequencing.

In some embodiments, the complex produces fewer indels in a target sequence that does not comprise the canonical PAM (5′-NGG-3′) at its 3′ end as compared to the amount of indels produced by a complex comprising Streptococcus pyogenes Cas9 as provided by SEQ ID NO: 9. In some embodiments, the complex produces fewer indels in a target sequence having a 3′ end that is not directly adjacent to the canonical PAM sequence (5′-NGG-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold lower as compared to the amount of indels produced by a complex comprising Streptococcus pyogenes Cas9 as provided by SEQ ID NO: 9 on the same target sequence. In some embodiments, the 3′ end of the target sequence is directly adjacent to a sequence selected from the group consisting of NGT, NGA, NGC, and NNG, wherein N is an A, G, T, or C. In some embodiments, the 3′ end of the target sequence is directly adjacent to a sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA. In some embodiments, indels are measured using high-throughput sequencing.

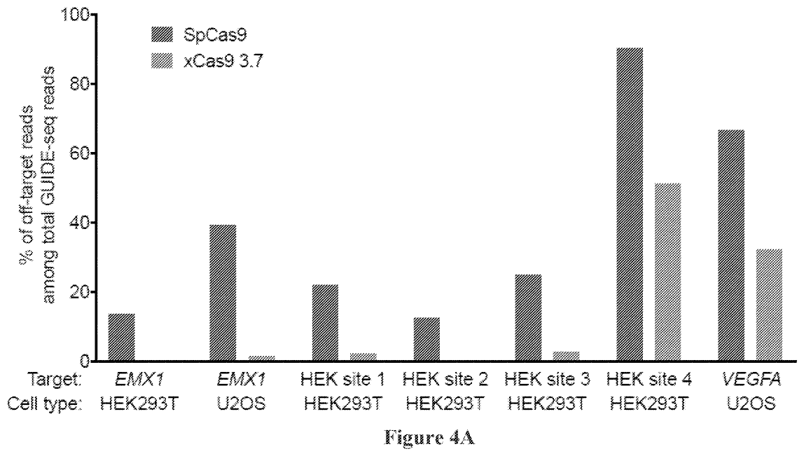

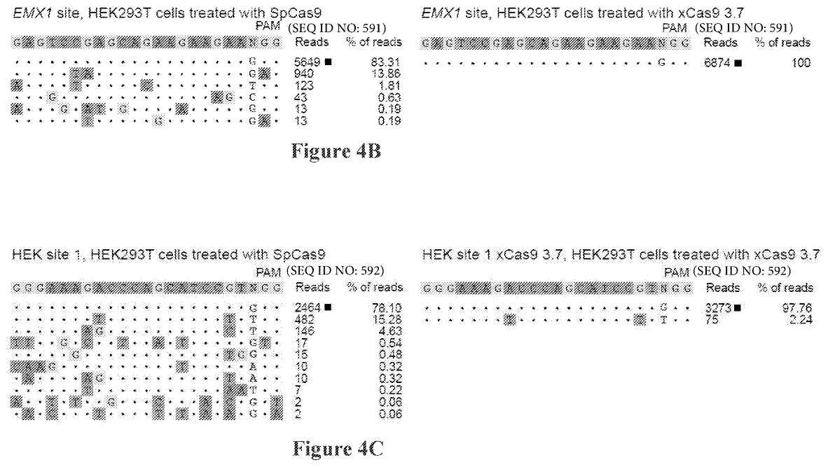

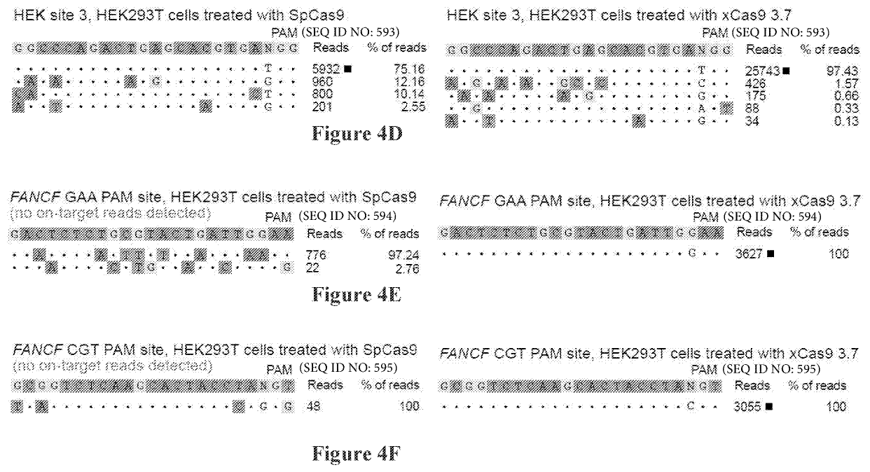

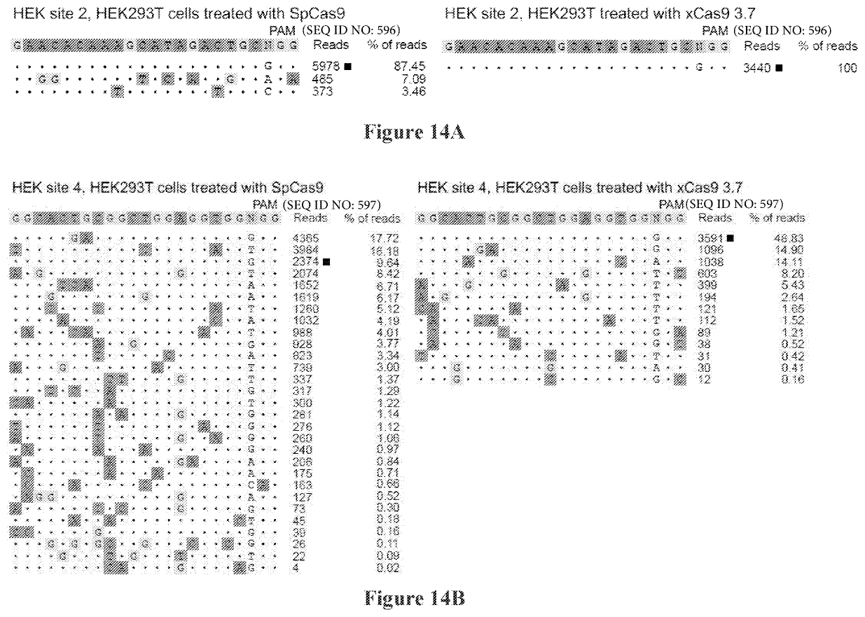

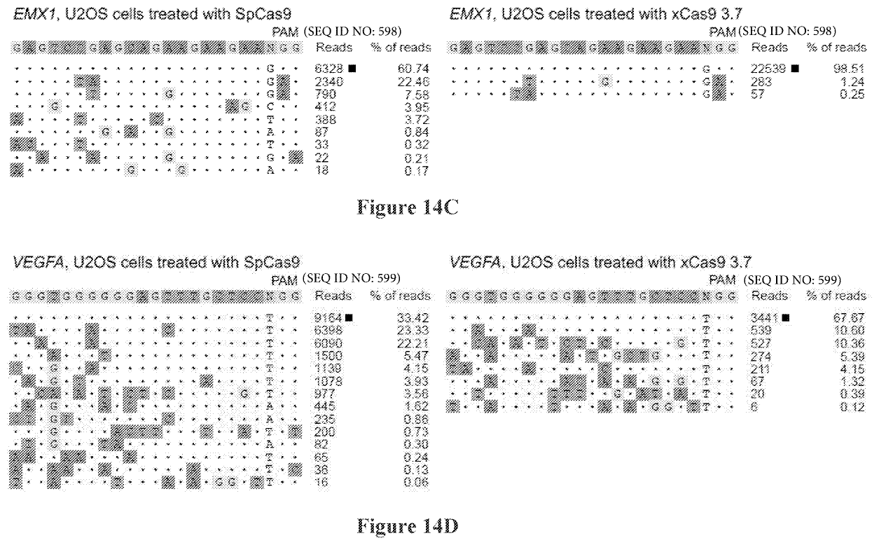

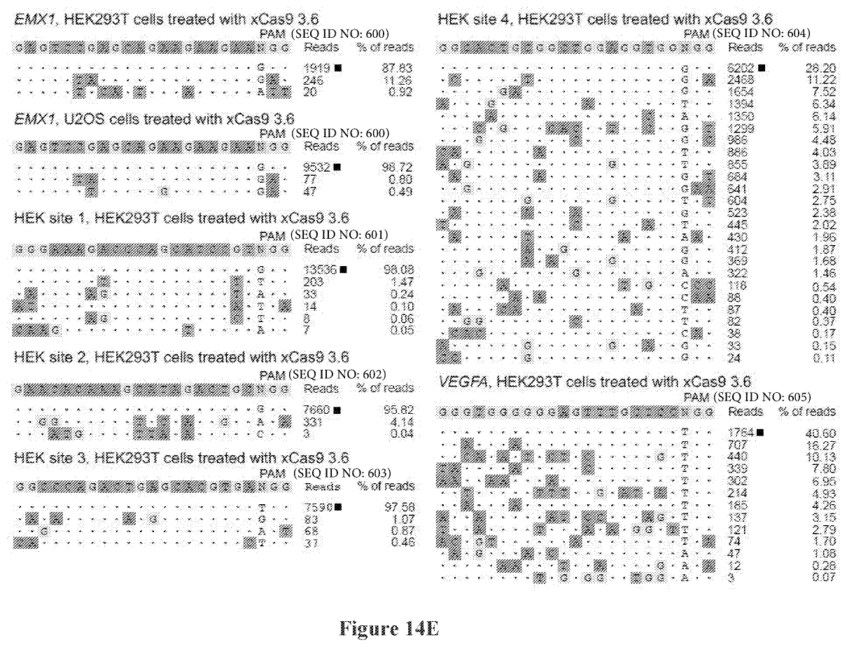

In some embodiments, the complex exhibits a decreased off-target activity as compared to the off-target activity of a complex comprising Streptococcus pyogenes Cas9 as provided by SEQ ID NO: 9. In some embodiments, the off-target activity of the complex is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold decreased as compared to the off-target activity of a complex comprising Streptococcus pyogenes Cas9 as provided by SEQ ID NO: 9. In some embodiments, the off-target activity is determined using a genome-wide off-target analysis. In some embodiments, the off-target activity is determined using GUIDE-seq. See, e.g., Example 4, FIGS. 4A-4F and FIGS. 14A-14E.

### Nucleic Acid Editing Domains

Some aspects of this disclosure provide fusion proteins comprising a Cas9 domain as provided herein that is fused to a second protein, or a “fusion partner”, such as a nucleic acid editing domain, thus forming a fusion protein. In some embodiments, the nucleic acid editing domain is fused to the N-terminus of the Cas9 domain. In some embodiments, the nucleic acid editing domain is fused to the C-terminus of the Cas9 domain. In some embodiments, the Cas9 domain and the nucleic acid editing domain are fused to each other via a linker. Suitable strategies for generating fusion proteins according to aspects of this disclosure using linkers or without the use of linkers will also be apparent to those of skill in the art in view of the instant disclosure and the knowledge in the art. For example, Gilbert et al., CRISPR-mediated modular RNA-guided regulation of transcription in eukaryotes. Cell. 2013; 154(2):442-51, showed that C-terminal fusions of Cas9 with VP64 using 2 NLS's as a linker (SPKKKRKVEAS, SEQ ID NO: 522), can be employed for transcriptional activation. Mali et al., CAS9 transcriptional activators for target specificity screening and paired nickases for cooperative genome engineering. Nat Biotechnol. 2013; 31(9):833-8, reported that C-terminal fusions with VP64 without linker can be employed for transcriptional activation. Maeder et al., CRISPR RNA-guided activation of endogenous human genes. Nat Methods. 2013; 10: 977-979, reported that C-terminal fusions with VP64 using a Gly4Ser (SEQ ID NO: 5) linker can be used as transcriptional activators. Recently, dCas9-FokI nuclease fusions have successfully been generated and exhibit improved enzymatic specificity as compared to the parental Cas9 enzyme (In Guilinger J P, Thompson D B, Liu D R. Fusion of catalytically inactive Cas9 to FokI nuclease improves the specificity of genome modification. Nat. Biotechnol. 2014; 32(6): 577-82, and in Tsai S Q, Wyvekens N, Khayter C, Foden J A, Thapar V, Reyon D, Goodwin M J, Aryee M J, Joung J K. Dimeric CRISPR RNA-guided FokI nucleases for highly specific genome editing. Nat Biotechnol. 2014; 32(6):569-76. PMID: 24770325 a SGSETPGTSESATPES (SEQ ID NO: 306) or a GGGGS. (SEQ ID NO: 301) linker was used in FokI-dCas9 fusion proteins, respectively).

In some embodiments, the second protein in the fusion protein (i.e., the fusion partner) comprises a nucleic acid editing domain. Such a nucleic acid editing domain may be, without limitation, a nuclease, a nickase, a recombinase, a deaminase, a methyltransferase, a methylase, an acetylase, or an acetyltransferase. Non-limiting exemplary nucleic acid editing domains that may be used in accordance with this disclosure include cytidine deaminases and adenosine deaminases. In some embodiments, the nucleic acid editing domain is a deaminase domain. In some embodiments, the nucleic acid editing domain is a nuclease domain. In some embodiments, the nuclease domain is a FokI DNA cleavage domain. In some embodiments, this disclosure provides dimers of the fusion proteins provided herein, e.g., dimers of fusion proteins may include a dimerizing nuclease domain. In some embodiments, the nucleic acid editing domain is a nickase domain. In some embodiments, the nucleic acid editing domain is a recombinase domain. In some embodiments, the nucleic acid editing domain is a methyltransferase domain. In some embodiments, the nucleic acid editing domain is a methylase domain. In some embodiments, the nucleic acid editing domain is an acetylase domain. In some embodiments, the nucleic acid editing domain is an acetyltransferase domain. Additional nucleic acid editing domains would be apparent to a person of ordinary skill in the art based on this disclosure and knowledge in the field and are within the scope of this disclosure. In other embodiments, the second protein comprises a domain that modulates transcriptional activity. Such transcriptional modulating domains may be, without limitation, a transcriptional activator or transcriptional repressor domain.

In some embodiments, the deaminase domain is a cytidine deaminase domain. A cytidine deaminase domain may also be referred to interchangeably as a cytosine deaminase domain. In some embodiments, the cytidine deaminase catalyzes the hydrolytic deamination of cytidine (C) or deoxycytidine (dC) to uridine (U) or deoxyuridine (dU), respectively. In some embodiments, the cytidine deaminase domain catalyzes the hydrolytic deamination of cytosine (C) to uracil (U). In some embodiments, the cytidine deaminase catalyzes the hydrolytic deamination of cytidine or cytosine in deoxyribonucleic acid (DNA). Without wishing to be bound by any particular theory, fusion proteins comprising a cytidine deaminase are useful inter alia for targeted editing, referred to herein as “base editing,” of nucleic acid sequences in vitro and in vivo.

One exemplary suitable type of cytidine deaminase is a cytidine deaminase, for example, of the APOBEC family. The apolipoprotein B mRNA-editing complex (APOBEC) family of cytidine deaminase enzymes encompasses eleven proteins that serve to initiate mutagenesis in a controlled and beneficial manner (see, e.g., Conticello S G. The AID/APOBEC family of nucleic acid mutators. Genome Biol. 2008; 9(6):229). One family member, activation-induced cytidine deaminase (AID), is responsible for the maturation of antibodies by converting cytosines in ssDNA to uracils in a transcription-dependent, strand-biased fashion (see, e.g., Reynaud C A, et al. What role for AID: mutator, or assembler of the immunoglobulin mutasome? Nat Immunol. 2003; 4(7):631-638). The apolipoprotein B editing complex 3 (APOBEC3) enzyme provides protection to human cells against a certain HIV-1 strain via the deamination of cytosines in reverse-transcribed viral ssDNA (see, e.g., Bhagwat A S. DNA-cytosine deaminases: from antibody maturation to antiviral defense. DNA Repair (Amst). 2004; 3(1):85-89). These proteins all require a Zn²⁺-coordinating motif (His-X-Glu-X23-26-Pro-Cys-X24-Cys; SEQ ID NO: 800) and bound water molecule for catalytic activity. The Glu residue acts to activate the water molecule to a zinc hydroxide for nucleophilic attack in the deamination reaction. Each family member preferentially deaminates at its own particular “hotspot”, ranging from WRC (W is A or T, R is A or G) for hAID, to TTC for hAPOBEC3F (see, e.g., Navaratnam N and Sarwar R. An overview of cytidine deaminases. Int J Hematol. 2006; 83(3):195-200). A recent crystal structure of the catalytic domain of APOBEC3G revealed a secondary structure comprised of a five-stranded j-sheet core flanked by six α-helices, which is believed to be conserved across the entire family (see, e.g., Holden L G, et al. Crystal structure of the anti-viral APOBEC3G catalytic domain and functional implications. Nature. 2008; 456(7218):121-4). The active center loops have been shown to be responsible for both ssDNA binding and in determining “hotspot” identity (see, e.g., Chelico L, et al. Biochemical basis of immunological and retroviral responses to DNA-targeted cytosine deamination by activation-induced cytidine deaminase and APOBEC3G. J Biol Chem. 2009; 284(41). 27761-5). Overexpression of these enzymes has been linked to genomic instability and cancer, thus highlighting the importance of sequence-specific targeting (see, e.g., Pham P, et al. Reward versus risk: DNA cytidine deaminases triggering immunity and disease. Biochemistry. 2005; 44(8):2703-15).

Some aspects of this disclosure relate to the recognition that the activity of cytidine deaminase enzymes such as APOBEC enzymes can be directed to a specific site in genomic DNA. Without wishing to be bound by any particular theory, advantages of using a nucleic acid programmable binding protein (e.g., a Cas9 domain) as a recognition agent include (1) the sequence specificity of nucleic acid programmable binding protein (e.g., a Cas9 domain) can be easily altered by simply changing the sgRNA sequence; and (2) the nucleic acid programmable binding protein (e.g., a Cas9 domain) may bind to its target sequence by denaturing the dsDNA, resulting in a stretch of DNA that is single-stranded and therefore a viable substrate for the deaminase. It should be understood that other catalytic domains of napDNAbps, or catalytic domains from other nucleic acid editing proteins, can also be used to generate fusion proteins with Cas9, and that the disclosure is not limited in this regard.

In view of the results provided herein regarding the nucleotides that can be targeted by Cas9:deaminase fusion proteins, a person of ordinary skill in the art will be able to design suitable guide RNAs to target the fusion proteins to a target sequence that comprises a nucleotide to be deaminated.

In some embodiments, the cytidine deaminase is an apolipoprotein B mRNA-editing complex (APOBEC) family deaminase. In some embodiments, the cytidine deaminase is an APOBEC1 deaminase. In some embodiments, the cytidine deaminase is an APOBEC2 deaminase. In some embodiments, the cytidine deaminase is an APOBEC3 deaminase. In some embodiments, the cytidine deaminase is an APOBEC3A deaminase. In some embodiments, the cytidine deaminase is an APOBEC3B deaminase. In some embodiments, the cytidine deaminase is an APOBEC3C deaminase. In some embodiments, the cytidine deaminase is an APOBEC3D deaminase. In some embodiments, the cytidine deaminase is an APOBEC3E deaminase. In some embodiments, the cytidine deaminase is an APOBEC3F deaminase. In some embodiments, the cytidine deaminase is an APOBEC3G deaminase. In some embodiments, the cytidine deaminase is an APOBEC3H deaminase. In some embodiments, the cytidine deaminase is an APOBEC4 deaminase. In some embodiments, the cytidine deaminase is an activation-induced deaminase (AID). In some embodiments, the cytidine deaminase is a vertebrate cytidine deaminase. In some embodiments, the cytidine deaminase is an invertebrate cytidine deaminase. In some embodiments, the cytidine deaminase is a human, chimpanzee, gorilla, monkey, cow, dog, rat, or mouse deaminase. In some embodiments, the cytidine deaminase is a human cytidine deaminase. In some embodiments, the cytidine deaminase is a rat cytidine deaminase, e.g., rAPOBEC1. In some embodiments, the cytidine deaminase is a Petromyzon marinus cytidine deaminase 1 (pmCDA1). In some embodiments, the cytidine deaminase is a human APOBEC3G (SEQ ID NO: 359). In some embodiments, the cytidine deaminase is a fragment of the human APOBEC3G (SEQ ID NO: 388). In some embodiments, the deaminase is a human APOBEC3G variant comprising a D316R and D317R mutation (SEQ ID NO: 387). In some embodiments, the deaminase is a fragment of the human APOBEC3G and comprising mutations corresponding to the D316R and D317R mutations in SEQ ID NO: 359 (SEQ ID NO: 389).

In some embodiments, the nucleic acid editing domain is at least 80%, at least 85%, at least 90%, at least 92%, at least 95%, at least 96%, at least 97%, at least 98%, at least 99%, or at least 99.5% identical to the deaminase domain of any one of SEQ ID NOs: 350-389. In some embodiments, the nucleic acid editing domain comprises the amino acid sequence of any one of SEQ ID NOs: 350-389.

Some exemplary suitable nucleic-acid editing domains, e.g., cytidine deaminases and cytidine deaminase domains that can be fused to napDNAbps (e.g., Cas9 domains) according to aspects of this disclosure are provided below. It should be understood that, in some embodiments, the active domain of the respective sequence can be used, e.g., the domain without a localizing signal (nuclear localization sequence, without nuclear export signal, cytoplasmic localizing signal).

Human AID:

(SEQ ID NO: 350)
MDSLLMNRRKFLYQFKNVRWAKGRRETYLCYVVKRRDSATSFSLDFGYLRN
KNGCHVELLFLRYISDWDLDPGRCYRVTWFTSWSPCYDCARHVADFLRGNP
NLSLRIFTARLYFCEDRKAEPEGLRRLHRAGVQIAIMTFKDYFYCWNTFVE
NHERTFKAWEGLHENSVRLSRQLRRILLPLYEVDDLRDAFRTLGL
(underline: nuclear localization sequence; double
underline: nuclear export signal)

Mouse AID:

(SEQ ID NO: 351)
MDSLLMKQKKFLYHFKNVRWAKGRHETYLCYVVKRRDSATSCSLDFGHLRN
KSGCHVELLFLRYISDWDLDPGRCYRVTWFTSWSPCYDCARHVAEFLRWNP
NLSLRIFTARLYFCEDRKAEPEGLRRLHRAGVQIGIMTFKDYFYCWNTFVE
NRERTFKAWEGLHENSVRLTRQLRRILLPLYEVDDLRDAFRMLGF
(underline: nuclear localization sequence; double
underline: nuclear export signal)

Dog AID:

(SEQ ID NO: 352)
MDSLLMKQRKFLYHFKNVRWAKGRHETYLCYVVKRRDSATSFSLDFGHLRN
KSGCHVELLFLRYISDWDLDPGRCYRVTWFTSWSPCYDCARHVADFLRGYP
NLSLRIFAARLYFCEDRKAEPEGLRRLHRAGVQIAIMTFKDYFYCWNTFVE
NREKTFKAWEGLHENSVRLSRQLRRILLPLYEVDDLRDAFRTLGL
(underline: nuclear localization sequence; double
underline: nuclear export signal)

Bovine AID:

(SEQ ID NO: 353)
MDSLLKKQRQFLYQFKNVRWAKGRHETYLCYVVKRRDSPTSFSLDFGHLRN
KAGCHVELLFLRYISDWDLDPGRCYRVTWFTSWSPCYDCARHVADFLRGYP
NLSLRIFTARLYFCDKERKAEPEGLRRLHRAGVQIAIMTFKDYFYCWNTFV
ENHERTFKAWEGLHENSVRLSRQLRRILLPLYEVDDLRDAFRTLGL
(underline: nuclear localization sequence; double
underline: nuclear export signal)

Rat AID:

(SEQ ID NO: 374)
MAVGSKPKAALVGPHWERERIWCFLCSTGLGTQQTGQTSRWLRPAATQDPV
SPPRSLLMKQRKFLYHFKNVRWAKGRHETYLCYVVKRRDSATSFSLDFGYL
RNKSGCHVELLFLRYISDWDLDPGRCYRVTWFTSWSPCYDCARHVADFLRG
NPNLSLRIFTARLTGWGALPAGLMSPARPSDYFYCWNTFVENHERTFKAWE
GLHENSVRLSRRLRRILLPLYEVDDLRDAFRTLGL
(underline: nuclear localization sequence; double
underline: nuclear export signal)

Mouse APOBEC-3:

(SEQ ID NO: 354)
MGPFCLGCSHRKCYSPIRNLISQETFKFHFKNLGYAKGRKDTFLCYEVTRK
DCDSPVSLHHGVFKNKDNIHAEICFLYWFHDKVLKVLSPREEFKITWYMSW
SPCFECAEQIVRFLATHHNLSLDIFSSRLYNVQDPETQQNLCRLVQEGAQV
AAMDLYEFKKCWKKFVDNGGRRFRPWKRLLTNFRYQDSKLQEILRPCYIPV
PSSSSSTLSNICLTKGLPETRFCVEGRRMDPLSEEEFYSQFYNQRVKHLCY
YHRMKPYLCYQLEQFNGQAPLKGCLLSEKGKQHAEILFLDKIRSMELSQVT
ITCYLTWSPCPNCAWQLAAFKRDRPDLILHIYTSRLYFHWKRPFQKGLCSL
WQSGILVDVMDLPQFTDCWTNFVNPKRPFWPWKGLEIISRRTQRRLRRIKE
SWGLQDLVNDFGNLQLGPPMS
(italic: nucleic acid editing domain)

Rat APOBEC-3:

(SEQ ID NO: 355)
MGPFCLGCSHRKCYSPIRNLISQETFKFHFKNLRYAIDRKDTFLCYEVTRK
DCDSPVSLHHGVFKNKDNIHAEICFLYWFHDKVLKVLSPREEFKITWYMSW
SPCFECAEQVLRFLATHHNLSLDIFSSRLYNIRDPENQQNLCRLVQEGAQV
AAMDLYEFKKCWKKFVDNGGRRFRPWKKLLTNFRYQDSKLQEILRPCYIPV
PSSSSSTLSNICLTKGLPETRFCVERRRVHLLSEEEFYSQFYNQRVKHLCY
YHGVKPYLCYQLEQFNGQAPLKGCLLSEKGKQHAEILFLDKIRSMELSQVI
ITCYLTWSPCPNCAWQLAAFKRDRPDLILHIYTSRLYFHWKRPFQKGLCSL
WQSGILVDVMDLPQFTDCWTNFVNPKRPFWPWKGLEIISRRTQRRLHRIKE
SWGLQDLVNDFGNLQLGPPMS
(italic: nucleic acid editing domain)

Rhesus Macaque APOBEC-3G:

(SEQ ID NO: 356)
MVEPMDPRTFVSNFNNRPILSGLNTVWLCCEVKTKDPSGPPLDAKIFQGKV
YSKAKYHPEMRFLRWFHKWRQLHHDQEYKVTWYVSWSPCTRCANSVATFLA
KDPKVTLTIFVARLYYFWKPDYQQALRILCQKRGGPHATMKIMNYNEFQDC
WNKFVDGRGKPFKPRNNLPKHYTLLQATLGELLRHLMDPGTFTSNFNNKPW
VSGQHETYLCYKVERLHNDTWVPLNQHRGFLRNQAPNIHGFPKGRHAELCF
LDLIPFWKLDGQQYRVTCFTSWSPCFSCAQEMAKFISNNEHVSLCIFAARI
YDDQGRYQEGLRALHRDGAKIAMMNYSEFEYCWDTFVDRQGRPFQPWDGLD
EHSQALSGRLRAI

(italic: nucleic acid editing domain; underline: cytoplasmic localization signal)

Chimpanzee APOBEC-3G:

(SEQ ID NO: 357)
MKPHFRNPVERMYQDTFSDNFYNRPILSHRNTVWLCYEVKTKGPSRPPLDA
KIFRGQVYSKLKYHPEMRFFHWFSKWRKLHRDQEYEVTWYISWSPCTKCTR
DVATFLAEDPKVTLTIFVARLYYFWDPDYQEALRSLCQKRDGPRATMKIMN
YDEFQHCWSKFVYSQRELFEPWNNLPKYYILLHIMLGEILRHSMDPPTFTS
NFNNELWVRGRHETYLCYEVERLHNDTWVLLNQRRGFLCNQAPHKHGFLEG
RHAELCFLDVIPFWKLDLHODYRVTCFTSWSPCFSCAQEMAKFISNNKHVS
LCIFAARIYDDQGRCQEGLRTLAKAGAKISIMTYSEFKHCWDTFVDHQGCP
FQPWDGLEEHSQALSGRLRAILQNQGN
(italic: nucleic acid editing domain; underline:
cytoplasmic localization signal)

Green Monkey APOBEC-3G:

(SEQ ID NO: 358)
MNPQIRNMVEQMEPDIFVYYFNNRPILSGRNTVWLCYEVKTKDPSGPPLDA
NIFQGKLYPEAKDHPEMKFLHWFRKWRQLHRDQEYEVTWYVSWSPCTRCAN
SVATFLAEDPKVTLTIFVARLYYFWKPDYQQALRILCQERGGPHATMKIMN
YNEFQHCWNEFVDGQGKPFKPRKNLPKHYTLLHATLGELLRHVMDPGTFTS
NFNNKPWVSGQRETYLCYKVERSHNDTWVLLNQHRGFLRNQAPDRHGFPKG
RHAELCFLDLIPFWKLDDQQYRVTCFTSWSPCFSCAQKMAKFISNNKHVSL
CIFAARIYDDQGRCQEGLRTLHRDGAKIAVMNYSEFEYCWDTFVDRQGRPF
QPWDGLDEHSQALSGRLRAI
(italic: nucleic acid editing domain; underline:
cytoplasmic localization signal)

Human APOBEC-3G:

(SEQ ID NO: 359)
MKPHFRNTVERMYRDTFSYNFYNRPILSRRNTVWLCYEVKTKGPSRPPLDA
KIFRGQVYSELKYHPEMRFFHWFSKWRKLHRDQEYEVTWYISWSPCTKCTR
DMATFLAEDPKVTLTIFVARLYYFWDPDYQEALRSLCQKRDGPRATMKIMN
YDEFQHCWSKFVYSQRELFEPWNNLPKYYILLHIMLGEILRHSMDPPTFTF
NFNNEPWVRGRHETYLCYEVERMHNDTWVLLNQRRGFLCNQAPHKHGFLEG
RHAELCFLDVIPFWKLDLDQDYRVTCFTSWSPCFSCAQEMAKFISKNKHVS
LCIFTARIYDDQGRCQEGLRTLAEAGAKISIMTYSEFKHCWDTFVDHQGCP
FQPWDGLDEHSQDLSGRLRAILQNQEN
(italic: nucleic acid editing domain; underline:
cytoplasmic localization signal)

Human APOBEC-3F:

(SEQ ID NO: 360)
MKPHFRNTVERMYRDTFSYNFYNRPILSRRNTVWLCYEVKTKGPSRPRLDA
KIFRGQVYSQPEHHAEMCFLSWFCGNQLPAYKCFQITWFVSWTPCPDCVAK
LAEFLAEHPNVTLTISAARLYYYWERDYRRALCRLSQAGARVKIMDDEEFA
YCWENFVYSEGQPFMPWYKFDDNYAFLHRTLKEILRNPMEAMYPHIFYFHF
KNLRKAYGRNESWLCFTMEVVKHHSPVSWKRGVFRNQVDPETHCHAERCFL
SWFCDDILSPNTNYEVTWYTSWSPCPECAGEVAEFLARHSNVNLTIFTARL
YYFWDTDYQEGLRSLSQEGASVEIMGYKDFKYCWENFVYNDDEPFKPWKGL
KYNFLFLDSKLQEILE
(italic: nucleic acid editing domain)

Human APOBEC-3B:

(SEQ ID NO: 361)
MNPQIRNPMERMYRDTFYDNFENEPILYGRSYTWLCYEVKIKRGRSNLLWD
TGVFRGQVYFKPQYHAEMCFLSWFCGNQLPAYKCFQITWFVSWTPCPDCVA
KLAEFLSEHPNVTLTISAARLYYYWERDYRRALCRLSQAGARVTIMDYEEF
AYCWENFVYNEGQQFMPWYKFDENYAFLHRTLKEILRYLMDPDTFTFNFNN
DPLVLRRRQTYLCYEVERLDNGTWVLMDQHMGFLCNEAKNLLCGFYGRHAE
LRFLDLVPSLQLDPAQIYRVTWFISWSPCFSWGCAGEVRAFLQENTHVRLR
IFAARIYDYDPLYKEALQMLRDAGAQVSIMTYDEFEYCWDTFVYRQGCPFQ
PWDGLEEHSQALSGRLRAILQNQGN
(italic: nucleic acid editing domain)

Rat APOBEC-3B:

(SEQ ID NO: 378)
MQPQGLGPNAGMGPVCLGCSHRRPYSPIRNPLKKLYQQTFYFHFKNVRYA
WGRKNNFLCYEVNGMDCALPVPLRQGVFRKQGHIHAELCFIYWFHDKVLR
VLSPMEEFKVTWYMSWSPCSKCAEQVARFLAAHRNLSLAIFSSRLYYYLR
NPNYQQKLCRLIQEGVHVAAMDLPEFKKCWNKFVDNDGQPFRPWMRLRIN
FSFYDCKLQEIFSRMNLLREDVFYLQFNNSHRVKPVQNRYYRRKSYLCYQ
LERANGQEPLKGYLLYKKGEQHVEILFLEKMRSMELSQVRITCYLTWSPC
PNCARQLAAFKKDHPDLILRIYTSRLYFYWRKKFQKGLCTLWRSGIHVDV
MDLPQFADCWTNFVNPQRPFRPWNELEKNSWRIQRRLRRIKESWGL

Bovine APOBEC-3B:

(SEQ ID NO: 379)
DGWEVAFRSGTVLKAGVLGVSMTEGWAGSGHPGQGACVWTPGTRNTMNLL
REVLFKQQFGNQPRVPAPYYRRKTYLCYQLKQRNDLTLDRGCFRNKKQRH
AEIRFIDKINSLDLNPSQSYKIICYITWSPCPNCANELVNFITRNNHLKL
EIFASRLYFHWIKSFKMGLQDLQNAGISVAVMTHTEFEDCWEQFVDNQSR
PFQPWDKLEQYSASIRRRLQRILTAPI

Chimpanzee APOBEC-3B:

(SEQ ID NO: 380)
MNPQIRNPMEWMYQRTFYYNFENEPILYGRSYTWLCYEVKIRRGHSNLLW
DTGVFRGQMYSQPEHHAEMCFLSWFCGNQLSAYKCFQITWFVSWTPCPDC
VAKLAKFLAEHPNVTLTISAARLYYYWERDYRRALCRLSQAGARVKIMDD
EEFAYCWENFVYNEGQPFMPWYKFDDNYAFLHRTLKEIIRHLMDPDTFTF
NFNNDPLVLRRHQTYLCYEVERLDNGTWVLMDQHMGFLCNEAKNLLCGFY
GRHAELRFLDLVPSLQLDPAQIYRVTWFISWSPCFSWGCAGQVRAFLQEN
THVRLRIFAARIYDYDPLYKEALQMLRDAGAQVSIMTYDEFEYCWDTFVY
RQGCPFQPWDGLEEHSQALSGRLRAILQVRASSLCMVPHRPPPPPQSPGP
CLPLCSEPPLGSLLPTGRPAPSLPFLLTASFSFPPPASLPPLPSLSLSPG
HLPVPSFHSLTSCSIQPPCSSRIRETEGWASVSKEGRDLG

Human APOBEC-3C:

(SEQ ID NO: 362)
MNPQIRNPMKAMYPGTFYFQFKNLWEANDRNETWLCFTVEGIKRRSVVSW
KTGVFRNQVDSETHCHAERCELSWFCDDILSPNTKYQVTWYTSWSPCPDC
AGEVAEFLARHSNVNLTIFTARLYYFQYPCYQEGLRSLSQEGVAVEIMDY
EDFKYCWENFVYNDNEPFKPWKGLKTNFRLLKRRLRESLQ
(italic: nucleic acid editing domain)

Gorilla APOBEC3C:

(SEQ ID NO: 375)
MNPQIRNPMKAMYPGTFYFQFKNLWEANDRNETWLCFTVEGIKRRSVVSW
KTGVFRNQVDSETHCHAERCFLSWFCDDILSPNTNYQVTWYTSWSPCPEC
AGEVAEFLARHSNVNLTIFTARLYYFQDTDYQEGLRSLSQEGVAVKIMDY
KDFKYCWENFVYNDDEPFKPWKGLKYNFRFLKRRLQEILE
(italic: nucleic acid editing domain)

Human APOBEC-3A:

(SEQ ID NO: 363)
MEASPASGPRHLMDPHIFTSNFNNGIGRHKTYLCYEVERLDNGTSVKMDQ
HRGFLHNQAKNLLCGFYGRHAELRFLDLVPSLQLDPAQIYRVTWFISWSP
CFSWGCAGEVRAFLQENTHVRLRIFAARIYDYDPLYKEALQMLRDAGAQV
SIMTYDEFKHCWDTFVDHQGCPFQPWDGLDEHSQALSGRLRAILQNQGN
(italic: nucleic acid editing domain)

Rhesus Macaque APOBEC-3A:

(SEQ ID NO: 376)
MDGSPASRPRHLMDPNTFTFNFNNDLSVRGRHQTYLCYEVERLDNGTWVP
MDERRGFLCNKAKNVPCGDYGCHVELRELCEVPSWQLDPAQTYRVTWFIS
WSPCFRRGCAGQVRVFLQENKHVRLRIFAARIYDYDPLYQEALRTLRDAG
AQVSIMTYEEFKHCWDTFVDRQGRPFQPWDGLDEHSQALSGRLRAILQNQ
GN
(italic: nucleic acid editing domain)

Bovine APOBEC-3A:

(SEQ ID NO: 377)
MDEYTFTENFNNQGWPSKTYLCYEMERLDGDATIPLDEYKGFVRNKGLDQ
PEKPCHAELYFLGKIHSWNLDRNQHYRLTCFISWSPCYDCAQKLTTFLKE
NHHISLHILASRIYTHNRFGCHQSGLCELQAAGARITIMTFEDFKHCWET
FVDHKGKPFQPWEGLNVKSQALCTELQAILKTQQN
(italic: nucleic acid editing domain)

Human APOBEC-3H:

(SEQ ID NO: 364)
MALLTAETFRLQFNNKRRLRRPYYPRKALLCYQLTPQNGSTPTRGYFENK
KKCHAEICFINEIKSMGLDETQCYQVTCYLTWSPCSSCAWELVDFIKAHD
HLNLGIFASRLYYHWCKPQQKGLRLLCGSQVPVEVMGFPKFADCWENFVD
HEKPLSFNPYKMLEELDKNSRAIKRRLERIKIPGVRAQGRYMDILCDAEV
(italic: nucleic acid editing domain)

Rhesus Macaque APOBEC-3H:

(SEQ ID NO: 381)
MALLTAKTFSLQFNNKRRVNKPYYPRKALLCYQLTPQNGSTPTRGHLKNK
KKDHAEIRFINKIKSMGLDETQCYQVTCYLTWSPCPSCAGELVDFIKAHR
HLNLRIFASRLYYHWRPNYQEGLLLLCGSQVPVEVMGLPEFTDCWENFVD
HKEPPSFNPSEKLEELDKNSQAIKRRLERIKSRSVDVLENGLRSLQLGPV
TPSSSIRNSR

Human APOBEC-3D:

(SEQ ID NO: 365)
MNPQIRNPMERMYRDTFYDNFENEPILYGRSYTWLCYEVKIKRGRSNLLW
DTGVFRGPVLPKRQSNHRQEVYFRFENHAEMCFLSWFCGNRLPANRRFQI
TWFVSWNPCLPCVVKVTKFLAEHPNVTLTISAARLYYYRDRDWRWVLLRL
HKAGARVKIMDYEDFAYCWENFVCNEGQPFMPWYKFDDNYASLHRTLKEI
LRNPMEAMYPHIFYFHFKNLLKACGRNESWLCFTMEVTKHHSAVFRKRGV
FRNQVDPETHCHAERCFLSWFCDDILSPNTNYEVTWYTSWSPCPECAGEV
AEFLARHSNVNLTIFTARLCYFWDTDYQEGLCSLSQEGASVKIMGYKDFV
SCWKNFVYSDDEPFKPWKGLQTNFRLLKRRLREILQ
(italic: nucleic acid editing domain)

Human APOBEC-1:

(SEQ ID NO: 366)
MTSEKGPSTGDPTLRRRIEPWEFDVFYDPRELRKEACLLYEIKWGMSRKI
WRSSGKNTTNHVEVNFIKKFTSERDFHPSMSCSITWFLSWSPCWECSQAI
REFLSRHPGVTLVIYVARLFWHMDQQNRQGLRDLVNSGVTIQIMRASEYY
HCWRNFVNYPPGDEAHWPQYPPLWMMLYALELHCIILSLPPCLKISRRWQ
NHLTFFRLHLQNCHYQTIPPHILLATGLIHPSVAWR

Mouse APOBEC-1:

(SEQ ID NO: 367)
MSSETGPVAVDPTLRRRIEPHEFEVFFDPRELRKETCLLYEINWGGRHSV
WRHTSQNTSNHVEVNFLEKFTTERYFRPNTRCSITWFLSWSPCGECSRAI
TEFLSRHPYVTLFIYIARLYHHTDQRNRQGLRDLISSGVTIQIMTEQEYC
YCWRNFVNYPPSNEAYWPRYPHLWVKLYVLELYCIILGLPPCLKILRRKQ
PQLTFFTITLQTCHYQRIPPHLLWATGLK

Rat APOBEC-1:

(SEQ ID NO: 368)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK

Human APOBEC-2:

(SEQ ID NO: 382)
MAQKEEAAVATEAASQNGEDLENLDDPEKLKELIELPPFEIVTGERLPAN
FFKFQFRNVEYSSGRNKTFLCYVVEAQGKGGQVQASRGYLEDEHAAAHAE
EAFFNTILPAFDPALRYNVTWYVSSSPCAACADRIIKTLSKTKNLRLLIL
VGRLFMWEEPEIQAALKKLKEAGCKLRIMKPQDFEYVWQNFVEQEEGESK
AFQPWEDIQENFLYYEEKLADILK

Mouse APOBEC-2:

(SEQ ID NO: 383)
MAQKEEAAEAAAPASQNGDDLENLEDPEKLKELIDLPPFEIVTGVRLPVN
FFKFQFRNVEYSSGRNKTFLCYVVEVQSKGGQAQATQGYLEDEHAGAHAE
EAFFNTILPAFDPALKYNVTWYVSSSPCAACADRILKTLSKTKNLRLLIL
VSRLFMWEEPEVQAALKKLKEAGCKLRIMKPQDFEYIWQNFVEQEEGESK
AFEPWEDIQENFLYYEEKLADILK

Rat APOBEC-2:

(SEQ ID NO: 384)
MAQKEEAAEAAAPASQNGDDLENLEDPEKLKELIDLPPFEIVTGVRLPVN
FFKFQFRNVEYSSGRNKTFLCYVVEAQSKGGQVQATQGYLEDEHAGAHAE
EAFFNTILPAFDPALKYNVTWYVSSSPCAACADRILKTLSKTKNLRLLIL
VSRLFMWEEPEVQAALKKLKEAGCKLRIMKPQDFEYLWQNFVEQEEGESK
AFEPWEDIQENFLYYEEKLADILK

Bovine APOBEC-2:

(SEQ ID NO: 385)
MAQKEEAAAAAEPASQNGEEVENLEDPEKLKELIELPPFEIVTGERLPAH
YFKFQFRNVEYSSGRNKTFLCYVVEAQSKGGQVQASRGYLEDEHATNHAE
EAFFNSIMPTFDPALRYMVTWYVSSSPCAACADRIVKTLNKTKNLRLLIL
VGRLFMWEEPEIQAALRKLKEAGCRLRIMKPQDFEYIWQNFVEQEEGESK
AFEPWEDIQENFLYYEEKLADILK

Petromyzon marinus CDA1 (pmCDA1):

(SEQ ID NO: 386)
MTDAEYVRIHEKLDIYTFKKQFFNNKKSVSHRCYVLFELKRRGERRACFW
GYAVNKPQSGTERGIHAEIFSIRKVEEYLRDNPGQFTINWYSSWSPCADC
AEKILEWYNQELRGNGHTLKIWACKLYYEKNARNQIGLWNLRDNGVGLNV
MVSEHYQCCRKIFIQSSHNQLNENRWLEKTLKRAEKRRSELSIMIQVKIL
HTTKSPAV

Human APOBEC3G D316R_D317R:

(SEQ ID NO: 387)
MKPHFRNTVERMYRDTFSYNFYNRPILSRRNTVWLCYEVKTKGPSRPPLD
AKIFRGQVYSELKYHPEMRFFHWFSKWRKLHRDQEYEVTWYISWSPCTKC
TRDMATFLAEDPKVTLTIFVARLYYFWDPDYQEALRSLCQKRDGPRATMK
IMNYDEFQHCWSKFVYSQRELFEPWNNLPKYYILLHIMLGEILRHSMDPP
TFTFNFNNEPWVRGRHETYLCYEVERMHNDTWVLLNQRRGFLCNQAPHKH
GFLEGRHAELCFLDVIPFWKLDLDQDYRVTCFTSWSPCFSCAQEMAKFIS
KNKHVSLCIFTARIYRRQGRCQEGLRTLAEAGAKISIMTYSEFKHCWDTF
VDHQGCPFQPWDGLDEHSQDLSGRLRAILQNQEN

Human APOBEC3G Chain A:

(SEQ ID NO: 388)
MDPPTFTFNFNNEPWVRGRHETYLCYEVERMHNDTWVLLNQRRGFLCNQA
PHKHGFLEGRHAELCFLDVIPFWKLDLDQDYRVTCFTSWSPCFSCAQEMA
KFISKNKHVSLCIFTARIYDDQGRCQEGLRTLAEAGAKISIMTYSEFKHC
WDTFVDHQGCPFQPWDGLDEHSQDLSGRLRAILQ

Human APOBEC3G Chain A D120R_D121R:

(SEQ ID NO: 389)
MDPPTFTFNFNNEPWVRGRHETYLCYEVERMHNDTWVLLNQRRGFLCNQA
PHKHGFLEGRHAELCFLDVIPFWKLDLDQDYRVTCFTSWSPCFSCAQEMA
KFISKNKHVSLCIFTARIYRRQGRCQEGLRTLAEAGAKISIMTYSEFKHC
WDTFVDHQGCPFQPWDGLDEHSQDLSGRLRAILQ

In some embodiments, the deaminase domain is an adenosine deaminase domain. In some embodiments, the adenosine deaminase catalyzes the hydrolytic deamination of adenosine (A) or deoxyadenosine (dA) to inosine (I) or deoxyinosine (dl), respectively. In some embodiments, the adenosine deaminase catalyzes the hydrolytic deamination of adenine or adenosine in deoxyribonucleic acid (DNA). For example, the adenosine may be converted to an inosine residue, which typically base pairs with a cytosine residue. Without wishing to be bound by any particular theory, fusion proteins comprising an adenosine deaminase are useful inter alia for targeted editing, referred to herein as “base editing,” of nucleic acid sequences in vitro and in vivo. The adenosine deaminase may be derived from any suitable organism (e.g., E. coli). In some embodiments, the adenine deaminase is a naturally-occurring adenosine deaminase that includes one or more mutations (i.e., a adenosine deaminase) corresponding to any of the mutations provided herein (e.g., mutations in ecTadA). One of ordinary skill in the art will be able to identify the corresponding residue in any homologous protein and in the respective encoding nucleic acid by methods well known in the art, e.g., by sequence alignment and determination of homologous residues. Accordingly, one of ordinary skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In some embodiments, the adenosine deaminase comprises the amino acid sequence of any one of SEQ ID NOs: 400-458. In some embodiments, the adenosine deaminase consists of the amino acid sequence of any one of SEQ ID NOs: 400-458. In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 400-458. Additional adenosine deaminase domains that may be suitable for use in the present invention are provided and described in Gaudelli N M, et al. (2017) Programmable base editing of A⋅T to G⋅C in genomic DNA without DNA cleavage, Nature, 551(23); 464-471; the entire contents of which is incorporated herein by reference. It should be appreciated that adenosine deaminases provided herein may include one or more mutations (e.g., any of the mutations provided herein), or may not include any mutations (i.e., a wild-type adensosine demainase). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 400-458 or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in SEQ ID NOs: 400-458, or any of the adenosine deaminases provided herein.

It should be appreciated that the adenosine deaminase (e.g., a first or second adenosine deaminase) may comprise one or more of the mutations provided in any of the adenosine deaminases (e.g., ecTadA adenosine deaminases) shown in Example 2. In some embodiments, the adenosine deaminase comprises the combination of mutations of any of the adenosine deaminases (e.g., ecTadA adenosine deaminases) shown in Example 2. For example, the adenosine deaminase may comprise the mutations W23R, H36L, P48A, R51L, L84F, A106V, D108N, H123Y, S146C, D147Y, R152P, E155V, I156F, and K157N (relative to SEQ ID NO: 400), which is also referred to as ABE7.10. In some embodiments, the adenosine deaminase may comprise the mutations H36L, R51L, L84F, A106V, D108N, H123Y, S146C, D147Y, E155V, I156F, and K157N (relative to SEQ ID NO: 400). In some embodiments, the adenosine deaminase comprises any of the following combination of mutations relative to SEQ ID NO: 400, where each mutation of a combination is separated by a “_” and each combination of mutations is between parentheses: (A106V_D108N), (R107C_D108N), (H8Y_D108N_S127S_D147Y_Q154H), (H8Y_R24W_D108N_N127S_D147Y_E155V), (D108N_D147Y_E155V), (H8Y_D108N_S127S), (H8Y_D108N_N127S_D147Y_Q154H), (A106V_D108N_D147Y_E155V), (D108Q_D147Y_E155V), (D108M_D147Y_E155V), (D108L_D147Y_E155V), (D108K_D147Y_E155V), (D108I_D147Y_E155V), (D108F_D147Y_E155V), (A106V_D108N_D147Y), (A106V_D108M_D147Y_E155V), (E59A_A106V_D108N_D147Y_E155V), (E59A cat dead_A106V_D108N_D147Y_E155V), (L84F_A106V_D108N_H123Y_D147Y_E155V_I156Y), (L84F_A106V_D108N_H123Y_D147Y_E155V_I156F), (D103A_D014N), (G22P_D103A_D104N), (G22P_D103A_D104N_S138A), (D103A_D104N_S138A), (R26G_L84F_A106V_R107H_D108N_H123Y_A142N_A143D_D147Y_E155V_I156F), (E25G_R26G_L84F_A106V_R107H_D108N_H123Y_A142N_A143D_D147Y_E155V_I156F), (E25D_R26G_L84F_A106V_R107K_D108N_H123Y_A142N_A143G_D147Y_E155V_I156F), (R26Q_L84F_A106V_D108N_H123Y_A142N_D147Y_E155V_I156F), (E25M_R26G_L84F_A106V_R107P_D108N_H123Y_A142N_A143D_D147Y_E155V_I156F), (R26C_L84F_A106V_R107H_D108N_H123Y_A142N_D147Y_E155V_I156F), (L84F_A106V_D108N_H123Y_A142N_A143L_D147Y_E155V_I156F), (R26G_L84F_A106V_D108N_H123Y_A142N_D147Y_E155V_I156F), (E25A_R26G_L84F_A106V_R107N_D108N_H123Y_A142N_A143E_D147Y_E155V_I15 6F), (R26G_L84F_A106V_R107H_D108N_H123Y_A142N_A143D_D147Y_E155V_I156F), (A106V_D108N_A142N_D147Y_E155V), (R26G_A106V_D108N_A142N_D147Y_E155V), (E25D_R26G_A106V_R107K_D108N_A142N_A143G_D147Y_E155V), (R26G_A106V_D108N_R107H_A142N_A143D_D147Y_E155V), (E25D_R26G_A106V_D108N_A142N_D147Y_E155V), (A106V_R107K_D108N_A142N_D147Y_E155V), (A106V_D108N_A142N_A143G_D147Y_E155V), (A106V_D108N_A142N_A143L_D147Y_E155V), (H36L_R51L_L84F_A106V_D108N_H123Y_S146C_D147Y_E155V_I156F_K157N), (H36L_P48S_R51L_L84F_A106V_D108N_H123Y_S146C_D147Y_E155V_I156F_K157N), (H36L_P48S_R51L_L84F_A106V_D108N_H123Y_A142N_S146C_D147Y_E155V_I156F_K157N), (W23L_H36L_P48A_R51L_L84F_A106V_D108N_H123Y_A142N_S146C_D147Y_E155V_I156F_K157N), (W23L_H36L_P48A_R51L_L84F_A106V_D108N_H123Y_A142N_S146C_D147Y_R152P_E155V_I156F_K157N), (N37T_P48T_M70L_L84F_A106V_D108N_H123Y_D147Y_I49V_E155V_I156F), (N37S_L84F_A106V_D108N_H123Y_D147Y_E155V_I156F_K161T), (H36L_L84F_A106V_D108N_H123Y_D147Y_Q154H_E155V_I156F), (N72S_L84F_A106V_D108N_H123Y_S146R_D147Y_E155V_I156F), (H36L_P48L_L84F_A106V_D108N_H123Y_E134G_D147Y_E155V_I156F), (H36L_L84F_A106V_D108N_H123Y_D147Y_E155V_I156F_K157N), (H36L_L84F_A106V_D108N_H123Y_S146C_D147Y_E155V_I156F), (L84F_A106V_D108N_H123Y_S146R_D147Y_E155V_I156F_K161T), (N37S_R51H_D77G_L84F_A106V_D108N_H123Y_D147Y_E155V_I156F), (R51L_L84F_A106V_D108N_H123Y_D147Y_E155V_I156F_K157N), (D24G_Q71R_L84F_H96L_A106V_D108N_H123Y_D147Y_E155V_I156F_K160E), (H36L_G67V_L84F_A106V_D108N_H123Y_S146T_D147Y_E155V_I156F), (Q71L_L84F_A106V_D108N_H123Y_L137M_A143E_D147Y_E155V_I156F), (E25G_L84F_A106V_D108N_H123Y_D147Y_E155V_I156F_Q159L), (L84F_A91T_F104I_A106V_D108N_H123Y_D147Y_E155V_I156F), (N72D_L84F_A106V_D108N_H123Y_G125A_D147Y_E155V_I156F), (P48S_L84F_S97C_A106V_D108N_H123Y_D147Y_E155V_I156F), (W23G_L84F_A106V_D108N_H123Y_D147Y_E155V_I156F), (D24G_P48L_Q71R_L84F_A106V_D108N_H123Y_D147Y_E155V_I156F_Q159L), (L84F_A106V_D108N_H123Y_A142N_D147Y_E155V_I156F), (H36L_R51L_L84F_A106V_D108N_H123Y_A 142N_S146C_D147Y_E155V_I156F_K157N), (N37S_L84F_A106V_D108N_H123Y_A142N_D147Y_E155V_I156F_K161T), (L84F_A106V_D108N_D147Y_E155V_I156F), (R51L_L84F_A106V_D108N_H123Y_S146C_D147Y_E155V_I156F_K157N_K161T), (L84F_A106V_D108N_H123Y_S146C_D147Y_E155V_I156F_K161T), (L84F_A106V_D108N_H123Y_S146C_D147Y_E155V_I156F_K157N_K160E_K161T), (L84F_A106V_D108N_H123Y_S146C_D147Y_E155V_I156F_K157N_K160E), (R74Q L84F_A106V_D108N_H123Y_D147Y_E155V_I156F), (R74A_L84F_A106V_D108N_H123Y_D147Y_E155V_I156F), (L84F_A106V_D108N_H123Y_D147Y_E155V_I156F), (R74Q_L84F_A106V_D108N_H123Y_D147Y_E155V_I156F), (L84F_R98Q_A106V_D108N_H123Y_D147Y_E155V_I156F), (L84F_A106V_D108N_H123Y_R129Q_D147Y_E155V_I156F), (P48S_L84F_A106V_D108N_H123Y_A142N_D147Y_E155V_I156F), (P48S_A142N), (P48T_I49V_L84F_A106V_D108N_H123Y_A142N_D147Y_E155V_I156F_L157N), (P48T_I49V_A142N), (H36L_P48S_R51L_L84F_A106V_D108N_H123Y_S146C_D147Y_E155V_I156F_K157N), (H36L_P48S_R51L_L84F_A106V_D108N_H123Y_S146C_A142N_D147Y_E155V_I156F_K157N), (H36L_P48T_I49V_R51L_L84F_A106V_D108N_H123Y_S146C_D147Y_E155V_I156F_K157N), (H36L_P48T_I49V_R51L_L84F_A106V_D108N_H123Y_A142N_S146C_D147Y_E155V_I156F_K157N), (H36L_P48A_R51L_L84F_A106V_D108N_H123Y_S146C_D147Y_E155V_I156F_K157N), (H36L_P48A_R51L_L84F_A106V_D108N_H123Y_A142N_S146C_D147Y_E155V_I156F_K157N), (H36L_P48A_R51L_L84F_A106V_D108N_H123Y_S146C_A142N_D147Y_E155V_I156F_K157N), (W23L_H36L_P48A_R51L_L84F_A106V_D108N_H123Y_S146C_D147Y_E155V_I156F_K157N), (W23R_H36L_P48A_R51L_L84F_A106V_D108N_H123Y_S146C_D147Y_E155V_I156F_K157N), (W23L_H36L_P48A_R51L_L84F_A106V_D108N_H123Y_S146R_D147Y_E155V_I156F_K161T), (H36L_P48A_R51L_L84F_A106V_D108N_H123Y_S146C_D147Y_R152H_E155V_I156F_K157N), (H36L_P48A_R51L_L84F_A106V_D108N_H123Y_S146C_D147Y_R152P_E155V_I156F_K157N), (W23L_H36L_P48A_R51L_L84F_A106V_D108N_H123Y_S146C_D147Y_R152P_E155V_I156F_K157N), (W23L_H36L_P48A_R51L_L84F_A106V_D108N_H123Y_A142A_S146C_D147Y_E155V_I156F_K157N), (W23L_H36L_P48A_R51L_L84F_A106V_D108N_H123Y_A142A_S146C_D147Y_R152P_E155V_I156F_K157N), (W23L_H36L_P48A_R51L_L84F_A106V_D108N_H123Y_S146R_D147Y_E155V_I156F_K161T), (W23R_H36L_P48A_R51L_L84F_A106V_D108N_H123Y_S146C_D147Y_R152P_E155V_I156F_K157N), (H36L_P48A_R51L_L84F_A106V_D108N_H123Y_A142N_S146C_D147Y_R152P_E155V_I156F_K157N).

In some embodiments, fusion proteins as provided herein comprise the full-length amino acid of a nucleic acid editing enzyme, e.g., one of the sequences provided above. In other embodiments, however, fusion proteins as provided herein do not comprise a full-length sequence of a nucleic acid editing enzyme, but only a fragment thereof. For example, in some embodiments, a fusion protein provided herein comprises a napDNAbp and a fragment of a nucleic acid editing enzyme, e.g., wherein the fragment comprises a nucleic acid editing domain. Exemplary amino acid sequences of nucleic acid editing domains are shown in the sequences above, and additional suitable sequences of such domains will be apparent to those of ordinary skill in the art based on this disclosure and knowledge in the field.

Additional suitable nucleic-acid editing enzyme sequences, e.g., deaminase enzyme and domain sequences, that can be used according to aspects of this invention, e.g., that can be fused to a napDNAbp (e.g., a nuclease-inactive Cas9 domain), will be apparent to those of ordinary skill in the art based on this disclosure. In some embodiments, such additional enzyme sequences include deaminase enzyme or deaminase domain sequences that are at least 70%, at least 75%, at least 80%, at least 85%, at least 90%, at least 95%, at least 96%, at least 97%, at least 98%, or at least 99% similar to the sequences provided herein. Additional suitable napDNAbps (e.g., Cas9 domains), variants, and sequences will also be apparent to those of ordinary skill in the art. Examples of such additional suitable Cas9 domains include, but are not limited to, D10A, D10A/D839A/H840A, and D10A/D839A/H840A/N863A mutant domains (see, e.g., Prashant et al., CAS9 transcriptional activators for target specificity screening and paired nickases for cooperative genome engineering. Nature Biotechnology. 2013; 31(9): 833-838; the entire contents of which are incorporated herein by reference). In some embodiments, the Cas9 comprises a histidine residue at position 840 of the amino acid sequence provided in SEQ ID NO: 10, or a corresponding mutation in any of the amino acid sequences provided in SEQ ID NOs: 11-260. The presence of the catalytic residue H840 restores the acvitity of the Cas9 to cleave the non-edited strand containing a G opposite the targeted C. Restoration of H840 does not result in the cleavage of the target strand containing the C.

### High Fidelity Cas9

Some aspects of the disclosure provide high fidelity Cas9 domains. In some embodiments, high fidelity Cas9 domains have decreased electrostatic interactions between the Cas9 domain and a sugar-phosphate backbone of a DNA, as compared to a wild-type Cas9 domain. In some embodiments, any of the Cas9 domains provided herein comprise one or more mutations that decrease the association between the Cas9 domain and a sugar-phosphate backbone of a DNA. In some embodiments, any of the Cas9 domains provided herein comprise one or more mutations that decrease the association between the Cas9 domain and a sugar-phosphate backbone of a DNA by at least 5%, at least 10%, at least 15%, at least 20%, at least 25%, at least 30%, at least 35%, at least 40%, at least 45%, at least 50%, at least 60%, at least 70%, at least 80%, at least 90%, or at least 95%. In some embodiments, any of the Cas9 domains provided herein comprise one or more of a N497X, a R661X, a Q695X, and/or a Q926X mutation of the amino acid sequence provided in SEQ ID NO: 9, or a corresponding mutation in any of the amino acid sequences provided in SEQ ID NOs: 10-262, wherein X is any amino acid. In some embodiments, any of the Cas9 domains provided herein comprise one or more of a N497A, a R661A, a Q695A, and/or a Q926A mutation of the amino acid sequence provided in SEQ ID NO: 9, or a corresponding mutation in any of the amino acid sequences provided in SEQ ID NOs: 10-262. In some embodiments, the Cas9 domain comprises a D10A mutation of the amino acid sequence provided in SEQ ID NO: 9, or a corresponding mutation in any of the amino acid sequences provided in SEQ ID NOs: 10-262. In some embodiments, the Cas9 domain comprises the amino acid sequence as set forth in SEQ ID NO: 296. High fidelity Cas9 domains have been described in the art and would be apparent to the skilled artisan. For example, high fidelity Cas9 domains have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each are incorporated herein by reference. It should be appreciated that, based on the present disclosure and knowledge in the art, that mutations in any Cas9 domain may be generated to make high fidelity Cas9 domains that have decreased electrostatic interactions between the Cas9 domain and a sugar-phosphate backbone of a DNA, as compared to a wild-type Cas9 domain.

Cas9 domain where mutations relative to Cas9 of SEQ ID NO: 9 are shown in bold and underlines.

(SEQ ID NO: 296)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMT [symbol] FDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWG [symbol] LSRKLINGIRDKQSGKTILDFLKSDGFANRNFM [symbol] LIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETR [symbol] ITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

Cas9 Domains with Reduced PAM Exclusivity

Some aspects of the disclosure provide Cas9 domains that have different PAM specificities. Typically, Cas9 domains, such as Cas9 from S. pyogenes (spCas9), require a canonical NGG PAM sequence to bind a particular nucleic acid region. This may limit the ability to of the Cas9 domain to bind to a particular nucleotide sequence within a genome. Accordingly, in some embodiments, any of the Cas proteins provided herein may be capable of binding a nucleotide sequence that does not contain a canonical (e.g., NGG) PAM sequence. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises the amino acid sequence SEQ ID NO: 297. In some embodiments, the SaCas9 comprises a N579X mutation of SEQ ID NO: 297, or a corresponding mutation in any of the amino acid sequences provided in SEQ ID NOs: 9-262, wherein X is any amino acid except for N. In some embodiments, the SaCas9 comprises a N579A mutation of SEQ ID NO: 297, or a corresponding mutation in any of the amino acid sequences provided in SEQ ID NOs: 10-262. In some embodiments, the SaCas9 domain, the SaCas9d protein, or the SaCas9n protein can bind to a nucleic acid sequence having a non-canonical PAM. In some embodiments, the SaCas9 domain, the SaCas9d protein, or the SaCas9n protein can bind to a nucleic acid sequence having a NNGRRT PAM sequence. In some embodiments, the SaCas9 domain comprises one or more of a E781X, N967X, or R1014X mutation of SEQ ID NO: 297, or a corresponding mutation in any of the amino acid sequences provided in SEQ ID NOs: 10-262, wherein X is any amino acid. In some embodiments, the SaCas9 domain comprises one or more of a E781K, N967K, or R1014H mutation of SEQ ID NO: 297, or one or more corresponding mutation in any of the amino acid sequences provided in SEQ ID NOs: 10-262. In some embodiments, the SaCas9 domain comprises a E781K, a N967K, and a R1014H mutation of SEQ ID NO: 297, or corresponding mutations in any of the amino acid sequences provided in SEQ ID NOs: 10-262. It should be appreciated that these mutations may be combined with any of the other mutations provided herein

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 297-299. In some embodiments, the Cas9 domain of any of the fusion proteins provided herein comprises the amino acid sequence of any one of SEQ ID NOs: 297-299. In some embodiments, the Cas9 domain of any of the fusion proteins provided herein consists of the amino acid sequence of any one of SEQ ID NOs: 297-299.

Exemplary SaCas9 Sequence

KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEE [symbol] SKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG
(SEQ ID NO: 297; Residue N579 of SEQ ID NO: 297,
which is underlined and in bold, may be mutated
(e.g., to a A579) to yield a SaCas9 nickase)

Exemplary SaCas9n Sequence

KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEE [symbol] SKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG
(SEQ ID NO: 298; Residue A579 of SEQ ID NO: 298,
which can be mutated from N579 of SEQ ID NO: 297
to yield a SaCas9 nickase, is underlined and in
bold)

Exemplary SaKKH Cas9

KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEE [symbol] SKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRKLINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYKNDLIKINGELYRVIGVNNDLLNRIEVNMIDITY
REYLENMNDKRPPHIIKTIASKTQSIKKYSTDILGNLYEVKSKKHPQIIK
KG
(SEQ ID NO: 299; Residue A579 of SEQ ID NO: 299,
which can be mutated from N579 of SEQ ID NO: 297
to yield a SaCas9 nickase, is underlined and in
bold. Residues K781, K967, and H1014 of SEQ ID
NO: 299, which can be mutated from E781, N967,
and R1014 of SEQ ID NO: 297 to yield a SaKKH
Cas9 are underlined and in italics)

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises the amino acid sequence SEQ ID NO: 9. In some embodiments, the SpCas9 comprises a D10X mutation of SEQ ID NO: 9, or a corresponding mutation in any of the amino acid sequences provided in SEQ ID NOs: 10-262, wherein X is any amino acid except for D. In some embodiments, the SpCas9 comprises a D10A mutation of SEQ ID NO: 9, or a corresponding mutation in any of the amino acid sequences provided in SEQ ID NOs: 10-262. In some embodiments, the SpCas9 domain, the SpCas9d protein, or the SpCas9n protein can bind to a nucleic acid sequence having a non-canonical PAM. In some embodiments, the SpCas9 domain, the SpCas9d protein, or the SpCas9n protein can bind to a nucleic acid sequence having a NGG, a NGA, or a NGCG PAM sequence. In some embodiments, the SpCas9 domain comprises one or more of a D1 135X, R1335X, and T1337X mutation of SEQ ID NO: 9, or a corresponding mutation in any of the amino acid sequences provided in SEQ ID NOs: 10-262, wherein X is any amino acid. In some embodiments, the SpCas9 domain comprises one or more of a D1135E, R1335Q, and T1337R mutation of SEQ ID NO: 9, or a corresponding mutation in any of the amino acid sequences provided in SEQ ID NOs: 10-262. In some embodiments, the SpCas9 domain comprises a D1135E, a R1335Q, and a T1335R mutation of SEQ ID NO: 9, or corresponding mutations in any of the amino acid sequences provided in SEQ ID NOs: 10-262. In some embodiments, the SpCas9 domain comprises one or more of a D1135X, R1335X, and T1337X mutation of SEQ ID NO: 9, or a corresponding mutation in any of the amino acid sequences provided in SEQ ID NOs: 10-262, wherein X is any amino acid. In some embodiments, the SpCas9 domain comprises one or more of a D1135V, R1335Q, and T1337R mutation of SEQ ID NO: 9, or a corresponding mutation in any of the amino acid sequences provided in SEQ ID NOs: 10-262. In some embodiments, the SpCas9 domain comprises a D1135V, a R1335Q, and a T1337R mutation of SEQ ID NO: 9, or corresponding mutations in any of the amino acid sequences provided in SEQ ID NOs: 10-262. In some embodiments, the SpCas9 domain comprises one or more of a D1135X, G1218X, R1335X, and T1337X mutation of SEQ ID NO: 9, or a corresponding mutation in any of the amino acid sequences provided in SEQ ID NOs: 10-262, wherein X is any amino acid. In some embodiments, the SpCas9 domain comprises one or more of a D1135V, G1218R, R1335Q, and T1337R mutation of SEQ ID NO: 9, or a corresponding mutation in any of the amino acid sequences provided in SEQ ID NOs: 10-262. In some embodiments, the SpCas9 domain comprises a D1135V, a G1218R, a R1335Q, and a T1337R mutation of SEQ ID NO: 9, or corresponding mutations in any of the amino acid sequences provided in SEQ ID NOs: 10-262. It should be appreciated that these mutations may be combined with any of the other mutations provided herein

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 9, 270-273. In some embodiments, the Cas9 domain of any of the fusion proteins provided herein comprises the amino acid sequence of any one of SEQ ID NOs: 9 or 270-273. In some embodiments, the Cas9 domain of any of the fusion proteins provided herein consists of the amino acid sequence of any one of SEQ ID NOs: 9 or 270-273.

Exemplary SpCas9n

(SEQ ID NO: 270)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

Exemplary SpEQR Cas9

DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGF [symbol] SPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRK [symbol] Y [symbol] STKEVLDATLIHQS
ITGLYETRIDLSQLGGD
(SEQ ID NO: 271; Residues E1134, Q1334, and R1336
of SEQ ID NO: 271, which can be mutated from
D1134, R1334, and T1336 of SEQ ID NO: 9 to yield
a SpEQR Cas9, are underlined and in bold)

Exemplary SpVQR Cas9

DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGF [symbol] SPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRK [symbol] Y [symbol] STKEVLDATLIHQS
ITGLYETRIDLSQLGGD
(SEQ ID NO: 272; Residues V1134, Q1334, and R1336
of SEQ ID NO: 272, which can be mutated from
D1134, R1334, and T1336 of SEQ ID NO: 9 to yield
a SpVQR Cas9, are underlined and in bold)

Exemplary SpVRER Cas9

DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGF [symbol] SPTVAYSVLVVAKVEK
GKSKKLKSVKELLGITIMERSSFEKNPIDFLEAKGYKEVKKDLIIKLPKY
SLFELENGRKRMLASA [symbol] ELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRK [symbol] Y [symbol] STKEVLDATLIHQS
ITGLYETRIDLSQLGGD
(SEQ ID NO: 273; Residues V1134, R1217, Q1334, and
R1336 of SEQ ID NO: 313, which can be mutated from
D1134, G1217, R1334, and T1336 of SEQ ID NO: 9 to
yield a SpVRER Cas9, are underlined and in bold)

### Methods of Using Cas9 Fusion Proteins

Some aspects of this disclosure provide methods of using the Cas9 domains, fusion proteins, or complexes provided herein.

In one aspect, provided herein are methods comprising contacting a nucleic acid molecule (a) with any of the Cas9 domains or fusion proteins provided herein, and with at least one guide RNA, wherein the guide RNA is about 15-100 nucleotides long and comprises a sequence of at least 10 contiguous nucleotides that is complementary to a target sequence in the nucleic acid molecule; or (b) with a Cas9 domain, a fusion protein comprising a Cas9 domain, or a complex comprising a Cas9 domain, wherein the Cas9 domain is associated with at least one gRNA as provided herein. In some embodiments, the nucleic acid is present in a cell. In some embodiments, the nucleic acid is present in a subject. In some embodiments, the contacting is in vitro. In some embodiments, the contacting is in vivo in a subject.

In another aspect, provided herein are methods comprising contacting a cell (a) with any of the Cas9 domains or fusion proteins provided herein, and with at least one guide RNA, wherein the guide RNA is about 15-100 nucleotides long and comprises a sequence of at least 10 contiguous nucleotides that is complementary to a target sequence in the nucleic acid molecule; or (b) with a Cas9 domain, a fusion protein comprising a Cas9 domain, or a complex comprising a Cas9 domain, wherein the Cas9 domain is associated with at least one gRNA as provided herein. In some embodiments, the contacting is in vitro. In some embodiments, the contacting is in vivo in a subject. In some embodiments, the cell is a prokaryotic cell. In some embodiments, the prokaryotic cell is a bacterium. In some embodiments, the bacterium is E. coli. In some embodiments, the cell is a eukaryotic cell. In some embodiments, the eukaryotic cell is a mammalian cell. In some embodiments, the mammalian cell is a human cell. In some embodiments, the cell is a plant or fungal cell.

In another aspect, provided herein are methods for administering to a subject (a) any of the Cas9 domains or fusion proteins provided herein, and at least one guide RNA, wherein the guide RNA is about 15-100 nucleotides long and comprises a sequence of at least 10 contiguous nucleotides that is complementary to a target sequence in the nucleic acid molecule; or (b) a Cas9 domain, a fusion protein comprising a Cas9 domain, or a complex comprising a Cas9 domain, wherein the Cas9 domain is associated with at least one gRNA as provided herein. In some embodiments, an effective amount of the Cas9 domain, fusion protein, or complex is administered to the subject. In some embodiments, the effective amount is an amount effective for treating a disease or disorder, wherein the disease comprises one or more point mutations in a nucleic acid sequence associated with the disease or disorder.

In some embodiments, the 3′ end of the target sequence is not immediately adjacent to the canonical PAM sequence (5′-NGG-3′). In some embodiments, the 3′ end of the target sequence is directly adjacent to a sequence selected from the group consisting of NGT, NGA, NGC, and NNG, wherein N is an A, G, T, or C. In some embodiments, the 3′ end of the target sequence is directly adjacent to a sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA.

In some embodiments, the target sequence comprises a sequence associated with a disease or disorder. In some embodiments, the target sequence comprises a point mutation associated with a disease or disorder. In some embodiments, the activity of the Cas9 domain, the Cas9 fusion protein, or the complex results in a correction of the point mutation. In some embodiments, the target sequence comprises a T→C point mutation associated with a disease or disorder, wherein the deamination of the mutant C base results in a sequence that is not associated with a disease or disorder. In some embodiments, the target sequence comprises a A→G, wherein deamination of the C that is base-paired to the mutant G base results in a sequence that is not associated with a disease or disorder. In some embodiments, the target sequence encodes a protein and wherein the point mutation is in a codon and results in a change in the amino acid encoded by the mutant codon as compared to the wild-type codon. In some embodiments, the deamination of the mutant C results in a change of the amino acid encoded by the mutant codon. In some embodiments, the deamination of the mutant C results in the codon encoding the wild-type amino acid. In some embodiments, the target DNA sequence comprises a G→A point mutation associated with a disease or disorder, and wherein the deamination of the mutant A base results in a sequence that is not associated with a disease or disorder. In some embodiments, the target DNA sequence comprises a C→T point mutation associated with a disease or disorder, wherein deamination of the A that is base-paired with the mutant T results in a sequence that is not associated with a disease or disorder. In some embodiments, the target DNA sequence encodes a protein and wherein the point mutation is in a codon and results in a change in the amino acid encoded by the mutant codon as compared to the wild-type codon. In some embodiments, the deamination of the mutant A results in a change of the amino acid encoded by the mutant codon. In some embodiments, the deamination of the mutant A results in the codon encoding the wild-type amino acid. In some embodiments, the contacting is in vivo in a subject. In some embodiments, the subject has or has been diagnosed with a disease or disorder. In some embodiments, the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNB1 protein, a mutant HRAS protein, or a mutant p53 protein. In some embodiments, the target sequence comprises a sequence located in a genomic locus. In some embodiments, the genomic locus is a HEK site. In some embodiments, the HEK site is HEK site 3 or HEK site 4. In some embodiments, the HEK site comprises a CGG, GGG, TGT, GGT, AGC, CGC, TGC, AGA, or TGA PAM sequence. In some embodiments, the genomic locus is EMX1. In some embodiments, the EMX1 locus comprises a GGG or CAA PAM sequence. In some embodiments, the genomic locus is VEGFA. In some embodiments, the VEGFA locus comprises a AGT, GGC, GGA, or GAT PAM sequence. In some embodiments, the genomic locus is FANCF. In some embodiments, the FANCF locus comprises a CGT, GAA, GAT, TGG, AGT, TGT, GGT, CGC, TGC, GGC, AGA, or TGA PAM sequence.

Some embodiments provide methods for using the Cas9 DNA editing fusion proteins provided herein. In some embodiments, the fusion protein is used to introduce a point mutation into a nucleic acid by deaminating a target nucleobase, e.g., a C or A residue. In some embodiments, the deamination of the target nucleobase results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ a fusion protein comprising a Cas9 domain (e.g., a base editor) to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provide herein is to restore the function of a dysfunctional gene via genome editing. The Cas9-deaminase fusion proteins provided herein can be validated for gene editing-based human therapeutics in vitro, e.g., by correcting a disease-associated mutation in human cell culture. It will be understood by the skilled artisan that the fusion proteins provided herein, e.g., the fusion proteins comprising a Cas9 domain and a cytidine deaminase domain can be used to correct any single T→C or A→G point mutation. In the first case, deamination of the mutant C back to U corrects the mutation, and in the latter case, deamination of the C that is base-paired with the mutant G, followed by a round of replication, corrects the mutation. The fusion proteins comprising a Cas9 domain and one or more adenosine deaminase domains can be used to correct any single G→A or C→T point mutation. In the first case, deamination of the mutant A to I corrects the mutation, and in the latter case, deamination of the A that is base-paired with the mutant T, followed by a round of replication, corrects the mutation.

An exemplary disease-relevant mutation that can be corrected by the provided fusion proteins in vitro or in vivo is the H1047R (A3140G) polymorphism in the PI3KCA protein. The phosphoinositide-3-kinase, catalytic alpha subunit (PI3KCA) protein acts to phosphorylate the 3-OH group of the inositol ring of phosphatidylinositol. The PI3KCA gene has been found to be mutated in many different carcinomas, and thus it is considered to be a potent oncogene.⁵⁰ In fact, the A3140G mutation is present in several NCI-60 cancer cell lines, such as, for example, the HCT116, SKOV3, and T47D cell lines, which are readily available from the American Type Culture Collection (ATCC).⁵¹

In some embodiments, a cell carrying a mutation to be corrected, e.g., a cell carrying a point mutation, e.g., an A3140G point mutation in exon 20 of the PI3KCA gene, resulting in a H1047R substitution in the PI3KCA protein, is contacted with an expression construct encoding a Cas9 deaminase fusion protein and an appropriately designed sgRNA targeting the fusion protein to the respective mutation site in the encoding PI3KCA gene. Control experiments can be performed where the sgRNAs are designed to target the fusion enzymes to non-C residues that are within the PI3KCA gene. Genomic DNA of the treated cells can be extracted, and the relevant sequence of the PI3KCA genes PCR amplified and sequenced to assess the activities of the fusion proteins in human cell culture.

It will be understood that the example of correcting point mutations in PI3KCA is provided for illustration purposes and is not meant to limit the instant disclosure. The skilled artisan will understand that the instantly disclosed DNA-editing fusion proteins can be used to correct other point mutations and mutations associated with other cancers and with diseases other than cancer including other proliferative diseases.

The successful correction of point mutations in disease-associated genes and alleles opens up new strategies for gene correction with applications in therapeutics and basic research. Site-specific single-base modification systems like the disclosed fusions of Cas9 domains and deaminase domains also have applications in “reverse” gene therapy, where certain gene functions are purposely suppressed or abolished. In these cases, site-specifically mutating Trp (TGG), Gln (CAA and CAG), or Arg (CGA) residues to premature stop codons (TAA, TAG, TGA) can be used to abolish protein function in vitro, ex vivo, or in vivo.

The instant disclosure provides methods for the treatment of a subject diagnosed with a disease associated with or caused by a point mutation that can be corrected by a fusion protein comprising a Cas9 domain and nucleic acid editing domain (e.g., a deaminase domain) provided herein. For example, in some embodiments, a method is provided that comprises administering to a subject having such a disease, e.g., a cancer associated with a PI3KCA point mutation as described above, an effective amount of a Cas9 deaminase fusion protein that corrects the point mutation or introduces a deactivating mutation into the disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

The instant disclosure provides methods for the treatment of additional diseases or disorders, e.g., diseases or disorders that are associated or caused by a point mutation that can be corrected by deaminase-mediated gene editing. Some such diseases are described herein, and additional suitable diseases that can be treated with the strategies and fusion proteins provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation, cystic fibrosis (see, e.g., Schwank et al., Functional repair of CFTR by CRISPR/Cas9 in intestinal stem cell organoids of cystic fibrosis patients. Cell stem cell. 2013; 13: 653-658; and Wu et. al., Correction of a genetic disease in mouse via use of CRISPR-Cas9. Cell stem cell. 2013; 13: 659-662, neither of which uses a deaminase fusion protein to correct the genetic defect); phenylketonuria—e.g., phenylalanine to serine mutation at position 835 (mouse) or 240 (human) or a homologous residue in phenylalanine hydroxylase gene (T>C mutation)—see, e.g., McDonald et al., Genomics. 1997; 39:402-405; Bernard-Soulier syndrome (BSS)—e.g., phenylalanine to serine mutation at position 55 or a homologous residue, or cysteine to arginine at residue 24 or a homologous residue in the platelet membrane glycoprotein IX (T>C mutation)—see, e.g., Noris et al., British Journal of Haematology. 1997; 97: 312-320, and Ali et al., Hematol. 2014; 93: 381-384; epidermolytic hyperkeratosis (EHK)—e.g., leucine to proline mutation at position 160 or 161 (if counting the initiator methionine) or a homologous residue in keratin 1 (T>C mutation)—see, e.g., Chipev et al., Cell. 1992; 70: 821-828, see also accession number P04264 in the UNIPROT database at www[dot]uniprot[dot]org; chronic obstructive pulmonary disease (COPD)—e.g., leucine to proline mutation at position 54 or 55 (if counting the initiator methionine) or a homologous residue in the processed form of α₁-antitrypsin or residue 78 in the unprocessed form or a homologous residue (T>C mutation)—see, e.g., Poller et al., Genomics. 1993; 17: 740-743, see also accession number P01011 in the UNIPROT database; Charcot-Marie-Toot disease type 4J—e.g., isoleucine to threonine mutation at position 41 or a homologous residue in FIG. 4 (T>C mutation)—see, e.g., Lenk et al., PLoS Genetics. 2011; 7: e1002104; neuroblastoma (NB)—e.g., leucine to proline mutation at position 197 or a homologous residue in Caspase-9 (T>C mutation)—see, e.g., Kundu et al., 3 Biotech. 2013, 3:225-234; von Willebrand disease (vWD)—e.g., cysteine to arginine mutation at position 509 or a homologous residue in the processed form of von Willebrand factor, or at position 1272 or a homologous residue in the unprocessed form of von Willebrand factor (T>C mutation)—see, e.g., Lavergne et al., Br. J. Haematol. 1992, see also accession number P04275 in the UNIPROT database; 82: 66-72; myotonia congenital—e.g., cysteine to arginine mutation at position 277 or a homologous residue in the muscle chloride channel gene CLCN1 (T>C mutation)—see, e.g., Weinberger et al., The J. of Physiology. 2012; 590: 3449-3464; hereditary renal amyloidosis—e.g., stop codon to arginine mutation at position 78 or a homologous residue in the processed form of apolipoprotein AII or at position 101 or a homologous residue in the unprocessed form (T>C mutation)—see, e.g., Yazaki et al., Kidney Int. 2003; 64: 11-16; dilated cardiomyopathy (DCM)—e.g., tryptophan to Arginine mutation at position 148 or a homologous residue in the FOXD4 gene (T>C mutation), see, e.g., Minoretti et. al., Int. J. of Mol. Med. 2007; 19: 369-372; hereditary lymphedema—e.g., histidine to arginine mutation at position 1035 or a homologous residue in VEGFR3 tyrosine kinase (A>G mutation), see, e.g., Irrthum et al., Am. J. Hum. Genet. 2000; 67: 295-301; familial Alzheimer's disease—e.g., isoleucine to valine mutation at position 143 or a homologous residue in presenilin1 (A>G mutation), see, e.g., Gallo et. al., J. Alzheimer's disease. 2011; 25: 425-431; Prion disease—e.g., methionine to valine mutation at position 129 or a homologous residue in prion protein (A>G mutation)—see, e.g., Lewis et. al., J. of General Virology. 2006; 87: 2443-2449; chronic infantile neurologic cutaneous articular syndrome (CINCA)—e.g., Tyrosine to Cysteine mutation at position 570 or a homologous residue in cryopyrin (A>G mutation)—see, e.g., Fujisawa et. al. Blood. 2007; 109: 2903-2911; and desmin-related myopathy (DRM)—e.g., arginine to glycine mutation at position 120 or a homologous residue in αB crystallin (A>G mutation)—see, e.g., Kumar et al., J. Biol. Chem. 1999; 274: 24137-24141. The entire contents of all references and database entries is incorporated herein by reference.

The instant disclosure provides lists of genes comprising pathogenic T>C or A>G mutations, which may be corrected using a fusion protein comprising a Cas9 domain and a cytidine deaminase domain provided herein. Provided herein, are the names of these genes, their respective SEQ ID NOs, their gene IDs, and sequences flanking the mutation site. See Table 5 and Table 18. Without wishing to be bound by any particular theory, the mutations provided in Table 5 and Table 18 may be corrected using the Cas9 fusions provided herein, which are able to bind to target sequences lacking the canonical PAM sequence. In some embodiments, a Cas9-deaminase fusion protein demostrates activity on non-canonical PAMs and therefore can correct all the pathogenic T>C or A>G mutations listed in Table 5 and Table 18 (SEQ ID NOs: 674-2539 and 3144-5083), respectively. In some embodiments, a Cas9-deaminase fusion protein recognizes canonical PAMs and therefore can correct the pathogenic T>C or A>G mutations with canonical PAMs, e.g., 5′-NGG-3′. It should be appreciated that a skilled artisan would understand how to design an RNA (e.g., a gRNA) to target any of the Cas9 domains or fusion proteins provided herein to any target sequence in order to correct any of the mutations provided herein, for example, the mutations provided in Table 5 or Table 18. It will be apparent to those of skill in the art that in order to target a Cas9:effector domain fusion protein as disclosed herein to a target site, e.g., a site comprising a point mutation to be edited, it is typically necessary to co-express the Cas9:effector domain fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:effector domain fusion protein. In some embodiments, the guide RNA comprises a structure 5′-[guide sequence]-guuuuagagcuagaaauagcaaguuaaaauaaggcuaguccguuaucaacuugaaaaaguggcaccgagucggugcuuu uu-3′ (SEQ ID NO: 285), wherein the guide sequence comprises a sequence that is complementary to the target sequence. The guide sequence is typically 20 nucleotides long. The sequences of suitable guide RNAs for targeting Cas9:effector domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Exemplary guide RNA structures, including guide RNA backbone sequences, are described, for example, in Jinek M, et al. (2012) A programmable dual-RNA-guided DNA endonuclease in adaptive bacterial immunity. Science, 337, 816-812; Mali P, et al. (2013) Cas9 as a versatile tool for engineering biology. Nature Methods, 10, 957-963; Li J F, et al. (2013) Multiplex and homologous recombination-mediated genome editing in Arabidopsis and Nicotiana benthamiana using guide RNA and Cas9. Nature Biotech, 31, 688-691; Hwang W Y, et al. (2013) Efficient in vivo genome editing using RNA-guided nucleases. Nat Biotechnol, 31, 227-229; Cong L, et al. (2013) Multiplex genome engineering using CRIPSR/Cas systems. Science, 339, 819-823; Cho S W, et al. (2013) Targeted genome engineering in human cells with the Cas9 RNA-guided endonuclease. Nat Biotechnol, 31, 230-232; Jinek M J, et al. (2013) RNA-programmed genome editing in human cells. eLIFE, 2:e00471; DiCarlo J E, et al. (2013) Genome engineering in Saccharomyces cerevisiae using CRISPR-Cas systems. Nucl Acids Res, 41, 4336-4343; Qi L S, et al. (2013) Repruposing CRISPR as an RNA-Guided Platform for Sequence-Specific Control of Gene Expression. Cell, 152, 1173-1183; and Briner A E, et al. (2014) Guide RNA functional modules direct Cas9 activity and orthogonality. Mol Cell, 56, 333-339; each of which is incorporated herein by reference.

### Pharmaceutical Compositions

Other aspects of the present disclosure relate to pharmaceutical compositions comprising any of the Cas9 domains, fusion proteins, or the fusion protein-gRNA complexes described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a composition of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Kits, Vectors, Cells

Some aspects of this disclosure provide kits comprising a nucleic acid construct, comprising (a) a nucleotide sequence encoding a Cas9 domain or a fusion protein comprising a Cas9 domain as provided herein; and (b) a heterologous promoter that drives expression of the sequence of (a). In some embodiments, the kit further comprises an expression construct encoding a guide RNA backbone, wherein the construct comprises a cloning site positioned to allow the cloning of a nucleic acid sequence identical or complementary to a target sequence into the guide RNA backbone.

Some aspects of this disclosure provide polynucleotides encoding a Cas9 domain or a fusion protein comprising a Cas9 domain as provided herein. Some aspects of this disclosure provide vectors comprising such polynucleotides. In some embodiments, the vector comprises a heterologous promoter driving expression of polynucleotide.

In one aspect, provided herein are methods comprising contacting a cell with a kit provided herein. In another aspect, provided herein are methods comprising contacting a cell with a vector provided herein. In some embodiments, the vector is transfected into the cell. In some embodiments, the vector is transfected into the cell using a suitable transfection reaction. Transfection reactions may be carried out, for example, using electroporation, heat shock, or a composition comprising a catioinic lipid. Cationic lipids suitable for the transfection of nucleic acid molecules are provided in, for example, Patent Publication WO2015/035136, published Mar. 12, 2015, entitled “Delivery System for Functional Nucleases”; the entire contents of which is incorporated by reference herein.

Some aspects of this disclosure provide cells comprising a Cas9 domain, a fusion protein, a nucleic acid molecule, and/or a vector as provided herein.

The description of exemplary embodiments of the reporter systems (e.g., GFP) herein is provided for illustration purposes only and not meant to be limiting. Additional reporter systems, e.g., variations of the exemplary systems described in detail above, are also embraced by this disclosure.

### EXAMPLES

In order that the invention described herein may be more fully understood, the following examples are set forth. The synthetic examples described in this application are offered to illustrate the compounds and methods provided herein and are not to be construed in any way as limiting their scope.

### Example 1: Cas9 Variants without PAM Restrictions

The beneficial mutations conferring Cas9 activity on noncanonical PAM sequences were mapped to a S. pyogenes wild-type sequence. Below is an exemplary Cas9 sequence (S. pyogenes Cas9 with D10 and H840 residues marked with an asterisk following the respective amino acid residues, SEQ ID NO: 9). The D10 and H840 residues of SEQ ID NO: 9 may be mutated to generate a nuclease inactive Cas9 (e.g., to D10A and H840A) or to generate a nickase Cas9 (e.g., to D10A with H840; or to D10 with H840A). The HNH domain (bold and underlined) and the RuvC domain (boxed) are identified. The residues found mutated in the clones isolated from the various PACE experiments, amino acid residues 122, 137, 182, 262, 294, 409, 480, 543, 660, 694, 1219, and 1329 are identified with an asterisk following the respective amino acid residue.

(SEQ ID NO:9)
MDKKYSIGLD*IGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNI*VDEVAYHEKYPTIYH*LRKKLVDSTD
KADLRLIYLALAHMIKFRGHFLIEGDLNPDNSDVD*KLFIQLVQTYNQLFEENPINASGVDAKAILSARLSKSRR
LENLIAQLPGEKKNGLFGNLIALSLGLTPNFKSNFDLAEDA*KLQLSKDTYDDDLDNLLAQIGDQYADLFLAAK*
NLSDAILLSDILRVNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGS*IPHQIHLGELHAILRRQEDFYPFLKDNREKIEKI
LTFRIPYYVGPLARGNSRFAWMTRKSEETITPWNFEE*VVDKGASAQSFIERMTNFDKNLPNEKVLPKHSLLYEY
FTVYNELTKVKYVTEGMRKPAFLSGE*QKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQLKRRRYTGWG*RLSRK

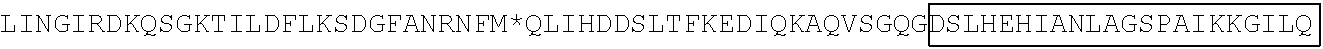

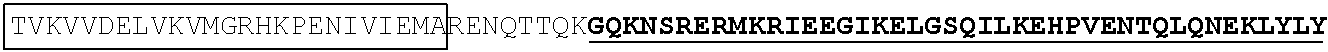

YLQNGRDMYVDQELDINRLSDYDVDH*IVPQSFLKDDSIDNKVLTRSDKNRGKSDNVPSEEVVKKMKNYWRQLLN

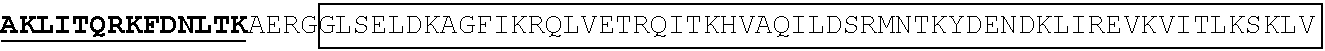

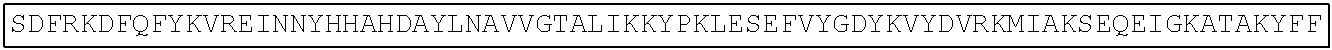

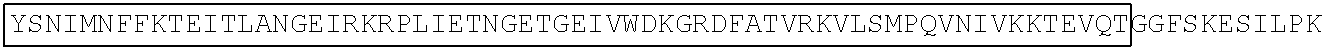

RNSDKLIARKKDWDPKKYGGFDSPTVAYSVLVVAKVEKGKSKKLKSVKELLGITIMERSSFEKNPIDFLEAKGYK
EVKKDLIIKLPKYSLFELENGRKRMLASAGE*LQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPAAFKYFDT*TIDRKRY
TSTKEVLDATLIHQSITGLYETRIDLSQLGGD

The beneficial mutations conferring as activity on noncanonical PAM sequences were mapped to additional exemplary wild-type Cas9 sequences. The HNH domain (bold and underlined) and the RuvC domain (boxed) are identified. The residues homologous to the residues found mutated in SEQ ID NO: 9, amino acid residues 122, 137, 182, 262, 294, 409, 480, 543, 660, 694, 1219, and 1329 are identified with an asterisk following the respective amino acid residue. In addition, amino acid residues 10 and 840, which are mutated in dCas9 domain variants, are also identified by an asterisk.

(SEQ ID NO: 262)
MDKKYSIGLD*IGTNSVGWAVITDDYKVPSKKFKVLGNTDRHSIKKNLIGALLFGSGETAEATRLKRTARRRYTR
RKNRICYLQEIFSNEMAKVDDSFFHRLEESFLVEEDKKHERHPIFGNI*VDEVAYHEKYPTIYH*LRKKLADSTD
KADLRLIYLALAHMIKFRGHFLIEGDLNPDNSDVD*KLFIQLVQIYNQLFEENPINASRVDAKAILSARLSKSRR
LENLIAQLPGEKRNGLFGNLIALSLGLTPNFKSNFDLAEDA*KLQLSKDTYDDDLDNLLAQIGDQYADLFLAAK*
NLSDAILLSDILRVNSEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGS*IPHQIHLGELHAILRRQEDFYPFLKDNREKIEKI
LTFRIPYYVGPLARGNSRFAWMTRKSEETITPWNFEE*VVDKGASAQSFIERMTNFDKNLPNEKVLPKHSLLYEY
FTVYNELTKVKYVTEGMRKPAFLSGE*QKKAIVDLLFKTNRKVTVKQLKEDYFKKIECFDSVEISGVEDRFNASL
GAYHDLLKIIKDKDFLDNEENEDILEDIVLTLTLFEDRGMIEERLKTYAHLFDDKVMKQLKRRRYTGWG*RLSRK

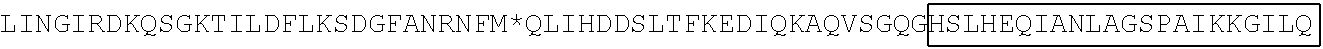

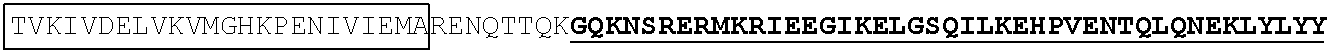

LQNGRDMYVDQELDINRLSDYDVDH*IVPQSFIKDDSIDNKVLIRSDKNRGKSDNVPSEEVVKKMKNYWRQLLNA

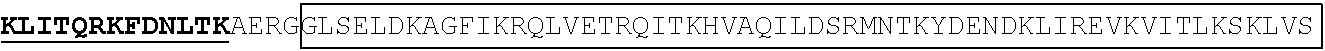

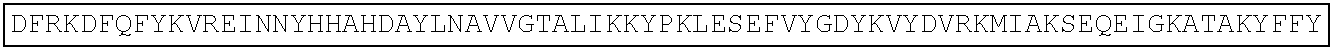

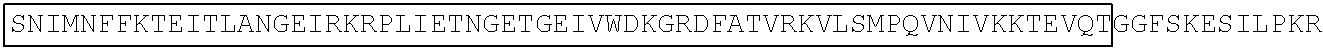

NSDKLIARKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGE*LQKGNELALPSKYVNFLYLASHYEKLKGSPEDNEQKQLFVEQHK
HYLDEIIEQISEFSKRVILADANLDKVLSAYNKHRDKPIREQAENIIHLFTLTNLGAPAAFKYFDT*TIDRKRYT
STKEVLDATLIHQSITGLYETRIDLSQLGGD
(SEQ ID NO: 263)
MDKKYSIGLA*IGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNI*VDEVAYHEKYPTIYH*LRKKLVDSTD
KADLRLIYLALAHMIKFRGHFLIEGDLNPDNSDVD*KLFIQLVQTYNQLFEENPINASGVDAKAILSARLSKSRR
LENLIAQLPGEKKNGLFGNLIALSLGLTPNFKSNFDLAEDA*KLQLSKDTYDDDLDNLLAQIGDQYADLFLAAK*
NLSDAILLSDILRVNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGS*IPHQIHLGELHAILRRQEDFYPFLKDNREKIEKI
LTFRIPYYVGPLARGNSRFAWMTRKSEETITPWNFEE*VVDKGASAQSFIERMTNFDKNLPNEKVLPKHSLLYEY
FTVYNELTKVKYVTEGMRKPAFLSGE*QKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLFDDKVMKQLKRRRYTGWG*RLSRK

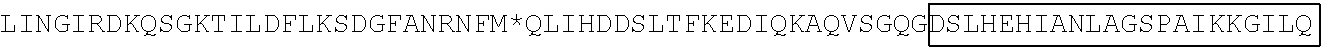

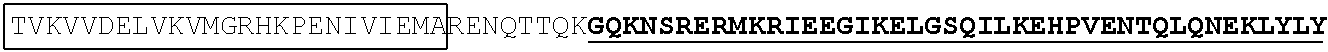

YLQNGRDMYVDQELDINRLSDYDVDH*IVPQSFLKDDSIDNKVLIRSDKNRGKSDNVPSEEVVKKMKNYWRQLLN

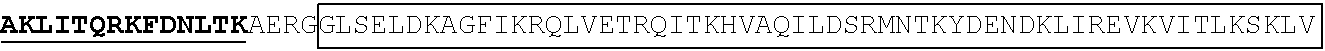

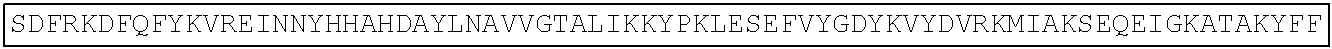

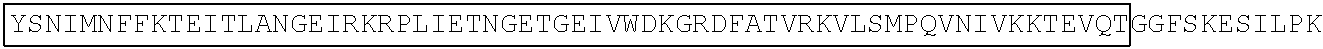

RNSDKLIARKKDWDPKKYGGFDSPTVAYSVLVVAKVEKGKSKKLKSVKELLGITIMERSSFEKNPIDFLEAKGYK
EVKKDLIIKLPKYSLFELENGRKRMLASAGE*LQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPAAFKYFDT*TIDRKRY
TSTKEVLDATLIHQSITGLYETRIDLSQLGGD
>gi|504540549|ref|WP_014727651.1| type II CRISPR RNA-guided endonuclease
Cas9 [Streptococcus thermophilus]
(SEQ ID NO: 260)
MTKPYSIGLD*IGTNSVGWAVITDNYKVPSKKMKVLGNTSKKYIKKNLLGVLLFDSGITAEGRRLKRTARRRYTR
RRNRILYLQEIFSTEMATLDDAFFQRLDDSFLVPDDKRDSKYPIFGNL*VEEKAYHDEFPTIYH*LRKYLADSTK
KADLRLVYLALAHMIKYRGHFLIEGEFNSKNNDIQ*KNFQDFLDTYNAIFESDLSLENSKQLEEIVKDKISKLEK
KDRILKLFPGEKNSGIFSEFLKLIVGNQADFRKCFNLDEKA*SLHFSKESYDEDLETLLGYIGDDYSDVFLKAK*
KLYDAILLSGFLTVTDNETEAPLSSAMIKRYNEHKEDLALLKEYIRNISLKTYNEVFKDDTKNGYAGYIDGKTNQ
EDFYVYLKNLLAEFEGADYFLEKIDREDFLRKQRTFDNGS*IPYQIHLQEMRAILDKQAKFYPFLAKNKERIEKI
LTFRIPYYVGPLARGNSDFAWSIRKRNEKITPWNFED*VIDKESSAEAFINRMTSFDLYLPEEKVLPKHSLLYET
FNVYNELTKVRFIAESMRDYQFLDSK*QKKDIVRLYFKDKRKVTDKDIIEYLHAIYGYDGIELKGIEKQFNSSLS
TYHDLLNIINDKEFLDDSSNEAIIEEIIHTLTIFEDREMIKQRLSKFENIFDKSVLKKLSRRHYTGWG*KLSAKL

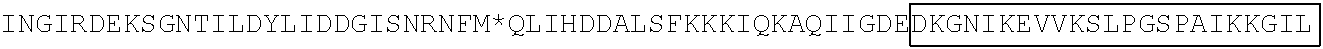

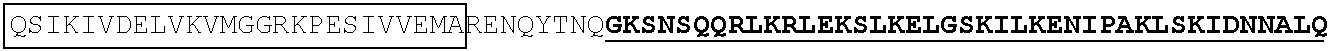

NDRLYLYYLQNGKDMYTGDDLDIDRLSNYDIDH*IIPQAFLKDNSIDNKVINSSASNRGKSDDFPSLEVVKKRKT

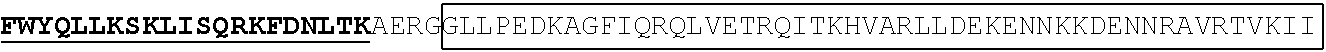

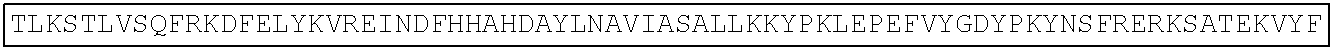

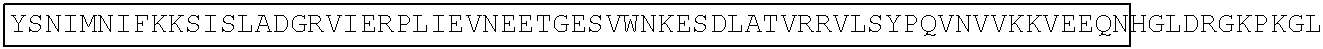

FNANLSSKPKPNSNENLVGAKEYLDPKKYGGYAGISNSFAVLVKGTIEKGAKKKITNVLEFQGISILDRINYRKD
KLNFLLEKGYKDIELIIELPKYSLFELSDGSRRMLASILSTNNKRGE*IHKGNQIFLSQKFVKLLYHAKRISNTI
NENHRKYVENHKKEFEELFYYILEFNENYVGAKKNGKLLNSAFQSWQNHSIDELCSSFIGPTGSERKGLFELTSR
GSAADFEFLGV*KIPRYRDYTPSSLLKDATLIHQSVTGLYETRIDLAKLGEG
>gi 924443546 | Staphylococcus Aureus Cas9
(SEQ ID NO: 261)
GSHMKRNYILGLD*IGITSVGYGIIDYETRDVIDAGVRLFKEANVENNEGRRSKRGARRLKRRRRHRIQRVKKLL
FDYNLLTDHSELSGINP*YEARVKGLSQKLSEEEFSAALLHLAKRRGVHNVNEVEEDTGNELST*KEQISRNSKA
LEEKYVAELQLERLKKDGEVRGSINRFKTSDYVKEAKQLLKVQKAYHQLDQSFIDTYIDLLETRRTYYEGPGEGS
PFGWKDIKEWYEMLMGHCTYFPEELRSVKYAYNADLYNALNDLNNLVITRDENEKLEYY*EKFQIIENVFKQKKK
PTLKQIAKEILVNEEDIKGYRVTSTGKPEFTNLKVYHDIKDITARKEIIENAELLDQIAKILTIYQSSEDIQEEL

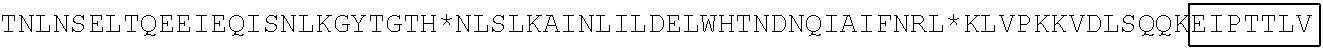

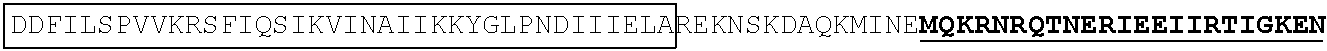

AKYLIEKIKLHDMQEGKCLYSLEAIPLEDLLNNPFNYEVDH*IIPRSVSFDNSFNNKVINKQEEASKKGNRTPFQ

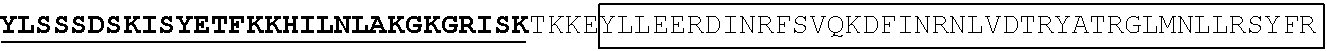

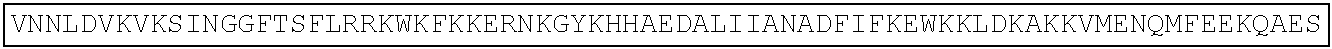

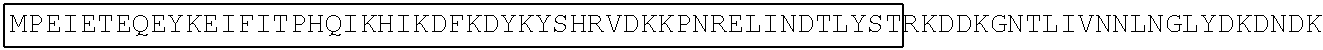

LKKLINKSPEKLLMYHHDPQTYQKLKLIMEQYGDEKNPLYKYYEETGNYLTKYSKKDNGPVIKKIKYYGNKLNAH
LDITDDYPNS*RNKVVKLSLKPYRFDVYLDNGVYKFVTVKNLDVIKKENYYEVNSKAYEEAKKLKKISNQAEFIA
SFYNNDLIKINGELYRVIGVNNDLLNRIEVNMIDITYREYLEN*MNDKRPPRIIKTIASKTQSIKKYSTDILGNL
YEVKSKKHPQIIKKG

This disclosure provides Cas9 variants in which one or more of the amino acid residues identified by an asterisk are mutated as described herein. In some embodiments, the D10 and H840 residues are mutated, e.g., to an alanine residue, and the Cas9 variants provided include one or more additional mutations of the amino acid residues identified by an asterisk as provided herein. In some embodiments, the D10 residue is mutated, e.g., to an alanine residue, and the Cas9 variants provided include one or more additional mutations of the amino acid residues identified by an asterisk as provided herein.

A number of Cas9 sequences from various species were aligned to determine whether corresponding homologous amino acid residues can be identified in other Cas9 domains, allowing the generation of Cas9 variants with corresponding mutations of the homologous amino acid residues. The alignment was carried out using the NCBI Constraint-based Multiple Alignment Tool (COBALT(accessible at st-va.ncbi.nlm.nih.gov/tools/cobalt), with the following parameters. Alignment parameters: Gap penalties −11,−1; End-Gap penalties −5,−1. CDD Parameters: Use RPS BLAST on; Blast E-value 0.003; Find Conserved columns and Recompute on. Query Clustering Parameters: Use query clusters on; Word Size 4; Max cluster distance 0.8; Alphabet Regular.

An exemplary alignment of four Cas9 sequences is provided below. The Cas9 sequences in the alignment are: Sequence 1 (S1): SEQ ID NO: 10|WP_010922251|gi 499224711|type II CRISPR RNA-guided endonuclease Cas9 [Streptococcus pyogenes]; Sequence 2 (S2): SEQ ID NO: 11|WP_039695303|gi 746743737|type II CRISPR RNA-guided endonuclease Cas9 [Streptococcus gallolyticus]; Sequence 3 (S3): SEQ ID NO: 12|WP_045635197|gi 782887988|type II CRISPR RNA-guided endonuclease Cas9 [Streptococcus mitis]; Sequence 4 (S4): SEQ ID NO: 13|5AXW_A|gi 924443546|Staphylococcus Aureus Cas9. The HNH domain (bold and underlined) and the RuvC domain (boxed) are identified for each of the four sequences. Amino acid residues 10, 122, 137, 182, 262, 294, 409, 480, 543, 660, 694, 840, 1219, and 1329 in S1 and the homologous amino acids in the aligned sequences are identified with an asterisk following the respective amino acid residue. A similar approach can be employed to determine homologous amino acid residues suitable for mutation based on the amino acid mutations of Cas9 domains identified herein.

| | | | |
|---|---|---|---|
| S1 | 1 | --MDKK-YSIGLD*IGTNSVGWAVITDEYKVPSKKFKVLGNTDRHSIKKNLI--GALLFDSG--ETAEATRLKRTARRRYT | 73 |
| S2 | 1 | --MTKKNYSIGLD*IGTNSVGWAVITDDYKVPAKKMKVLGNTDKKYIKKNLL--GALLFDSG--ETAEATRLKRTARRRYT | 74 |
| S3 | 1 | --M-KKGYSIGLD*IGTNSVGFAVITDDYKVPSKKMKVLGNTDKRFIKKNLI--GALLFDEG--TTAEARRLKRTARRRYT | 73 |
| S4 | 1 | GSHMKRNYILGLD*IGITSVGYGII--DYET-----------------RDVIDAGVRLFKEANVENNEGRRSKRGARRLKR | 61 |
| S1 | 74 | RRKNRICYLQEIFSNEMAKVDDSFFHRLEESFLVEEDKKHERHPIFGNI*VDEVAYHEKYPTIYH*LRKKLVDSTDKADLRL | 153 |
| S2 | 75 | RRKNRLRYLQEIFANEIAKVDESFFQRLDESFLTDDDKTFDSHPIFGNK*AEEDAYHQKFPTIYH*LRKHLADSSEKADLRL | 154 |
| S3 | 74 | RRKNRLRYLQEIFSEEMSKVDSSFFHRLDDSFLIPEDKRESKYPIFATL*TEEKEYHKQFPTIYH*LRKQLADSKEKTDLRL | 153 |
| S4 | 62 | RRRHRIQRVKKLL--------------FDYNLLTD---------------------HSELSGINP*YEARVKGLSQKLSEEE | 107 |
| S1 | 154 | IYLALAHMIKFRGHFLIEGDLNPDNSDVD*KLFIQLVQTYNQLFEENPINASGVDAKAILSARLSKSRRLENLIAQLPGEK | 233 |
| S2 | 155 | VYLALAHMIKFRGHFLIEGELNAENTDVQ*KIFADFVGVYNRTFDDSHLSEITVDVASILTEKISKSRRLENLIKYYPTEK | 234 |
| S3 | 154 | IYLALAHMIKYRGHFLYEEAFDIKNNDIQ*KIFNEFISIYDNTFEGSSLSGQNAQVEAIFTDKISKSAKRERVLKLFPDEK | 233 |
| S4 | 108 | FSAALLHLAKRRG-----------------------VHNVNEVEEDT---------------------------------- | 131 |
| S1 | 234 | KNGLFGNLIALSLGLTPNFKSNFDLAEDA*KLQLSKDTYDDDLDNLLAQIGDQYADLFLAAK*NLSDAILLSDILRVNTEIT | 313 |
| S2 | 235 | KNTLFGNLIALALGLQPNFKTNFKLSEDA*KLQFSKDTYEEDLEELLGKIGDDYADLFTSAK*NLYDAILLSGILTVDDNST | 314 |
| S3 | 234 | STGLFSEFLKLIVGNQADFKKHFDLEDKA*PLQFSKDTYDEDLENLLGQIGDDFTDLFVSAK*KLYDAILLSGILTVTDPST | 313 |
| S4 | 132 | -----GNELS------------------T*KEQISRN--------------------------------------------- | 144 |
| S1 | 314 | KAPLSASMIKRYDEHHQDLTLLKALVRQQLPEKYKEIFFDQSKNGYAGYIDGGASQEEFYKFIKPILEKM--DGTEELLV | 391 |
| S2 | 315 | KAPLSASMIKRYVEHHEDLEKLKEFIKANKSELYHDIFKDKNKNGYAGYIENGVKQDEFYKYLKNILSKIKIDGSDYFLD | 394 |
| S3 | 314 | KAPLSASMIERYENHQNDLAALKQFIKNNLPEKYDEVFSDQSKDGYAGYIDGKTTQETFYKYIKNLLSKF--EGTDYFLD | 391 |
| S4 | 145 | ----SKALEEKYVAELQ-------------------------------------------------LERLKKDG------ | 165 |
| S1 | 392 | KLNREDLLRKQRTFDNGS*IPHQIHLGELHAILRRQEDFYPFLKDNREKIEKILTFRIPYYVGPLARGNSRFAWMTRKSEE | 471 |
| S2 | 395 | KIEREDFLRKQRTFDNGS*IPHQIHLQEMHAILRRQGDYYPFLKEKQDRIEKILTFRIPYYVGPLVRKDSRFAWAEYRSDE | 474 |
| S3 | 392 | KIEREDFLRKQRTFDNGS*IPHQIHLQEMNAILRRQGEYYPFLKDNKEKIEKILTFRIPYYVGPLARGNRDFAWLTRNSDE | 471 |
| S4 | 166 | --EVRGSINRFKTSD---------YVKEAKQLLKVQKAYHQLDQSFIDTYIDLLETRRTYYEGP--GEGSPFGW------K | 227 |
| S1 | 472 | TITPWNFEE*VVDKGASAQSFIERMTNFDKNLPNEKVLPKHSLLYEYFTVYNELTKVKYVTEGMRKPAFLSGE*QKKAIVDL | 551 |
| S2 | 475 | KITPWNFDK*VIDKEKSAEKFITRMTLNDLYLPEEKVLPKHSHVYETYAVYNELTKIKYVNEQGKE-SFFDSN*MKQEIFDH | 553 |
| S3 | 472 | AIRPWNFEE*IVDKASSAEDFINKMTNYDLYLPEEKVLPKHSLLYETFAVYNELTKVKFIAEGLRDYQFLDSG*QKKQIVNQ | 551 |
| S4 | 228 | DIKEW----------------YEMLMGHCTYFPEELRSVKYAYNADLYNALNDLNNLVITRDENEK---LEYY*EKFQIIEN | 289 |
| S1 | 552 | LFKTNRKVTVKQLKEDYFKKIECFDSVEISGVEDR---FNASLGTYHDLLKIIKDKDFLDNEENEDILEDIVLTLTLFED | 628 |
| S2 | 554 | VFKENRKVTKEKLLNYLNKEFFEYRIKDLIGLDKENKSFNASLGTYHDLKKIL-DKAFLDDKVNEEVIEDIIKTLTLFED | 632 |
| S3 | 552 | LFKENRKVTEKDIIHYLHN-VDGYDGIELKGIEKQ---FNASLSTYHDLLKIIKDKEFMDDAKNEAILENIVHTLTIFED | 627 |
| S4 | 290 | VFKQKKKPTLKQIAKEILVNEEDIKGYRVTSTGKPEF---TNLKVYHDIKDITARKEII---ENAELLDQIAKILTIYQS | 363 |
| 51 | 629 | REMIEERLKTYAHLFDDKVMKQLKR-RRYTGWG*RLSRKLINGIRDKQSGKTILDFLKSDGFANRNFM*QLIHDDSLTFKED | 707 |
| S2 | 633 | KDMIHERLQKYSDIFTANQLKKLER-RHYTGWG*RLSYKLINGIRNKENNKTILDYLIDDGSANRNFM*QLINDDTLPFKQI | 711 |
| S3 | 628 | REMIKQRLAQYDSLFDEKVIKALTR-RHYTGWG*KLSAKLINGICDKQTGNTILDYLIDDGKINRNFM*QLINDDGLSFKEI | 706 |
| S4 | 364 | SEDIQEELTNLNSELTQEEIEQISNLKGYTGTH*NLSLKAINLILDE------LWHTNDNQIAIFNRL*KLVP--------- | 428 |
| 51 | 708 | 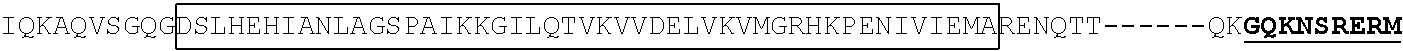 | 781 |
| S2 | 712 | 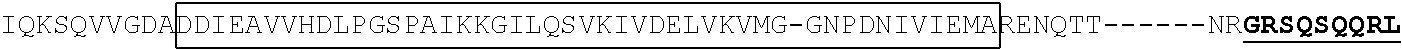 | 784 |
| S3 | 707 | 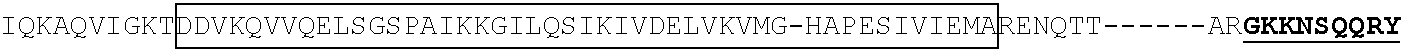 | 779 |
| S4 | 429 | 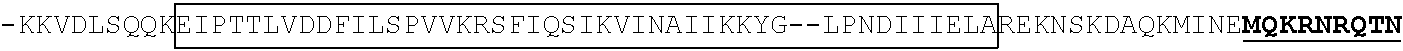 | 505 |
| 51 | 782 | KRIEEGIKELGSQIL-------KEHPVENTQLQNEKLYLYYLQNGRDMYVDQELDINRLSD----YDVDH*IVPQSFLKDD | 850 |
| S2 | 785 | KKLQNSLKELGSNILNEEKPSYIEDKVENSHLQNDQLFLYYIQNGKDMYTGDELDIDHLSD----YDIDH*IIPQAFIKDD | 860 |
| S3 | 780 | KRIEDSLKILASGL---DSNILKENPTDNNQLQNDRLFLYYLQNGKDMYTGEALDINQLSS----YDIDH*IIPQAFIKDD | 852 |
| S4 | 506 | ERIEEIIRTTGK---------------ENAKYLIEKIKLHDMQEGKCLYSLEAIPLEDLLNNPFNYEVDH*IIPRSVSFDN | 570 |
| 51 | 851 | 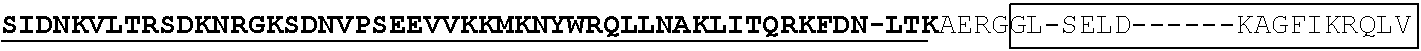 | 922 |
| S2 | 861 | 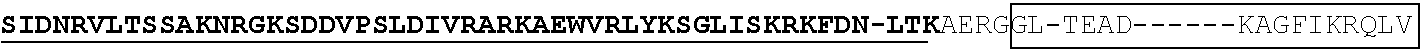 | 932 |
| S3 | 853 | 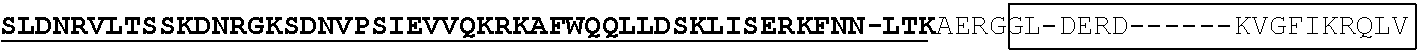 | 924 |
| S4 | 571 | 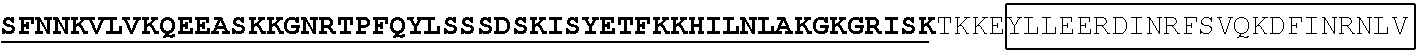 | 650 |
| 51 | 923 | 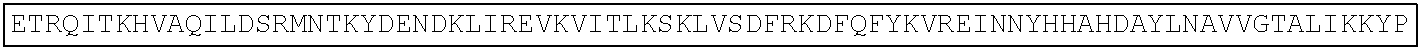 | 1002 |
| S2 | 933 | 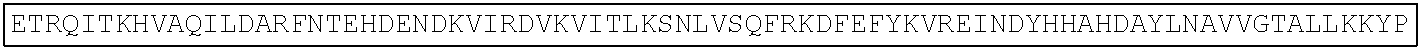 | 1012 |
| S3 | 925 | 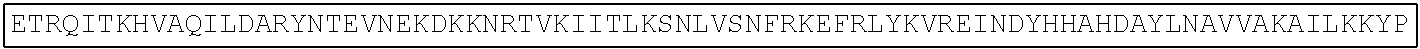 | 1004 |
| S4 | 651 | 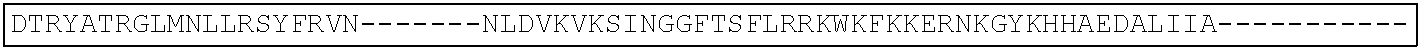 | 712 |
| 51 | 1003 | 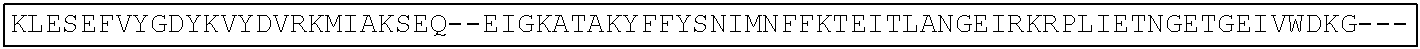 | 1077 |
| S2 | 1013 | 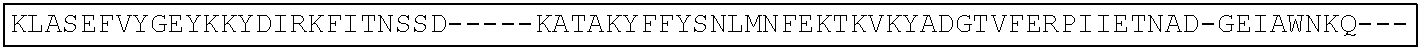 | 1083 |
| S3 | 1005 | 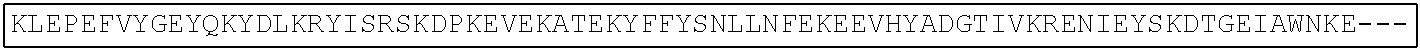 | 1081 |
| S4 | 713 | 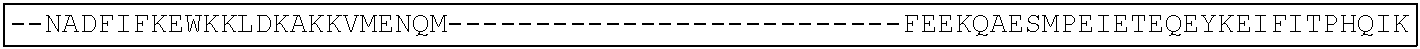 | 764 |
| 51 | 1078 | 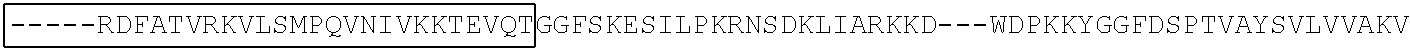 | 1149 |
| S2 | 1084 | 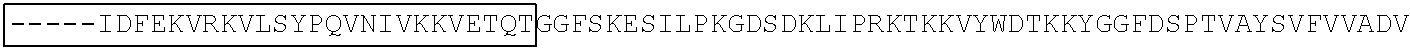 | 1158 |
| S3 | 1082 | 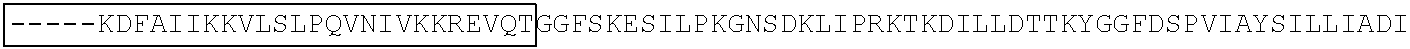 | 1156 |
| S4 | 765 | 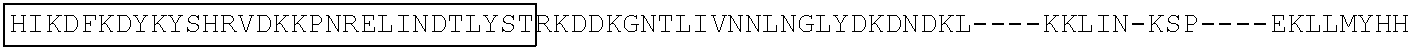 | 835 |
| 51 | 1150 | EKGKSKKLKSVKELLGITIMERSSFEKNPI-DFLEAKG-----YKEVKKDLIIKLPKYSLFELENGRKRMLASAGE*LQKG | 1223 |
| S2 | 1159 | EKGKAKKLKTVKELVGISIMERSFFEENPV-EFLENKG-----YHNIREDKLIKLPKYSLFEFEGGRRRLLASASE*LQKG | 1232 |
| S3 | 1157 | EKGKAKKLKTVKTLVGITIMEKAAFEENPI-TFLENKG-----YHNVRKENILCLPKYSLFELENGRRRLLASAKE*LQKG | 1230 |
| S4 | 836 | DPQTYQKLK--------LIMEQYGDEKNPLYKYYEETGNYLTKYSKKDNGPVIKKIKYYGNKLNAHLDITDDYPNS*RNKV | 907 |
| S1 | 1224 | NELALPSKYVNFLYLASHYEKLKGSPEDNEQKQLFVEQHKHYLDEIIEQISEFSKRVILADANLDKVLSAYNKH------ | 1297 |
| S2 | 1233 | NEMVLPGYLVELLYHAHRADNF-----NSTEYLNYVSEHKKEFEKVLSCVEDFANLYVDVEKNLSKIRAVADSM------ | 1301 |
| S3 | 1231 | NEIVLPVYLTTLLYHSKNVHKL-----DEPGHLEYIQKHRNEFKDLLNLVSEFSQKYVLADANLEKIKSLYADN------ | 1299 |
| S4 | 908 | VKLSLKPYRFD-VYLDNGVYKFV-----TVKNLDVIK--KENYYEVNSKAYEEAKKLKKISNQAEFIASFYNNDLIKING | 979 |
| 51 | 1298 | RDKPIREQAENIIHLFTLTNLGAPAAFKYFDT*TIDRKRYTSTKEVLDATLIHQSIT--------GLYETRI----DLSQL | 1365 |
| S2 | 1302 | DNFSIEEISNSFINLLTLTALGAPADFNFLGE*KIPRKRYTSTKECLNATLIHQSIT--------GLYETRI----DLSKL | 1369 |
| S3 | 1300 | EQADIEILANSFINLLTFTALGAPAAFKFFGK*DIDRKRYTTVSEILNATLIHQSIT--------GLYETWI----DLSKL | 1367 |
| S4 | 980 | ELYRVIGVNNDLLNRIEVNMIDITYR-EYLEN*MNDKRPPRIIKTIASKT---QSIKKYSTDILGNLYEVKSKKHPQIIKK | 1055 |
| 51 | 1366 | GGD | 1368 |
| S2 | 1370 | GEE | 1372 |
| S3 | 1368 | GED | 1370 |
| S4 | 1056 | G-- | 1056 |

The alignment demonstrates that amino acid sequences and amino acid residues that are homologous to a reference Cas9 amino acid sequence or amino acid residue can be identified across Cas9 sequence variants, including, but not limited to Cas9 sequences from different species, by identifying the amino acid sequence or residue that aligns with the reference sequence or the reference residue using alignment programs and algorithms known in the art. This disclosure provides Cas9 variants in which one or more of the amino acid residues identified by an asterisk in SEQ ID NO: 9 are mutated as described herein. The residues in Cas9 sequences other than SEQ ID NO: 9 that correspond to the residues identified in SEQ ID NO: 9 by an asterisk are referred to herein as “homologous” or “corresponding” residues. Such homologous residues can be identified by sequence alignment, e.g., as described above, and by identifying the sequence or residue that aligns with the reference sequence or residue. Similarly, mutations in Cas9 sequences other than SEQ ID NO: 9 that correspond to mutations identified in SEQ ID NO: 9 herein, e.g., mutations of residues 10, 122, 137, 182, 262, 294, 409, 480, 543, 660, 694, 840, 1219, and 1329 in SEQ ID NO: 9, are referred to herein as “homologous” or “corresponding” mutations. For example, the mutations corresponding to the D10A mutation in S1 for the four aligned sequences above are D10A for S2, D9A for S3, and D13A for S4; the corresponding mutations for H840A in S1 are H850A for S2, H842A for S3, and H560 for S4; the corresponding mutation for X1219V in S1 are X1228V for S2, X1226 for S3, and X903V for S4, and so on.

A total of 250 Cas9 sequences (SEQ ID NOs: 10-262) from different species were aligned using the same algorithm and alignment parameters outlined above, and is provided in Patent Publication No. WO2017/070633, published Apr. 27, 2017, entitled “Evolved Cas9 domains For Gene Editing”; the entire contents of which are incorporated herein by reference. Additional suitable Cas9 homologues, as well as performing alignments of homologues, will be apparent to those of ordinary skill in the art based on this disclosure and knowledge in the field, and are within the scope of the present disclosure.

Cas9 variants with one or more mutations in amino acid residues homologous to amino acid residues 122, 137, 182, 262, 294, 409, 480, 543, 660, 694, 1219, and 1329 of SEQ ID NO: 9 are provided herein. In some embodiments, the Cas9 variants provided herein comprise mutations corresponding to the D10A and the H840A mutations in SEQ ID NO: 9, resulting in a nuclease-inactive dCas9, and at least one, at least two, at least three, at least four, at least five, at least six, or at least seven mutations of amino acid residues homologous to amino acid residues 122, 137, 182, 262, 294, 409, 480, 543, 660, 694, 1219, and 1329 of SEQ ID NO: 9.

Cas9 variants with one or more mutations in amino acid residues homologous to amino acid residues 122, 137, 182, 262, 294, 409, 480, 543, 660, 694, 1219, and 1329 of SEQ ID NO: 9 are provided herein. In some embodiments, the Cas9 variants provided herein comprise mutations corresponding to the D10A mutations in SEQ ID NO: 9, resulting in a partially nuclease-inactive dCas9, wherein the Cas9 can nick the non-target strand but not the targeted strand, and at least one, at least two, at least three, at least four, at least five, at least six, or at least seven mutations of amino acid residues homologous to amino acid residues 122, 137, 182, 262, 294, 409, 480, 543, 660, 694, 1219, and 1329 of SEQ ID NO: 9.

| | | |
|---|---|---|
| WP_010922251.1 | type II CRISPR RNA-guided endonuclease Cas9 [Streptococcus pyogenes] | SEQ ID NO: 10 |
| WP_039695303.1 | type II CRISPR RNA-guided endonuclease Cas9 [Streptococcus gallolyticus] | SEQ ID NO: 11 |
| WP_045635197.1 | type II CRISPR RNA-guided endonuclease Cas9 [Streptococcus mitis] | SEQ ID NO: 12 |
| 5AXW_A | Cas9, Chain A, Crystal Structure [Staphylococcus Aureus] | SEQ ID NO: 13 |
| WP_009880683.1 | type II CRISPR RNA-guided endonuclease Cas9 [Streptococcus pyogenes] | SEQ ID NO: 14 |
| WP_010922251.1 | type II CRISPR RNA-guided endonuclease Cas9 [Streptococcus pyogenes] | SEQ ID NO: 15 |
| WP_011054416.1 | type II CRISPR RNA-guided endonuclease Cas9 [Streptococcus pyogenes] | SEQ ID NO: 16 |
| WP_011284745.1 | type II CRISPR RNA-guided endonuclease Cas9 [Streptococcus pyogenes] | SEQ ID NO: 17 |
| WP_011285506.1 | type II CRISPR RNA-guided endonuclease Cas9 [Streptococcus pyogenes] | SEQ ID NO: 18 |
| WP_011527619.1 | type II CRISPR RNA-guided endonuclease Cas9 [Streptococcus pyogenes] | SEQ ID NO: 19 |
| WP_012560673.1 | type II CRISPR RNA-guided endonuclease Cas9 [Streptococcus pyogenes] | SEQ ID NO: 20 |
| WP_014407541.1 | type II CRISPR RNA-guided endonuclease Cas9 [Streptococcus pyogenes] | SEQ ID NO: 21 |
| WP_020905136.1 | type II CRISPR RNA-guided endonuclease Cas9 [Streptococcus pyogenes] | SEQ ID NO: 22 |
| WP_023080005.1 | type II CRISPR RNA-guided endonuclease Cas9 [Streptococcus pyogenes] | SEQ ID NO: 23 |
| WP_023610282.1 | type II CRISPR RNA-guided endonuclease Cas9 [Streptococcus pyogenes] | SEQ ID NO: 24 |
| WP_030125963.1 | type II CRISPR RNA-guided endonuclease Cas9 [Streptococcus pyogenes] | SEQ ID NO: 25 |
| WP_030126706.1 | type II CRISPR RNA-guided endonuclease Cas9 [Streptococcus pyogenes] | SEQ ID NO: 26 |
| WP_031488318.1 | type II CRISPR RNA-guided endonuclease Cas9 [Streptococcus pyogenes] | SEQ ID NO: 27 |
| WP_032460140.1 | type II CRISPR RNA-guided endonuclease Cas9 [Streptococcus pyogenes] | SEQ ID NO: 28 |
| WP_032461047.1 | type II CRISPR RNA-guided endonuclease Cas9 [Streptococcus pyogenes] | SEQ ID NO: 29 |
| WP_032462016.1 | type II CRISPR RNA-guided endonuclease Cas9 [Streptococcus pyogenes] | SEQ ID NO: 30 |
| WP_032462936.1 | type II CRISPR RNA-guided endonuclease Cas9 [Streptococcus pyogenes] | SEQ ID NO: 31 |
| WP_032464890.1 | type II CRISPR RNA-guided endonuclease Cas9 [Streptococcus pyogenes] | SEQ ID NO: 32 |
| WP_033888930.1 | type II CRISPR RNA-guided endonuclease Cas9 [Streptococcus pyogenes] | SEQ ID NO: 33 |
| WP_038431314.1 | type II CRISPR RNA-guided endonuclease Cas9 [Streptococcus pyogenes] | SEQ ID NO: 34 |
| WP_038432938.1 | type II CRISPR RNA-guided endonuclease Cas9 [Streptococcus pyogenes] | SEQ ID NO: 35 |
| WP_038434062.1 | type II CRISPR RNA-guided endonuclease Cas9 [Streptococcus pyogenes] | SEQ ID NO: 36 |
| BAQ51233.1 | CRISPR-associated protein, Csn1 family [Streptococcus pyogenes] | SEQ ID NO: 37 |
| KGE60162.1 | hypothetical protein MGAS2111_0903 [Streptococcus pyogenes MGAS2111] | SEQ ID NO: 38 |
| KGE60856.1 | CRISPR-associated endonuclease protein [Streptococcus pyogenes SS1447] | SEQ ID NO: 39 |
| WP_002989955.1 | MULTISPECIES: type II CRISPR RNA-guided endonuclease Cas9 [Streptococcus] | SEQ ID NO: 40 |
| WP_003030002.1 | MULTISPECIES: type II CRISPR RNA-guided endonuclease Cas9 [Streptococcus] | SEQ ID NO: 41 |
| WP_003065552.1 | MULTISPECIES: type II CRISPR RNA-guided endonuclease Cas9 [Streptococcus] | SEQ ID NO: 42 |
| WP_001040076.1 | type II CRISPR RNA-guided endonuclease Cas9 [Streptococcus agalactiae] | SEQ ID NO: 43 |
| WP_001040078.1 | type II CRISPR RNA-guided endonuclease Cas9 [Streptococcus agalactiae] | SEQ ID NO: 44 |
| WP_001040080.1 | type II CRISPR RNA-guided endonuclease Cas9 [Streptococcus agalactiae] | SEQ ID NO: 45 |
| WP_001040081.1 | type II CRISPR RNA-guided endonuclease Cas9 [Streptococcus agalactiae] | SEQ ID NO: 46 |
| WP_001040083.1 | type II CRISPR RNA-guided endonuclease Cas9 [Streptococcus agalactiae] | SEQ ID NO: 47 |
| WP_001040085.1 | type II CRISPR RNA-guided endonuclease Cas9 [Streptococcus agalactiae] | SEQ ID NO: 48 |
| WP_001040087.1 | type II CRISPR RNA-guided endonuclease Cas9 [Streptococcus agalactiae] | SEQ ID NO: 49 |
| WP_001040088.1 | type II CRISPR RNA-guided endonuclease Cas9 [Streptococcus agalactiae] | SEQ ID NO: 50 |
| WP_001040089.1 | type II CRISPR RNA-guided endonuclease Cas9 [Streptococcus agalactiae] | SEQ ID NO: 51 |
| WP_001040090.1 | type II CRISPR RNA-guided endonuclease Cas9 [Streptococcus agalactiae] | SEQ ID NO: 52 |
| WP_001040091.1 | type II CRISPR RNA-guided endonuclease Cas9 [Streptococcus agalactiae] | SEQ ID NO: 53 |
| WP_001040092.1 | type II CRISPR RNA-guided endonuclease Cas9 [Streptococcus agalactiae] | SEQ ID NO: 54 |
| WP_001040094.1 | type II CRISPR RNA-guided endonuclease Cas9 [Streptococcus agalactiae] | SEQ ID NO: 55 |
| WP_001040095.1 | type II CRISPR RNA-guided endonuclease Cas9 [Streptococcus agalactiae] | SEQ ID NO: 56 |
| WP_001040096.1 | type II CRISPR RNA-guided endonuclease Cas9 [Streptococcus agalactiae] | SEQ ID NO: 57 |
| WP_001040097.1 | type II CRISPR RNA-guided endonuclease Cas9 [Streptococcus agalactiae] | SEQ ID NO: 58 |
| WP_001040098.1 | type II CRISPR RNA-guided endonuclease Cas9 [Streptococcus agalactiae] | SEQ ID NO: 59 |
| WP_001040099.1 | type II CRISPR RNA-guided endonuclease Cas9 [Streptococcus agalactiae] | SEQ ID NO: 60 |
| WP_001040100.1 | type II CRISPR RNA-guided endonuclease Cas9 [Streptococcus agalactiae] | SEQ ID NO: 61 |
| WP_001040104.1 | type II CRISPR RNA-guided endonuclease Cas9 [Streptococcus agalactiae] | SEQ ID NO: 62 |
| WP_001040105.1 | type II CRISPR RNA-guided endonuclease Cas9 [Streptococcus agalactiae] | SEQ ID NO: 63 |
| WP_001040106.1 | type II CRISPR RNA-guided endonuclease Cas9 [Streptococcus agalactiae] | SEQ ID NO: 64 |
| WP_001040107.1 | type II CRISPR RNA-guided endonuclease Cas9 [Streptococcus agalactiae] | SEQ ID NO: 65 |
| WP_001040108.1 | type II CRISPR RNA-guided endonuclease Cas9 [Streptococcus agalactiae] | SEQ ID NO: 66 |
| WP_001040109.1 | type II CRISPR RNA-guided endonuclease Cas9 [Streptococcus agalactiae] | SEQ ID NO: 67 |
| WP_001040110.1 | type II CRISPR RNA-guided endonuclease Cas9 [Streptococcus agalactiae] | SEQ ID NO: 68 |
| WP_015058523.1 | type II CRISPR RNA-guided endonuclease Cas9 [Streptococcus agalactiae] | SEQ ID NO: 69 |
| WP_017643650.1 | type II CRISPR RNA-guided endonuclease Cas9 [Streptococcus agalactiae] | SEQ ID NO: 70 |
| WP_017647151.1 | type II CRISPR RNA-guided endonuclease Cas9 [Streptococcus agalactiae] | SEQ ID NO: 71 |
| WP_017648376.1 | type II CRISPR RNA-guided endonuclease Cas9 [Streptococcus agalactiae] | SEQ ID NO: 72 |
| WP_017649527.1 | type II CRISPR RNA-guided endonuclease Cas9 [Streptococcus agalactiae] | SEQ ID NO: 73 |
| WP_017771611.1 | type II CRISPR RNA-guided endonuclease Cas9 [Streptococcus agalactiae] | SEQ ID NO: 74 |
| WP_017771984.1 | type II CRISPR RNA-guided endonuclease Cas9 [Streptococcus agalactiae] | SEQ ID NO: 75 |
| CFQ25032.1 | CRISPR-associated protein [Streptococcus agalactiae] | SEQ ID NO: 76 |
| CFV16040.1 | CRISPR-associated protein [Streptococcus agalactiae] | SEQ ID NO: 77 |
| KLJ37842.1 | CRISPR-associated protein Csn1 [Streptococcus agalactiae] | SEQ ID NO: 78 |
| KLJ72361.1 | CRISPR-associated protein Csn1 [Streptococcus agalactiae] | SEQ ID NO: 79 |
| KLL20707.1 | CRISPR-associated protein Csn1 [Streptococcus agalactiae] | SEQ ID NO: 80 |
| KLL42645.1 | CRISPR-associated protein Csn1 [Streptococcus agalactiae] | SEQ ID NO: 81 |
| WP_047207273.1 | type II CRISPR RNA-guided endonuclease Cas9 [Streptococcus agalactiae] | SEQ ID NO: 82 |
| WP_047209694.1 | type II CRISPR RNA-guided endonuclease Cas9 [Streptococcus agalactiae] | SEQ ID NO: 83 |
| WP_050198062.1 | type II CRISPR RNA-guided endonuclease Cas9 [Streptococcus agalactiae] | SEQ ID NO: 84 |
| WP_050201642.1 | type II CRISPR RNA-guided endonuclease Cas9 [Streptococcus agalactiae] | SEQ ID NO: 85 |
| WP_050204027.1 | type II CRISPR RNA-guided endonuclease Cas9 [Streptococcus agalactiae] | SEQ ID NO: 86 |
| WP_050881965.1 | type II CRISPR RNA-guided endonuclease Cas9 [Streptococcus agalactiae] | SEQ ID NO: 87 |
| WP_050886065.1 | type II CRISPR RNA-guided endonuclease Cas9 [Streptococcus agalactiae] | SEQ ID NO: 88 |
| AHN30376.1 | CRISPR-associated protein Csn1 [Streptococcus agalactiae 138P] | SEQ ID NO: 89 |
| EAO78426.1 | reticulocyte binding protein [Streptococcus agalactiae H36B] | SEQ ID NO: 90 |
| CCW42055.1 | CRISPR-associated protein, SAG0894 family [Streptococcus agalactiae ILRI112] | SEQ ID NO: 91 |
| WP_003041502.1 | type II CRISPR RNA-guided endonuclease Cas9 [Streptococcus anginosus] | SEQ ID NO: 92 |
| WP_037593752.1 | type II CRISPR RNA-guided endonuclease Cas9 [Streptococcus anginosus] | SEQ ID NO: 93 |
| WP_049516684.1 | CRISPR-associated protein Csn1 [Streptococcus anginosus] | SEQ ID NO: 94 |
| GAD46167.1 | hypothetical protein ANG6_0662 [Streptococcus anginosus T5] | SEQ ID NO: 95 |
| WP_018363470.1 | type II CRISPR RNA-guided endonuclease Cas9 [Streptococcus caballi] | SEQ ID NO: 96 |
| WP_003043819.1 | type II CRISPR RNA-guided endonuclease Cas9 [Streptococcus canis] | SEQ ID NO: 97 |
| WP_006269658.1 | type II CRISPR RNA-guided endonuclease Cas9 [Streptococcus constellatus] | SEQ ID NO: 98 |
| WP_048800889.1 | type II CRISPR RNA-guided endonuclease Cas9 [Streptococcus constellatus] | SEQ ID NO: 99 |
| WP_012767106.1 | type II CRISPR RNA-guided endonuclease Cas9 [Streptococcus dysgalactiae] | SEQ ID NO: 100 |
| WP_014612333.1 | type II CRISPR RNA-guided endonuclease Cas9 [Streptococcus dysgalactiae] | SEQ ID NO: 101 |
| WP_015017095.1 | type II CRISPR RNA-guided endonuclease Cas9 [Streptococcus dysgalactiae] | SEQ ID NO: 102 |
| WP_015057649.1 | type II CRISPR RNA-guided endonuclease Cas9 [Streptococcus dysgalactiae] | SEQ ID NO: 103 |
| WP_048327215.1 | type II CRISPR RNA-guided endonuclease Cas9 [Streptococcus dysgalactiae] | SEQ ID NO: 104 |
| WP_049519324.1 | CRISPR-associated protein Csn1 [Streptococcus dysgalactiae] | SEQ ID NO: 105 |
| WP_012515931.1 | type II CRISPR RNA-guided endonuclease Cas9 [Streptococcus equi] | SEQ ID NO: 106 |
| WP_021320964.1 | type II CRISPR RNA-guided endonuclease Cas9 [Streptococcus equi] | SEQ ID NO: 107 |
| WP_037581760.1 | type II CRISPR RNA-guided endonuclease Cas9 [Streptococcus equi] | SEQ ID NO: 108 |
| WP_004232481.1 | type II CRISPR RNA-guided endonuclease Cas9 [Streptococcus equinus] | SEQ ID NO: 109 |
| WP_009854540.1 | type II CRISPR RNA-guided endonuclease Cas9 [Streptococcus gallolyticus] | SEQ ID NO: 110 |
| WP_012962174.1 | type II CRISPR RNA-guided endonuclease Cas9 [Streptococcus gallolyticus] | SEQ ID NO: 111 |
| WP_039695303.1 | type II CRISPR RNA-guided endonuclease Cas9 [Streptococcus gallolyticus] | SEQ ID NO: 112 |
| WP_014334983.1 | type II CRISPR RNA-guided endonuclease Cas9 [Streptococcus infantarius] | SEQ ID NO: 113 |
| WP_003099269.1 | type II CRISPR RNA-guided endonuclease Cas9 [Streptococcus iniae] | SEQ ID NO: 114 |
| AHY15608.1 | CRISPR-associated protein Csn1 [Streptococcus iniae] | SEQ ID NO: 115 |
| AHY17476.1 | CRISPR-associated protein Csn1 [Streptococcus iniae] | SEQ ID NO: 116 |
| ESR09100.1 | hypothetical protein IUSA1_08595 [Streptococcus iniae IUSA1] | SEQ ID NO: 117 |
| AGM98575.1 | CRISPR-associated protein Cas9/Csn1, subtype II/NMEMI [Streptococcus iniae SF1] | SEQ ID NO: 118 |
| ALF27331.1 | CRISPR-associated protein Csn1 [Streptococcus intermedius] | SEQ ID NO: 119 |
| WP_018372492.1 | type II CRISPR RNA-guided endonuclease Cas9 [Streptococcus massiliensis] | SEQ ID NO: 120 |
| WP_045618028.1 | type II CRISPR RNA-guided endonuclease Cas9 [Streptococcus mitis] | SEQ ID NO: 121 |
| WP_045635197.1 | type II CRISPR RNA-guided endonuclease Cas9 [Streptococcus mitis] | SEQ ID NO: 122 |
| WP_002263549.1 | type II CRISPR RNA-guided endonuclease Cas9 [Streptococcus mutans] | SEQ ID NO: 123 |
| WP_002263887.1 | type II CRISPR RNA-guided endonuclease Cas9 [Streptococcus mutans] | SEQ ID NO: 124 |
| WP_002264920.1 | type II CRISPR RNA-guided endonuclease Cas9 [Streptococcus mutans] | SEQ ID NO: 125 |
| WP_002269043.1 | type II CRISPR RNA-guided endonuclease Cas9 [Streptococcus mutans] | SEQ ID NO: 126 |
| WP_002269448.1 | type II CRISPR RNA-guided endonuclease Cas9 [Streptococcus mutans] | SEQ ID NO: 127 |
| WP_002271977.1 | type II CRISPR RNA-guided endonuclease Cas9 [Streptococcus mutans] | SEQ ID NO: 128 |
| WP_002272766.1 | type II CRISPR RNA-guided endonuclease Cas9 [Streptococcus mutans] | SEQ ID NO: 129 |
| WP_002273241.1 | type II CRISPR RNA-guided endonuclease Cas9 [Streptococcus mutans] | SEQ ID NO: 130 |
| WP_002275430.1 | type II CRISPR RNA-guided endonuclease Cas9 [Streptococcus mutans] | SEQ ID NO: 131 |
| WP_002276448.1 | type II CRISPR RNA-guided endonuclease Cas9 [Streptococcus mutans] | SEQ ID NO: 132 |
| WP_002277050.1 | type II CRISPR RNA-guided endonuclease Cas9 [Streptococcus mutans] | SEQ ID NO: 133 |
| WP_002277364.1 | type II CRISPR RNA-guided endonuclease Cas9 [Streptococcus mutans] | SEQ ID NO: 134 |
| WP_002279025.1 | type II CRISPR RNA-guided endonuclease Cas9 [Streptococcus mutans] | SEQ ID NO: 135 |
| WP_002279859.1 | type II CRISPR RNA-guided endonuclease Cas9 [Streptococcus mutans] | SEQ ID NO: 136 |
| WP_002280230.1 | type II CRISPR RNA-guided endonuclease Cas9 [Streptococcus mutans] | SEQ ID NO: 137 |
| WP_002281696.1 | type II CRISPR RNA-guided endonuclease Cas9 [Streptococcus mutans] | SEQ ID NO: 138 |
| WP_002282247.1 | type II CRISPR RNA-guided endonuclease Cas9 [Streptococcus mutans] | SEQ ID NO: 139 |
| WP_002282906.1 | type II CRISPR RNA-guided endonuclease Cas9 [Streptococcus mutans] | SEQ ID NO: 140 |
| WP_002283846.1 | type II CRISPR RNA-guided endonuclease Cas9 [Streptococcus mutans] | SEQ ID NO: 141 |
| WP_002287255.1 | type II CRISPR RNA-guided endonuclease Cas9 [Streptococcus mutans] | SEQ ID NO: 142 |
| WP_002288990.1 | type II CRISPR RNA-guided endonuclease Cas9 [Streptococcus mutans] | SEQ ID NO: 143 |
| WP_002289641.1 | type II CRISPR RNA-guided endonuclease Cas9 [Streptococcus mutans] | SEQ ID NO: 144 |
| WP_002290427.1 | type II CRISPR RNA-guided endonuclease Cas9 [Streptococcus mutans] | SEQ ID NO: 145 |
| WP_002295753.1 | type II CRISPR RNA-guided endonuclease Cas9 [Streptococcus mutans] | SEQ ID NO: 146 |
| WP_002296423.1 | type II CRISPR RNA-guided endonuclease Cas9 [Streptococcus mutans] | SEQ ID NO: 147 |
| WP_002304487.1 | type II CRISPR RNA-guided endonuclease Cas9 [Streptococcus mutans] | SEQ ID NO: 148 |
| WP_002305844.1 | type II CRISPR RNA-guided endonuclease Cas9 [Streptococcus mutans] | SEQ ID NO: 149 |
| WP_002307203.1 | type II CRISPR RNA-guided endonuclease Cas9 [Streptococcus mutans] | SEQ ID NO: 150 |
| WP_002310390.1 | type II CRISPR RNA-guided endonuclease Cas9 [Streptococcus mutans] | SEQ ID NO: 151 |
| WP_002352408.1 | type II CRISPR RNA-guided endonuclease Cas9 [Streptococcus mutans] | SEQ ID NO: 152 |
| WP_012997688.1 | type II CRISPR RNA-guided endonuclease Cas9 [Streptococcus mutans] | SEQ ID NO: 153 |
| WP_014677909.1 | type II CRISPR RNA-guided endonuclease Cas9 [Streptococcus mutans] | SEQ ID NO: 154 |
| WP_019312892.1 | type II CRISPR RNA-guided endonuclease Cas9 [Streptococcus mutans] | SEQ ID NO: 155 |
| WP_019313659.1 | type II CRISPR RNA-guided endonuclease Cas9 [Streptococcus mutans] | SEQ ID NO: 156 |
| WP_019314093.1 | type II CRISPR RNA-guided endonuclease Cas9 [Streptococcus mutans] | SEQ ID NO: 157 |
| WP_019315370.1 | type II CRISPR RNA-guided endonuclease Cas9 [Streptococcus mutans] | SEQ ID NO: 158 |
| WP_019803776.1 | type II CRISPR RNA-guided endonuclease Cas9 [Streptococcus mutans] | SEQ ID NO: 159 |
| WP_019805234.1 | type II CRISPR RNA-guided endonuclease Cas9 [Streptococcus mutans] | SEQ ID NO: 160 |
| WP_024783594.1 | type II CRISPR RNA-guided endonuclease Cas9 [Streptococcus mutans] | SEQ ID NO: 161 |
| WP_024784288.1 | type II CRISPR RNA-guided endonuclease Cas9 [Streptococcus mutans] | SEQ ID NO: 162 |
| WP_024784666.1 | type II CRISPR RNA-guided endonuclease Cas9 [Streptococcus mutans] | SEQ ID NO: 163 |
| WP_024784894.1 | type II CRISPR RNA-guided endonuclease Cas9 [Streptococcus mutans] | SEQ ID NO: 164 |
| WP_024786433.1 | type II CRISPR RNA-guided endonuclease Cas9 [Streptococcus mutans] | SEQ ID NO: 165 |
| WP_049473442.1 | CRISPR-associated protein Csn1 [Streptococcus mutans] | SEQ ID NO: 166 |
| WP_049474547.1 | CRISPR-associated protein Csn1 [Streptococcus mutans] | SEQ ID NO: 167 |
| EMC03581.1 | hypothetical protein SMU69_09359 [Streptococcus mutans NLML4] | SEQ ID NO: 168 |
| WP_000428612.1 | type II CRISPR RNA-guided endonuclease Cas9 [Streptococcus oralis] | SEQ ID NO: 169 |
| WP_000428613.1 | type II CRISPR RNA-guided endonuclease Cas9 [Streptococcus oralis] | SEQ ID NO: 170 |
| WP_049523028.1 | CRISPR-associated protein Csn1 [Streptococcus parasanguinis] | SEQ ID NO: 171 |
| WP_003107102.1 | type II CRISPR RNA-guided endonuclease Cas9 [Streptococcus parauberis] | SEQ ID NO: 172 |
| WP_054279288.1 | type II CRISPR RNA-guided endonuclease Cas9 [Streptococcus phocae] | SEQ ID NO: 173 |
| WP_049531101.1 | CRISPR-associated protein Csn1 [Streptococcus pseudopneumoniae] | SEQ ID NO: 174 |
| WP_049538452.1 | CRISPR-associated protein Csn1 [Streptococcus pseudopneumoniae] | SEQ ID NO: 175 |
| WP_049549711.1 | CRISPR-associated protein Csn1 [Streptococcus pseudopneumoniae] | SEQ ID NO: 176 |
| WP_007896501.1 | type II CRISPR RNA-guided endonuclease Cas9 [Streptococcus pseudoporcinus] | SEQ ID NO: 177 |
| EFR44625.1 | CRISPR-associated protein, Csn1 family [Streptococcus pseudoporcinus SPIN 20026] | SEQ ID NO: 178 |
| WP_002897477.1 | type II CRISPR RNA-guided endonuclease Cas9 [Streptococcus sanguinis] | SEQ ID NO: 179 |
| WP_002906454.1 | type II CRISPR RNA-guided endonuclease Cas9 [Streptococcus sanguinis] | SEQ ID NO: 180 |
| WP_009729476.1 | type II CRISPR RNA-guided endonuclease Cas9 [Streptococcus sp. F0441] | SEQ ID NO: 181 |
| CQR24647.1 | CRISPR-associated protein [Streptococcus sp. FF10] | SEQ ID NO: 182 |
| WP_000066813.1 | type II CRISPR RNA-guided endonuclease Cas9 [Streptococcus sp. M334] | SEQ ID NO: 183 |
| WP_009754323.1 | type II CRISPR RNA-guided endonuclease Cas9 [Streptococcus sp. taxon 056] | SEQ ID NO: 184 |
| WP_044674937.1 | type II CRISPR RNA-guided endonuclease Cas9 [Streptococcus suis] | SEQ ID NO: 185 |
| WP_044676715.1 | type II CRISPR RNA-guided endonuclease Cas9 [Streptococcus suis] | SEQ ID NO: 186 |
| WP_044680361.1 | type II CRISPR RNA-guided endonuclease Cas9 [Streptococcus suis] | SEQ ID NO: 187 |
| WP_044681799.1 | type II CRISPR RNA-guided endonuclease Cas9 [Streptococcus suis] | SEQ ID NO: 188 |
| WP_049533112.1 | CRISPR-associated protein Csn1 [Streptococcus suis] | SEQ ID NO: 189 |
| WP_029090905.1 | type II CRISPR RNA-guided endonuclease Cas9 [Brochothrix thermosphacta] | SEQ ID NO: 190 |
| WP_006506696.1 | type II CRISPR RNA-guided endonuclease Cas9 [Catenibacterium mitsuokai] | SEQ ID NO: 191 |
| AIT42264.1 | Cas9hc:NLS:HA [Cloning vector pYB196] | SEQ ID NO: 192 |
| WP_034440723.1 | type II CRISPR endonuclease Cas9 [Clostridiales bacterium 55-All] | SEQ ID NO: 193 |
| AKQ21048.1 | Cas9 [CRISPR-mediated gene targeting vector p(bh5p68-Cas9)] | SEQ ID NO: 194 |
| WP_004636532.1 | type II CRISPR RNA-guided endonuclease Cas9 [Dolosigranulum pigrum] | SEQ ID NO: 195 |
| WP_002364836.1 | MULTISPECIES: type II CRISPR RNA-guided endonuclease Cas9 [Enterococcus] | SEQ ID NO: 196 |
| WP_016631044.1 | MULTISPECIES: type II CRISPR RNA-guided endonuclease Cas9 [Enterococcus] | SEQ ID NO: 197 |
| EM575795.1 | hypothetical protein H318_06676 [Enterococcus durans IPLA 655] | SEQ ID NO: 198 |
| WP_002373311.1 | type II CRISPR RNA-guided endonuclease Cas9 [Enterococcus faecalis] | SEQ ID NO: 199 |
| WP_002378009.1 | type II CRISPR RNA-guided endonuclease Cas9 [Enterococcus faecalis] | SEQ ID NO: 200 |
| WP_002407324.1 | type II CRISPR RNA-guided endonuclease Cas9 [Enterococcus faecalis] | SEQ ID NO: 201 |
| WP_002413717.1 | type II CRISPR RNA-guided endonuclease Cas9 [Enterococcus faecalis] | SEQ ID NO: 202 |
| WP_010775580.1 | type II CRISPR RNA-guided endonuclease Cas9 [Enterococcus faecalis] | SEQ ID NO: 203 |
| WP_010818269.1 | type II CRISPR RNA-guided endonuclease Cas9 [Enterococcus faecalis] | SEQ ID NO: 204 |
| WP_010824395.1 | type II CRISPR RNA-guided endonuclease Cas9 [Enterococcus faecalis] | SEQ ID NO: 205 |
| WP_016622645.1 | type II CRISPR RNA-guided endonuclease Cas9 [Enterococcus faecalis] | SEQ ID NO: 206 |
| WP_033624816.1 | type II CRISPR RNA-guided endonuclease Cas9 [Enterococcus faecalis] | SEQ ID NO: 207 |
| WP_033625576.1 | type II CRISPR RNA-guided endonuclease Cas9 [Enterococcus faecalis] | SEQ ID NO: 208 |
| WP_033789179.1 | type II CRISPR RNA-guided endonuclease Cas9 [Enterococcus faecalis] | SEQ ID NO: 209 |
| WP_002310644.1 | type II CRISPR RNA-guided endonuclease Cas9 [Enterococcus faecium] | SEQ ID NO: 210 |
| WP_002312694.1 | type II CRISPR RNA-guided endonuclease Cas9 [Enterococcus faecium] | SEQ ID NO: 211 |
| WP_002314015.1 | type II CRISPR RNA-guided endonuclease Cas9 [Enterococcus faecium] | SEQ ID NO: 212 |
| WP_002320716.1 | type II CRISPR RNA-guided endonuclease Cas9 [Enterococcus faecium] | SEQ ID NO: 213 |
| WP_002330729.1 | type II CRISPR RNA-guided endonuclease Cas9 [Enterococcus faecium] | SEQ ID NO: 214 |
| WP_002335161.1 | type II CRISPR RNA-guided endonuclease Cas9 [Enterococcus faecium] | SEQ ID NO: 215 |
| WP_002345439.1 | type II CRISPR RNA-guided endonuclease Cas9 [Enterococcus faecium] | SEQ ID NO: 216 |
| WP_034867970.1 | type II CRISPR RNA-guided endonuclease Cas9 [Enterococcus faecium] | SEQ ID NO: 217 |
| WP_047937432.1 | type II CRISPR RNA-guided endonuclease Cas9 [Enterococcus faecium] | SEQ ID NO: 218 |
| WP_010720994.1 | type II CRISPR RNA-guided endonuclease Cas9 [Enterococcus hirae] | SEQ ID NO: 219 |
| WP_010737004.1 | type II CRISPR RNA-guided endonuclease Cas9 [Enterococcus hirae] | SEQ ID NO: 220 |
| WP_034700478.1 | type II CRISPR RNA-guided endonuclease Cas9 [Enterococcus hirae] | SEQ ID NO: 221 |
| WP_007209003.1 | type II CRISPR RNA-guided endonuclease Cas9 [Enterococcus italicus] | SEQ ID NO: 222 |
| WP_023519017.1 | type II CRISPR RNA-guided endonuclease Cas9 [Enterococcus mundtii] | SEQ ID NO: 223 |
| WP_010770040.1 | type II CRISPR RNA-guided endonuclease Cas9 [Enterococcus phoeniculicola] | SEQ ID NO: 224 |
| WP_048604708.1 | type II CRISPR RNA-guided endonuclease Cas9 [Enterococcus sp. AM1] | SEQ ID NO: 225 |
| WP_010750235.1 | type II CRISPR RNA-guided endonuclease Cas9 [Enterococcus villorum] | SEQ ID NO: 226 |
| AII16583.1 | Cas9 endonuclease [Expression vector pCas9] | SEQ ID NO: 227 |
| WP_029073316.1 | type II CRISPR RNA-guided endonuclease Cas9 [Kandleria vitulina] | SEQ ID NO: 228 |
| WP_031589969.1 | type II CRISPR RNA-guided endonuclease Cas9 [Kandleria vitulina] | SEQ ID NO: 229 |
| KDA45870.1 | CRISPR-associated protein Cas9/Csn1, subtype II/NMEMI [Lactobacillus animalis] | SEQ ID NO: 230 |
| WP_039099354.1 | type II CRISPR RNA-guided endonuclease Cas9 [Lactobacillus curvatus] | SEQ ID NO: 231 |
| AKP02966.1 | hypothetical protein ABB45_04605 [Lactobacillus farciminis] | SEQ ID NO: 232 |
| WP_010991369.1 | type II CRISPR RNA-guided endonuclease Cas9 [Listeria innocual] | SEQ ID NO: 233 |
| WP_033838504.1 | type II CRISPR RNA-guided endonuclease Cas9 [Listeria innocual] | SEQ ID NO: 234 |
| EHN60060.1 | CRISPR-associated protein, Csn1 family [Listeria innocua ATCC 33091] | SEQ ID NO: 235 |
| EFR89594.1 | crispr-associated protein, Csn1 family [Listeria innocua FSL S4-378] | SEQ ID NO: 236 |
| WP_038409211.1 | type II CRISPR RNA-guided endonuclease Cas9 [Listeria ivanovii] | SEQ ID NO: 237 |
| EFR95520.1 | crispr-associated protein Csn1 [Listeria ivanovii FSL F6-596] | SEQ ID NO: 238 |
| WP_003723650.1 | type II CRISPR RNA-guided endonuclease Cas9 [Listeria monocytogenes] | SEQ ID NO: 239 |
| WP_003727705.1 | type II CRISPR RNA-guided endonuclease Cas9 [Listeria monocytogenes] | SEQ ID NO: 240 |
| WP_003730785.1 | type II CRISPR RNA-guided endonuclease Cas9 [Listeria monocytogenes] | SEQ ID NO: 241 |
| WP_003733029.1 | type II CRISPR RNA-guided endonuclease Cas9 [Listeria monocytogenes] | SEQ ID NO: 242 |
| WP_003739838.1 | type II CRISPR RNA-guided endonuclease Cas9 [Listeria monocytogenes] | SEQ ID NO: 243 |
| WP_014601172.1 | type II CRISPR RNA-guided endonuclease Cas9 [Listeria monocytogenes] | SEQ ID NO: 244 |
| WP_023548323.1 | type II CRISPR RNA-guided endonuclease Cas9 [Listeria monocytogenes] | SEQ ID NO: 245 |
| WP_031665337.1 | type II CRISPR RNA-guided endonuclease Cas9 [Listeria monocytogenes] | SEQ ID NO: 246 |
| WP_031669209.1 | type II CRISPR RNA-guided endonuclease Cas9 [Listeria monocytogenes] | SEQ ID NO: 247 |
| WP_033920898.1 | type II CRISPR RNA-guided endonuclease Cas9 [Listeria monocytogenes] | SEQ ID NO: 248 |
| AKI42028.1 | CRISPR-associated protein [Listeria monocytogenes] | SEQ ID NO: 249 |
| AKI50529.1 | CRISPR-associated protein [Listeria monocytogenes] | SEQ ID NO: 250 |
| EFR83390.1 | crispr-associated protein Csn1 [Listeria monocytogenes FSL F2-208] | SEQ ID NO: 251 |
| WP_046323366.1 | type II CRISPR RNA-guided endonuclease Cas9 [Listeria seeligeri] | SEQ ID NO: 252 |
| AKE81011.1 | Cas9 [Plant multiplex genome editing vector pYLCRISPR/Cas9Pubi-H] | SEQ ID NO: 253 |
| CUO82355.1 | Uncharacterized protein conserved in bacteria [Roseburia hominis] | SEQ ID NO: 254 |
| WP_033162887.1 | type II CRISPR RNA-guided endonuclease Cas9 [Sharpea azabuensis] | SEQ ID NO: 255 |
| AGZ01981.1 | Cas9 endonuclease [synthetic construct] | SEQ ID NO: 256 |
| AKA60242.1 | nuclease deficient Cas9 [synthetic construct] | SEQ ID NO: 257 |
| AK540380.1 | Cas9 [Synthetic plasmid pFC330] | SEQ ID NO: 258 |
| 4UN5_B | Cas9, Chain B, Crystal Structure | SEQ ID NO: 259 |

Additional suitable Cas9 sequences in which amino acid residues homologous to residues 51, 86, 115, 108, 141, 175, 217, 230, 257, 261, 262, 267, 274, 284, 294, 331, 319, 324, 341, 388, 405, 409, 435, 461, 466, 480, 510, 522, 543, 548, 593, 653, 673, 694, 711, 712, 715, 772, 777, 798, 811, 839, 847, 955, 967, 991, 1063, 1139, 1199, 1207, 1219, 1224, 1227, 1229, 1256, 1264, 1296, 1318, 1356, and/or 1362 of SEQ ID NO: 9 can be identified are known to those of skill in the art. See, e.g., Supplementary Table S2 and Supplementary Figure S2 of Fonfara et al., Phylogeny of Cas9 determines functional exchangeability of dual-RNA and Cas9 among orthologous type II CRISPR-Cas systems, Nucl. Acids Res. 2013, doi: 10.1093/nar/gkt1074, which are incorporated herein by reference in their entirety. Cas9 variants of the sequences provided herein or known in the art comprising one or more mutations, e.g., at least one, at least two, at least three, at least four, at least five, at least six, or at least seven mutations as provided herein, e.g., of one or more amino acid residue that is homologous to amino acid residues 51, 86, 115, 108, 141, 175, 217, 230, 257, 261, 262, 267, 274, 284, 294, 331, 319, 324, 341, 388, 405, 409, 435, 461, 466, 480, 510, 522, 543, 548, 593, 653, 673, 694, 711, 712, 715, 772, 777, 798, 811, 839, 847, 955, 967, 991, 1063, 1139, 1199, 1207, 1219, 1224, 1227, 1229, 1256, 1264, 1296, 1318, 1356, and/or 1362 in SEQ ID NO: 9 are provided by this disclosure, for example, Cas9 variants comprising a A262T, K294R, S409I, E480K, E543D, M694I, and/or E1219V mutation.

### Example 2: Exemplary Adenosine Deaminases and ecTadA Domains

Some aspects of this disclosure relate to the use of adenosine deaminase domains, such as, for example, in a fusion protein comprising a Cas9 domain and a nucleic acid editing domain, wherein the nucleic acid editing domain is an adenosine deaminase. In some embodiments, the adenosine deaminase comprises an amino acid sequence that is at least 60%, 65%, 70%, 75%, 80%, 85%, 90%, 95, 98%, 99%, or 99.5% identical to any one of SEQ ID NOs: 400-458, or any of the adenosine deaminases provided herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 400-458, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 166, identical contiguous amino acid residues as compared to any one of the amino acid sequences set forth in SEQ ID NOs: 400-458, or any of the adenosine deaminases provided herein. In some embodiments, the adenosine deaminase comprises the amino acid sequence of any one of SEQ ID NOs: 400-458, or any of the adenosine deaminases provided herein. In some embodiments, the adenosine deaminase consists of the amino acid sequence of any one of SEQ ID NOs: 400-458, or any of the adenosine deaminases provided herein. The ecTadA sequences provided below are from ecTadA (SEQ ID NO: 400), absent the N-terminal methionine (M). The saTadA sequences provided below are from saTadA (SEQ ID NO: 402), absent the N-terminal methionine (M). For clarity, the amino acid numbering scheme used to identify the various amino acid mutations is derived from ecTadA (SEQ ID NO: 400) for E. coli TadA and saTadA (SEQ ID NO: 402) for S. aureus TadA. Amino acid mutations, relative to SEQ ID NO: 400 (ecTadA) or SEQ DI NO: 402 (saTadA), are indicated by underlining. Exemplary adenosine deaminase domains (e.g., ecTadA) and their use in base editors are described in Gaudelli et al. (2017) Programmable base editing of A⋅T to G⋅C in genomic DNA without DNA cleavage. Nature, 551; 464-471; which is incorporated by reference herein in its entirety.

ecTadA

(SEQ ID NO: 409)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD

ecTadA (D108N)

(SEQ ID NO: 410)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARNAKTGAAGSLMDVLHHPGMNHRVEITEGILADECAALLSDFFRM
RRQEIKAQKKAQSSTD

ecTadA (D108G)

(SEQ ID NO: 411)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARGAKTGAAGSLMDVLHHPGMNHRVEITEGILADECAALLSDFFRM
RRQEIKAQKKAQSSTD

ecTadA (D108V)

(SEQ ID NO: 412)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARVAKTGAAGSLMDVLHHPGMNHRVEITEGILADECAALLSDFFRM
RRQEIKAQKKAQSSTD

ecTadA (H8Y, D108N, and N127S)

(SEQ ID NO: 413)
SEVEFSYEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARNAKTGAAGSLMDVLHHPGMSHRVEITEGILADECAALLSDFFRM
RRQEIKAQKKAQSSTD

ecTadA (H8Y, D108N, N127S, and E155D)

(SEQ ID NO: 414)
SEVEFSYEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARNAKTGAAGSLMDVLHHPGMSHRVEITEGILADECAALLSDFFRM
RRQDIKAQKKAQSSTD

ecTadA (H8Y, D108N, N127S, and E155G)

(SEQ ID NO: 415)
SEVEFSYEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARNAKTGAAGSLMDVLHHPGMSHRVEITEGILADECAALLSDFFRM
RRQGIKAQKKAQSSTD

ecTadA (H8Y, D108N, N127S, and E155V)

(SEQ ID NO: 416)
SEVEFSYEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARNAKTGAAGSLMDVLHHPGMSHRVEITEGILADECAALLSDFFRM
RRQVIKAQKKAQSSTD

ecTadA (A106V, D108N, D147Y, and E155V)

(SEQ ID NO: 417)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGVRNAKTGAAGSLMDVLHHPGMNHRVEITEGILADECAALLSYFFRM
RRQVIKAQKKAQSSTD

ecTadA (L84F, A106V, D108N, H123Y, D147Y, E155V, I156F)

(SEQ ID NO: 418)
SEVEFSHEYWMRHALTLAKRAWDEREVPVGAVLVHNNRVIGEGWNRPIGR
HDPTAHAEIMALRQGGLVMQNYRLIDATLYVTFEPCVMCAGAMIHSRIGR
VVFGVRNAKTGAAGSLMDVLHYPGMNHRVEITEGILADECAALLSYFFRM
RRQVFKAQKKAQSSTD

ecTadA (S2A, I49F, A106V, D108N, D147Y, E155V)

(SEQ ID NO: 419)
AEVEFSHEYWMRHALTLAKRAWDEREVPVGAVLVHNNRVIGEGWNRPFGR
HDPTAHAEIMALRQGGLVMQNYRLIDATLYVTLEPCVMCAGAMIHSRIGR
VVFGVRNAKTGAAGSLMDVLHHPGMNHRVEITEGILADECAALLSYFFRM
RRQVIKAQKKAQSSTD

ecTadA (H8Y, A106T, D108N, N127S, K160S)

(SEQ ID NO: 420)
SEVEFSYEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGTRNAKTGAAGSLMDVLHHPGMSHRVEITEGILADECAALLSDFFRM
RRQEIKAQSKAQSSTD

ecTadA (R26G, L84F, A106V, R107H, D108N, H123Y, A142N, A143D, D147Y, E155V, I156F)

(SEQ ID NO: 421)
SEVEFSHEYWMRHALTLAKRAWDEGEVPVGAVLVHNNRVIGEGWNRPIGR
HDPTAHAEIMALRQGGLVMQNYRLIDATLYVTFEPCVMCAGAMIHSRIGR
VVFGVHNAKTGAAGSLMDVLHYPGMNHRVEITEGILADECNDLLSYFFRM
RRQVFKAQKKAQSSTD

ecTadA (E25G, R26G, L84F, A106V, R107H, D108N, H123Y, A142N, A143D, D147Y, E155V, I156F)

(SEQ ID NO: 422)
SEVEFSHEYWMRHALTLAKRAWDGGEVPVGAVLVHNNRVIGEGWNRPIGR
HDPTAHAEIMALRQGGLVMQNYRLIDATLYVTFEPCVMCAGAMIHSRIGR
VVFGVHNAKTGAAGSLMDVLHYPGMNHRVEITEGILADECNDLLSYFFRM
RRQVFKAQKKAQSSTD

ecTadA (E25D, R26G, L84F, A106V, R107K, D108N, H123Y, A142N, A143G, D147Y, E155V, I156F)

(SEQ ID NO: 423)
SEVEFSHEYWMRHALTLAKRAWDDGEVPVGAVLVHNNRVIGEGWNRPIGR
HDPTAHAEIMALRQGGLVMQNYRLIDATLYVTFEPCVMCAGAMIHSRIGR
VVFGVKNAKTGAAGSLMDVLHYPGMNHRVEITEGILADECNGLLSYFFRM
RRQVFKAQKKAQSSTD

ecTadA (R26Q, L84F, A106V, D108N, H123Y, A142N, D147Y, E155V, 1156F)

(SEQ ID NO: 424)
SEVEFSHEYWMRHALTLAKRAWDEQEVPVGAVLVHNNRVIGEGWNRPIGR
HDPTAHAEIMALRQGGLVMQNYRLIDATLYVTFEPCVMCAGAMIHSRIGR
VVFGVRNAKTGAAGSLMDVLHYPGMNHRVEITEGILADECNALLSYFFRM
RRQVFKAQKKAQSSTD

ecTadA (E25M, R26G, L84F, A106V, R107P, D108N, H123Y, A142N, A143D, D147Y, E155V, I156F)

(SEQ ID NO: 425)
SEVEFSHEYWMRHALTLAKRAWDMGEVPVGAVLVHNNRVIGEGWNRPIGR
HDPTAHAEIMALRQGGLVMQNYRLIDATLYVTFEPCVMCAGAMIHSRIGR
VVFGVPNAKTGAAGSLMDVLHYPGMNHRVEITEGILADECNDLLSYFFRM
RRQVFKAQKKAQSSTD

ecTadA (R26C, L84F, A106V, R107H, D108N, H123Y, A142N, D147Y, E155V, I156F)

(SEQ ID NO: 426)
SEVEFSHEYWMRHALTLAKRAWDECEVPVGAVLVHNNRVIGEGWNRPIGR
HDPTAHAEIMALRQGGLVMQNYRLIDATLYVTFEPCVMCAGAMIHSRIGR
VVFGVHNAKTGAAGSLMDVLHYPGMNHRVEITEGILADECNALLSYFFRM
RRQVFKAQKKAQSSTD

ecTadA (L84F, A106V, D108N, H123Y, A142N, A143L, D147Y, E155V, I156F)

(SEQ ID NO: 427)
SEVEFSHEYWMRHALTLAKRAWDEREVPVGAVLVHNNRVIGEGWNRPIGR
HDPTAHAEIMALRQGGLVMQNYRLIDATLYVTFEPCVMCAGAMIHSRIGR
VVFGVRNAKTGAAGSLMDVLHYPGMNHRVEITEGILADECNLLLSYFFRM
RRQVFKAQKKAQSSTD

ecTadA (R26G, L84F, A106V, D108N, H123Y, A142N, D147Y, E155V, I156F)

(SEQ ID NO: 428)
SEVEFSHEYWMRHALTLAKRAWDEGEVPVGAVLVHNNRVIGEGWNRPIGR
HDPTAHAEIMALRQGGLVMQNYRLIDATLYVTFEPCVMCAGAMIHSRIGR
VVFGVRNAKTGAAGSLMDVLHYPGMNHRVEITEGILADECNALLSYFFRM
RRQVFKAQKKAQSSTD

ecTadA (E25A, R26G, L84F, A106V, R107N, D108N, H123Y, A142N, A143E, D147Y, E155V, I156F)

(SEQ ID NO: 429)
SEVEFSHEYWMRHALTLAKRAWDAGEVPVGAVLVHNNRVIGEGWNRPIGR
HDPTAHAEIMALRQGGLVMQNYRLIDATLYVTFEPCVMCAGAMIHSRIGR
VVFGVNNAKTGAAGSLMDVLHYPGMNHRVEITEGILADECNELLSYFFRM
RRQVFKAQKKAQSSTD

ecTadA (L84F, A106V, D108N, H123Y, D147Y, E155V, I156F)

(SEQ ID NO: 430)
SEVEFSHEYWMRHALTLAKRAWDEREVPVGAVLVHNNRVIGEGWNRPIGRH
DPTAHAEIMALRQGGLVMQNYRLIDATLYVTFEPCVMCAGAMIHSRIGRVV
FGVRNAKTGAAGSLMDVLHYPGMNHRVEITEGILADECAALLSYFFRMRRQ
VFKAQKKAQSSTD

ecTadA (N37T, P48T, L84F, A106V, D108N, H123Y, D147Y, E155V, I156F)

(SEQ ID NO: 431)
SEVEFSHEYWMRHALTLAKRAWDEREVPVGAVLVHTNRVIGEGWNRTIGRH
DPTAHAEIMALRQGGLVMQNYRLIDATLYVTFEPCVMCAGAMIHSRIGRVV
FGVRNAKTGAAGSLMDVLHYPGMNHRVEITEGILADECAALLSYFFRMRRQ
VFKAQKKAQSSTD

ecTadA (N37S, L84F, A106V, D108N, H123Y, D147Y, E155V, I156F)

(SEQ ID NO: 432)
SEVEFSHEYWMRHALTLAKRAWDEREVPVGAVLVHSNRVIGEGWNRPIGRH
DPTAHAEIMALRQGGLVMQNYRLIDATLYVTFEPCVMCAGAMIHSRIGRVV
FGVRNAKTGAAGSLMDVLHYPGMNHRVEITEGILADECAALLSYFFRMRRQ
VFKAQKKAQSSTD

ecTadA (H36L, L84F, A106V, D108N, H123Y, D147Y, E155V, I156F)

(SEQ ID NO: 433)
SEVEFSHEYWMRHALTLAKRAWDEREVPVGAVLVLNNRVIGEGWNRPIGRH
DPTAHAEIMALRQGGLVMQNYRLIDATLYVTFEPCVMCAGAMIHSRIGRVV
FGVRNAKTGAAGSLMDVLHYPGMNHRVEITEGILADECAALLSYFFRMRRQ
VFKAQKKAQSSTD

ecTadA (L84F, A106V, D108N, H123Y, S146R, D147Y, E155V, I156F)

(SEQ ID NO: 434)
SEVEFSHEYWMRHALTLAKRAWDEREVPVGAVLVHNNRVIGEGWNRPIGRH
DPTAHAEIMALRQGGLVMQNYRLIDATLYVTFEPCVMCAGAMIHSRIGRVV
FGVRNAKTGAAGSLMDVLHYPGMNHRVEITEGILADECAALLRYFFRMRRQ
VFKAQKKAQSSTD

ecTadA (H36L, P48L, L84F, A106V, D108N, H123Y, D147Y, E155V, I156F)

(SEQ ID NO: 435)
SEVEFSHEYWMRHALTLAKRAWDEREVPVGAVLVLNNRVIGEGWNRLIGRH
DPTAHAEIMALRQGGLVMQNYRLIDATLYVTFEPCVMCAGAMIHSRIGRVV
FGVRNAKTGAAGSLMDVLHYPGMNHRVEITEGILADECAALLSYFFRMRRQ
VFKAQKKAQSSTD

ecTadA (H36L, L84F, A106V, D108N, H123Y, D147Y, E155V, K57N, I156F)

(SEQ ID NO: 436)
SEVEFSHEYWMRHALTLAKRAWDEREVPVGAVLVLNNRVIGEGWNRPIGRH
DPTAHAEIMALRQGGLVMQNYRLIDATLYVTFEPCVMCAGAMIHSRIGRVV
FGVRNAKTGAAGSLMDVLHYPGMNHRVEITEGILADECAALLSYFFRMRRQ
VFNAQKKAQSSTD

ecTadA (H36L, L84F, A106V, D108N, H123Y, S146C, D147Y, E155V, 1156F)

(SEQ ID NO: 437)
SEVEFSHEYWMRHALTLAKRAWDEREVPVGAVLVLNNRVIGEGWNRPIGRH
DPTAHAEIMALRQGGLVMQNYRLIDATLYVTFEPCVMCAGAMIHSRIGRVV
FGVRNAKTGAAGSLMDVLHYPGMNHRVEITEGILADECAALLCYFFRMRRQ
VFKAQKKAQSSTD

ecTadA (L84F, A106V, D108N, H123Y, S146R, D147Y, E155V, 1156F)

(SEQ ID NO: 438)
SEVEFSHEYWMRHALTLAKRAWDEREVPVGAVLVHNNRVIGEGWNRPIGRH
DPTAHAEIMALRQGGLVMQNYRLIDATLYVTFEPCVMCAGAMIHSRIGRVV
FGVRNAKTGAAGSLMDVLHYPGMNHRVEITEGILADECAALLRYFFRMRRQ
VFKAQKKAQSSTD

ecTadA (N37S, R51H, L84F, A106V, D108N, H123Y, D147Y, E155V, 1156F)

(SEQ ID NO: 439)
SEVEFSHEYWMRHALTLAKRAWDEREVPVGAVLVHSNRVIGEGWNRPIGHH
DPTAHAEIMALRQGGLVMQNYRLIDATLYVTFEPCVMCAGAMIHSRIGRVV
FGVRNAKTGAAGSLMDVLHYPGMNHRVEITEGILADECAALLSYFFRMRRQ
VFKAQKKAQSSTD

ecTadA (R51L, L84F, A106V, D108N, H123Y, D147Y, E155V, I156F, K157N)

(SEQ ID NO: 440)
SEVEFSHEYWMRHALTLAKRAWDEREVPVGAVLVHNNRVIGEGWNRPIGLH
DPTAHAEIMALRQGGLVMQNYRLIDATLYVTFEPCVMCAGAMIHSRIGRVV
FGVRNAKTGAAGSLMDVLHYPGMNHRVEITEGILADECAALLSYFFRMRRQ
VFNAQKKAQSSTD

ecTadA (R51H, L84F, A106V, D108N, H123Y, D147Y, E155V, I156F, K157N)

(SEQ ID NO: 441)
SEVEFSHEYWMRHALTLAKRAWDEREVPVGAVLVHNNRVIGEGWNRPIGHH
DPTAHAEIMALRQGGLVMQNYRLIDATLYVTFEPCVMCAGAMIHSRIGRVV
FGVRNAKTGAAGSLMDVLHYPGMNHRVEITEGILADECAALLSYFFRMRRQ
VFNAQKKAQSSTD

ecTadA (P48S)

(SEQ ID NO: 442)
SEVEFSHEYWMRHALTLAKRAWDEREVPVGAVLVHNNRVIGEGWNRSIGRH
DPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQ
EIKAQKKAQSSTD

ecTadA (P48T)

(SEQ ID NO: 443)
SEVEFSHEYWMRHALTLAKRAWDEREVPVGAVLVHNNRVIGEGWNRTIGRH
DPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQ
EIKAQKKAQSSTD

ecTadA (P48A)

(SEQ ID NO: 444)
SEVEFSHEYWMRHALTLAKRAWDEREVPVGAVLVHNNRVIGEGWNRAIGRH
DPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQ
EIKAQKKAQSSTD

ecTadA (A142N)

(SEQ ID NO: 445)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECNALLSDFFRM
RRQEIKAQKKAQSSTD

ecTadA (W23R)

(SEQ ID NO: 446)
SEVEFSHEYWMRHALTLAKRARDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD

ecTadA (W23L)

(SEQ ID NO: 447)
SEVEFSHEYWMRHALTLAKRALDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD

ecTadA (R152P)

(SEQ ID NO: 448)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
PRQEIKAQKKAQSSTD

ecTadA (R152H)

(SEQ ID NO: 449)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
HRQEIKAQKKAQSSTD

ecTadA (L84F, A106V, D108N, H123Y, D147Y, E155V, I156F)

(SEQ ID NO: 450)
SEVEFSHEYWMRHALTLAKRAWDEREVPVGAVLVHNNRVIGEGWNRPIGR
HDPTAHAEIMALRQGGLVMQNYRLIDATLYVTFEPCVMCAGAMIHSRIGR
VVFGVRNAKTGAAGSLMDVLHYPGMNHRVEITEGILADECAALLSYFFRM
RRQVFKAQKKAQSSTD

ecTadA (H36L, R51L, L84F, A106V, D108N, H123Y, S146C, D147Y, E155V, I156F, K157N)

(SEQ ID NO: 451)
SEVEFSHEYWMRHALTLAKRAWDEREVPVGAVLVLNNRVIGEGWNRPIGL
HDPTAHAEIMALRQGGLVMQNYRLIDATLYVTFEPCVMCAGAMIHSRIGR
VVFGVRNAKTGAAGSLMDVLHYPGMNHRVEITEGILADECAALLCYFFRM
RRQVFNAQKKAQSSTD

ecTadA (H36L, P48S, R51L, L84F, A106V, D108N, H123Y, S146C, D147Y, E155V, I156F, K157N)

(SEQ ID NO: 452)
SEVEFSHEYWMRHALTLAKRAWDEREVPVGAVLVLNNRVIGEGWNRSIGL
HDPTAHAEIMALRQGGLVMQNYRLIDATLYVTFEPCVMCAGAMIHSRIGR
VVFGVRNAKTGAAGSLMDVLHYPGMNHRVEITEGILADECAALLCYFFRM
RRQVFNAQKKAQSSTD

ecTadA (H36L, P48A, R51L, L84F, A106V, D108N, H123Y, S146C, D147Y, E155V, 1156F, K157N)

(SEQ ID NO: 453)
SEVEFSHEYWMRHALTLAKRAWDEREVPVGAVLVLNNRVIGEGWNRAIGL
HDPTAHAEIMALRQGGLVMQNYRLIDATLYVTFEPCVMCAGAMIHSRIGR
VVFGVRNAKTGAAGSLMDVLHYPGMNHRVEITEGILADECAALLCYFFRM
RRQVFNAQKKAQSSTD

ecTadA (W23L, H36L, P48A, R51L, L84F, A106V, D108N, H123Y, S146C, D147Y, R152P, E155V, I156F, K157N)

(SEQ ID NO: 454)
SEVEFSHEYWMRHALTLAKRALDEREVPVGAVLVLNNRVIGEGWNRAIGL
HDPTAHAEIMALRQGGLVMQNYRLIDATLYVTFEPCVMCAGAMIHSRIGR
VVFGVRNAKTGAAGSLMDVLHYPGMNHRVEITEGILADECAALLCYFFRM
PRQVFNAQKKAQSSTD

ecTadA (H36L, P48S, R51L, L84F, A106V, D108N, H123Y, A142N, S146C, D147Y, E155V, I156F, K157N)

(SEQ ID NO: 455)
SEVEFSHEYWMRHALTLAKRAWDEREVPVGAVLVLNNRVIGEGWNRSIGL
HDPTAHAEIMALRQGGLVMQNYRLIDATLYVTFEPCVMCAGAMIHSRIGR
VVFGVRNAKTGAAGSLMDVLHYPGMNHRVEITEGILADECNALLCYFFRM
RRQVFNAQKKAQSSTD

ecTadA (W23L, H36L, P48A, R51L, L84F, A106V, D108N, H123Y, A142N, S146C, D147Y, E155V, I156F, K157N)

(SEQ ID NO: 456)
SEVEFSHEYWMRHALTLAKRALDEREVPVGAVLVLNNRVIGEGWNRAIGL
HDPTAHAEIMALRQGGLVMQNYRLIDATLYVTFEPCVMCAGAMIHSRIGR
VVFGVRNAKTGAAGSLMDVLHYPGMNHRVEITEGILADECNALLCYFFRM
RRQVFNAQKKAQSSTD

ecTadA (W23L, H36L, P48A, R51L, L84F, A106V, D108N, H123Y, A142N, S146C, D147Y, R152P, E155V, I156F, K157N)

(SEQ ID NO: 457)
SEVEFSHEYWMRHALTLAKRALDEREVPVGAVLVLNNRVIGEGWNRAIGL
HDPTAHAEIMALRQGGLVMQNYRLIDATLYVTFEPCVMCAGAMIHSRIGR
VVFGVRNAKTGAAGSLMDVLHYPGMNHRVEITEGILADECNALLCYFFRM
PRQVFNAQKKAQSSTD

ecTadA (W23R, H36L, P48A, R51L, L84F, A106V, D108N, H123Y, S146C, D147Y, R152P, E155V, 1156F, K157N)

(SEQ ID NO: 458)
SEVEFSHEYWMRHALTLAKRALDEREVPVGAVLVLNNRVIGEGWNRAIGL
HDPTAHAEIMALRQGGLVMQNYRLIDATLYVTFEPCVMCAGAMIHSRIGR
VVFGVRNAKTGAAGSLMDVLHYPGMNHRVEITEGILADECAALLCYFFRM
PRQVFNAQKKAQSSTD

### Example 3: Diseases/Disorders Containing Nucleobase Changes that can be Corrected Using a Fusion Protein Provided Herein

In some embodiments, any of the fusion proteins provided herein may be used to correct any human gene mutation by changing a cytosine (C) to a thymine (T). Exemplary diseases that may be treated and/or exemplary mutations that may be corrected, and/or exemplary gRNAs that can be used to produce a mutation, such as a mutation associated with a disease or disorder, are provided in International Patent Application No., PCT/US2016/058345, filed Oct. 22, 2016, and published as Publication No. WO 2017/070633, published Apr. 27, 2017, entitled “Evolved Cas9 Proteins for Gene Editing” which is herein incorporated by reference in its entirety. For example, Table 5 includes human gene mutations that may be corrected by changing a cytosine (C) to a thymine (T). The gene name, gene symbol, and Gene ID are indicated.

In some embodiments, any of the fusion proteins provided herein may be used to correct any human gene mutation by changing a guanine (G) to an adenine (A). Exemplary diseases that may be treated and/or exemplary mutations that may be corrected, and/or exemplary gRNAs that can be used to produce a mutation, such as a mutation associated with a disease or disorder, are provided in International Patent Application No., PCT/US2016/058345, filed Oct. 22, 2016, and published as Publication No. WO 2017/070633, published Apr. 27, 2017, entitled “Evolved Cas9 Proteins for Gene Editing” which is herein incorporated by reference in its entirety. For example, Table 18 includes human gene mutations that may be corrected by changing a guanine (G) to adenine (A). The gene name, gene symbol, and Gene ID are indicated.

In some embodiments, any of the fusion proteins provided herein may be used to correct any human gene mutation by changing an adenine (A) to a guanine (G). Exemplary diseases that may be treated and/or exemplary mutations that may be corrected, and/or exemplary gRNAs that can be used to produce a mutation, such as a mutation associated with a disease or disorder, are provided in International Patent Application No., PCT/US2017/045381, filed Aug. 3, 2017, and published as Publication No. WO 2018/027078, published Feb. 8, 2018, entitled “ADENOSINE NUCLEOBASE EDITORS AND USES THEREOF” which is herein incorporated by reference in its entirety. For example, Table 2 includes gene symbols, gene names, and gRNAs that may be used.

### Example 4: Evolved Cas9 Variants with Broad PAM Compatibility and High DNA Specificity

A key limitation to the use of CRISPR-Cas9 domains for genome editing and other applications is the requirement that a protospacer adjacent motif (PAM) be present at the target site. For the most commonly used Cas9 from Streptococcus pyogenes (SpCas9), this PAM requirement is NGG. No natural or engineered Cas9 variants shown to function efficiently in mammalian cells offer a PAM less restrictive than NGG. Here, phage-assisted continuous evolution (PACE) is used to evolve an expanded PAM SpCas9 variant (xCas9) that can recognize a broad range of PAM sequences including NG, GAA, and GAT. The PAM compatibility of xCas9 is the broadest reported to date among Cas9s active in mammalian cells, and supports applications in human cells including targeted transcriptional activation, nuclease-mediated gene disruption, and both cytidine and adenine base editing. Remarkably, despite its broadened PAM compatibility, xCas9 has much greater DNA specificity than SpCas9, with substantially lower genome-wide off-target activity at all NGG target sites tested, as well as minimal off-target activity when targeting genomic sites with non-NGG PAMs. These findings expand the DNA targeting scope of CRISPR systems and establish that there is no necessary trade-off between Cas9 editing efficiency, PAM compatibility, and DNA specificity.

The clustered regularly interspaced short palindromic repeats (CRISPR)-associated protein 9 (Cas9) system has facilitated widely used genome manipulation capabilities including targeted gene disruption^{1,2}, transcriptional activation and repression³, epigenetic modification³, and direct conversion of a target base pair to a different base pair^{4,5} in a broad range of organisms and cell types⁶. CRISPR-Cas9 targets DNA in a manner that is programmed by an RNA (typically a single-guide RNA, or sgRNA⁷) that contains a “spacer” sequence complementary to the target DNA site, the “protospacer”. In addition to a protospacer that complements the sgRNA, a Cas9 target site must also contain a protospacer adjacent motif (PAM) sequence to support recognition by Cas9. The NGG PAM requirement of canonical SpCas9, which occurs on average only once in every ˜16 randomly chosen genomic loci, greatly limits the targeting scope of Cas9 especially for applications that require precise Cas9 positioning, such as base editing, which requires a PAM ˜15±2 nucleotides from the target base^{4,5}, and some forms of homology-directed repair, which are most efficient when DNA cleavage occurs ˜10-20 base pairs away from a desired alteration^{8,9}. These requirements limit the fraction of genomic DNA that can be targeted with CRISPR systems and highlight the need for more general genome editing tools.

To address this limitation, researchers have harnessed natural CRISPR nucleases with different PAM requirements and engineered existing systems to accept variants of naturally recognized PAMs. Other natural CRISPR nucleases shown to function efficiently in mammalian cells include Staphylococcus aureus Cas9 (SaCas9)¹⁰, Acidaminococcus sp. Cpf1¹¹, Lachnospiraceae bacterium Cpf1¹¹, Campylobacter jejuni Cas9¹², Streptococcus thermophilus Cas9¹³, and Neisseria meningitides Cas9¹⁴. None of these mammalian cell-compatible CRISPR nucleases, however, offer a PAM that occurs as frequently as that of SpCas9. While CRISPR nucleases engineered to accept additional PAM sequences^{15,16} also expand the scope of genomic targets available for Cas9-mediated manipulation, many target sequences remain inaccessible.

Here, phage-assisted continuous evolution (PACE) is used to rapidly generate Cas9 variants that accept an expanded range of PAM sequences. During PACE, host E. coli cells continuously dilute an evolving population of bacteriophages (selection phage, SP). Since dilution occurs faster than cell division but slower than phage replication, only the SP, and not the host cells, can accumulate mutations¹⁷. Each SP carries a gene to be evolved instead of a phage gene (gene III) that is required for the production of infectious progeny phage. SP containing desired gene variants trigger host-cell gene III expression from the accessory plasmid (AP) and the production of infectious SP that propagate the desired variants. Phage encoding inactive variants do not generate infectious progeny and are rapidly diluted out of the culture vessel (FIG. 1A). As phage replication can occur in as little as 10 minutes, PACE enables hundreds of generations of directed evolution to occur per week without researcher intervention¹⁷⁻²².

Evolution of Cas9 Variants with Expanded PAM Compatibility

To link Cas9 DNA recognition to phage propagation during PACE, a bacterial one-hybrid selection^{20,23,24} in which the SP encodes a catalytically dead SpCas9 (dCas9) fused to the ω subunit of bacterial RNA polymerase was developed. When this fusion binds an AP-encoded sgRNA and a PAM and protospacer upstream of gene III in the AP, RNA polymerase recruitment causes gene III expression and phage propagation (FIG. 1b). A library of all 64 possible NNN PAM sequences at the target protospacer in the AP, so that SP encoding Cas9 variants with broader PAM compatibility would replicate in a larger fraction of host cells and thus experience a fitness advantage, was generated.

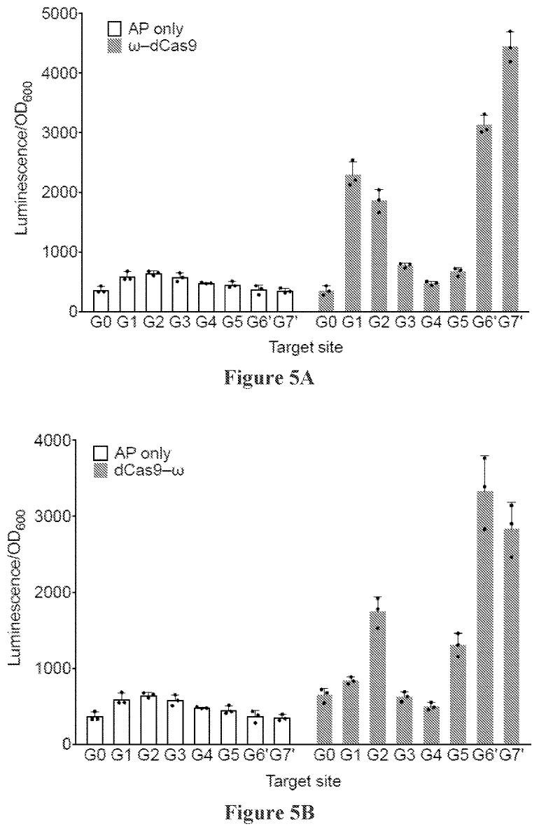

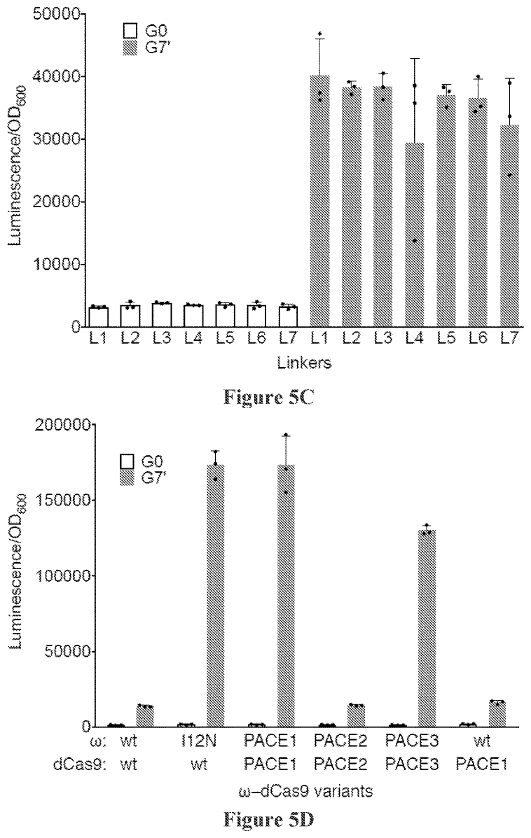

The relationship between Cas9 DNA binding and gene expression was optimized (FIGS. 5A-D). These studies revealed that (i) a fusion of the orientation N-ω-dCas9-C, (ii) a simple Ala-Ala fusion linker, and (iii) placement of the protospacer on the reverse complement strand 45 bp upstream of the −35 box together resulted in the strongest guide RNA-dependent gene expression activation of 13-fold (FIGS. 5A-D). Together, these results establish a linkage between Cas9 DNA binding activity and gene expression in a selection system suitable for PACE.

Using this selection, an SP encoding the ω-dCas9 fusion was allowed to self-optimize on host cells containing an AP with a canonical NGG PAM, resulting in enrichment of an I12N mutation in the ω subunit. Adding this single mutation to ω-dCas9 boosted activation from 13-fold to over 100-fold (FIG. 5D), representing early-stage optimization of ω-dCas9 during PACE prior to evolution for broadened PAM compatibility.

To evolve Cas9 variants with expanded PAM compatibility three AP libraries were generate, each containing a different sgRNA (Table 4) and corresponding protospacer upstream of an NNN PAM library, where N is an equimolar mixture of all four DNA bases. This design imposes selection pressure to recognize many different PAM sequences, as well as to maintain compatibility with different target DNA sequences. All three AP libraries were introduced into host E. coli cells harboring the mutagenesis plasmid MP6¹⁷. The resulting host cells were incubated overnight with SP containing ω(I12N)-dCas9. This phage-assisted non-continuous evolution (PANCE) system^{19,21} preferentially replicates Cas9 variants that bind a greater variety of PAM sequences, similar to PACE, but with lower stringency since there is no outflow of phage.

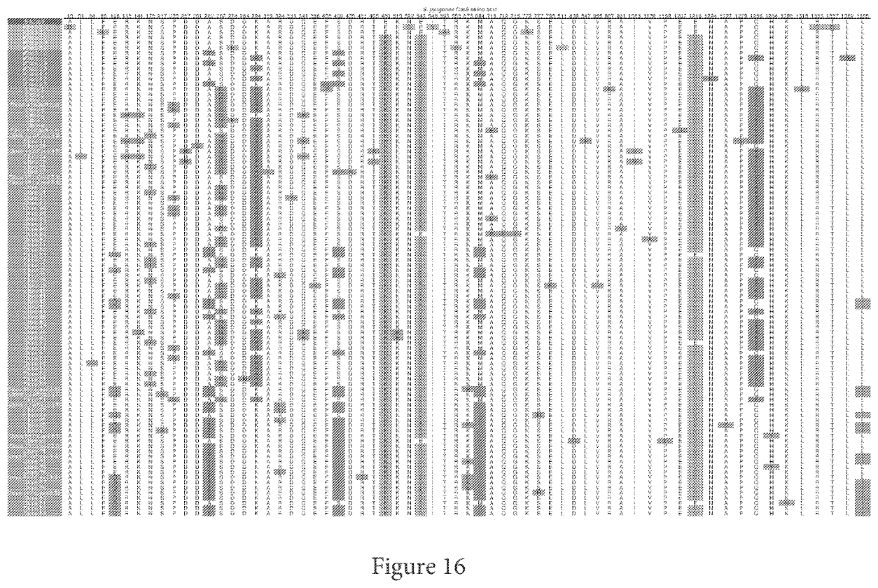

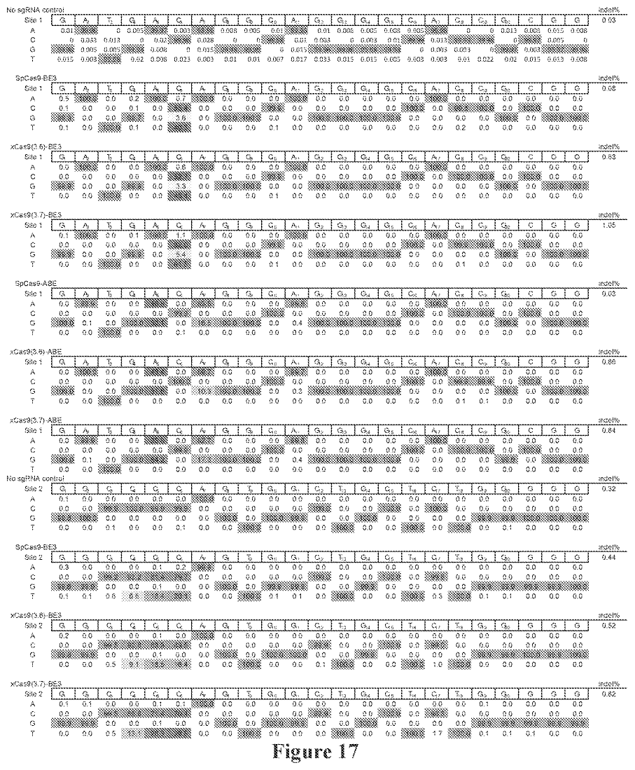

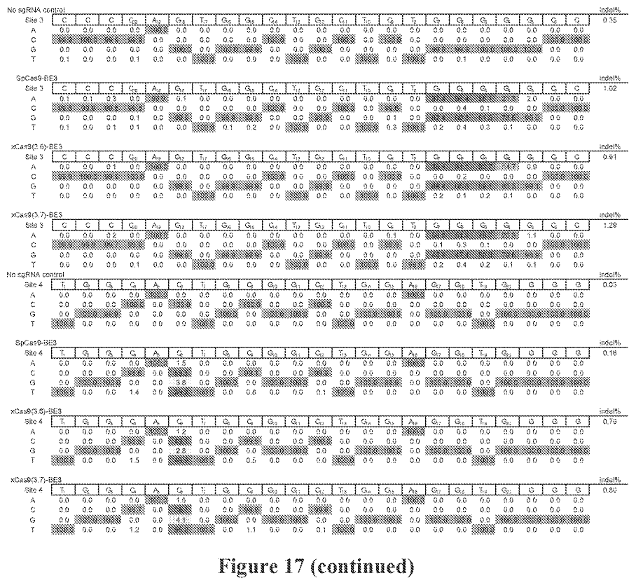

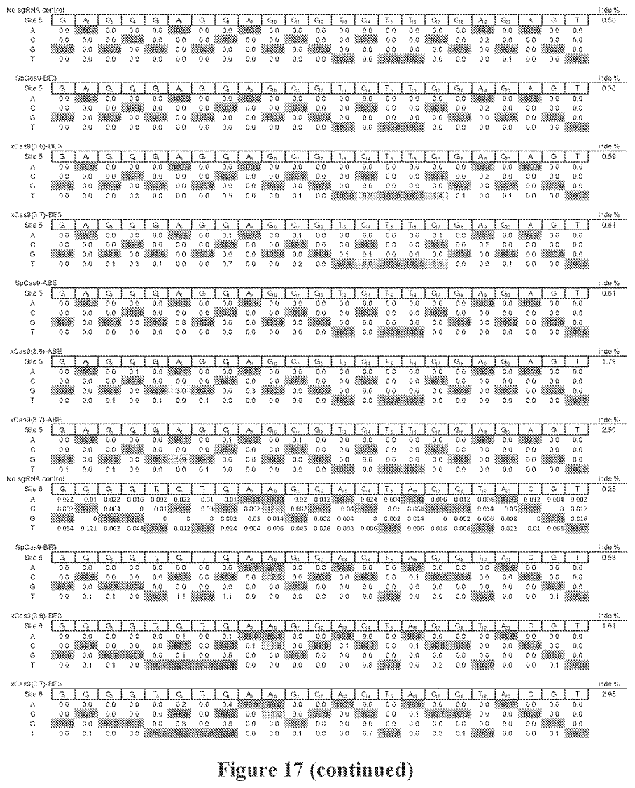

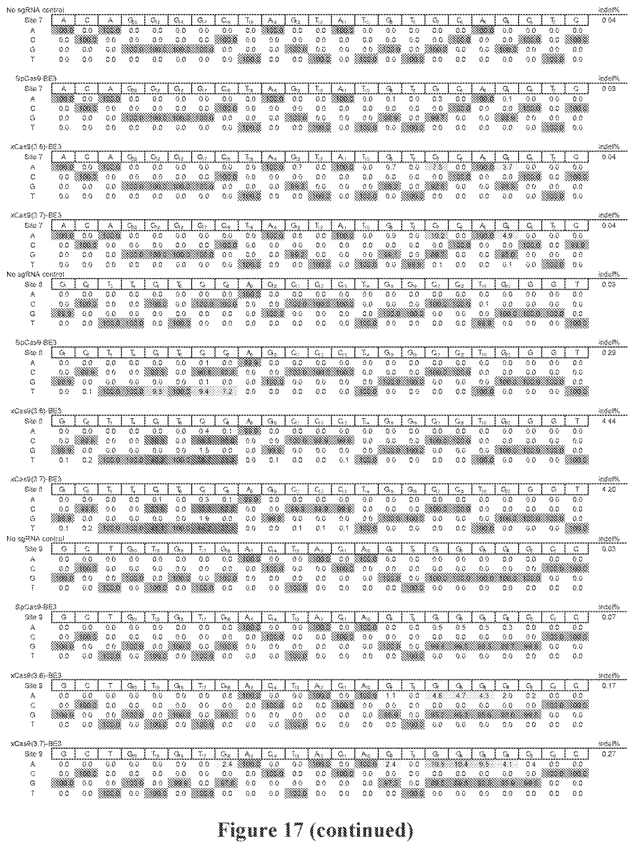

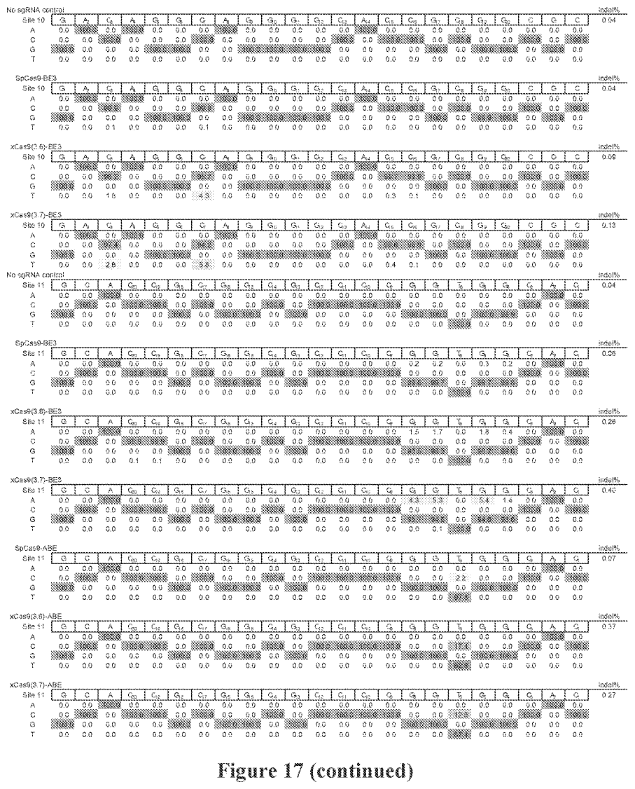

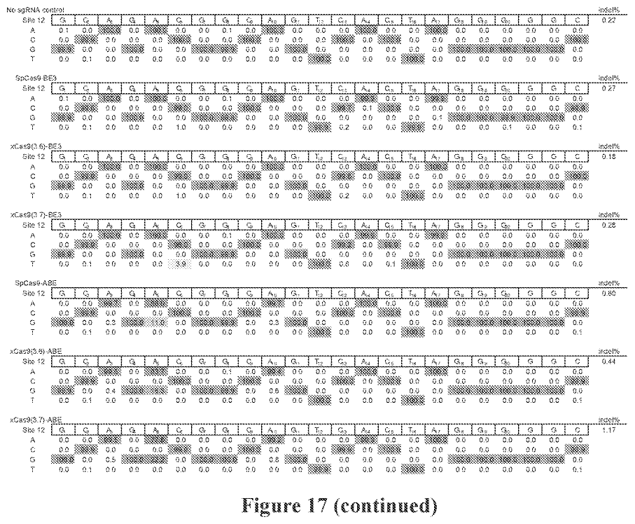

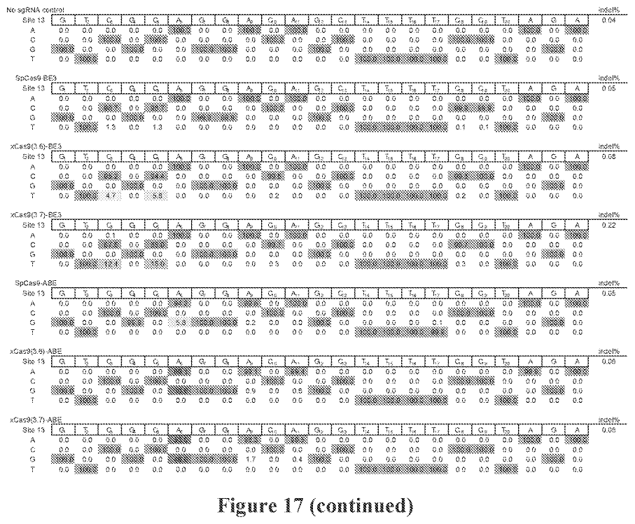

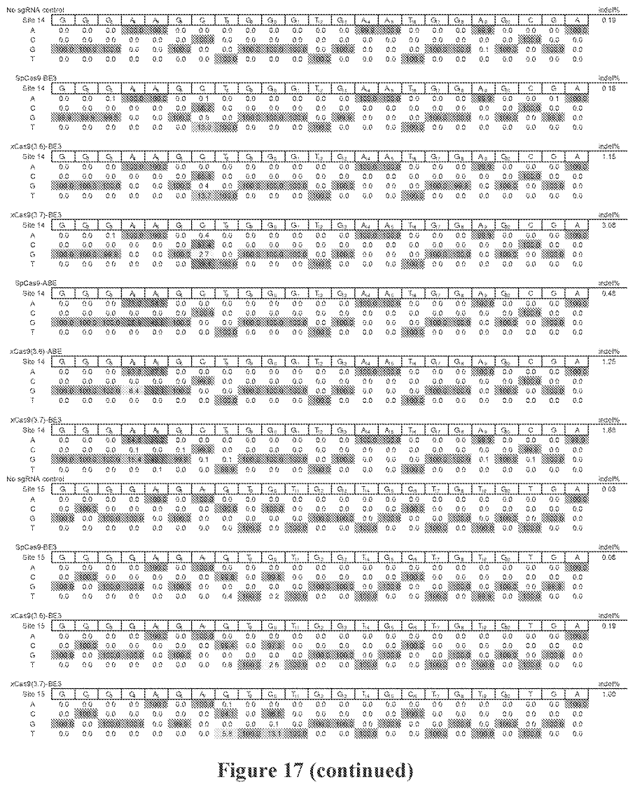

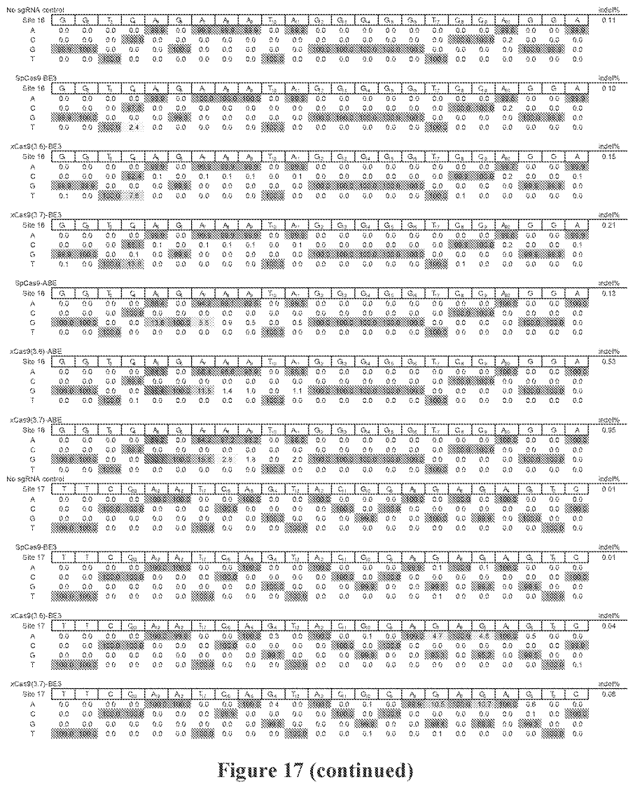

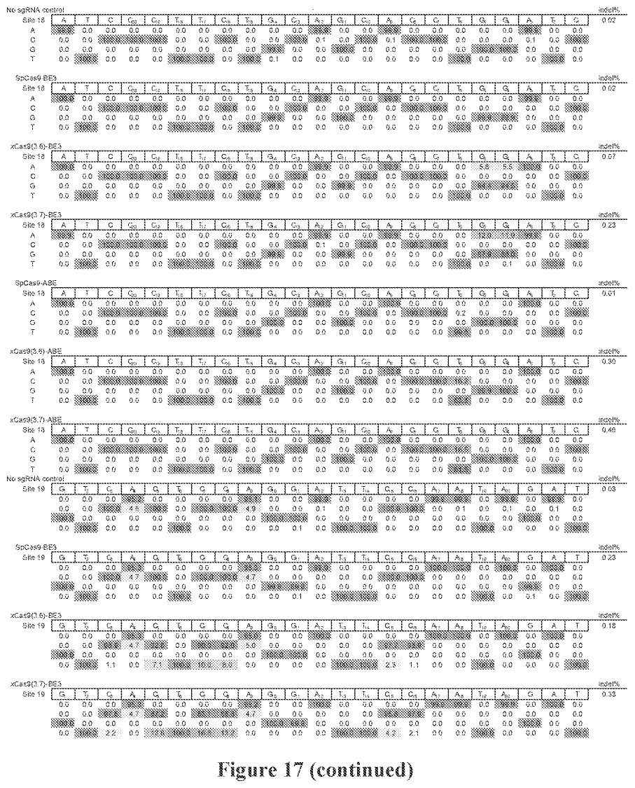

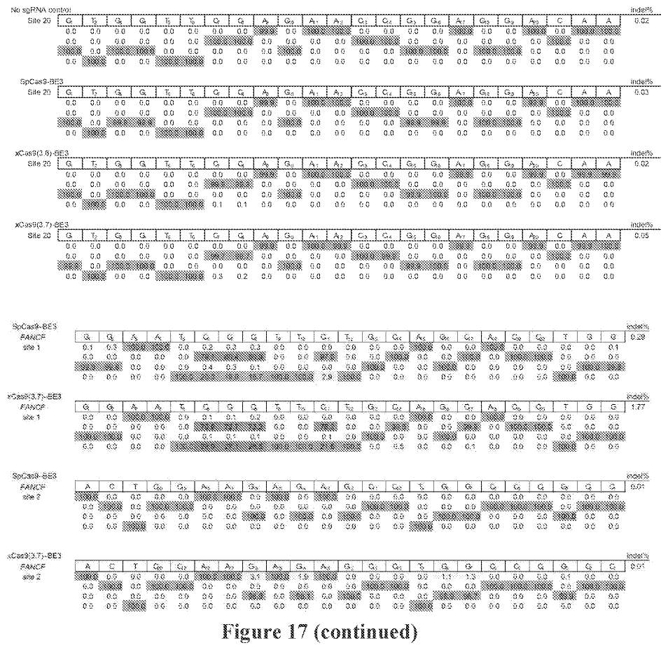

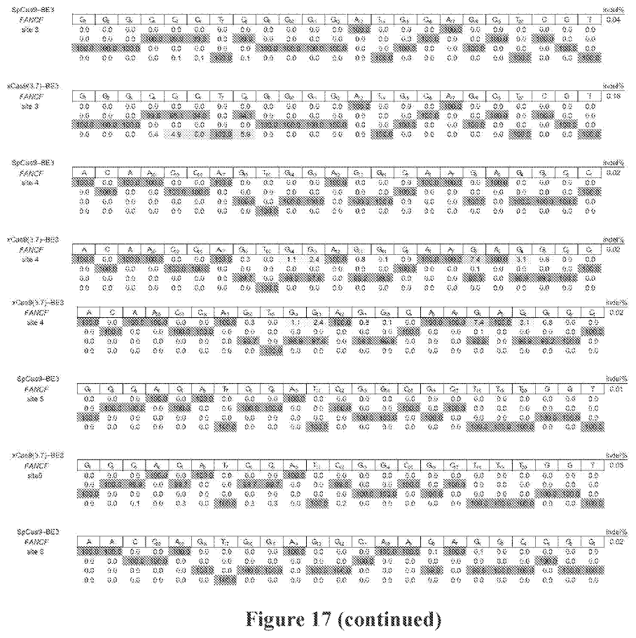

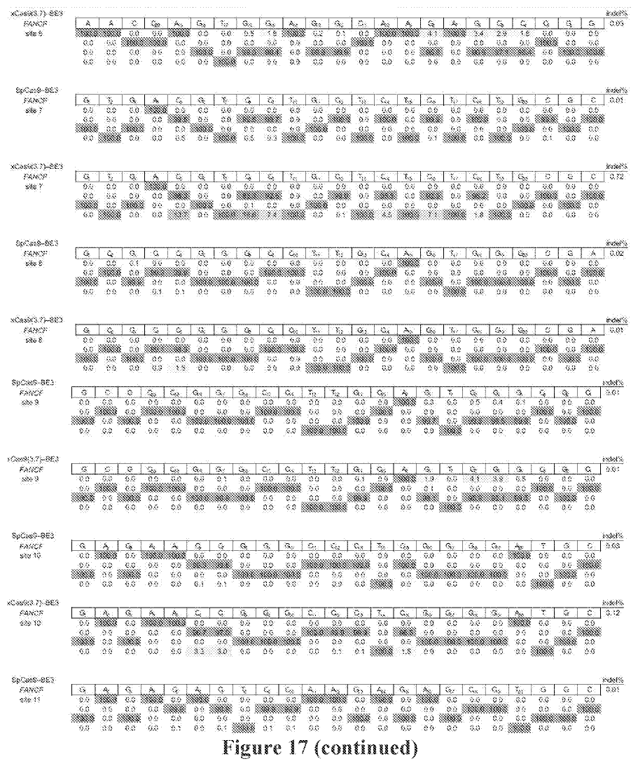

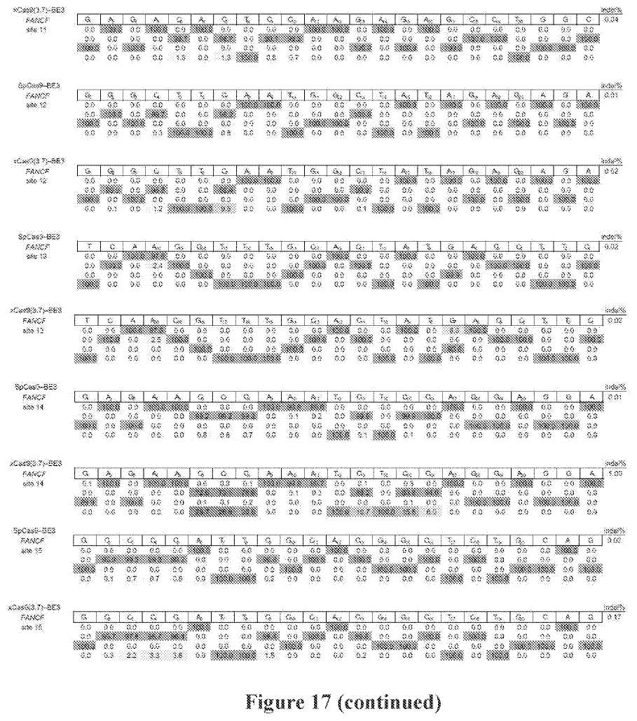

After 24 days of serial overnight propagation and 1:1000 dilution, five Cas9 clones were isolated for sequencing and characterization (xCas9 1.0-1.4). Notable recurring mutations include E480K, E543D, and E1219V (FIG. 1E and FIG. 16). E1219 is close in the SpCas9 crystal structure to R1333 and R1335 (FIG. 1C), two residues known to play a critical role in PAM recognition²⁵. The mixture of phage from the final PANCE pool were further evolved for 72 h in PACE on host cells containing the same AP libraries harboring NNN PAM sequences. Among individual Cas9 clones emerging from PACE (xCas9 2.0-2.6), E480K, E543D, and E1219V were present in all sequenced phage, along with additional mutations seen in multiple clones, including A262T, K294R, S409I, and M694I (FIG. 1E and FIG. 16). Finally, the resulting phage were continuously evolved in PACE for an additional 72 h on host cells containing three protospacer-sgRNA pairs and HHH PAM libraries, where H is A, C, or T, to favor Cas9 variants with activity on non-NGG PAMs.

Fourteen resulting evolved Cas9 variants (xCas9 3.0-3.13) containing consensus mutations (FIG. 1E and FIG. 16) emerged from two apparent evolution trajectories. While all phage shared E480K, E543D, and E1219V core mutations, xCas9 3.0-3.5 also all contained K294R and Q1256K mutations while xCas9 3.6-3.13 all contained A262T, S409I, and M694I mutations, with some of the latter also containing R324L. The Cas9 crystal structure predicts that R324L, S409I, and M694I lie near the DNA-sgRNA interface (FIG. 1D) and could play a role in mediating DNA sequence recognition and the switching of Cas9 from the open to the closed conformation upon target recognition^{26,27}. Since the entire Cas9 gene was subject to mutatgenesis during PACE, the mechanism of xCas9 may differ from that of engineered Cas9 variants that primarily mutated DNA-contacting residues^{15,16,28}.

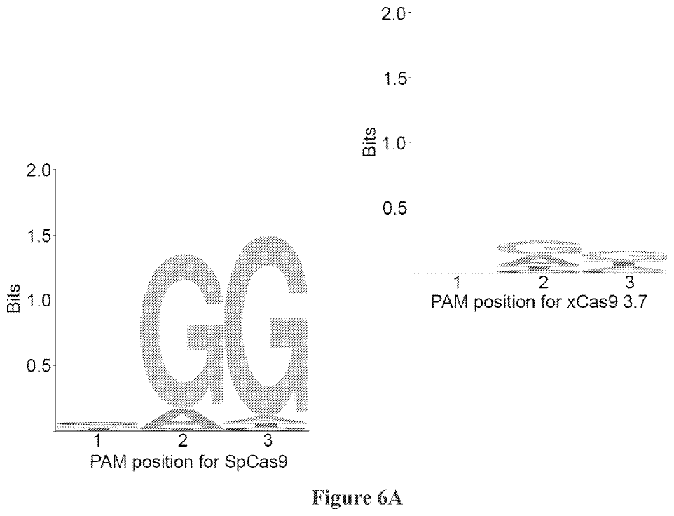

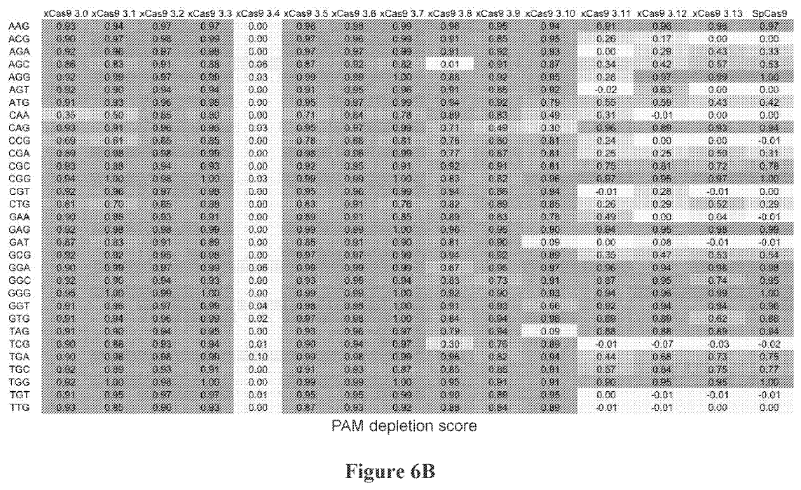

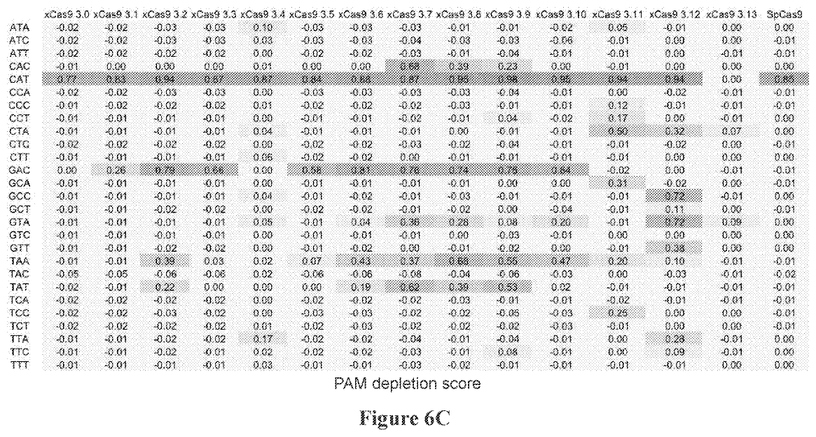

The evolved xCas9 variants were characterized in several contexts. First, the catalytic residues Asp 10 and His 840 were restored to test if xCas9 nucleases can cleave DNA even though they were evolved only for DNA binding. The xCas9 3.0-3.13 clones were tested in a PAM depletion assay^{15,16} in which they were given the opportunity to cleave a library of plasmids containing a protospacer and all possible NNN PAM sequences in an antibiotic resistance gene in bacterial cells. Plasmid cleavage results in the loss of spectinomycin resistance. This PAM depletion assay revealed that xCas9 3.0-3.3 and 3.5-3.9 cleave DNA site with NG, NNG, GAA, GAT, and CAA PAMs (FIGS. 6A-6D). The clone with the highest PAM depletion score, xCas9 3.7, depleted NG, NNG, GAA, GAT, and CAA PAMs≥100-fold compared to the starting library, while xCas9 3.6 showed the second highest average PAM depletion score.

Characterization of xCas9 Transcriptional Activators and Nucleases in Human Cells

To test if mutations evolved during PACE in bacteria are compatible with xCas9 function in mammalian cells, xCas9 variants were characterized for their activity and PAM compatibility in human cells in four contexts: transcriptional activation, genomic DNA cutting, cytidine base editing, and adenine base editing. To test for transcriptional activation, catalytically dead versions of xCas9 were fused to the transcriptional activator VP64-p65-Rta (dxCas9-VPR)²⁹. Plasmids encoding dxCas9-VPR, a GFP reporter downstream of a target protospacer, and a corresponding sgRNA were co-transfected into HEK293T cells²⁹. Target gene transcriptional activation was measured by cellular GFP fluorescence after three days. Three different target site PAM sets were tested: a single reporter with an NGG PAM, a reporter library containing a NNN PAM library, and a reporter library containing a NNNNN PAM library. In addition, two different protospacer sequences, reporter 1 and reporter 2, were tested with their corresponding sgRNAs.

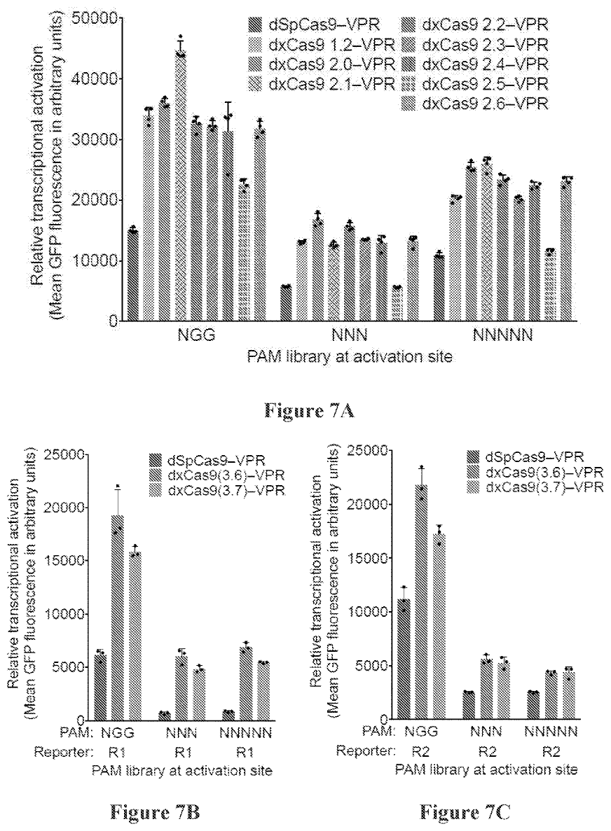

Most early-stage xCas9 variants outperformed wild-type SpCas9 on sites with NGG PAMs, as well as with NNN and NNNNN PAM libraries (FIG. 7A). For reporter 1 and reporter 2, respectively, xCas9 3.7 achieved 2.8- and 1.5-fold higher mean fluorescence for the NGG PAM, 7.9- and 2.1-fold higher mean fluorescence for the NNN PAM library, and 5.2- and 1.7-fold higher mean fluorescence for the NNNNN PAM library compared with SpCas9 (FIGS. 7B and 7C). The similar performances on NNN and NNNNN PAM libraries suggest that xCas9 3.7 did not evolve strong sequence preferences at nucleotides immediately downstream of the NNN PAM. The xCas9 3.6 variant showed similar results to those of xCas9 3.7 in this assay (FIGS. 7B and 7C).

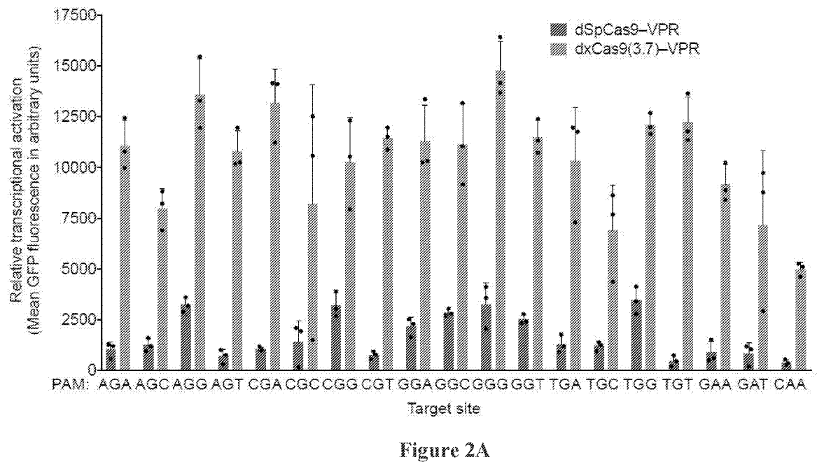

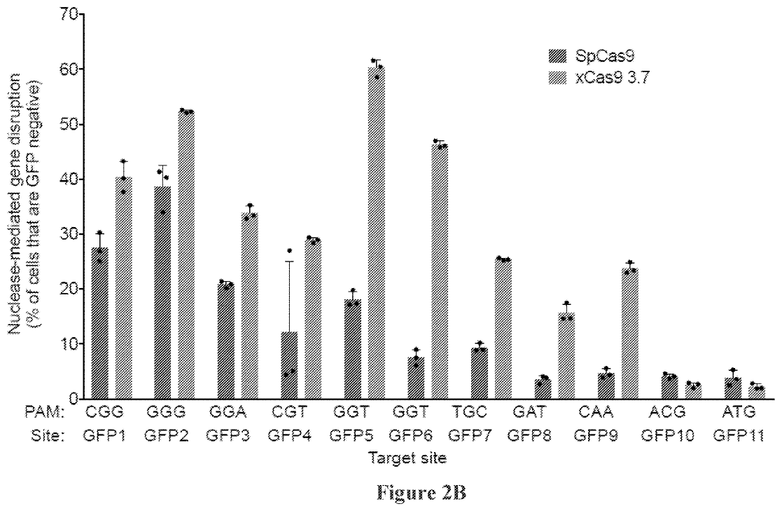

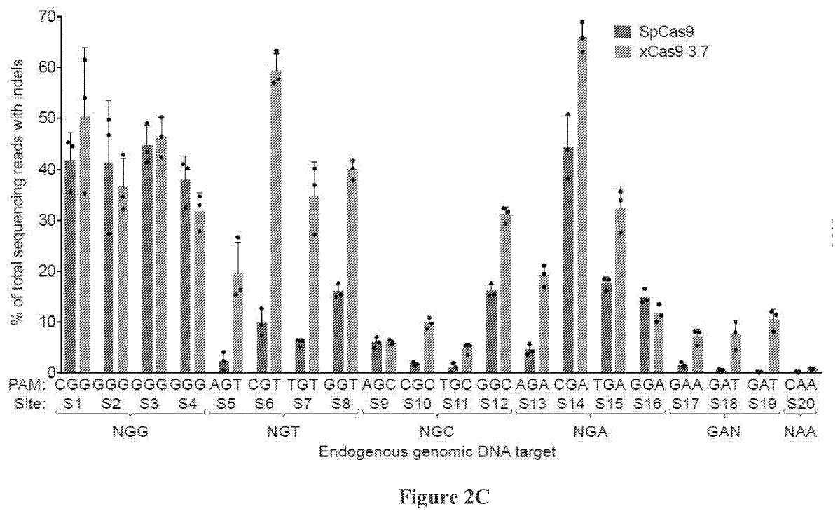

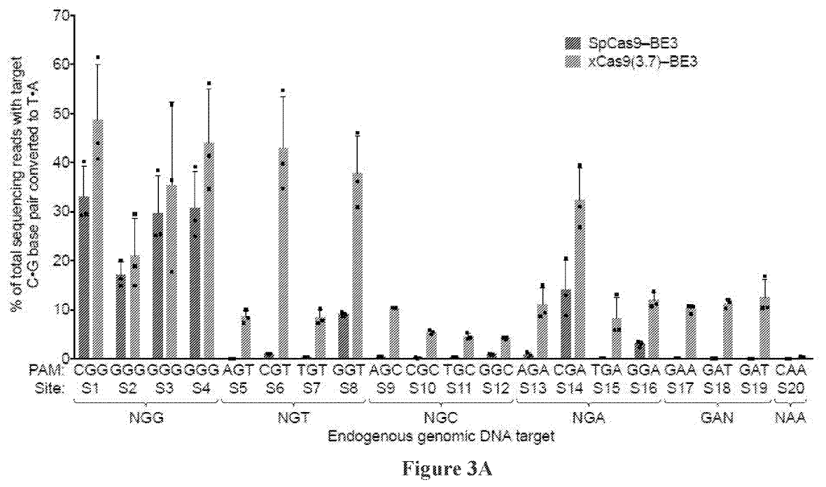

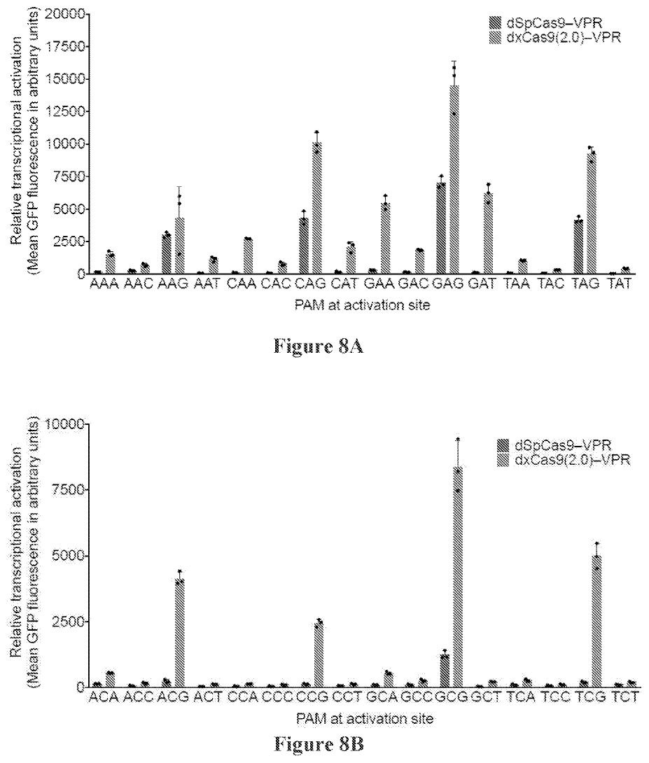

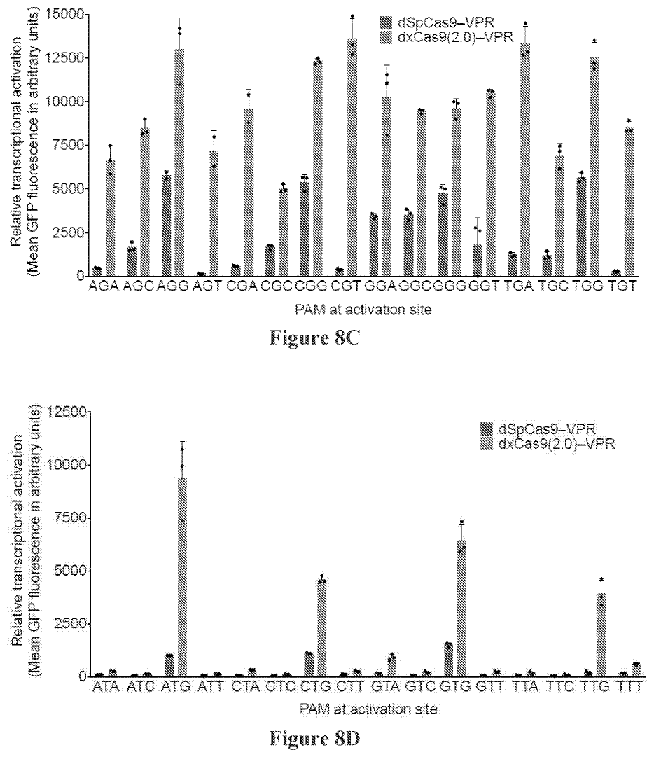

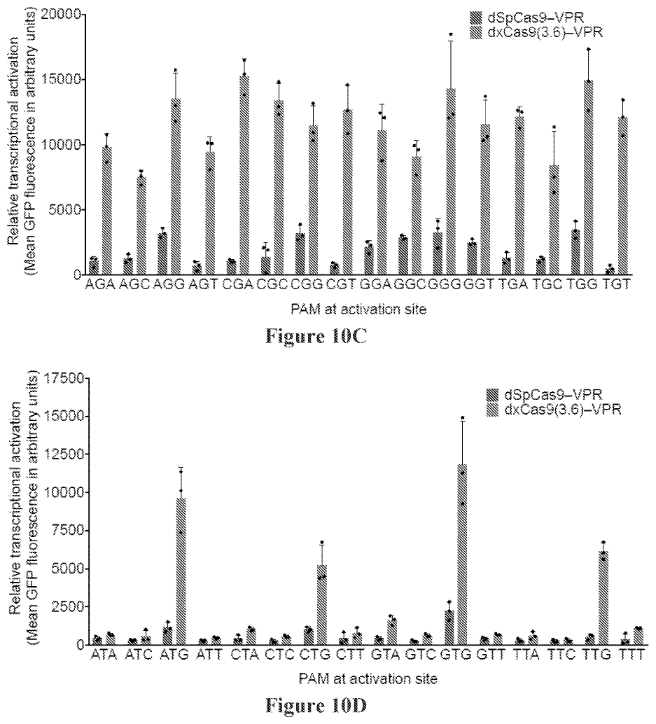

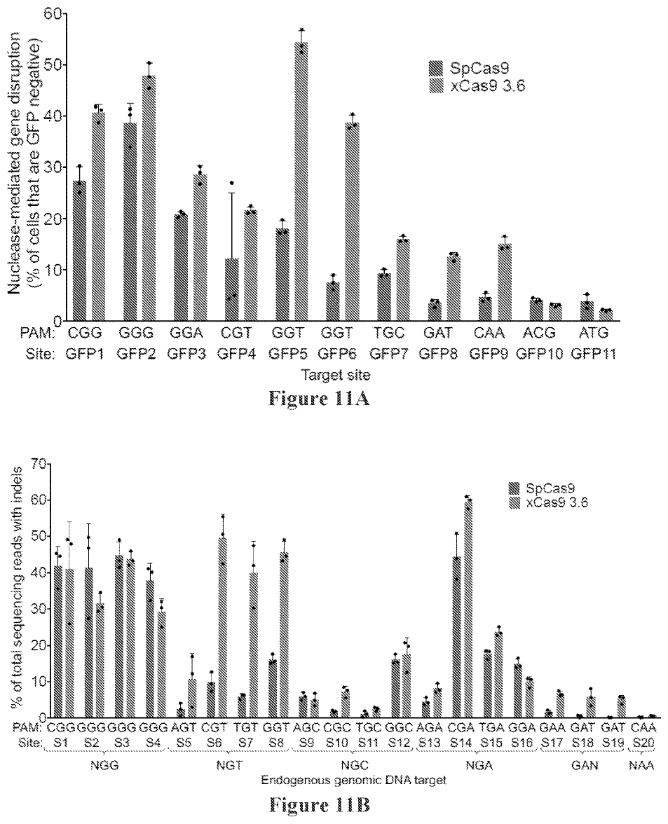

To dissect activity on individual PAM sequences, dxCas9-VPR transcriptional activators were tested on individual target sites containing each of the 64 possible three-nucleotide PAM sequences (FIG. 2A, FIGS. 8A-8D, FIGS. 9A-9E, and FIGS. 10A-10D) in HEK293T cells. Consistent with the PAM library results, dxCas9(3.7)-VPR showed broad improvements in transcriptional activity relative to dSpCas9-VPR across many individual non-NGG PAMs. Transcriptional activation by dxCas9(3.7)-VPR at sites containing NGT, NGA, NGC, NNG, GAA, and GAT PAMs averaged 56-91% of the average activity of dxCas9(3.7)-VPR on the four NGG PAM sites (FIG. 2A and FIGS. 9A-9E). The performance of xCas9 3.6 transcriptional activators was similar to that of xCas9 3.7 (FIGS. 10A-10D). To test if broadened transcriptional activation by xCas9 is limited to reporter plasmids that may be only partially chromatinized, the ability of dxCas9(3.7)-VPR to activate transcription of six endogenous genomic loci in human cells was also tested and a 3.3-fold average improved activation of the two NGG PAM sites and 39-fold average improved activation among the three NGN PAM sites was observed, but there was no improvement on the tested NNG site, relative to dSpCas9-VPR (FIG. 9E). Overall, these results establish that xCas9 is compatible with the dCas9-VPR architecture and can serve as potent transcriptional activators in human cells at a substantially expanded set of PAMs. Based on their strong performance in the PAM depletion assay and as transcriptional activators, we chose xCas9 3.7 and xCas9 3.6 for further characterization.

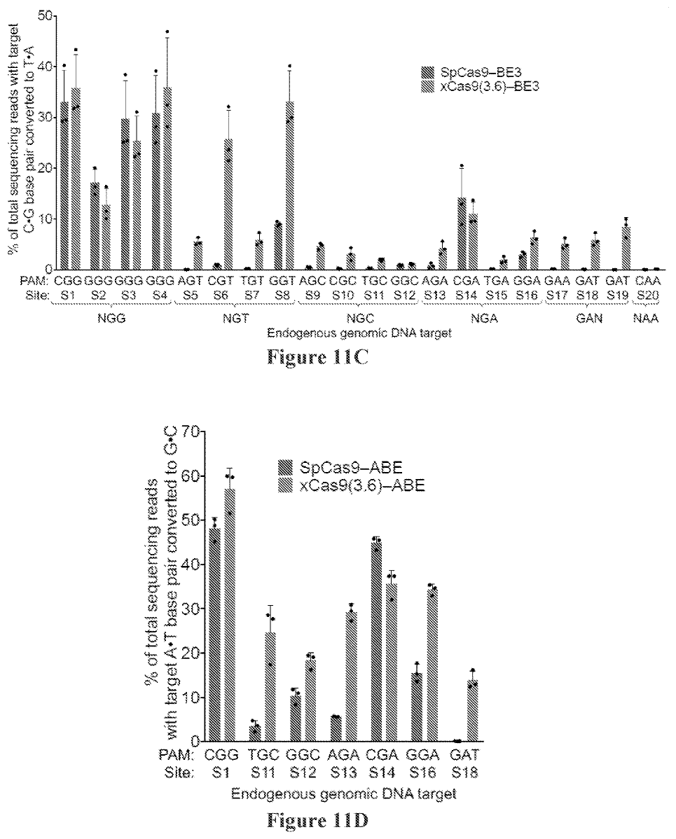

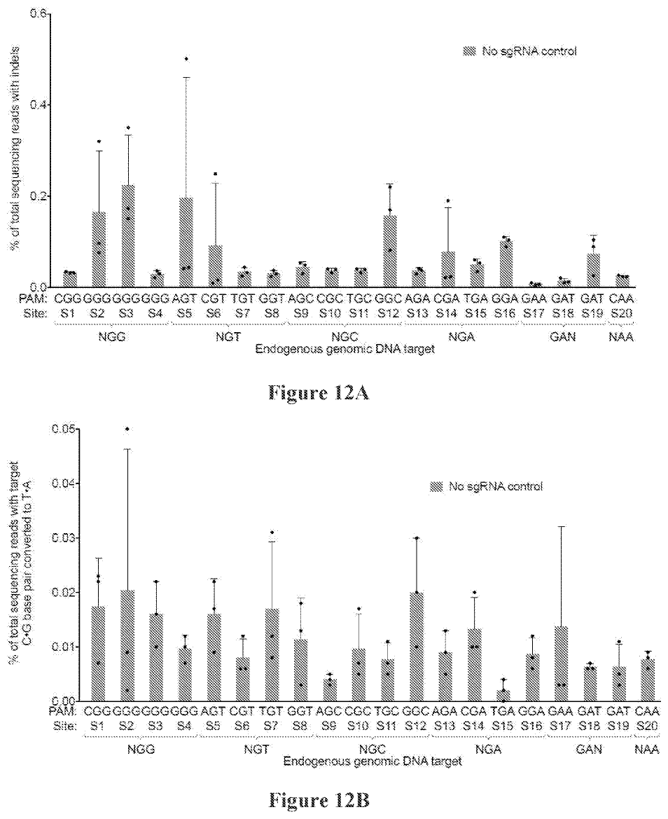

To test targeted genomic DNA cleavage in human cells xCas9 3.7 and 3.6 nuclease were expressed in a HEK293T cell line with a genomically integrated GFP gene and the loss of GFP fluorescence reflecting DNA cleavage and indel-mediated disruption of the target site was measured. Two NGG PAM sites, three NGT sites, two NGC sites, and one NGA, GAT, NCG, and NTG site are present in the GFP sequence; all were tested with SpCas9, xCas9 3.7, and xCas9 3.6. For NGG PAM sites, xCas9 3.7 modestly outperformed SpCas9, resulting in 46±2.0% compared to 33±3.4% GFP disruption, respectively (mean±SD of three independent replicates, FIG. 2B). For all tested non-NGG PAM sites, xCas9 3.7 showed substantially higher (1.6- to 6.4-fold) average apparent cleavage activity than SpCas9 (FIG. 2B). At all tested sites, xCas9 3.6 showed comparable or slightly lower GFP disruption percentages than xCas9 3.7 (FIG. 11A). Neither xCas9 3.7 nor 3.6 increased GFP loss relative to SpCas9 for either NNG PAM site tested, suggesting that the transcriptional activation observed at some NNG PAM sites by dxCas9-VPR activators, and the strong NNG PAM signal in the bacterial PAM depletion assay, do not necessarily translate to DNA cleavage at all NNG sites in mammalian cells. Target site dependence is also well-known for SpCas9³⁰.

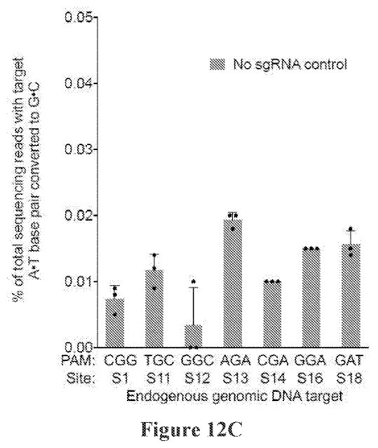

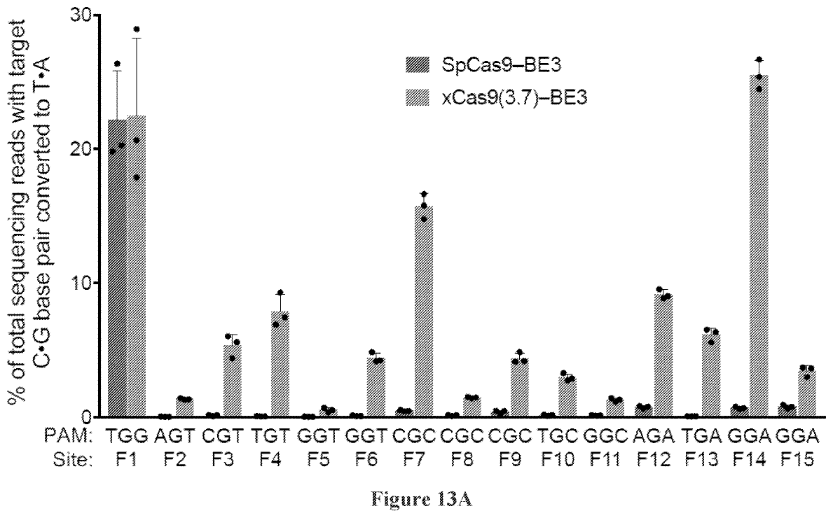

To further characterize DNA cleavage in human cells by xCas9 variants, we targeted endogenous genomic sites in HEK293T cells and measured indel formation by high-throughput sequencing (HTS). Twenty endogenous sites were tested covering four NGG PAM sites, all 12 possible NGT, NGC, and NGA PAMs, and GAT, GAA, and CAA PAMs (FIG. 2C). On the four NGG PAM sites tested, xCas9 3.7 showed comparable activity to SpCas9, averaging 41±6.4% indels compared to 41±6.0% for SpCas9 (FIG. 2C). All four NGT PAM sites showed much higher indel formation with xCas9 3.7 than with SpCas9, averaging 38±4.1% indels compared to 8.6±1.5% for SpCas9, a 4.5-fold increase. The four NGA PAM sites averaged 32±2.4% indels for xCas9 and 20±2.7% for SpCas9, a 1.6-fold increase, consistent with previous reports that NGA can serve as a secondary PAM for SpCas9³¹. While indel frequencies at the endogenous NGC PAM sites were more variable, ranging from 4.8-31%, xCas9 3.7 averaged 13±0.90% indels compared to 6.3±0.77% for SpCas9, a 2.1-fold increase. Among the three GAA and GAT sites tested, SpCas9 showed virtually no activity, averaging 1.4±1.3% indel formation, while xCas9 3.7 averaged 7.2±2.8% indel formation, a 5.2-fold increase. The xCas9 3.6 variant showed comparable or slightly lower indel frequencies for all sites tested (FIG. 11B). Negative control experiments lacking sgRNA plasmid resulted in no indels above background (FIGS. 12A-12C). Taken together, these results indicate that xCas9 3.7 nuclease mediates target gene disruption at NGG PAM sites with comparable efficiencies as wild-type SpCas9, but cleaves NG, GAA, and GAT PAM sites with substantially higher efficiencies than SpCas9. The greater PAM-dependent and protospacer-dependent variability of xCas9 nuclease-mediated gene disruption relative to transcriptional activation (FIGS. 2A-2C, FIGS. 9A-9E, FIGS. 10A-10D, and FIGS. 11A-11D) may reflect more extensive requirements for DNA cleavage than DNA binding^{27,32}, or differences in the chromatin state of plasmid (FIGS. 2A-2C, FIGS. 8A-8D, FIGS. 9A-9D, and FIGS. 10A-10D) versus genomic targets (FIGS. 2B and 2C, FIG. 9E, and FIGS. 11A-11D).

Base Editing by xCas9 Variants in Human Cells

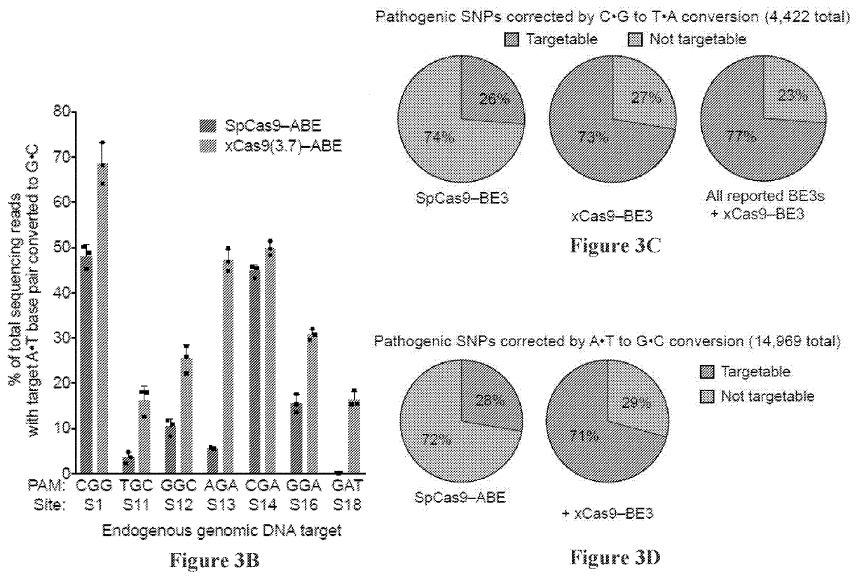

Base editing is a newer genome editing approach that uses a catalytically impaired Cas9 fused to a natural or laboratory-evolved nucleobase deaminase enzyme and, in some cases, a DNA glycosylase inhibitor to directly convert a target C⋅G to T⋅A, or a target A⋅T to G⋅C, without introducing double-stranded DNA breaks or requiring homology-directed repair^{45,33}. The suitability of a target site for base editing is highly dependent on the presence of a suitably positioned PAM, which must exist within a narrow window downstream of the target base pair (typically 15±2 nucleotides). The broad PAM compatibility of xCas9 variants thus has the potential to expand the DNA targeting scope of base editors (FIG. 3C).

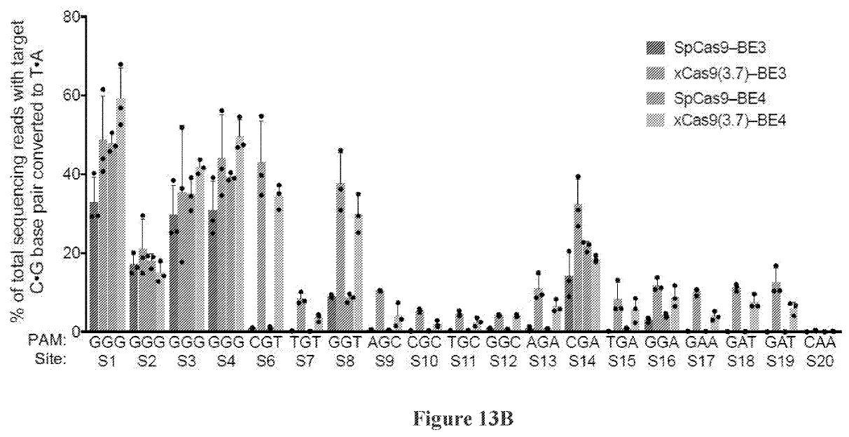

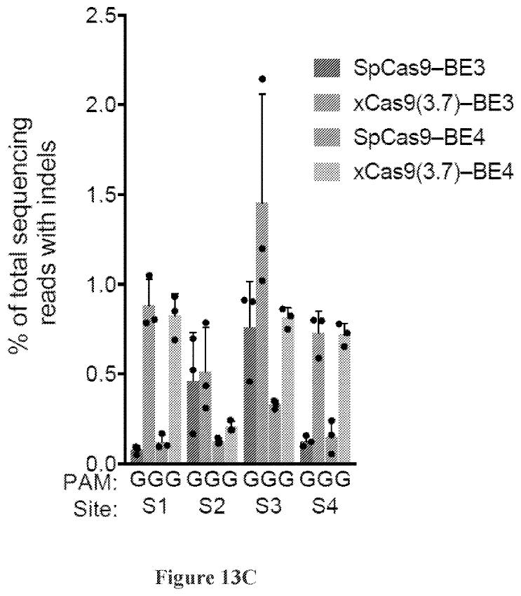

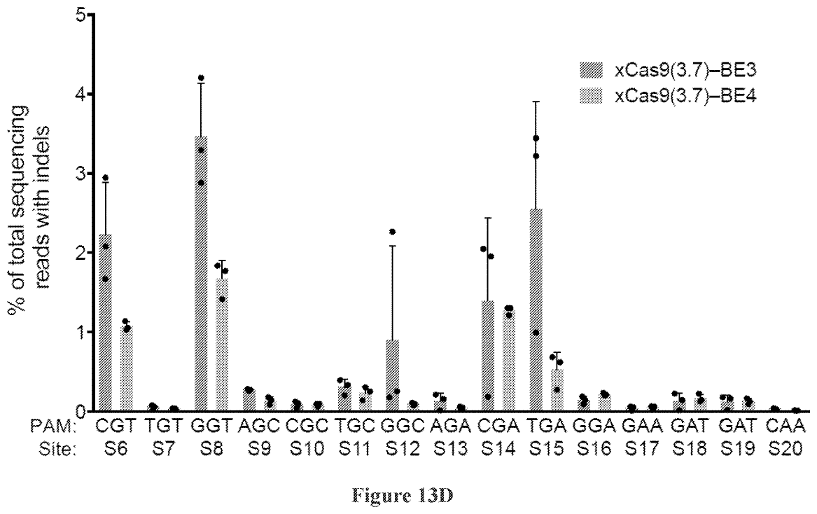

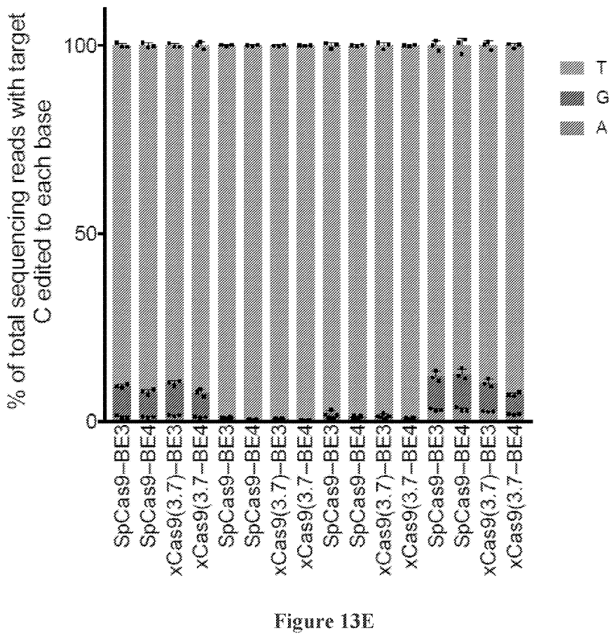

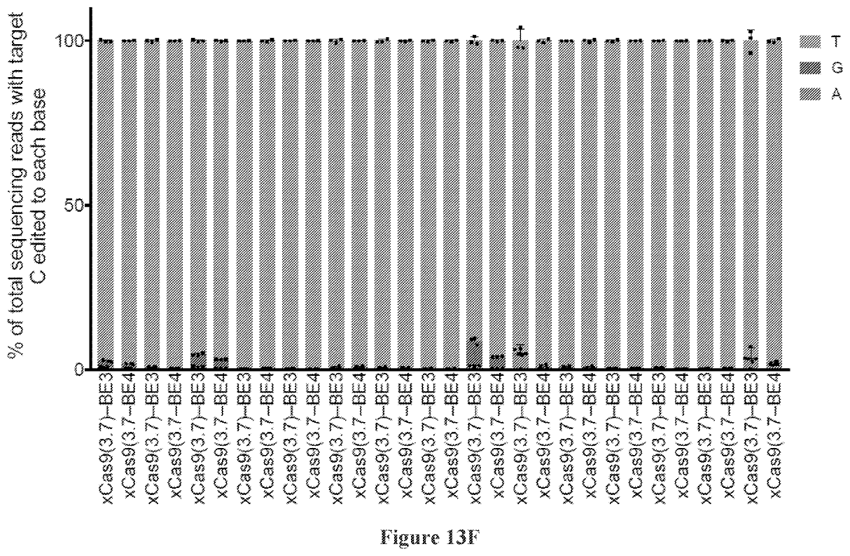

To evaluate C⋅G-to-T⋅A base editing activity of xCas9 variants, SpCas9 was substituted with xCas9 3.7 and 3.6 in the third-generation (BE3) base editor architecture⁴. Both xCas9-BE3s and SpCas9-BE3 were separately transfected into mammalian cells to compare editing efficiency on all 20 sites tested above for endogenous genomic DNA cleavage (FIG. 3A). At the four NGG PAM target sites tested, xCas9(3.7)-BE3 averaged 37±10% C⋅G to T⋅A conversion, while SpCas9-BE3 averaged 28±5.2% (FIG. 3A). At NG, GAA, and GAT PAM sites tested, xCas9(3.7)-BE3 resulted in substantially improved base editing, averaging 24±5.4% editing at NGT PAM sites, an 9.5-fold increase over that of SpCas9; 16±3.5% editing at NGA sites, a 3.5-fold increase over SpCas9; 6.2±0.34% editing at NGC sites, a 13-fold increase over SpCas9; 10±0.75% editing on the GAA PAM site, a >50-fold increase over SpCas9; and 12±1.5% editing on the GAT sites, a >100-fold increase over SpCas9 (FIG. 3A). The base editing efficiencies of xCas9(3.6)-BE3 were comparable to, or slightly worse than, those of xCas9(3.7)-BE3 (FIG. 11C). Cytosine base editing was also tested at an additional 15 endogenous genomic sites within the FANCF gene and observed similar large improvements for xCas9(3.7)-BE3 over SpCas9-BE3 (FIG. 13A). Overall, these results indicate that xCas9 variants are compatible with the BE3 architecture, and enable cytidine base editing of target sites that cannot be accessed by SpCas9-BE3 or, with the exception of NGA PAM sites³³, by any other previously reported base editors. The xCas9 3.7 was also tested in the BE4 architecture designed to reduce undesired byproducts³⁴, and fewer indels and higher product purities were observed, although with slightly lower editing efficiencies (FIGS. 13B-13F).

The recent development of an adenine base editor (ABE) enables programmable installation of A⋅T to G⋅C mutations⁵. No ABEs have been reported yet that can target non-NGG PAM sites, limiting its targeting scope. SpCas9 in ABE 7.105 was replaced with xCas9 3.7 and 3.6, and assayed the resulting xCas9-ABEs in HEK293T cells at the seven endogenous genomic sites tested above that contain an A in the targeting window of ABE (positions 4-8, counting the PAM as positions 21-23). At all seven of these sites, xCas9(3.7)-ABE resulted in higher base editing efficiencies than the original SpCas9-ABE (FIG. 3B). Average base editing efficiency at the NGG PAM site tested increased from 48±2.1% to 69±3.7%. At the GAT PAM site tested, xCas9(3.7)-ABE resulted in 16±1.5% base editing, while SpCas9-ABE yielded no detectable editing (≤0.1%), representing a >100-fold increase. On the two NGC and three NGA sites tested, xCas9(3.7)-ABE averaged 21±2.5% and 43±1.5% base editing, respectively, while SpCas9-ABE averaged 7.0±1.3% and 22±1.2%, respectively. Base editing by xCas9(3.7)-ABE was comparable to or higher than that of xCas9(3.6)-ABE (FIG. 11D). Collectively, these results establish that xCas9-ABE mediates adenine base editing at sites that cannot currently be accessed otherwise.

Improved DNA Specificity of xCas9 Variants in Human Cells

Because PAM recognition is a crucial component of Cas9 DNA specificity²⁷, the substantially broadened PAM compatibility of xCas9s would be expected to increase their off-target activity^{32,35}. Indeed, the engineered S. aureus KKH-SaCas9, which accepts an NNNRRT PAM instead of the native NNGRRT SaCas9 PAM, exhibits comparable or higher off-target editing than SaCas9¹⁵. Most of the xCas9 mutations are close to the PAM or to the DNA:sgRNA interface (FIGS. 1A-1E), raising the possibility that these mutations might alter the degree to which mismatches between the spacer and protospacer impede productive DNA binding or editing. Previous studies have demonstrated that some mutations near the Cas9:DNA interface can reduce off-target activity, in some cases without sacrificing on-target modification efficiency^{26,36,37}.

In order to test the off-target activity of xCas9 variants, GUIDE-seq, an unbiased genome-wide off-target analysis³², was performed on xCas9 3.7, xCas9 3.6, and SpCas9 in HEK293T and U2OS cells. Remarkably, for all five endogenous genomic NGG PAM sites tested in HEK293T cells and for both NGG PAM sites tested in U2OS cells, GUIDE-seq analysis revealed that xCas9 3.7 and 3.6 resulted in much lower off-target activity than SpCas9, as reflected by both the number of detected off-target sites, as well as the total modification frequency at each detected off-target site (FIGS. 4A-4F and FIGS. 14A-14E). For example, at the EMX1 target site in HEK293T cells, SpCas9 showed 5,649 on-target reads and a total of 1,132 off-target reads, while xCas9 3.7 showed 6,874 on-target reads but zero off-target reads (FIG. 4B). In U2OS cells, the off-target:on-target ratio for the same site was 0.65 for SpCas9 with 6,328 on-target reads, and 0.015 for xCas9 3.7 with 22,539 on-target reads, representing a 43-fold reduction in off-target modification (FIG. 14C). Likewise, for HEK sites 1, 2, and 3, xCas9 3.7 resulted in ≥100-fold lower off-target to on-target modification ratios (0.023, <0.001, and 0.028, respectively) compared to those of SpCas9 (0.28, 0.14, and 0.33, respectively) (FIGS. 4A-4F and FIGS. 14A-14E). For the known highly promiscuous target sites HEK site 4 and VEGFA³², the off-target:on-target ratios were 9.4 and 2.0, respectively, for SpCas9, but only 1.0 and 0.48 for xCas9 3.7, a 4.2- to 9.4-fold improvement (FIGS. 14A-14E). We observed these large improvements in DNA specificity for xCas9 3.7 and 3.6 even though, consistent with the above findings, xCas9 variants showed a much broader range of PAM sequences among detected off-target sites (FIGS. 4A-4F and FIGS. 14A-14E). These GUIDE-seq results were verified by HTS of many individual on-target and off-target sites from the genomic DNA of treated cells (FIGS. 15A-15D).

The off-target DNA specificity of xCas9 3.7 at two non-NGG PAM (GAA and CGT) sites with both SpCas9 and xCas9 3.7 in HEK293T cells was also evaluated. As expected, GUIDE-seq did not yield any on-target reads for SpCas9 at either of these non-NGG PAM sites, while xCas9 3.7 had 3,627 on-target reads for the GAA PAM site and 3,055 on-target reads for the CGT PAM site (FIGS. 4E and 4F). Importantly, neither site was accompanied by any detected off-target GUIDE-seq reads for xCas9 3.7, although potential off-target reads were detected for SpCas9 (FIGS. 4E and 4F). Collectively, these findings reveal that xCas9 3.7 and 3.6 offer greatly reduced off-target activity compared with wild-type SpCas9, despite their broader PAM compatibility. These results also establish that there is no necessary trade-off between Cas9-mediated editing efficiency, PAM compatibility, and DNA specificity, a key finding as natural and engineered genome editing agents advance into widespread applications including human clinical trials.

These results, together with the success of multiple independent efforts to create high-fidelity Cas9 variants^{26,36,37}, suggest that the DNA promiscuity of wild-type Cas9, which likely evolved to impede viral evasion, can readily be overcome by protein engineering or evolution. That xCas9 exhibits much higher DNA specificity than SpCas9 even though it was not explicitly selected for this property suggests that the off-target activity of wild-type SpCas9 may lie at a narrow fitness peak suitable for defending the much smaller bacterial genome but not optimal for genome editing in mammalian cells. These observations are therefore consistent with a model in which native SpCas9 is poised to become more specific, rather than less specific, upon mutation.

To our knowledge, the targeting scope of xCas9 is the broadest among Cas9 variants known to function efficiently in mammalian cells. Evolved xCas9 variants are also the first to offer improvements in targeting scope, activity, and DNA specificity in a single entity relative to wild-type SpCas9. While the efficacy of xCas9 on non-NGG PAMs varies based on application-here, transcriptional activation, DNA cleavage, or base editing—and on target site, the ability to access some NG, GAA, and GAT PAM sequences greatly expands the breadth of targets available for site-sensitive genome editing applications. Indeed, compared to SpCas9-BE3, xCas9(3.7)-BE3 increases the percentage of 4,422 pathogenic SNPs in the ClinVar database³⁸ that in principle could be targeted by C⋅G to T⋅A base editing from 26% to 73% (FIG. 3c). Likewise, xCas9(3.7)-ABE increases the fraction of 14,969 pathogenic ClinVar SNPs that could be targeted by A⋅T to G⋅C base editing from 28% to 71% (FIG. 3D). We anticipate that xCas9 and additional CRISPR enzyme variants with broadened PAM compatibilities may also expand the scope of other forms of nucleic acid editing, CRISPR-based screens, and epigenetic modification.

### Methods

General methods and cloning. DNA sequences used in this work are listed in the Supplementary Information. PCR was performed using Q5 Hot Start High-Fidelity DNA Polymerase (New England Biolabs) or Phusion U Green Multiplex PCR Master Mix (Thermo Fisher Scientific). PACE plasmids and phage were constructed by USER cloning or Gibson cloning (New England Biolabs). Cas9 genes and plasmid backbones for PACE were obtained from previously reported plasmids^{19,20} available from Addgene. Plasmids encoding dxCas9-VPR²⁹, xCas9 nucleases, xCas9-BE3⁴, xCas9-BE4³⁴, and xCas9-ABE⁵ were constructed by replacing SpCas9 using Gibson cloning. Plasmids for sgRNA expression were constructed using one-piece blunt-end ligation of a PCR product containing a variable 20-nucleotide sequence corresponding to the desired sgRNA targeted site. Primers and templates used in the synthesis of all sgRNA plasmids used in this work are listed in Supplementary Tables 8, 9, 11, 12, 15, and 16. All guide RNAs used in this study were transcribed from a U6 promoter, and natively started with a G at the 5′ end to avoid possible losses in activity caused by a mismatched 5′ guide terminus. PCR was performed using Q5 Hot Start High-Fidelity Polymerase (New England Biolabs) with the phosphorylated primers and the plasmid pFYF1320 (EGFP sgRNA expression plasmid) as a template according to the manufacturer's instructions. PCR products were analyzed by agarose gel electrophoresis, the band of the expected molecular weight was cut out, and the DNA was extracted using a Zymoclean Gel DNA Recovery Kit (Zymo Research) and ligated using T4 DNA Ligase (New England Biolabs) according to the manufacturer's instructions. DNA vector amplification was carried out using Machi competent cells (Thermo Fisher Scientific). All mammalian ABE constructs, sgRNA plasmids and bacterial constructs were transformed and stored as glycerol stocks at −80° C. in Machi T1R Competent Cells (Thermo Fisher Scientific), which are recA-. Molecular biology grade Hyclone water (GE Healthcare Life Sciences) was used in all assays and PCR reactions. All vectors used in evolution experiments and mammalian cell assays were purified using ZympPURE Plasmid Midiprep (Zymo Research Corporation), which includes endotoxin removal. Antibiotics were purchased from Gold Biotechnology.

Cell culture. HEK293T (ATCC CRL-3216) and U20S (ATCC-HTB-96) were maintained in Dulbecco's Modified Eagle's Medium plus GlutaMax (Thermo Fisher Scientific) supplemented with 10% (v/v) fetal bovine serum (FBS) at 37° C. with 5% CO2.

Transfections. HEK293T cells were seeded on 48-well poly-D-lysine-coated BioCoat plates (Corning) and transfected at approximately 85% confluency. For genomic DNA cutting or base editing, 750 ng of Cas9 or BE3 and 250 ng of sgRNA expression plasmids were transfected using 1.5 μL of Lipofectamine 2000 (Thermo Fisher Scientific) per well according to the manufacturer's protocol. For GFP activation, 200 ng of dCas9-VPR plasmid, 50 ng of sgRNA expression plasmid, 60 ng of GFP reporter plasmid, and 30 ng of iRFP expression plasmid were transfected using 1.5 μL of Lipofectamine 2000 (Thermo Fisher Scientific) per well according to the manufacturer's protocol. Endogenous gene activation was done similarly but with 200 ng of dCas9-VPR plasmid and 50 ng of sgRNA expression plasmid only.

GFP transcriptional activation assay. Transfected HEK293T cells were trypsinized and resuspended in Dulbecco's Modified Eagle's Medium plus GlutaMax (ThermoFisher) supplemented with 10% (v/v) fetal bovine serum (FBS). The cells were kept on ice and flow cytometry was performed using a LSRFortessa from BD Biosciences. Events were gated for iRFP positive cells to analyze transfected cell. The percentage of GFP-positive cells and the intensity of GFP fluorescence from each cell was collected.

RNA expression quantification for endogenous transcriptional activation assay. RNA was extracted from HEK293T cells using the Quick-RNA Plus Kit (Zymo Research). cDNA was synthesized using the iScript cDNA Synthesis Kit (Bio-Rad) and qPCR was performed on a Bio-Rad CFX96 Real-Time PCR Detection System using Q5 Polymerase (NEB) and SYBR Green (Lonza). Primers for qPCR are listed in Supplementary Table 10.

PAM depletion assay. Electrocompetent NEB 10-beta cells (New England Biolabs) were electroporated with two plasmids. The first plasmid expresses Cas9 (inducible by anhydrotetracycline, ATc), the sgRNA (inducible by arabinose), and a spectinomycin resistance gene. The second plasmid contains the target protospacer and a kanamycin resistance gene. After incubation with SOC outgrowth medium (New England Biolabs) for 1 hour the bacteria were plated on agar plates containing both spectinomycin and kanamycin along with ATc and arabinose inducers. After incubating overnight, the bacterial cells on the agar plates were scraped and the plasmids extracted using the ZymoPURE Plasmid Midiprep Kit (Zymo Research). The resulting post-selection DNA included all of the protospacer plasmids not cleaved by Cas9. The same region around the protospacer in the pre-selection library and the post-selection DNA was then amplified separately using NEBNext High-Fidelity PCR Polymerase (New England Biolabs) with flanking HTS primer pairs listed in the Supplementary Table 7. Illumina barcoding PCR reaction was assembled with NEBNext High-Fidelity PCR Polymerase. PCR products were purified by electrophoresis with a 2% agarose gel using a QIAquick Gel Extraction Kit, eluting with 15 μL of water. DNA concentration was quantified with the KAPA Library Quantification Kit-Illumina (KAPA Biosystems) and sequenced on an Illumina MiSeq instrument according to the manufacturer's protocols.

High-throughput DNA sequencing of genomic DNA samples. Transfected cells were harvested after 3 days (BE3 and BE4) or 5 days (DNA cutting and ABE). Media was removed and cells were washed with 1x PBS solution (Thermo Fisher Scientific). Genomic DNA was extracted by addition of 100 μL freshly prepared lysis buffer (10 mM Tris-HCl, pH 7.0, 0.05% SDS, 25 μg/mL Proteinase K (Thermo Fisher Scientific)) directly into each well of the tissue culture plate. The plate was incubated at 37° C. for 1 h. The genomic DNA mixture was transferred to a 96-well PCR plate and incubated at 80° C. for 15 min to denature enzymes. Genomic regions of interest were amplified by PCR with flanking HTS primer pairs listed in the Supplementary Table 13. Each 25 μL PCR reaction was assembled with Phusion Hot Start II High-Fidelity DNA Polymerase (NEB) according to manufacturer's instructions using 1.0 μM of each forward and reverse primer and 1 μL of genomic DNA extract. PCR reactions were carried out as follows: 95° C. for 3 min, then 30 cycles of [98° C. for 30 s, 60° C. for 20 s, and 72° C. for 1 min], followed by a final 72° C. extension for 5 min. PCR products were verified by comparison to DNA standards (1-kb Plus DNA Ladder) on a 2% agarose gel with ethidium bromide. Each 25-μL Illumina barcoding PCR reaction was assembled with Phusion DNA polymerase according to manufacturer's instructions using 0.5 μM of each unique forward and reverse Illumina barcoding primer pair and 1 μL of unpurified genomic amplification PCR reaction mixture. The barcoding reactions were carried out as follows: 98° C. for 2 min, then 8 cycles of [98° C. for 12 s, 61° C. for 25 s, and 72° C. for 30 s], followed by a final 72° C. extension for 1.5 min. PCR products were purified by electrophoresis with a 2% agarose gel using a QIAquick Gel Extraction Kit, eluting with 15 μL of water. DNA concentration was determined with the KAPA Library Quantification Kit-Illumina (KAPA Biosystems) and sequenced on an Illumina MiSeq instrument according to the manufacturer's protocols. Analysis was carried out using previously published Matlab code⁵, provided in Supplementary Notes 1 and 2.

Analysis of human disease-associated mutations in ClinVar database. Bioinformatic analysis of the ClinVar database was carried out in a manner similar to previously described analysis³³. The code is provided in Supplementary Note 3.

GUIDE-Seq. HEK293T cells were transfected with 750 ng of the Cas9 plasmid, 250 ng of the gRNA plasmid, and 20 pmol of GUIDE-seq dsODN. U20S cells were transfected with 750 ng of the Cas9 plasmid, 250 ng of the gRNA plasmid, and 100 pmol of GUIDE-seq dsODN. For both cell types, 20 μL of Solution SE (Lonza) was used along with a Lonza Nucleofector 4-D. Program CM-137 was used for HEK293T cells while program DN-100 was used for U20S cells. Genomic DNA was extracted using the Quick-DNA Miniprep Plus Kit (Zymo Research) following the manufacturer's protocol. The DNA was sheared to an average of 500 bp using a Covaris S220 focused ultrasonicator as previously described³². End repair, dA-tailing, adapter ligation, tag-specific PCR1, and tag-specific PCR2 were carried out using the primers and methods previously described³². DNA concentration was quantified with the KAPA Library Quantification Kit-Illumina (KAPA Biosystems) and sequenced on an Illumina MiSeq instrument according to the manufacturer's protocols. Analysis was carried out using the previously published Python code³².

Data availability. High-throughput sequencing data have been deposited in the NCBI Sequence Read Archive database under accession code SRP130166. Plasmids encoding xCas9 3.7 and 3.6 transcriptional activators, nucleases, BE3, BE4, and ABE variants will be available from Addgene.

### REFERENCES

1. Doudna, J. A. & Charpentier, E. Genome editing. The new frontier of genome engineering with CRISPR-Cas9. Science 346, 1258096, doi:10.1126/science.1258096 (2014).
2. Hsu, P. D., Lander, E. S. & Zhang, F. Development and applications of CRISPR-Cas9 for genome engineering. Cell 157, 1262-1278, doi:10.1016/j.cell.2014.05.010 (2014).
3. Mitsunobu, H., Teramoto, J., Nishida, K. & Kondo, A. Beyond Native Cas9: Manipulating Genomic Information and Function. Trends Biotechnol 35, 983-996, doi:10.1016/j.tibtech.2017.06.004 (2017).
4. Komor, A. C., Kim, Y. B., Packer, M. S., Zuris, J. A. & Liu, D. R. Programmable editing of a target base in genomic DNA without double-stranded DNA cleavage. Nature 533, 420-424, doi:10.1038/nature17946 (2016).
5. Gaudelli, N. M. et al. Programmable base editing of A*T to G*C in genomic DNA without DNA cleavage. Nature 551, 464-471, doi:10.1038/nature24644 (2017).
6. Komor, A. C., Badran, A. H. & Liu, D. R. CRISPR-Based Technologies for the Manipulation of Eukaryotic Genomes. Cell 168, 20-36, doi:10.1016/j.cell.2016.10.044 (2017).
7. Jinek, M. et al. A programmable dual-RNA-guided DNA endonuclease in adaptive bacterial immunity. Science 337, 816-821, doi:10.1126/science.1225829 (2012).
8. Findlay, G. M., Boyle, E. A., Hause, R. J., Klein, J. C. & Shendure, J. Saturation editing of genomic regions by multiplex homology-directed repair. Nature 513, 120-123, doi:10.1038/nature13695 (2014).
9. Yang, L. et al. Optimization of scarless human stem cell genome editing. Nucleic Acids Res 41, 9049-9061, doi:10.1093/nar/gkt555 (2013).
10. Ran, F. A. et al. In vivo genome editing using Staphylococcus aureus Cas9. Nature 520, 186-191, doi:10.1038/nature14299 (2015).
11. Zetsche, B. et al. Cpf1 is a single RNA-guided endonuclease of a class 2 CRISPR-Cas system. Cell 163, 759-771, doi:10.1016/j.cell.2015.09.038 (2015).
12. Kim, E. et al. In vivo genome editing with a small Cas9 orthologue derived from Campylobacter jejuni. Nat Commun 8, 14500, doi:10.1038/ncomms14500 (2017).
13. Muller, M. et al. Streptococcus thermophilus CRISPR-Cas9 Systems Enable Specific Editing of the Human Genome. Mol Ther 24, 636-644, doi:10.1038/mt.2015.218 (2016).
14. Lee, C. M., Cradick, T. J. & Bao, G. The Neisseria meningitidis CRISPR-Cas9 System Enables Specific Genome Editing in Mammalian Cells. Mol Ther 24, 645-654, doi:10.1038/mt.2016.8 (2016).
15. Kleinstiver, B. P. et al. Broadening the targeting range of Staphylococcus aureus CRISPR-Cas9 by modifying PAM recognition. Nat Biotechnol 33, 1293-1298, doi:10.1038/nbt.3404 (2015).
16. Kleinstiver, B. P. et al. Engineered CRISPR-Cas9 nucleases with altered PAM specificities. Nature 523, 481-485, doi:10.1038/nature14592 (2015).
17. Badran, A. H. & Liu, D. R. Development of potent in vivo mutagenesis plasmids with broad mutational spectra. Nat Commun 6, 8425, doi:10.1038/ncomms9425 (2015).
18. Esvelt, K. M., Carlson, J. C. & Liu, D. R. A system for the continuous directed evolution of biomolecules. Nature 472, 499-503, doi:10.1038/nature09929 (2011).
19. Badran, A. H. et al. Continuous evolution of Bacillus thuringiensis toxins overcomes insect resistance. Nature 533, 58-63, doi:10.1038/nature17938 (2016).
20. Hubbard, B. P. et al. Continuous directed evolution of DNA-binding proteins to improve TALEN specificity. Nat Methods 12, 939-942, doi:10.1038/nmeth.3515 (2015).
21. Bryson, D. I. et al. Continuous directed evolution of aminoacyl-tRNA synthetases. Nat Chem Biol, doi:10.1038/nchembio.2474 (2017).
22. Packer, M. S., Rees, H. A. & Liu, D. R. Phage-assisted continuous evolution of proteases with altered substrate specificity. Nat Commun 8, 956, doi:10.1038/s41467-017-01055-9 (2017).
23. Meng, X. & Wolfe, S. A. Identifying DNA sequences recognized by a transcription factor using a bacterial one-hybrid system. Nat Protoc 1, 30-45, doi:10.1038/nprot.2006.6 (2006).
24. Dove, S. L., Joung, J. K. & Hochschild, A. Activation of prokaryotic transcription through arbitrary protein-protein contacts. Nature 386, 627-630, doi:10.1038/386627a0 (1997).
25. Anders, C., Niewoehner, O., Duerst, A. & Jinek, M. Structural basis of PAM-dependent target DNA recognition by the Cas9 endonuclease. Nature 513, 569-573, doi:10.1038/nature13579 (2014).
26. Chen, J. S. et al. Enhanced proofreading governs CRISPR-Cas9 targeting accuracy. Nature 550, 407-410, doi:10.1038/nature24268 (2017).
27. Sternberg, S. H., LaFrance, B., Kaplan, M. & Doudna, J. A. Conformational control of DNA target cleavage by CRISPR-Cas9. Nature 527, 110-113, doi:10.1038/nature15544 (2015).
28. Gao, L. et al. Engineered Cpf1 variants with altered PAM specificities. Nat Biotechnol 35, 789-792, doi:10.1038/nbt.3900 (2017).
29. Chavez, A. et al. Highly efficient Cas9-mediated transcriptional programming. Nat Methods 12, 326-328, doi:10.1038/nmeth.3312 (2015).
30. Doench, J. G. et al. Rational design of highly active sgRNAs for CRISPR-Cas9-mediated gene inactivation. Nat Biotechnol 32, 1262-1267, doi:10.1038/nbt.3026 (2014).
31. Zhang, Y. et al. Comparison of non-canonical PAMs for CRISPR/Cas9-mediated DNA cleavage in human cells. Sci Rep 4, 5405, doi:10.1038/srep05405 (2014).
32. Tsai, S. Q. et al. GUIDE-seq enables genome-wide profiling of off-target cleavage by CRISPR-Cas nucleases. Nat Biotechnol 33, 187-197, doi:10.1038/nbt.3117 (2015).
33. Kim, Y. B. et al. Increasing the genome-targeting scope and precision of base editing with engineered Cas9-cytidine deaminase fusions. Nat Biotechnol 35, 371-376, doi:10.1038/nbt.3803 (2017).
34. Komor, A. C. et al. Improved base excision repair inhibition and bacteriophage Mu Gam protein yields C:G-to-T:A base editors with higher efficiency and product purity. Sci Adv 3, eaao4774, doi:10.1126/sciadv.aao4774 (2017).
35. Pattanayak, V., Ramirez, C. L., Joung, J. K. & Liu, D. R. Revealing off-target cleavage specificities of zinc-finger nucleases by in vitro selection. Nat Methods 8, 765-770, doi:10.1038/nmeth.1670 (2011).
36. Slaymaker, I. M. et al. Rationally engineered Cas9 nucleases with improved specificity. Science 351, 84-88, doi:10.1126/science.aad5227 (2016).
37. Kleinstiver, B. P. et al. High-fidelity CRISPR-Cas9 nucleases with no detectable genome-wide off-target effects. Nature 529, 490-495, doi:10.1038/nature16526 (2016).
38. Landrum, M. J. et al. ClinVar: public archive of relationships among sequence variation and human phenotype. Nucleic Acids Res 42, D980-985, doi:10.1093/nar/gkt1113 (2014).
39. Crooks, G. E., Hon, G., Chandonia, J. M. & Brenner, S. E. WebLogo: a sequence logo generator. Genome Res 14, 1188-1190, doi:10.1101/gr.849004 (2004).
40. Humbert O, Davis L, Maizels N. Targeted gene therapies: tools, applications, optimization. Crit Rev Biochem Mol.2012; 47(3):264-81. PMID: 22530743.
41. Perez-Pinera P, Ousterout D G, Gersbach C A. Advances in targeted genome editing. Curr Opin Chem Biol. 2012; 16(3-4):268-77. PMID: 22819644.
42. Urnov F D, Rebar E J, Holmes M C, Zhang H S, Gregory P D. Genome editing with engineered zinc finger nucleases. Nat Rev Genet. 2010; 11(9):636-46. PMID: 20717154.
43. Joung J K, Sander J D. TALENs: a widely applicable technology for targeted genome editing. Nat Rev Mol Cell Biol. 2013; 14(1):49-55. PMID: 23169466.
44. Charpentier E, Doudna J A. Biotechnology: Rewriting a genome. Nature. 2013; 495, (7439):50-1. PMID: 23467164.
45. Pan Y, Xia L, Li A S, Zhang X, Sirois P, Zhang J, Li K. Biological and biomedical applications of engineered nucleases. Mol Biotechnol. 2013; 55(1):54-62. PMID: 23089945.
46. De Souza, N. Primer: genome editing with engineered nucleases. Nat Methods. 2012; 9(1):27. PMID: 22312638.
47. Santiago Y, Chan E, Liu P Q, Orlando S, Zhang L, Urnov F D, Holmes M C, Guschin D, Waite A, Miller J C, Rebar E J, Gregory P D, Klug A, Collingwood T N. Targeted gene knockout in mammalian cells by using engineered zinc-finger nucleases. Proc Natl Acad Sci USA. 2008; 105(15):5809-14. PMID: 18359850.
48. Cargill M, Altshuler D, Ireland J, Sklar P, Ardlie K, Patil N, Lane C R, Lim E P, Kalyanaraman N, Nemesh J, Ziaugra L, Friedland L, Rolfe A, Warrington J, Lipshutz R, Daley G Q, Lander E S. Characterization of single-nucleotide polymorphisms in coding regions of human genes. Nat Genet. 1999; 22(3):231-8. PMID: 10391209.
49. Jansen R, van Embden J D, Gaastra W, Schouls L M. Identification of genes that are associated with DNA repeats in prokaryotes. Mol Microbiol. 2002; 43(6):1565-75. PMID: 11952905.
50. Mali P, Esvelt K M, Church G M. Cas9 as a versatile tool for engineering biology. Nat Methods. 2013; 10(10):957-63. PMID: 24076990.
51. Jore M M, Lundgren M, van Duijin E, Bultema J B, Westra E R, Waghmare S P, Wiedenheft B, Pul U, Wurm R, Wagner R, Beijer M R, Barendregt A, Shou K, Snijders A P, Dickman M J, Doudna J A, Boekema E J, Heck A J, van der Oost J, Brouns S J. Structural basis for CRISPR RNA-guided DNA recognition by Cascade. Nat Struct Mol Biol. 2011; 18(5):529-36. PMID: 21460843.
52. Horvath P, Barrangou R. CRISPR/Cas, the immune system of bacteria and archaea. Science. 2010; 327(5962):167-70. PMID: 20056882.
53. Wiedenheft B, Sternberg S H, Doudna J A. RNA-guided genetic silencing systems in bacteria and archaea. Nature. 2012; 482(7385):331-8. PMID: 22337052.
54. Gasiunas G, Siksnys V. RNA-dependent DNA endonuclease Cas9 of the CRISPR system: Holy Grail of genome editing? Trends Microbiol. 2013; 21(11):562-7. PMID: 24095303.
55. Qi L S, Larson M H, Gilbert L A, Doudna J A, Weissman J S, Arkin A P, Lim W A. Repurposing CRISPR as an RNA-guided platform for sequence-specific control of gene expression. Cell. 2013; 152(5):1173-83. PMID: 23452860.
56. Perez-Pinera P, Kocak D D, Vockley C M, Adler A F, Kabadi A M, Polstein L R, Thakore P I, Glass K A, Ousterout D G, Leong K W, Guilak F, Crawford G E, Reddy T E, Gersbach C A. RNA-guided gene activation by CRISPR-Cas9-based transcription factors. Nat Methods. 2013; 10(10):973-6. PMID: 23892895.
57. Mali P, Aach J, Stranges P B, Esvelt K M, Moosburner M, Kosuri S, Yang L, Church G M. CAS9 transcriptional activators for target specificity screening and paired nickases for cooperative genome engineering. Nat Biotechnol. 2013; 31(9):833-8. PMID: 23907171.
58. Gilbert L A, Larson M H, Morsut L, Liu Z, Brar G A, Torres S E, Stern-Ginossar N, Brandman O, Whitehead E H, Doudna J A, Lim W A, Weissman J S, Qi L S. CRISPR-mediated modular RNA-guided regulation of transcription in eukaryotes. Cell. 2013; 154(2):442-51. PMID: 23849981.
59. Larson M H, Gilbert L A, Wang X, Lim W A, Weissman J S, Qi L S. CRISPR interference (CRISPRi) for sequence-specific control of gene expression. Nat Protoc. 2013; 8(11):2180-96. PMID: 24136345.
60. Mali P, Yang L, Esvelt K M, Aach J, Guell M, DiCarlo J E, Norville J E, Church G M. RNA-guided human genome engineering via Cas9. Science. 2013; 339(6121):823-6. PMID: 23287722.
61. Cole-Strauss A, Yoon K, Xiang Y, Byrne B C, Rice M C, Gryn J, Holloman W K, Kmiec E B. Correction of the mutation responsible for sickle cell anemia by an RNA-DNA oligonucleotide. Science. 1996; 273(5280):1386-9. PMID: 8703073.
62. Tagalakis A D, Owen J S, Simons J P. Lack of RNA-DNA oligonucleotide (chimeraplast) mutagenic activity in mouse embryos. Mol Reprod Dev. 2005; 71(2):140-4. PMID: 15791601.
63. Ray A, Langer M. Homologous recombination: ends as the means. Trends Plant Sci. 2002; 7(10):435-40. PMID 12399177.
64. Britt A B, May G D. Re-engineering plant gene targeting. Trends Plant Sci. 2003; 8(2):90-5. PMID: 12597876.
65. Vagner V, Ehrlich S D. Efficiency of homologous DNA recombination varies along the Bacillus subtilis chromosome. J Bacteriol. 1988; 170(9):3978-82. PMID: 3137211.
66. Saleh-Gohari N, Helleday T. Conservative homologous recombination preferentially repairs DNA double-strand breaks in the S phase of the cell cycle in human cells. Nucleic Acids Res. 2004; 32(12):3683-8. PMID: 15252152.
67. Lombardo A, Genovese P, Beausejour C M, Colleoni S, Lee Y L, Kim K A, Ando D, Urnov F D, Galli C, Gregory P D, Holmes M C, Naldini L. Gene editing in human stem cells using zinc finger nucleases and integrase-defective lentiviral vector delivery. Nat Biotechnol. 2007; 25(11):1298-306. PMID: 17965707.

### Supplemental Information

TABLE 1
Target sites used to test sgRNAs for activation
of gene III expression in PACE.

| | | | Distance | |
|---|---|---|---|---|
| | | | from PAM | |
| | | | to -35 | SEQ ID |
| Label | Sequence | PAM | signal | NO |
| G0 | GTGAACCGCATCGAGCTGAA | n/a | Scrambled | SEQ ID |
| | | | | NO: 264 |
| G1 | AACACGCACGGTGTTACATT | AGG | 10 bp | SEQ ID |
| | | | | NO: 265 |
| G2 | TAGGGACCCTACAACACGCA | CGG | 22 bp | SEQ ID |
| | | | | NO: 266 |
| G3 | CGTTTTGCTGAGGAGACTTA | GGG | 44 bp | SEQ ID |
| | | | | NO: 267 |
| G4 | CTGGGCCTTTCGTTTTGCTG | AGG | 50 bp | SEQ ID |
| | | | | NO: 268 |
| G5 | AAAGGCTCAGTCGAAAGACT | GGG | 68 bp | SEQ ID |
| | | | | NO: 269 |
| G6′ | GTAACACCGTGCGTGTTGTA | CGG | 36 bp | SEQ ID |
| | | | | NO: 274 |
| G7′ | AGTCTCCTCAGCAAAACGAA | CGG | 65 bp | SEQ ID |
| | | | | NO: 275 |

TABLE 2
Linker optimization of ω-dCas9 constructs for
PACE from FIG. 5C.

| Label | Sequence | Length |
|---|---|---|
| L1 | AA (SEQ ID NO: 319) | 2 |
| L2 | EQKLISEEDL (SEQ ID NO: 320) | 10 |
| L3 | GGSGGSGGSGGSGGS (SEQ ID NO: 321) | 15 |
| L4 | GHGTGSTGSGSS (SEQ ID NO: 322) | 12 |
| L5 | MSRPDPA (SEQ ID NO: 323) | 7 |
| L6 | GSAGSAAGSGEF (SEQ ID NO: 324) | 12 |
| L7 | SGSETPGTSESATPES (SEQ ID NO: 325) | 16 |

TABLE 3
Mutations in ω-dCas9 variants from FIG. 5D.

| ω/dCas9 | Mutations relative to canonical ω-dCas9 |
|---|---|
| wt/wt | I12N(ω) |
| PACE1/PACE1 | I12N(ω), H112Y, L300F, Y449H, N802S, K1288E |
| PACE2/PACE2 | F574L, L641I |
| PACE3/PACE3 | Q30K(ω) |
| wt/PACE1 | H112Y, L300F, Y449H, D706D, N802S, K1288E |

TABLE 4
Target sites used in xCas9 PACE.

| Label | Sequence |
|---|---|
| G7′ | AGTCTCCTCAGCAAAACGAA (SEQ ID NO: 276) |
| CD13 | GGAGCCCACCGAGTACCTGG (SEQ ID NO: 277) |
| VEGFA | GACCCCCTCCACCCCGCCTC (SEQ ID NO: 278) |

TABLE 6
Plasmids used for Cas9 PACE and characterization.

| Plasmid | Antibiotic | | ORF1 | | ORF2 | | | |
|---|---|---|---|---|---|---|---|---|
| name | resistance | ORI | Promoter | Gene | Promoter | Gene | Comments | Figure(s) |
| PhJH008 | None | M13 f1 | P_{gIII} | ω-dCas9 | | | SP for PACE^{1.2} | 1B |
| MP4 | chlor^{R} | CloDF13 | p_{BAD} | dnaQ926, dam, | PC | araC | MP for PACE³ | 1B |
| | | | | seqA | | | | |
| MP6 | chlor^{R} | CloDF13 | p_{BAD} | dnaQ926, dam, | PC | araC | MP for PACE³ | 1B |
| | | | | seqA, emrR, | | | | |
| | | | | ugi, cda1 | | | | |
| pJH414 | carb^{R} | SC101 | P_{lacZ} | gIII, luxAB | P_{lacZ} (no LacO) | sgRNA | AP (G7′ NGG PAM) | 5A-5D |
| pJH447 | carb^{R} | SC101 | P_{lacZ} | gIII, luxAB | P_{lacZ} (no LacO) | sgRNA | AP (G7′ NNN PAM | 1B |
| | | | | | | | library) | |
| pJH451 | carb^{R} | SC101 | P_{lacZ} | gIII, luxAB | P_{lacZ} (no LacO) | sgRNA | AP (CD13 NNN PAM | 1B |
| | | | | | | | library) | |
| pJH453 | carb^{R} | SC101 | P_{lacZ} | gIII, luxAB | P_{lacZ} (no LacO) | sgRNA | AP (VEGFA NNN | 1B |
| | | | | | | | PAM library) | |
| pJH958 | spect^{R} | SC101 | Pₜₑₜ | xCas9 3.0 | Plac (no LacO) | sgRNA | PAM depletion | 6A-6C |
| pJH959 | spect^{R} | SC101 | Pₜₑₜ | xCas9 3.1 | Plac (no LacO) | sgRNA | PAM depletion | 6A-6C |
| pJH960 | spect^{R} | SC101 | Pₜₑₜ | xCas9 3.2 | Plac (no LacO) | sgRNA | PAM depletion | 6A-6C |
| pJH961 | spect^{R} | SC101 | Pₜₑₜ | xCas9 3.3 | Plac (no LacO) | sgRNA | PAM depletion | 6A-6C |
| pJH962 | spect^{R} | SC101 | Pₜₑₜ | xCas9 3.4 | Plac (no LacO) | sgRNA | PAM depletion | 6A-6C |
| pJH963 | spect^{R} | SC101 | Pₜₑₜ | xCas9 3.5 | Plac (no LacO) | sgRNA | PAM depletion | 6A-6C |
| pJH964 | spect^{R} | SC101 | Pₜₑₜ | xCas9 3.6 | Plac (no LacO) | sgRNA | PAM depletion | 6A-6C |
| pJH965 | spect^{R} | SC101 | Pₜₑₜ | xCas9 3.7 | Plac (no LacO) | sgRNA | PAM depletion | 6A-6C |
| pJH966 | spect^{R} | SC101 | Pₜₑₜ | xCas9 3.8 | Plac (no LacO) | sgRNA | PAM depletion | 6A-6C |
| pJH967 | spect^{R} | SC101 | Pₜₑₜ | xCas9 3.9 | Plac (no LacO) | sgRNA | PAM depletion | 6A-6C |
| pJH968 | spect^{R} | SC101 | Pₜₑₜ | xCas9 3.10 | Plac (no LacO) | sgRNA | PAM depletion | 6A-6C |
| pJH969 | spect^{R} | SC101 | Pₜₑₜ | xCas9 3.11 | Plac (no LacO) | sgRNA | PAM depletion | 6A-6C |
| pJH970 | spect^{R} | SC101 | Pₜₑₜ | xCas9 3.12 | Plac (no LacO) | sgRNA | PAM depletion | 6A-6C |
| pJH971 | spect^{R} | SC101 | Pₜₑₜ | xCas9 3.13 | Plac (no LacO) | sgRNA | PAM depletion | 6A-6C |
| pJH306 | carb^{R} | BR322 | CMV | dSpCas9-VPR | | | GFP activation⁴ | 2A, 7A-7C, 8A-8D, |
| | | | | | | | | 9A-9E, 10A-10D |
| pJH599 | carb^{R} | BR322 | CMV | dxCas9(2.0)-VPR | | | GFP activation | 7C, 8A-8D |
| pJH11172 | carb^{R} | BR322 | CMV | dxCas9(3.6)-VPR | | | GFP activation | 7A, 7B, 10A-10D |
| pJH11173 | carb^{R} | BR322 | CMV | dxCas9(3.7)-VPR | | | GFP activation | 2A, 7A, 7B, 9A-9E |
| pJH407 | carb^{R} | BR322 | CMV | SpCas9 | | | DNA cutting | 2B, 2C, 4A-4F, |
| | | | | | | | | 11A-11D, 12A-12C, |
| | | | | | | | | 13A-13F, 14A-14E |
| pJH1009 | carb^{R} | BR322 | CMV | xCas9 3.6 | | | DNA cutting | 11 A, 11B, 13A-13F, |
| | | | | | | | | 14B, 14C |
| pJH1010 | carb^{R} | BR322 | CMV | xCas9 3.7 | | | DNA cutting | 2B, 2C, 4A-4F, |
| | | | | | | | | 13A-13F, 14A |
| pJH1054 | carb^{R} | BR322 | CMV | SpCas9-BE3 | | | C•G to T•A base | 3A |
| | | | | | | | editor⁵ | |
| pJH1056 | carb^{R} | BR322 | CMV | xCas9(3.6)-BE3 | | | C•G to T•A base | 11C |
| | | | | | | | editor | |
| pJH11171 | carb^{R} | BR322 | CMV | xCas9(3.7)-BE3 | | | C•G to T•A base | 3A |
| | | | | | | | editor | |
| pJH11174 | carb^{R} | BR322 | CMV | SpCas9-ABE | | | A•T to G•C base | 3B |
| | | | | | | | editor⁶ | |
| pJH11175 | carb^{R} | BR322 | CMV | xCas9(3.6)-ABE | | | A•T to G•C base | 11D |
| | | | | | | | editor | |
| pJH11176 | carb^{R} | BR322 | CMV | xCas9(3.7)-ABE | | | A•T to G•C base | 3B |
| | | | | | | | editor | |

TABLE 7
Primers used for PAM depletion assay HTS.

| Primer name | Sequence |
|---|---|
| PAM depletion | ACACTCTTTCCCTACACGACGCTCTTCCGATCTN |
| fwd 1 | NNNTTATATAGGCCTCCCACCGTAC |
| | (SEQ ID NO: 279) |
| PAM depletion | TGGAGTTCAGACGTGTGCTCTTCCGATCTACAGG |
| rev 1 | GTAATAGTGTCCCCTC |
| | (SEQ ID NO: 280) |
| PAM depletion | ACACTCTTTCCCTACACGACGCTCTTCCGATCTN |
| fwd 2 | NNNCTTATATAGGCCTCCCACCG |
| | (SEQ ID NO: 281) |
| PAM depletion | TGGAGTTCAGACGTGTGCTCTTCCGATCTTGCTC |
| rev 2 | GAGCGGCCGCC |
| | (SEQ ID NO: 282) |

TABLE 8
Target sequences used for GFP transcriptional
activation assays.

| Site | PAM | Protospacer sequence |
|---|---|---|
| R1 | GGG | GTCCCCTCCACCCCACAGTG |
| | | (SEQ ID NO: 283) |
| R2 | TGG | GGGGCCACTAGGGACAGGAT |
| | | (SEQ ID NO: 284) |

TABLE 9
Target sites used for endogenous gene
transcriptional activation.

| Gene | PAM | Protospacer sequence |
|---|---|---|
| NEUROD1 | GGG | GTCTCTAACTGGCGACAGAT |
| | | (SEQ ID NO: 285) |
| ASCL1 | GGG | GGGAGAAAGGAACGGGAGGG |
| | | (SEQ ID NO: 286) |
| MIAT | CGC | GGCAAGCGTTGCCATGACAG |
| | | (SEQ ID NO: 287) |
| NEUROD1 | AGT | GGGTAAAAACAGGTCCGCGG |
| | | (SEQ ID NO: 288) |
| RHOXF2 | GGC | GCTCTCCCTCATCCGGGCAA |
| | | (SEQ ID NO: 289) |
| MIAT | CTG | GCGGGTGGGGATGCGGGAGG |
| | | (SEQ ID NO: 290) |

TABLE 10
Primers used for endogenous gene transcriptional
activation analysis.

| Site | Primer sequence |
|---|---|
| NEUROD1 fwd | GGATGACGATCAAAAGCCCAA |
| | (SEQ ID NO: 291) |
| NEUROD1 rev | GCGTCTTAGAATAGCAAGGCA |
| | (SEQ ID NO: 293) |
| ASCL1 fwd | CGCGGCCAACAAGAAGATG |
| | (SEQ ID NO: 311) |
| ASCL1 rev | CGACGAGTAGGATGAGACCG |
| | (SEQ ID NO: 312) |
| MIAT fwd | TGGCTGGGGTTTGAACCTTT |
| | (SEQ ID NO: 313) |
| MIAT rev | AGGAAGCTGTTCCAGACTGC |
| | (SEQ ID NO: 326) |
| RHOXF2 fwd | GGCAAGAAGCATGAATGTGA |
| | (SEQ ID NO: 327) |
| RHOXF2 rev | TGTCTCCTCCATTTGGCTCT |
| | (SEQ ID NO: 328) |

TABLE 11
Target sites used in the GFP disruption assay.
An RNA sequence complementary to the protospacer
sequence in the table would be used in a gRNA to
target a Cas9 domain or a fusion protein
comprising a Cas9 domain to the sequence. sgRNA
plasmids were generated with reverse primer
GGTGTTTCGTCCTTTCCACAAGATATATAAAG (SEQ ID NO: 329)
and forward primer
(N)₂₀GTTTTAGAGCTAGAAATAGCAAGTTAAAATAAGGC (SEQ ID
NO: 330), where (N)₂₀ is the protospacer sequence.

| Site | PAM | Protospacer sequence |
|---|---|---|
| GFP1 | CGG | GACCAGGATGGGCACCACCC |
| | | (SEQ ID NO: 331) |
| GFP2 | GGG | GTGGTCACGAGGGTGGGCCA |
| | | (SEQ ID NO: 332) |
| GFP3 | GGA | GTGCCCATCCTGGTCGAGC |
| | | (SEQ ID NO: 333) |
| GFP4 | CGT | GTGACCACCTTCACCTACGG |
| | | (SEQ ID NO: 334) |
| GFP5 | GGT | GTGGTGCAGATGAACTTCAG |
| | | (SEQ ID NO: 335) |
| GFP6 | GGT | GAAGTCGTGCTGCTTCATGT |
| | | (SEQ ID NO: 336) |
| GFP7 | TGC | GGAGCTGTTCACCGGGGTGG |
| | | (SEQ ID NO: 337) |
| GFP8 | GAT | GAGTTCGTGACCGCCGCCGG |
| | | (SEQ ID NO: 338) |
| GFP9 | CAA | GTGAACTTCAAGACCCGCCA |
| | | (SEQ ID NO: 339) |
| GFP10 | ACG | GGTAGTGGTCGGCGAGCTGC |
| | | (SEQ ID NO: 340) |
| GFP11 | ATG | GCTGCACGCTGCCGTCCTCG |
| | | (SEQ ID NO: 341) |

TABLE 12
Genomic sites used for targeted genome editing. An RNA sequence
complementary to the protospacer sequence in the table would be used
in a gRNA to target a Cas9 domain or a fusion protein comprising a
Cas9 domain to the sequence. sgRNA plasmids were generated with
reverse primer GGTGTTTCGTCCTTTCCACAAGATATATAAAG (SEQ ID NO: 329)
and forward primer (N)₂₀GTTTTAGAGCTAGAAATAGCAAGTTAAAATAAGGC
(SEQ ID NO: 330), where (N)₂₀ is the protospacer sequence.

| Site | Genomic locus | PAM | Protospacer sequence |
|---|---|---|---|
| 1 | HEK site 4 | CGG | GATGACAGGCAGGGGCACCG (SEQ ID NO: 342) |
| 2 | EMX1 | GGG | GGCCCCAGTGGCTGCTCTGG (SEQ ID NO: 343) |
| 3 | EMX1 | GGG | GGCCCCCAGAGCAGCCACTG (SEQ ID NO: 344) |
| 4 | HEK site 4 | GGG | GGCACTGCGGCTGGAGGTGG (SEQ ID NO: 345) |
| 5 | VEGFA | AGT | GAGCGAGCAGCGTCTTCGAG (SEQ ID NO: 346) |
| 6 | FANCF | CGT | GCGGTCTCAAGCACTACCTA (SEQ ID NO: 347) |
| 7 | HEK site 3 | TGT | GAGCTGCACATACTAGCCCC (SEQ ID NO: 348) |
| 8 | HEK site 3 | GGT | GCTTCTCCAGCCCTGGCCTG (SEQ ID NO: 349) |
| 9 | HEK site 4 | AGC | GGCCCCCACTGTAGTCACAC (SEQ ID NO: 369) |
| 10 | HEK site 4 | CGC | GACAGGCAGGGGCACCGCGG (SEQ ID NO: 370) |
| 11 | HEK site 4 | TGC | GTGCCACCGGGGCGCCGCGG (SEQ ID NO: 371) |
| 12 | VEGFA | GGC | GCAGACGGCAGTCACTAGGG (SEQ ID NO: 372) |
| 13 | HEK site 3 | AGA | GTCGCAGGACAGCTTTTCCT (SEQ ID NO: 373) |
| 14 | VEGFA | CGA | GGGAAGCTGGGTGAATGGAG (SEQ ID NO: 390) |
| 15 | HEK site 4 | TGA | GCGGAGACTCTGGTGCTGTG (SEQ ID NO: 391) |
| 16 | VEGFA | GGA | GGTCAGAAATAGGGGGTCCA (SEQ ID NO: 392) |
| 17 | FANCF | GAA | GACTCTCTGCGTACTGATTG (SEQ ID NO: 393) |
| 18 | FANCF | GAT | GATCCAGGTGCTGCAGAAGG (SEQ ID NO: 394) |
| 19 | VEGFA | GAT | GTCACTCCAGGATTCCAATA (SEQ ID NO: 395) |
| 20 | EMX1 | CAA | GTGGTTCCAGAACCGGAGGA (SEQ ID NO: 396) |
| F1 | FANCF | TGG | GGAATCCCTTCTGCAGC (SEQ ID NO: 397) |
| F2 | FANCF | AGT | GGCGGGCCAGGCTCTCTTGG (SEQ ID NO: 398) |
| F3 | FANCF | CGT | GGGCCCTCGGGGATGCAGCT (SEQ ID NO: 399) |
| F4 | FANCF | TGT | GCCCTCTTGCCTCCACTGGT (SEQ ID NO: 459) |
| F5 | FANCF | GGT | GCCACATCCATCGGCGCTTT (SEQ ID NO: 460) |
| F6 | FANCF | GGT | GCCGCCCTCTTGCCTCCACT (SEQ ID NO: 461) |
| F7 | FANCF | CGC | GTGACGTCCTGCTCTCTCTG (SEQ ID NO: 462) |
| F8 | FANCF | CGC | GCGCCGGGCCTTGCAGTGGG (SEQ ID NO: 463) |
| F9 | FANCF | CGC | GCGCCCACTGCAAGGCCCGG (SEQ ID NO: 464) |
| F10 | FANCF | TGC | GAGAACCGGGCCCTCGGGGA (SEQ ID NO: 465) |
| F11 | FANCF | GGC | GAGACACTCCAAGAGAGCCT (SEQ ID NO: 466) |
| F12 | FANCF | AGA | GCGCTTCAATGGCTATAGAG (SEQ ID NO: 467) |
| F13 | FANCF | TGA | GAAGGTCATAGTGCAAACGT (SEQ ID NO: 468) |
| F14 | FANCF | GGA | GAGAACCCAAATCTCCAGGA (SEQ ID NO: 469) |
| F15 | FANCF | GGA | GGCCCCATTCGCACGGCTCT (SEQ ID NO: 470) |

TABLE 13
Primers for mammalian cell genomic DNA amplification.

| Primer name | Sequence |
|---|---|
| VEGFA fwd 1 | ACACTCTTTCCCTACACGACGCTCTTCCGATCTNNNNGCCCATTCCCTCTTTAGCCA |
| | (SEQ ID NO: 471) |
| VEGFA rev 1 | TGGAGTTCAGACGTGTGCTCTTCCGATCTCTCAACCCCACACGCACA |
| | (SEQ ID NO: 472) |
| VEGFA fwd 2 | ACACTCTTTCCCTACACGACGCTCTTCCGATCTNNNNCCACCACAGGGAAGCTGG |
| | (SEQ ID NO: 473) |
| VEGFA rev 2 | TGGAGTTCAGACGTGTGCTCTTCCGATCTCTCAACCCCACACGCACA |
| | (SEQ ID NO: 474) |
| VEGFA fwd 3 | ACACTCTTTCCCTACACGACGCTCTTCCGATCTNNNNGTCAGAGGGACACACTGTGG |
| | (SEQ ID NO: 475) |
| VEGFA rev 3 | TGGAGTTCAGACGTGTGCTCTTCCGATCTAATGGGCTTTGGAAAGGGGG |
| | (SEQ ID NO: 476) |
| VEGFA fwd 4 | ACACTCTTTCCCTACACGACGCTCTTCCGATCTNNNNGAGGACGTGTGTGTCTGTGT |
| | (SEQ ID NO: 477) |
| VEGFA rev 4 | TGGAGTTCAGACGTGTGCTCTTCCGATCTATATTGAAGGGGGCAGGGGA |
| | (SEQ ID NO: 478) |
| VEGFA fwd 5 | ACACTCTTTCCCTACACGACGCTCTTCCGATCTNNNNTGGATCGCTTTTCCGAGCTT |
| | (SEQ ID NO: 479) |
| VEGFA rev 5 | TGGAGTTCAGACGTGTGCTCTTCCGATCTGACGTCACAGTGACCGAGG |
| | (SEQ ID NO: 480) |
| VEGFA fwd 6 | ACACTCTTTCCCTACACGACGCTCTTCCGATCTNNNNCACAACCAGTGGAGGCAAGA |
| | (SEQ ID NO: 481) |
| VEGFA rev 6 | TGGAGTTCAGACGTGTGCTCTTCCGATCTCCAGGCTCTCTTGGAGTGTC |
| | (SEQ ID NO: 482) |
| VEGFA fwd 7 | ACACTCTTTCCCTACACGACGCTCTTCCGATCTNNNNACCTGGTGCAGCAACTCTTT |
| | (SEQ ID NO: 483) |
| VEGFA rev 7 | TGGAGTTCAGACGTGTGCTCTTCCGATCTGCCTGGGTCTTCATCAGAGAG |
| | (SEQ ID NO: 484) |
| VEGFA fwd 8 | ACACTCTTTCCCTACACGACGCTCTTCCGATCTNNNNGGACGCCGATGAGGAGAC |
| | (SEQ ID NO: 485) |
| VEGFA rev 8 | TGGAGTTCAGACGTGTGCTCTTCCGATCTTTGTTCTGAGGCAAGCGCTC |
| | (SEQ ID NO: 486) |
| VEGFA fwd 9 | ACACTCTTTCCCTACACGACGCTCTTCCGATCTNNNNGCTAGTCCACTGGCTTCTGG |
| | (SEQ ID NO: 487) |
| VEGFA rev 9 | TGGAGTTCAGACGTGTGCTCTTCCGATCTATTGTGCAACTCCTCCCAGG |
| | (SEQ ID NO: 488) |
| EMX1 fwd | ACACTCTTTCCCTACACGACGCTCTTCCGATCTNNNNCAGCTCAGCCTGAGTGTTGA |
| | (SEQ ID NO: 489) |
| EMX1 rev | TGGAGTTCAGACGTGTGCTCTTCCGATCTCTCGTGGGTTTGTGGTTGC |
| | (SEQ ID NO: 490) |
| FANCF fwd 1 | ACACTCTTTCCCTACACGACGCTCTTCCGATCTNNNNCATTGCAGAGAGGCGTATCA |
| | (SEQ ID NO: 491) |
| FANCF rev 1 | TGGAGTTCAGACGTGTGCTCTTCCGATCTGGGGTCCCAGGTGCTGAC |
| | (SEQ ID NO: 492) |
| FANCF fwd 2 | ACACTCTTTCCCTACACGACGCTCTTCCGATCTNNNNGAATCCCTTCTGCAGCACCT |
| | (SEQ ID NO: 493) |
| FANCF rev 2 | TGGAGTTCAGACGTGTGCTCTTCCGATCTGATGGATGTGGCGCAGGTAG |
| | (SEQ ID NO: 494) |
| HEK site 4 | ACACTCTTTCCCTACACGACGCTCTTCCGATCTNNNNGAACCCAGGTAGCCAGAGAC |
| fwd | (SEQ ID NO: 495) |
| HEK site 4 | TGGAGTTCAGACGTGTGCTCTTCCGATCTTCCTTTCAACCCGAACGGAG |
| rev | (SEQ ID NO: 496) |
| HEK site 3 | ACACTCTTTCCCTACACGACGCTCTTCCGATCTNNNNATGTGGGCTGCCTAGAAAGG |
| fwd | (SEQ ID NO: 497) |
| HEK site 3 | TGGAGTTCAGACGTGTGCTCTTCCGATCTCCCAGCCAAACTTGTCAACC |
| rev | (SEQ ID NO: 498) |
| EMX1 on- | ACACTCTTTCCCTACACGACGCTCTTCCGATCTNNNNGGAGGACAAAGTACAAACGGC |
| target fwd | (SEQ ID NO: 499) |
| EMX1 on- | TGGAGTTCAGACGTGTGCTCTTCCGATCTATCGATGTCCTCCCCATTGG |
| target rev | (SEQ ID NO: 504) |
| EMX1 off- | ACACTCTTTCCCTACACGACGCTCTTCCGATCTNNNNCGGCCTTTGCAAATAGAGCC |
| target 1 fwd | (SEQ ID NO: 505) |
| EMX1 off- | TGGAGTTCAGACGTGTGCTCTTCCGATCTTTGGCCTTTGTAGGAAAACACC |
| target 1 rev | (SEQ ID NO: 506) |
| HEK site 1 | ACACTCTTTCCCTACACGACGCTCTTCCGATCTNNNNAGCCAGGACCTGACATGCAG |
| on-target fwd | (SEQ ID NO: 507) |
| HEK site 1 | TGGAGTTCAGACGTGTGCTCTTCCGATCTCTTCCTCTTTCCCCTGTAGT |
| on-target rev | (SEQ ID NO: 508) |
| HEK site 1 | ACACTCTTTCCCTACACGACGCTCTTCCGATCTNNNNATGTCAACTCTGGAGCTGAT |
| off-target 1 | (SEQ ID NO: 509) |
| fwd | |
| HEK site 1 | TGGAGTTCAGACGTGTGCTCTTCCGATCTTCTCCTGGCTAGAAATCAGC |
| off-target 1 | (SEQ ID NO: 512) |
| rev | |
| HEK site 2 | ACACTCTTTCCCTACACGACGCTCTTCCGATCTNNNNCCTGGCTGAGCTAACTGTGA |
| on-target fwd | (SEQ ID NO: 513) |
| HEK site 2 | TGGAGTTCAGACGTGTGCTCTTCCGATCTCCAGCCCCATCTGTCAAACT |
| on-target rev | (SEQ ID NO: 514) |
| HEK site 2 | ACACTCTTTCCCTACACGACGCTCTTCCGATCTNNNNGCTCTCAGCTCAGCCTGAGT |
| off-target 1 | (SEQ ID NO: 516) |
| fwd | |
| HEK site 2 | TGGAGTTCAGACGTGTGCTCTTCCGATCTTTGGAGGTGACATCGATGTC |
| off-target 1 | (SEQ ID NO: 517) |
| rev | |
| HEK site 2 | ACACTCTTTCCCTACACGACGCTCTTCCGATCTNNNNTGGGTTCAAAAACAAACAGAGAA |
| off-target 2 | (SEQ ID NO: 518) |
| fwd | |
| HEK site 2 | TGGAGTTCAGACGTGTGCTCTTCCGATCTTTTAATGCTCCCACACCATTTTT |
| off-target 2 | (SEQ ID NO: 519) |
| rev | |
| HEK site 3 | ACACTCTTTCCCTACACGACGCTCTTCCGATCTNNNNGGAAACGCCCATGCAATTAGT |
| on-target fwd | (SEQ ID NO: 523) |
| HEK site 3 | TGGAGTTCAGACGTGTGCTCTTCCGATCTGAGCTGCACATACTAGCCCC |
| on-target rev | (SEQ ID NO: 524) |
| HEK site 3 | ACACTCTTTCCCTACACGACGCTCTTCCGATCTNNNNTCTTGCTGAGATGTGGGCAG |
| off-target 1 | (SEQ ID NO: 525) |
| fwd | |
| HEK site 3 | TGGAGTTCAGACGTGTGCTCTTCCGATCTTCAGGAGTACACAGAGGTCC |
| off-target 1 | (SEQ ID NO: 526) |
| rev | |
| HEK site 3 | ACACTCTTTCCCTACACGACGCTCTTCCGATCTNNNNATTAACTGCACCAGTGGGCA |
| off-target 2 | (SEQ ID NO: 527) |
| fwd | |
| HEK site 3 | TGGAGTTCAGACGTGTGCTCTTCCGATCTTGGTGTGCCTGTCACTGTAC |
| off-target 2 | (SEQ ID NO: 528) |
| rev | |
| HEK site 3 | ACACTCTTTCCCTACACGACGCTCTTCCGATCTNNNNGGGAAATAGGCCCTCTTCCT |
| xCas9 off- | (SEQ ID NO: 529) |
| target 1 fwd | |
| HEK site 3 | TGGAGTTCAGACGTGTGCTCTTCCGATCTGGAATCCCATGGGCACCATG |
| xCas9 off- | (SEQ ID NO: 545) |
| target 1 rev | |
| HEK site 3 | ACACTCTTTCCCTACACGACGCTCTTCCGATCTNNNNCTCAGTCTGAGAAAGTGGCG |
| xCas9 off- | (SEQ ID NO: 546) |
| target 2 fwd | |
| HEK site 3 | TGGAGTTCAGACGTGTGCTCTTCCGATCTAAGAACAAAGGCTGATGCGT |
| xCas9 off- | (SEQ ID NO: 547) |
| target 2 rev | |
| HEK site 4 | ACACTCTTTCCCTACACGACGCTCTTCCGATCTNNNNGGGTCCAAAGCAGGATGACA |
| on-target fwd | (SEQ ID NO: 548) |
| HEK site 4 | TGGAGTTCAGACGTGTGCTCTTCCGATCTCACACAGCACCAGAGTCTCC |
| on-target rev | (SEQ ID NO: 549) |
| HEK site 4 | ACACTCTTTCCCTACACGACGCTCTTCCGATCTNNNNAGCAGGTCCTTCATGGCAAG |
| off-target 1 | (SEQ ID NO: 550) |
| fwd | |
| HEK site 4 | TGGAGTTCAGACGTGTGCTCTTCCGATCTCCTCCATATCCCTGCACCTC |
| off-target 1 | (SEQ ID NO: 551) |
| rev | |
| HEK site 4 | ACACTCTTTCCCTACACGACGCTCTTCCGATCTNNNNTGCTCTCCAGAGTGGTCCAG |
| off-target 2 | (SEQ ID NO: 552) |
| fwd | |
| HEK site 4 | TGGAGTTCAGACGTGTGCTCTTCCGATCTTCCTCCTCGGAGTCCTCAAG |
| off-target 2 | (SEQ ID NO: 553) |
| rev | |
| HEK site 4 | ACACTCTTTCCCTACACGACGCTCTTCCGATCTNNNNAGCAGAGCAAGTCTCACATTT |
| off-target 3 | (SEQ ID NO: 554) |
| fwd | |
| HEK site 4 | TGGAGTTCAGACGTGTGCTCTTCCGATCTACCTTGTGATCTGTCCGTCTC |
| off-target 3 | (SEQ ID NO: 555) |
| rev | |
| HEK site 4 | ACACTCTTTCCCTACACGACGCTCTTCCGATCTNNNNCCGGATGTTCTTTCTGGGCT |
| off-target 4 | (SEQ ID NO: 556) |
| fwd | |
| HEK site 4 | TGGAGTTCAGACGTGTGCTCTTCCGATCTCTCGGTTCCTCCACAACACA |
| off-target 4 | (SEQ ID NO: 557) |
| rev | |
| HEK site 4 | ACACTCTTTCCCTACACGACGCTCTTCCGATCTNNNNGCATTACTCACCGGACCTCC |
| off-target 5 | (SEQ ID NO: 558) |
| fwd | |
| HEK site 4 | TGGAGTTCAGACGTGTGCTCTTCCGATCTCACGGGAAGGACAGGAGAAG |
| off-target 5 | (SEQ ID NO: 559) |
| rev | |
| HEK site 4 | ACACTCTTTCCCTACACGACGCTCTTCCGATCTNNNNGGTCCCCAGAGCTTAGTTCA |
| xCas9 off- | (SEQ ID NO: 560) |
| target 1 fwd | |
| HEK site 4 | TGGAGTTCAGACGTGTGCTCTTCCGATCTCGGATGCAATGAGCCAGGA |
| xCas9 off- | (SEQ ID NO: 561) |
| target 1 rev | |
| HEK site 4 | ACACTCTTTCCCTACACGACGCTCTTCCGATCTNNNNGTCACCCGGAATACCGGTCT |
| xCas9 off- | (SEQ ID NO: 562) |
| target 2 fwd | |
| HEK site 4 | TGGAGTTCAGACGTGTGCTCTTCCGATCTCCGTCTCTGTCATCATGCTG |
| xCas9 off- | (SEQ ID NO: 563) |
| target 2 rev | |

TABLE 15
Genomic sites used for GUIDE-seq analysis. An RNA
sequence complementary to the protospacer sequence
in the table would be used in a gRNA to target a
Cas9 domain or a fusion protein comprising a Cas9
domain to the sequence.

| Site | PAM | Protospacer sequence |
|---|---|---|
| EMX1 site | GGG | GAGTCCGAGCAGAAGAAGAA |
| | | (SEQ ID NO: 564) |
| HEK site 1 | GGG | GGGAAAGACCCAGCATCCGT |
| | | (SEQ ID NO: 565) |
| HEK site 2 | GGG | GAACACAAAGCATAGACTGC |
| | | (SEQ ID NO: 566) |
| HEK site 3 | GGG | GGCCCAGACTGAGCACGTGA |
| | | (SEQ ID NO: 567) |
| HEK site 4 | GGG | GGCACTGCGGCTGGAGGTGG |
| | | (SEQ ID NO: 568) |
| VEGFA site 1 | TGG | GGGTGGGGGGAGTTTGCTCC |
| | | (SEQ ID NO: 569) |
| FANCF GAA PAM site | GAA | GACTCTCTGCGTACTGATTG |
| | | (SEQ ID NO: 570) |
| FANCF CGT PAM site | CGT | GCGGTCTCAAGCACTACCTA |
| | | (SEQ ID NO: 571) |

sgRNA plasmids were generated with reverse primer GGTGTTTCGTCCTTTCCACAAGATATATAAAG (SEQ ID NO: 329) and forward primer (N)₂₀GTTTTAGAGCTAGAAATAGCAAGTTAAAATAAGGC (SEQ ID NO: 330), where (N)₂₀ is the protospacer sequence.

TABLE 16
List of GUIDE-seq off target sites, with corresponding chromosomal
location, PAM, and protospacer sequences. An RNA sequence
complementary to the protospacer sequence in the table would be used
in a gRNA to target a Cas9 domain or a fusion protein comprising a
Cas9 domain to the sequence.

| Site | Chromosome | PAM | Protospacer sequence |
|---|---|---|---|
| EMX1 off-target 1 | 15 | GAG | GAGTCTAAGCAGAAGAAGAA |
| | | | (SEQ ID NO: 572) |
| HEK site 1 off-target | 1 | TGG | GGGAAAGTCCCAGCATCCTT |
| 1 | | | (SEQ ID NO: 573) |
| HEK site 2 off-target | 2 | AGA | GAGGACAAAGTACAAACGGC |
| 1 | | | (SEQ ID NO: 574) |
| HEK site 2 off-target | 4 | CGG | GAACACAATGCATAGATTGC |
| 2 | | | (SEQ ID NO: 575) |
| HEK site 3 off-target | 7 | GGG | GACACAGACTGGGCACGTGA |
| 1 | | | (SEQ ID NO: 576) |
| HEK site 3 off-target | 15 | TGG | CACCCAGACTGAGCACGTGC |
| 2 | | | (SEQ ID NO: 577) |
| HEK site 3 xCas9 | 15 | CGG | AGGCAAAACGCACCACGTGA |
| off-target 1 | | | (SEQ ID NO: 578) |
| HEK site 3 xCas9 | 12 | AGT | GGGCCAGACTGAGCACGTGA |
| off-target 2 | | | (SEQ ID NO: 579) |
| HEK site 4 off-target | 10 | GGG | GGCACGACGGCTGGAGGTGG |
| 1 | | | (SEQ ID NO: 580) |
| HEK site 4 off-target | 20 | GGG | GGCACTGCGGCTGGAGGTGG |
| 2 | | | (SEQ ID NO: 581) |
| HEK site 4 off-target | 7 | GGG | GGCACTGCGGCTGGAGGTGG |
| 3 | | | (SEQ ID NO: 582) |
| HEK site 4 off-target | 10 | GGG | GGCACTGCGGCTGGAGGTGG |
| 4 | | | (SEQ ID NO: 583) |
| HEK site 4 off-target | 15 | GGG | GGCACTGCGGCTGGAGGTGG |
| 5 | | | (SEQ ID NO: 584) |
| HEK site 4 xCas9 | 5 | GGG | GGCACTGCGGCTGGAGGTGG |
| off-target 1 | | | (SEQ ID NO: 585) |
| HEK site 4 xCas9 | 13 | GGG | GGCACTGCGGCTGGAGGTGG |
| off-target 2 | | | (SEQ ID NO: 586) |

TABLE 17
GUIDE-seq HTS files contained in NCBI sequencing read archive,
and the corresponding GUIDE-seq data within each file.

| File name | Data |
|---|---|
| Guideseq 1 | EMX1 SpCas9 in HEK293T cells |
| Guideseq 3 | EMX1 xCas9 3.6 in HEK293T cells |
| Guideseq 1 | EMX1 xCas9 3.7 in HEK293T cells |
| Guideseq 5 | EMX1 SpCas9 in U2OS cells |
| Guideseq 5 | EMX1 xCas9 3.6 in U2OS cells |
| Guideseq 5 | EMX1 xCas9 3.7 in U2OS cells |
| Guideseq 3 | HEK site 1 SpCas9 in HEK293T cells |
| Guideseq 3 | HEK site 1 xCas9 3.6 in HEK293T cells |
| Guideseq 3 | HEK site 1 xCas9 3.7 in HEK293T cells |
| Guideseq 3 | HEK site 2 SpCas9 in HEK293T cells |
| Guideseq 3 | HEK site 2 xCas9 3.6 in HEK293T cells |
| Guideseq 3 | HEK site 2 xCas9 3.7 in HEK293T cells |
| Guideseq 3 | HEK site 3 SpCas9 in HEK293T cells |
| Guideseq 3 | HEK site 3 xCas9 3.6 in HEK293T cells |
| Guideseq 3 | HEK site 3 xCas9 3.7 in HEK293T cells |
| Guideseq 1 | HEK site 4 SpCas9 in HEK293T cells |
| Guideseq 1 | HEK site 4 xCas9 3.7 in HEK293T cells |
| Guideseq 3 | HEK site 4 xCas9 3.6 in HEK293T cells |
| Guideseq 2 | FANCF GAA xCas9 3.7 in HEK293T cells |
| Guideseq 4 | FANCF NGT xCas9 3.7 in HEK293T cells |
| Guideseq 6 | VEGFA site 1 SpCas9 in U2OS cells |
| Guideseq 6 | VEGFA site 1 xCas9 3.6 in U2OS cells |
| Guideseq 6 | VEGFA site 1 xCas9 3.7 in U2OS cells |

### DNA Sequences of PACE Selection Components

AP construct (ω(I12N)-linker(2aa)-dCas9):

(SEQ ID NO: 530)
GCACGCGTAACTGTTCAGGACGCTGTAGAGAAAAATGGTAACCGTTTTGACCTGGTACTGGTCGCCGCGCGTCGC
GCTCGTCAGATGCAGGTAGGCGGAAAGGATCCGCTGGTACCGGAAGAAAACGATAAAACCACTGTAATCGCGCTG
CGCGAAATCGAAGAAGGTCTGATCAACAACCAGATCCTCGACGTTCGCGAACGCCAGGAACAGCAAGAGCAGGAA
GCCGCTGAATTACAAGCCGTTACCGCTATTGCTGAAGGTCGTCGTGCTGCAGACAAGAAGTACTCCATTGGGCTC
GCTATCGGCACAAACAGCGTCGGCTGGGCCGTCATTACGGACGAGTACAAGGTGCCGAGCAAAAAATTCAAAGTT
CTGGGCAATACCGATCGCCACAGCATAAAGAAGAACCTCATTGGCGCCCTCCTGTTCGACTCCGGGGAGACGGCC
GAAGCCACGCGGCTCAAAAGAACAGCACGGCGCAGATATACCCGCAGAAAGAATCGGATCTGCTACCTGCAGGAG
ATCTTTAGTAATGAGATGGCTAAGGTGGATGACTCTTTCTTCCATAGGCTGGAGGAGTCCTTTTTGGTGGAGGAG
GATAAAAAGCACGAGCGCCACCCAATCTTTGGCAATATCGTGGACGAGGTGGCGTACCATGAAAAGTACCCAACC
ATATATCATCTGAGGAAGAAGCTTGTAGACAGTACTGATAAGGCTGACTTGCGGTTGATCTATCTCGCGCTGGCG
CATATGATCAAATTTCGGGGACACTTCCTCATCGAGGGGGACCTGAACCCAGACAACAGCGATGTCGATAAACTC
TTTATCCAACTGGTTCAGACTTACAATCAGCTTTTCGAAGAGAACCCGATCAACGCATCCGGAGTTGACGCCAAA
GCAATCCTGAGCGCTAGGCTGTCCAAATCCCGGCGGCTCGAAAACCTCATCGCACAGCTCCCTGGGGAGAAGAAG
AACGGCCTGTTTGGTAATCTTATCGCCCTGTCACTCGGGCTGACCCCCAACTTTAAATCTAACTTCGACCTGGCC
GAAGATGCCAAGCTTCAACTGAGCAAAGACACCTACGATGATGATCTCGACAATCTGCTGGCCCAGATCGGCGAC
CAGTACGCAGACCTTTTTTTGGCGGCAAAGAACCTGTCAGACGCCATTCTGCTGAGTGATATTCTGCGAGTGAAC
ACGGAGATCACCAAAGCTCCGCTGAGCGCTAGTATGATCAAGCGCTATGATGAGCACCACCAAGACTTGACTTTG
CTGAAGGCCCTTGTCAGACAGCAACTGCCTGAGAAGTACAAGGAAATTTTCTTCGATCAGTCTAAAAATGGCTAC
GCCGGATACATTGACGGCGGAGCAAGCCAGGAGGAATTTTACAAATTTATTAAGCCCATCTTGGAAAAAATGGAC
GGCACCGAGGAGCTGCTGGTAAAGCTTAACAGAGAAGATCTGTTGCGCAAACAGCGCACTTTCGACAATGGAAGC
ATCCCCCACCAGATTCACCTGGGCGAACTGCACGCTATCCTCAGGCGGCAAGAGGATTTCTACCCCTTTTTGAAA
GATAACAGGGAAAAGATTGAGAAAATCCTCACATTTCGGATACCCTACTATGTAGGCCCCCTCGCCCGGGGAAAT
TCCAGATTCGCGTGGATGACTCGCAAATCAGAAGAGACCATCACTCCCTGGAACTTCGAGGAAGTCGTGGATAAG
GGGGCCTCTGCCCAGTCCTTCATCGAAAGGATGACTAACTTTGATAAAAATCTGCCTAACGAAAAGGTGCTTCCT
AAACACTCTCTGCTGTACGAGTACTTCACAGTTTATAACGAGCTCACCAAGGTCAAATACGTCACAGAAGGGATG
AGAAAGCCAGCATTCCTGTCTGGAGAGCAGAAGAAAGCTATCGTGGACCTCCTCTTCAAGACGAACCGGAAAGTT
ACCGTGAAACAGCTCAAAGAAGACTATTTCAAAAAGATTGAATGTTTCGACTCTGTTGAAATCAGCGGAGTGGAG
GATCGCTTCAACGCATCCCTGGGAACGTATCACGATCTCCTGAAAATCATTAAAGACAAGGACTTCCTGGACAAT
GAGGAGAACGAGGACATTCTTGAGGACATTGTCCTCACCCTTACGTTGTTTGAAGATAGGGAGATGATTGAAGAA
CGCTTGAAAACTTACGCTCATCTCTTCGACGACAAAGTCATGAAACAGCTCAAGAGGCGCCGATATACAGGATGG
GGGCGGCTGTCAAGAAAACTGATCAATGGGATCCGAGACAAGCAGAGTGGAAAGACAATCCTGGATTTTCTTAAG
TCCGATGGATTTGCCAACCGGAACTTCATGCAGTTGATCCATGATGACTCTCTCACCTTTAAGGAGGACATCCAG
AAAGCACAAGTTTCTGGCCAGGGGGACAGTCTTCACGAGCACATCGCTAATCTTGCAGGTAGCCCAGCTATCAAA
AAGGGAATACTGCAGACCGTTAAGGTCGTGGATGAACTCGTCAAAGTAATGGGAAGGCATAAGCCCGAGAATATC
GTTATCGAGATGGCCCGAGAGAACCAAACTACCCAGAAGGGACAGAAGAACAGTAGGGAAAGGATGAAGAGGATT
GAAGAGGGTATAAAAGAACTGGGGTCCCAAATCCTTAAGGAACACCCAGTTGAAAACACCCAGCTTCAGAATGAG
AAGCTCTACCTGTACTACCTGCAGAACGGCAGGGACATGTACGTGGATCAGGAACTGGACATCAATCGGCTCTCC
GACTACGACGTGGACGCTATCGTGCCCCAGTCTTTTCTCAAAGATGATTCTATTGATAATAAAGTGTTGACAAGA
TCCGATAAAAACAGAGGGAAGAGTGATAACGTCCCCTCAGAAGAAGTTGTCAAGAAAATGAAAAATTATTGGCGG
CAGCTGCTGAACGCCAAACTGATCACACAACGGAAGTTCGATAATCTGACTAAGGCTGAACGAGGTGGCCTGTCT
GAGTTGGATAAAGCCGGCTTCATCAAAAGGCAGCTTGTTGAGACACGCCAGATCACCAAGCACGTGGCCCAAATT
CTCGATTCACGCATGAACACCAAGTACGATGAAAATGACAAACTGATTCGAGAGGTGAAAGTTATTACTCTGAAG
TCTAAGCTGGTCTCAGATTTCAGAAAGGACTTTCAGTTTTATAAGGTGAGAGAGATCAACAATTACCACCATGCG
CATGATGCCTACCTGAATGCAGTGGTAGGCACTGCACTTATCAAAAAATATCCCAAGCTTGAATCTGAATTTGTT
TACGGAGACTATAAAGTGTACGATGTTAGGAAAATGATCGCAAAGTCTGAGCAGGAAATAGGCAAGGCCACCGCT
AAGTACTTCTTTTACAGCAATATTATGAATTTTTTCAAGACCGAGATTACACTGGCCAATGGAGAGATTCGGAAG
CGACCACTTATCGAAACAAACGGAGAAACAGGAGAAATCGTGTGGGACAAGGGTAGGGATTTCGCGACAGTCCGG
AAGGTCCTGTCCATGCCGCAGGTGAACATCGTTAAAAAGACCGAAGTACAGACCGGAGGCTTCTCCAAGGAAAGT
ATCCTCCCGAAAAGGAACAGCGACAAGCTGATCGCACGCAAAAAAGATTGGGACCCCAAGAAATACGGCGGATTC
GATTCTCCTACAGTCGCTTACAGTGTACTGGTTGTGGCCAAAGTGGAGAAAGGGAAGTCTAAAAAACTCAAAAGC
GTCAAGGAACTGCTGGGCATCACAATCATGGAGCGATCAAGCTTCGAAAAAAACCCCATCGACTTTCTCGAGGCG
AAAGGATATAAAGAGGTCAAAAAAGACCTCATCATTAAGCTTCCCAAGTACTCTCTCTTTGAGCTTGAAAACGGC
CGGAAACGAATGCTCGCTAGTGCGGGCGAGCTGCAGAAAGGTAACGAGCTGGCACTGCCCTCTAAATACGTTAAT
TTCTTGTATCTGGCCAGCCACTATGAAAAGCTCAAAGGGTCTCCCGAAGATAATGAGCAGAAGCAGCTGTTCGTG
GAACAACACAAACACTACCTTGATGAGATCATCGAGCAAATAAGCGAATTCTCCAAAAGAGTGATCCTCGCCGAC
GCTAACCTCGATAAGGTGCTTTCTGCTTACAATAAGCACAGGGATAAGCCCATCAGGGAGCAGGCAGAAAACATT
ATCCACTTGTTTACTCTGACCAACTTGGGCGCGCCTGCAGCCTTCAAGTACTTCGACACCACCATAGACAGAAAG
CGGTACACCTCTACAAAGGAGGTCCTGGACGCCACACTGATTCATCAGTCAATTACGGGGCTCTATGAAACAAGA
ATCGACCTCTCTCAGCTCGGTGGAGAC

### SP:

(SEQ ID NO: 531)
CCGTTCGTTTTGCTGAGGAGACTTAGGGACCCTACAACACGCACGGTGTT
ACATTAGGCACCCCGGGCTTTACACTTTATGCTTCCGGCTCGTATGTTGT
GTCGACCGACCTGCAGGTGCAGTAAAGGAAAAAAAAA

Matlab Script for Base Calling

```
function basecall( )
%cycle through fastq files for different samples
WTnuc=‘GAATCCCTTCTGCAGCACCTGGATCGCTTTTCCGAGCTTCTGGCGGTCTCAAGCACTAC
CTACGTCAGCACCTGGGACCCCGCCACCGTGCGCCGGGCCTTGCAGTGGGCGCGCTACCTGC
GCCACATCCA’;(SEQ ID NO: 587)
files=dir(‘*.fastq’);
for d=1:9
  filename=files(d).name;
  %read fastq file
  [header,seqs,qscore] = fastqread(filename);
  seqsLength = length(seqs); % number of sequences
  seqsFile = strrep(filename,‘.fastq’,″); % trims off .fastq
  %create a directory with the same name as fastq file
  if exist(seqsFile,‘dir’);
    error(‘Directory already exists. Please rename or move it before moving on.’);
  end
  mkdir(seqsFile); % make directory
  wtLength = length(WTnuc); % length of wildtype sequence
  %% aligning back to the wildtype nucleotide sequence
  %
  % AIN is a matrix of the nucleotide alignment
  window=1:wthength;
  sBLength = length(seqs); % number of sequences
  % counts number of skips
  nSkips = 0;
  ALN=repmat(‘ ’,[sBLength wthength]);
  % iterate through each sequencing read
  for i = 1:sBLength
    %If you only have forward read fastq files leave as is
    %If you have R1 foward and R2 is reverse fastq files uncomment the
    %next four lines of code and the subsequent end statement
% if mod(d,2)==0;
% reverse = seqrcomplement(seqs{i});
% [score,alignment,start] = swalign(reverse,WTnuc,‘Alphabet’,‘NT’);
% else
    [score,alignment,start] =swalign(seqs{i},WTnuc,‘Alphabet’,‘NT’);
% end
    % length of the sequencing read
    len = length(alignment(3,:));
    % if there is a gap in the alignment , skip = 1 and we will
    % throw away the entire read
    skip = 0;
    for j = 1:len
      if (alignment(3,j) == ‘-’ || alignment(1,j) == ‘-’)
        skip = 1;
        break;
      end
      %in addition if the qscore for any given base in the read is
      %below 31 the nucleotide is turned into an N (fastq qscores that are not letters)
      if isletter(qscore{i}(start(1)+j−1))
      else
        alignment(1,j) = ‘N’;
      end
    end
    if skip == 0 && len>10
      ALN(i, start(2):(start(2)+length(alignment)−1))+32alignment(1,:);
    end
  end
  % with the alignment matrices we can simply tally up the occurrences of
  % each nucleotide at each column in the alignment these
  % tallies ignore bases annotated as N
  % due to low qscores
  TallyNTD=zeros(5,wthength);
  FreqNTD=zeros(4,wtLength);
  SUM=zeros(1,wthength);
  for i=1:wthength
    TallyNTD(:,i)=[sum(ALN(:,i)==‘A’),sum(ALN(:,i)==‘C’),sum(ALN(:,i)==‘G’),sum(ALN(:,i)==‘T’),
sum(ALN(:,i)==‘N’)];
  end
  for i=1:wthength
    FreqNTD(:,0=100*TallyNTD(1:4,i)/sum(TallyNTD(1:4,i));
  end
  for i=1:wthength
    SUM(:,i)=sum(TallyNTD(1:4,i));
  end
  % we then save these tally matrices in the respective folder for
  % further processing
  save(strcat(seqsFile, ‘/TallyNTD’), ‘TallyNTD’);
  dlmwrite(strcat(seqsFile, ‘/TallyNTD.csv’), TallyNTD, ‘precision’, ‘%.3f’, ‘newline’, ‘pc’);
  save(strcat(seqsFile, ‘/FreqNTD’), ‘FreqNTD’);
  dlmwrite(strcat(seqsFile, ‘/FreqNTD.csv’), FreqNTD, ‘precision’, ‘%.3f’, ‘newline’, ‘pc’);
  fid = fopen(‘FrequencySummary.csv’, ‘a’);
  fprintf(fid, ‘\n \n’);
  fprintf(fid, filename);
  fprintf(fid, ‘\n \n’);
  dlmwrite(‘FrequencySummary.csv’, FreqNTD, ‘precision’, ‘%.3f’, ‘newline’, ‘pc’, ‘-append’);
  dlmwrite(‘FrequencySummary.csv’, SUM, ‘precision’, ‘%.3f’, ‘newline’, ‘pc’, ‘-append’);
end
```

Matlab Script for Indel Analysis.

```
function indelcall( )
%cycle through fastq files for different samples
s11048 =′GCCCATTCCCTCTTTAGCCAGAGCCGGGGTGTGCAGACGGCAGTCACTAGGGGGCGC
TCGGCCACCACAGGGAAGCTGGGTGAATGGAGCGAGCAGCGTCTTCGAGAGTGAGGACGTGTG
TGTCTGTGTGGGTGAGTGAGTGTGTGCGTGTGGGGTTGAG′; (SEQ ID NO: 588)
s11150=′GCCCATTCCCTCTTTAGCCAGAGCCGGGGTGTGCAGACGGCAGTCACTAGGGGGCGC
TCGGCCACCACAGGGAAGCTGGGTGAATGGAGCGAGCAGCGTCTTCGAGAGTGAGGACGTGTG
TGTCTGTGTGGGTGAGTGAGTGTGTGCGTGTGGGGTTGAG′; (SEQ ID NO: 589)
s11048is=34
s11150i5=71
WTnuc=s11048;
%WTnuc=′CGGTGGGAGGTCTATATAAGCAGAGCTGGTTTAGTGAACCGTCAGATCCGCTAGAGA
TCCGCGGCCGCTAATACGACTCACCCTAGGGAGAGCCGCCACCGTGGTGAGCAAGGGCGAGG
AGCTGTTCACCGGGGTGGTGCCCATCCTGGTCGAGCTGGACGGCGACGTAAACGGCCACAAGT
TCAGCGTGTCCGGCGAG′; (SEQ ID NO: 590)
%cycle through fastq files for different samples
files=dir(*.fastq′);
indelstart=s11048is;
width=30;
flank=10;
for d=1:9
  filename=files(d).name;
  %read fastq file
  [header,seqs,qscore] = fastqread(filename);
  seqsLength = length(seqs); % number of sequences
  seqsFile = strcat(strrep(filename,‘.fastq’,″),‘_INDELS’); % trims off .fastq
  %create a directory with the same name as fastq file+_INDELS
  if exist(seqsFile,‘dir’);
    error(‘Directory already exists. Please rename or move it before moving on.’);
  end
  mkdir(seqsFile); % make directory
  wthength = length(WTnuc); % length of wildtype sequence
  sBLength = length(seqs); % number of sequences
  % initialize counters and cell arrays
  nSkips = 0;
  notINDEL=0;
  ins={ };
  dels={ };
  NumIns=0;
  NumDels=0;
  % iterate through each sequencing read
  for i = 1:sBLength
  %search for 10BP sequences that should flank both sides of the “INDEL WINDOW”
    windowstart=strfind(seqs{i},WTnuc(indelstart-flank:indelstart));
    windowend=strfind(seqs{i},WTnuc(indelstart+width:indelstart+width+flank));
    %if these flanks are found and more than half of base calls
    %are above Q31 THEN proceed OTHERWISE save as a skip
    if length(windowstart)==1 && length(windowend)==1 &&
    (sum(isletter(qscore{i}))/length(qscorelip)>=0.5
        %if the sequence length matches the INDEL window length save as
        %not INDEL
        if windowend-windowstart==width+flank
          notINDEL=notINDEL+1;
        %if the sequence is ONE or more baseslonger than the INDEL
        %window length save as an Insertion
        elseif windowend-windowstart>=width+flank+1
          NumIns=NumIns+1;
          ins{NumIns}=seqs{i};
      %if the sequence is ONE or more bases shorter than the INDEL
      %window length save as a Deletion
      elseif windowend-windowstart<=width+flank-1
        NumDels=NumDels+1;
        dels{NumDels}=seqs{i};
      end
    %keep track of skipped sequences that do not posess matching flank
    %sequences and do not pass quality cutoff
    else
      nSkips=nSkips+1;
    end
  end
  INDELrate=(NumIns+NumDels)/(NumIns+NumDels+notINDEL)*100.;
  FID = fopen(‘INDELSummary.csv’, ‘a’);
  fprintf(FID, ‘\n \n’);
  fprintf(FID, filename);
  fprintf(FID, ‘\n’);
  fprintf(FID, num2str(INDELrate));
  fid=fopen(strcat(seqsFile, ‘/summary.txt’), ‘wt’);
  fprintf(fid, ‘Skipped reads %i\n not INDEL %i\n Insertions %i\n Deletions %i\n INDEL percent %e\n’,
[nSkips, notINDEL, NumIns, NumDels,INDELrate]);
  fclose(fid);
  save(strcat(seqsFile, ‘/nSkips’), ‘nSkips’);
  save(strcat(seqsFile, ‘/notINDEL’), ‘notINDEL’);
  save(strcat(seqsFile, ‘NumIns’), ‘NumIns’);
  save(strcat(seqsFile, ‘/NumDels’), ‘NumDels’);
  save(strcat(seqsFile, ‘/INDELrate’), ‘INDELrate’);
  save(strcat(seqsFile, ‘/dels’), ‘dels’);
  C = dels;
  fid = fopen(strcat(seqsFile, ‘/dels.txt’), ‘wt’);
  fprintf(fid, ‘“%s”\n’, C{:});
  fclose(fid);
  save(strcat(seqsFile, ‘/ins’), ‘ins’);
  C = ins;
  fid = fopen(strcat(seqsFile, ‘/ins.txt’), ‘wt’);
  fprintf(fid, ‘“%s”\n’, C{:});
  fclose(fid);
end
```

Script for Clinvar SNP analysis for C⋅G to T⋅A base conversion and T⋅A to

G•C base conversion
import numpy as np
import pandas as pd
import regex
import re
from Bio import SeqIO
import Bio
###CHANGE PAM AND WINDOW INFO HERE###
PAM = ‘GAT’
windowstart = 4
windowend = 8
###CHANGE PAM AND WINDOW INFO HERE###
def RC(seq):

encoder = {‘A’:‘T’,‘T’:‘A’,‘C’:‘G’,‘G’:‘C’,‘N’:‘N’,‘R’:‘Y’,‘Y’:‘R’, ‘M’:‘K’, ‘K’:‘M’, ‘S’:‘S’, ‘W’:‘W’, ‘H’:‘D’,

‘B’:‘V’, ‘V’:‘B’, ‘D’:‘H’}

rc = “
for n in reversed(seq):

rc += encoder[n]

return rc

def create_PAM(pam):

encoder =

{‘A’:‘A’,‘T’:‘T’,‘G’:‘G’,‘C’:‘C’,‘R’:‘[A|G]’,‘Y’:‘[C|T]’,‘N’:‘[A|T|C|G]’,‘M’:‘[A|C]’,‘K’:‘[G|T]’,‘S’:‘[C|G]’,‘W’:‘[A|T]’,‘H’:‘[
A|C|T]’,‘B’:‘[C|G|T]’,‘V’:‘[A|C|G]’,‘D’:‘[A|G|T]’}

enc_pam = {‘f’:“,‘r’:”}
rc_pam = RC(pam)
for n,m in zip(pam, rc_pam):

enc_pam[‘f’] += encoder[n]
enc_pam[‘r’] += encoder[m]

return enc_pam

enc_pam = create_PAM(PAM)
windowlen=windowend−windowstart+1
lenpam=len(PAM)
#################################################################################
#################################################################################
##########################################
Clinvar=pd.read_csv(‘Clinvar_DB.csv’, encoding = “ISO-8859-1”)
Clinvar_size = len(Clinvar.index)
Clinvar_dedup = Clinvar.drop_duplicates(subset=‘RCVaccession’, keep=‘first’)
Clinvar_dedup_size = len(Clinvar_dedup.index)
print “size of the dedupped Clinvar Database:”, Clinvar_dedup_size
pathogenic = Clinvar_dedup[‘ClinicalSignificance’] == “Pathogenic”
Clinvar_Patho_df = Clinvar_dedup[pathogenic]
Clinvar_Patho_size = len(Clinvar_Patho_df.index)
print “Pathogenic entities in Clinvar:”, Clinvar_Patho_size
SNV = Clinvar_Patho_df[‘Type’] == “single nucleotide variant”
Clinvar_Patho_SNV = Clinvar_Patho_df[SNV]
Clinvar_Patho_SNV_size = len(Clinvar_Patho_SNV)
print “Pathogenic SNV's in Clinvar:”, Clinvar_Patho_SNV_size
BE_CtoT_editable = Clinvar_Patho_SNV[Clinvar_Patho_SNV[“Name”].str.contains(“T>C”)]
BE_GtoA_editable = Clinvar_Patho_SNV[Clinvar_Patho_SNV[“Name”].str.contains(“A>G”)]
ABE_TtoC_editable = Clinvar_Patho_SNV[Clinvar_Patho_SNV[“Name”].str.contains(“C>T”)]
ABE_AtoG_editable = Clinvar_Patho_SNV[Clinvar_Patho_SNV[“Name”].str.contains(“G>A”)]
AT_GC = Clinvar_Patho_SNV[Clinvar_Patho_SNV[“Name”].str.contains(“G>A|C>T”)] # C>T and G>A
in clinvar but A>G , T>C base editing #ABE
GC_AT = Clinvar_Patho_SNV[Clinvar_Patho_SNV[“Name”].str.contains(“A>G|T>C”)] # BE
# editing opportunities for transitions that can not yet be edited by base editing
CG_GC = Clinvar_Patho_SNV[Clinvar_Patho_SNV[“Name”].str.contains(“C>G|G>C”)]
AT_TA = Clinvar_Patho_SNV[Clinvar_Patho_SNV[“Name”].str.contains(“A>T|T>A”)]
GC_TA = Clinvar_Patho_SNV[Clinvar_Patho_SNV[“Name”].str.contains(“A>C|T>G”)]
AT_CG = Clinvar_Patho_SNV[Clinvar_Patho_SNV[“Name”].str.contains(“C>A|G>T”)]
GC_AT_size= len(GC_AT.index)
print “BE editable bases(GC_AT):”, GC_AT_size
AT_GC_size = len(AT_GC.index)
print “ABE editable bases(AT_GC):”, AT_GC_size
CG_GC_size = len(CG_GC.index)
print “CG_GC:”, CG_GC_size
AT_TA_size = len(AT_TA.index)
print “AT_TA:”, AT_TA_size
GC_TA_size = len(GC_TA.index)
print “GC_TA:”, GC_TA_size
AT_CG_size = len(AT_CG.index)
print “AT_CG:”, AT_CG_size
#open flanking sequence fasta files for all pathogenic human SNPs
#downloaded as fasta file from:
handle = open(“SNP_FASTA.txt”, “rU”)
flanks={ }
#save as a dictionary keyed on rsID as an Integer with values being 25nt of flanking sequence on
each side of the SNP
for record in SeqIO.parse(handle, “fasta”) :

flanks[int(record.id.split(“|”)[−1].strip(‘rs’))]=regex.findall(‘.{25}[\hat{ }A,T,C,G].{25}’, record.seq.tostring( ))

handle.close( )
#merge flanking sequences to the CtoT frame on rsID
F=pd.DataFrame({‘RS# (dbSNP)’: list(flanks.keys( )), ‘Flanks’: [x for x in flanks.values( )]})
BE_CtoT_editable = F.merge(BE_CtoT_editable, left_on=‘RS# (dbSNP)’, right_on=‘RS# (dbSNP)’,
how=‘right’)
BE_GtoA_editable = F.merge(BE_GtoA_editable, left_on=‘RS# (dbSNP)’, right_on=‘RS# (dbSNP)’,
how=‘right’)
ABE_TtoC_editable = F.merge(ABE_TtoC_editable, left_on=‘RS# (dbSNP)’, right_on=‘RS# (dbSNP)’,
how=‘right’)
ABE AtoG editable = F.merqe(ABE_AtoG_editable, left_on=‘RS# (dbSNP)’, right_on=‘RS# (dbSNP)’,
how=‘right’)
# clinvar may refer to the opposite strand that was used in dbSNP;
# we want to allow clinvar reference alleles A and T with alternate alleles G and C respectively
#BE_CtoT_editable.to_csv(‘BE_CtoT_editable.csv’, sep=‘\t’, encoding = ‘utf-8’)
#BE_GtoA_editable.to_csv(‘BE_GtoA_editable.csv’, sep=‘\t’, encoding = ‘utf-8’)
BE_CtoT_editable[‘gRNAs']=None
BE_CtoT_editable[‘gRNAall’]=None
for i in range(len(BE_CtoT_editable)):

if type(BE_CtoT_editable.iloc[i].Flanks)==list and BE_CtoT_editable.iloc[i].Flanks!=[ ]:

test=BE_CtoT_editable.iloc[i].Flanks[0]
# define a potential gRNA spacer for each window positioning
gRNAoptions=[test[(26−windowstart−j):(26−windowstart−j+lenpam+20)] for j in range(windowlen)]
#if there is an appropriate PAM placed for a given gRNA spacer
#save tuple of gRNA spacer, and the position of off-target Cs in the window
gRNA=[(gRNAoptions[k],[x.start( )+1 for x in re.finditer(‘C’,gRNAoptions[k]) if windowstart−

1<x.start( )+1<windowend+1]) for k in range(len(gRNAoptions)) if regex.match(enc_pam[‘f’],
gRNAoptions[k][−lenpam:])]

gRNAsingleC=[ ]
for g,c in gRNA:

#if the target C is the only C in the window save this as a single C site
if g[windowstart−1 :windowend].count(‘C’)==0:

gRNAsingleC.append(g)

#OPTIONAL uncomment the ELIF statement if you are interest in filtered based upon position

of off-target C

#if the target C is expected to be editted more efficiently than the off-target Cs, also save as a

single C Site

#elif all([p<priority[x] for x in c]):

#gRNAsingleC.append(g)

BE_CtoT_editable.gRNAs.iloc[i]=gRNAsingleC
BE_CtoT_editable.gRNAall.iloc[i]=[g for g,c in gRNA]

#merge in phenotypes based upon MedGen IDs; remove redundant columns
BE_CtoT_editable=BE_CtoT_editable[[‘RS# (dbSNP)’,‘GeneSymbol’, ‘gRNAs', ‘gRNAall’,‘Name’,
‘PhenotypeIDS’, ‘Origin’, ‘Reviewstatus', ‘NumberSubmitters', ‘LastEvaluated’]]
BE_CtoT_editable.drop(‘PhenotypeIDS’, inplace=True, axis=1)
BE_GtoA_editable[‘gRNAs']=None
BE_GtoA_editable[‘gRNAall’]=None
for i in range(len(BE_GtoA_editable)):

if type(BE_GtoA_editable.iloc[i].Flanks)==list and BE_GtoA_editable.iloc[i].Flanks!=[ ]:

test=BE_GtoA_editable.iloc[i].Flanks[0]
gRNAoptions=[test[(25+windowstart+j-20-lenpam):(25+windowstart+j)] for j in range(windowlen)]
gRNA=[(gRNAoptions[k],[20+lenpam-x.start( ) for x in re.finditer(‘G’,gRNAoptions[k]) if

windowstart−1 <20+lenpam−x.start( )<windowend+1]) for k in range(len(gRNAoptions)) if
regex.match(enc_pam[‘r’], gRNAoptions[k][:lenpam])]

gRNAsingleG=[ ]
for g,c in gRNA:

if g[20+lenpam−windowstart−windowlen+1:20+lenpam−windowstart+1 ].count(‘G’)==0:

gRNAsingleG.append(g)

#elif all([p<priority[x] for x in c]):

#gRNAsingleC.append(g)

BE_GtoA_editable.gRNAs.iloc[i]=gRNAsingleG
BE_GtoA_editable.gRNAall.iloc[i]=[g for g,c in gRNA]

BE_GtoA_editable=BE_GtoA_editable[[‘RS# (dbSNP)’,‘GeneSymbol’, ‘gRNAs', ‘gRNAall’,‘Name’,
‘PhenotypeIDS’, ‘Origin’, ‘Reviewstatus', ‘NumberSubmitters', ‘LastEvaluated’]]
BE_GtoA_editable.drop(‘PhenotypeIDS’, inplace=True, axis=1)
ABE_AtoG_editable[‘gRNAs']=None
ABE_AtoG_editable[‘gRNAall’]=None
for i in range(len(ABE_AtoG_editable)):

if type(ABE_AtoG_editable.iloc[i].Flanks)==list and ABE_AtoG_editable.iloc[i].Flanks!=[ ]:

test=ABE_AtoG_editable.iloc[i].Flanks[0]
# define a potential gRNA spacer for each window positioning
gRNAoptions=[test[(26−windowstart−j):(26−windowstart−j+lenpam+20)] for j in range(windowlen)]
#if there is an appropriate PAM placed for a given gRNA spacer
#save tuple of gRNA spacer, and the position of off-target Cs in the window
gRNA=[(gRNAoptions[k],[x.start( )+1 for x in re.finditer(‘A’,gRNAoptions[k]) ifwindowstart−

1<x.start( )+1<windowend+1]) for k in range(len(gRNAoptions)) if regex.match(enc_pam[‘f’],
gRNAoptions[k][−lenpam:])]

gRNAsingleA=[ ]
for g,c in gRNA:

#if the target C is the only C in the window save this as a single C site
if g[windowstart−1 :windowend].count(‘A’)==0:

gRNAsingleA.append(g)

#OPTIONAL uncomment the ELIF statement if you are interest in filtered based upon position

of off-target C

#if the target C is expected to be editted more efficiently than the off-target Cs, also save as a

single C Site

#elif all([p<priority[x] for x in c]):

#gRNAsingleC.append(g)

ABE_AtoG_editable.gRNAs.iloc[i]=gRNAsingleA
ABE_AtoG_editable.gRNAall.iloc[i]=[g for g,c in gRNA]

#merge in phenotypes based upon MedGen IDs; remove redundant columns
ABE_AtoG_editable=ABE_AtoG_editable[[‘RS# (dbSNP)’,‘GeneSymbol’, ‘gRNAs', ‘gRNAall’,‘Name’,
‘PhenotypeIDS’, ‘Origin’, ‘Reviewstatus', ‘NumberSubmitters', ‘LastEvaluated’]]
ABE_AtoG_editable.drop(‘PhenotypeIDS’, inplace=True, axis=1)
ABE_AtoG_editable.to_csv(‘ABE_AtoG_editable.csv’)
ABE_TtoC_editable[‘gRNAs']=None
ABE_TtoC_editable[‘gRNAall’]=None
for i in range(len(ABE_TtoC_editable)):

if type(ABE_TtoC_editable.iloc[i].Flanks)==list and ABE_TtoC_editable.iloc[i].Flanks!=[ ]:

test=ABE_TtoC_editable.iloc[i].Flanks[0]
gRNAoptions=[test[(25+windowstart+j−20−lenpam):(25+windowstart+j)] for j in range(windowlen)]
gRNA=[(gRNAoptions[k],[20+lenpam−x.start( ) for x in re.finditer(‘T’,gRNAoptions[k]) if

windowstart−1 <20+lenpam−x.start( )<windowend+1]) for k in range(len(gRNAoptions)) if
regex.match(enc_pam[‘r’], gRNAoptions[k][:lenpam])]

gRNAsingleT=[ ]
for g,c in gRNA:

if g[20+lenpam−windowstart−windowlen+1:20+lenpam−windowstart+1 ].count(‘T’)==0:

gRNAsingleT.append(g)

#elif all([p<priority[x] for x in c]):

#gRNAsingleC.append(g)

ABE_TtoC_editable.gRNAs.iloc[i]=gRNAsingleT
ABE_TtoC_editable.gRNAall.iloc[i]=[g for g,c in gRNA]

ABE_TtoC_editable=ABE_TtoC_editable[[‘RS# (dbSNP)’,‘GeneSymbol’, ‘gRNAs', ‘gRNAall’,‘Name’,
‘PhenotypeIDS’, ‘Origin’, ‘Reviewstatus', ‘NumberSubmitters', ‘LastEvaluated’]]
ABE_TtoC_editable.drop(‘PhenotypeIDS’, inplace=True, axis=1)
BE_CtoT_hasPAM=BE_CtoT_editable[[type(x)==list and x!=[ ] for x in BE_CtoT_editable.gRNAall]]
BE_CtoT_hasPAM.to_csv(‘BE_CtoT_hasPAM.csv’)
BE_CtoT_SingleC=BE_CtoT_editable[[type(x)==list and x!=[ ] for x in BE_CtoT_editable.gRNAs]]
BE_CtoT_SingleC.to_csv(‘BE_CtoT_SingleC.csv’)
BE_GtoA_hasPAM=BE_GtoA_editable[[type(x)==list and x!=[ ] for x in BE_GtoA_editable.gRNAall]]
BE_GtoA_hasPAM.to_csv(‘BE_GtoA_hasPAM.csv’)
BE_GtoA_SingleG=BE_GtoA_editable[[type(x)==list and x!=[ ] for x in BE_GtoA_editable.gRNAs]]
BE_GtoA_SingleG.to_csv(‘BE_GtoA_SingleG.csv’)
ABE_AtoG_hasPAM=ABE_AtoG_editable[[type(x)==list and x!=[ ] for x in
ABE_AtoG_editable.gRNAall]]
ABE_AtoG_hasPAM.to_csv(‘ABE_AtoG_hasPAM.csv’)
ABE_AtoG_SingleA=ABE_AtoG_editable[[type(x)==list and x!=[ ] for x in ABE_AtoG_editable.gRNAs]]
ABE_AtoG_SingleA.to_csv(‘ABE_AtoG_SingleA.csv’)
ABE_TtoC_hasPAM=ABE_TtoC_editable[[type(x)==list and x!=[ ] for x in
ABE_TtoC_editable.gRNAall]]
ABE_TtoC_hasPAM.to_csv(‘ABE_TtoC_hasPAM.csv’)
ABE_TtoC_SingleT=ABE_TtoC_editable[[type(x)==list and x!=[ ] for x in ABE_TtoC_editable.gRNAs]]
ABE_TtoC_SingleT.to_csv(‘ABE_TtoC_SingleT.csv’)
with open(“Summary.txt”, “w”) as text_file:

text_file.write(“BE3/4 \n”)
text_file.write(“single base %s \n” % (len(BE_CtoT_SingleC)+len(BE_GtoA_SingleG)))
text_file.write(“hasPAM %s \n” % (len(BE_CtoT_hasPAM)+len(BE_GtoA_hasPAM)))
text_file.write(“Pathogenic SNPs that can be targeted with BE %s \n” % (GC_AT_size))
text_file.write(“ABE \n”)
text_file.write(“single base %s \n” % (len(ABE_AtoG_SingleA)+len(ABE_TtoC_SingleT)))
text_file.write(“hasPAM %s \n” % (len(ABE_AtoG_hasPAM)+len(ABE_TtoC_hasPAM)))
text_file.write(“Pathogenic SNPs that can be targeted with ABE %s” % (AT_GC_size)

### SUPPLEMENTARY REFERENCES

1. Badran, A. H. et al. Continuous evolution of Bacillus thuringiensis toxins overcomes insect resistance. Nature 533, 58-63, doi:10.1038/nature17938 (2016).
2. Hubbard, B. P. et al. Continuous directed evolution of DNA-binding proteins to improve TALEN specificity. Nat Methods 12, 939-942, doi:10.1038/nmeth.3515 (2015).
3. Badran, A. H. & Liu, D. R. Development of potent in vivo mutagenesis plasmids with broad mutational spectra. Nat Commun 6, 8425, doi:10.1038/ncomms9425 (2015).
4. Chavez, A. et al. Highly efficient Cas9-mediated transcriptional programming. Nat Methods 12, 326-328, doi:10.1038/nmeth.3312 (2015).
5. Komor, A. C., Kim, Y. B., Packer, M. S., Zuris, J. A. & Liu, D. R. Programmable editing of a target base in genomic DNA without double-stranded DNA cleavage. Nature 533, 420-424, doi:10.1038/nature17946 (2016).
6. Komor, A. C. et al. Improved base excision repair inhibition and bacteriophage Mu Gam protein yields C:G-to-T:A base editors with higher efficiency and product purity. Sci Adv 3, eaao4774, doi:10.1126/sciadv.aao4774 (2017).
7. Gaudelli, N. M. et al. Programmable base editing of A*T to G*C in genomic DNA without DNA cleavage. Nature 551, 464-471, doi:10.1038/nature24644 (2017).

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. A Cas9 domain comprising an amino acid sequence that is at least 80% identical to the amino acid sequence of any one of SEQ ID NOs: 9-262,
wherein the Cas9 domain comprises at least one mutation in an amino acid residue selected from the group consisting of amino acid residues 51, 86, 115, 261, 274, 331, 319, 341, 388, 405, 435, 461, 510, 522, 548, 593, 653, 712, 715, 772, 777, 798, 811, 839, 847, 955, 967, 991, 1139, 1199, 1227, 1229, 1296, and 1318 of the amino acid sequence provided by SEQ ID NO: 9, or in a corresponding amino acid residue in any of the amino acid sequences provided in SEQ ID NO: 10-262, and
wherein the amino acid sequence of the Cas9 domain is not identical to the amino acid sequence of a naturally occurring Cas9 domain.

2. The Cas9 domain of claim 1, wherein the Cas9 domain comprises at least two, at least three, at least four, at least five, at least six, at least seven, at least eight, at least nine, at least ten, or more mutations in an amino acid residue selected from the group consisting of amino acid residues 51, 86, 115, 261, 274, 331, 319, 341, 388, 405, 435, 461, 510, 522, 548, 593, 653, 712, 715, 772, 777, 798, 811, 839, 847, 955, 967, 991, 1139, 1199, 1227, 1229, 1296, and 1318 of the amino acid sequence provided by SEQ ID NO: 9, or in a corresponding amino acid residue in any of the amino acid sequences provided in SEQ ID NO: 10-262.

3. The Cas9 domain of claim 1 or 2, wherein the Cas9 domain is at least 85%, at least 90%, at least 92%, at least 95%, at least 96%, at least 97%, at least 98%, at least 99%, or at least 99.5% identical to the amino acid sequence of a Cas9 as provided by any one of SEQ ID NOs: 9-262.

4. The Cas9 domain of any one of claims 1-3, wherein the amino acid sequence of the Cas9 domain comprises at least one mutation selected from the group consisting of X51I, X86L, X115H, X261G, X274E, X331Y, X319T, X341H, X388K, X405Y, X435N, X461I, X510E, X522D, X548V, X593A, X653S, X712K, X715V, X772R, X777N, X798K, X811I, X839G, X847F, X955I, X967K, X991V, X1139A, X1199T, X1227S, X1229S, X1296N, and X1318S of the amino acid sequence provided in SEQ ID NO: 9, or a corresponding mutation in any of the amino acid sequences provided in SEQ ID NOs: 10-262, wherein X represents any amino acid.

5. The Cas9 domain of claim 4, wherein the amino acid sequence of the Cas9 domain comprises at least two at least three, at least four, at least five, at least six, at least seven, at least eight, at least nine, at least ten, or more mutations selected from the group consisting of X51I, X86L, X115H, X261G, X274E, X331Y, X319T, X341H, X388K, X405Y, X435N, X461I, X510E, X522D, X548V, X593A, X653S, X712K, X715V, X772R, X777N, X798K, X811I, X839G, X847F, X955I, X967K, X991V, X1139A, X1199T, X1227S, X1229S, X1296N, and X1318S of the amino acid sequence provided in SEQ ID NO: 9, or a corresponding mutation in any of the amino acid sequences provided in SEQ ID NOs: 10-262, wherein X represents any amino acid.

6. The Cas9 domain of any one of claims 1-5, wherein the amino acid sequence of the Cas9 domain comprises at least one mutation selected from the group consisting of L51I, F86L, R115H, D261G, D274E, D331Y, A319T, Q341H, E388K, F405Y, D435N, R461I, K510E, N522D, I548V, T593A, R653S, Q712K, G715V, S777N, K772R, E798K, L811I, D839G, L847F, V955I, R967K, A991V, V1139A, P1199T, A1227S, P1229S, K1296N, and L1318S of the amino acid sequence provided in SEQ ID NO: 9, or a corresponding mutation in any of the amino acid sequences provided in SEQ ID NOs: 10-262.

7. The Cas9 domain of claim 6, wherein the amino acid sequence of the Cas9 domain comprises at least two at least three, at least four, at least five, at least six, at least seven, at least eight, or at least nine mutations selected from the group consisting of L51I, F86L, R115H, D261G, D274E, D331Y, A319T, Q341H, E388K, F405Y, D435N, R461I, K510E, N522D, I548V, T593A, R653S, Q712K, G715V, S777N, K772R, E798K, L811I, D839G, L847F, V955I, R967K, A991V, V1139A, P1199T, A1227S, P1229S, K1296N, and L1318S of the amino acid sequence provided in SEQ ID NO: 9, or a corresponding mutation in any of the amino acid sequences provided in SEQ ID NOs: 10-262.

8. The Cas9 domain of any one of claims 1-7 further comprising at least one mutation in an amino acid residue selected from the group consisting of amino acid residues 108, 141, 175, 217, 230, 257, 262, 267, 284, 294, 324, 405, 409, 466, 480, 543, 673, 694, 711, 1063, 1207, 1219, 1224, 1256, 1264, 1356, and 1362 of the amino acid sequence provided by SEQ ID NO: 9, or in a corresponding amino acid residue in any of the amino acid sequences provided in SEQ ID NO: 10-262.

9. The Cas9 domain of claim 8, wherein the Cas9 domain comprises at least two, at least three, at least four, at least five, at least six, at least seven, at least eight, at least nine, at least ten, or more mutations in an amino acid residue selected from the group consisting of amino acid residues 108, 141, 175, 217, 230, 257, 262, 267, 284, 294, 324, 405, 409, 466, 480, 543, 673, 694, 711, 1063, 1207, 1219, 1224, 1256, 1264, 1356, and 1362 of the amino acid sequence provided by SEQ ID NO: 9, or in a corresponding amino acid residue in any of the amino acid sequences provided in SEQ ID NO: 10-262.

10. The Cas9 domain of claim 8 or 9, wherein the amino acid sequence of the Cas9 domain comprises at least one mutation selected from the group consisting of X108G, X141Q, X175T, X217A, X230F, X230S, X257N, X262T, X267G, X284N, X294R, X324L, X405I, X409I, X466A, X480K, X543D, X673E, X694I, X711E, X1063V, X1207G, X1219V, X1224N, X1256K, X1264Y, X1356I, and X1362P of the amino acid sequence provided in SEQ ID NO: 9, or a corresponding mutation in any of the amino acid sequences provided in SEQ ID NOs: 10-262, wherein X represents any amino acid.

11. The Cas9 domain of claim 10, wherein the amino acid sequence of the Cas9 domain comprises at least two at least three, at least four, at least five, at least six, at least seven, at least eight, at least nine, at least ten, or more mutations selected from the group consisting of X108G, X141Q, X175T, X217A, X230F, X230S, X257N, X262T, X267G, X284N, X294R, X324L, X405I, X409I, X466A, X480K, X543D, X673E, X694I, X711E, X1063V, X1207G, X1219V, X1224N, X1256K, X1264Y, X1356I, and X1362P of the amino acid sequence provided in SEQ ID NO: 9, or a corresponding mutation in any of the amino acid sequences provided in SEQ ID NOs: 10-262, wherein X represents any amino acid.

12. The Cas9 domain of any one of claims 8-11, wherein the amino acid sequence of the Cas9 domain comprises at least one mutation selected from the group consisting of E108G, K141Q, N175T, S217A, P230F, P230S, D257N, A262T, S267G, D284N, K294R, R324L, F405I, S409I, T466A, E480K, E543D, K673E, M694I, A711E, I1063V, E1207G, E1219V, K1224N, Q1256K, H1264Y, L1356I, and L1362P of the amino acid sequence provided in SEQ ID NO: 9, or a corresponding mutation in any of the amino acid sequences provided in SEQ ID NOs: 10-262.

13. The Cas9 domain of claim 10, wherein the amino acid sequence of the Cas9 domain comprises at least two at least three, at least four, at least five, at least six, at least seven, at least eight, at least nine, at least ten, or more mutations selected from the group consisting of E108G, K141Q, N175T, S217A, P230F, P230S, D257N, A262T, S267G, D284N, K294R, R324L, F405I, S409I, T466A, E480K, E543D, K673E, M694I, A711E, I1063V, E1207G, E1219V, K1224N, Q1256K, H1264Y, L1356I, and L1362P of the amino acid sequence provided in SEQ ID NO: 9, or a corresponding mutation in any of the amino acid sequences provided in SEQ ID NOs: 10-262.

14. The Cas9 domain of any one of claims 1-13 further comprising at least one mutation in an amino acid residue selected from the group consisting of amino acid residues 23, 122, 137, 182, 394, 474, 554, 654, 660, 727, 763, 845, 847, 1100, 1135, 1218, 1224, 1333, 1335, and 1337 of the amino acid sequence provided by SEQ ID NO: 9, or in a corresponding amino acid residue in any of the amino acid sequences provided in SEQ ID NO: 10-262.

15. The Cas9 domain of claim 14, wherein the amino acid sequence of the Cas9 domain comprises at least one mutation selected from the group consisting of X23N, X122, X137, X182, X394H, X474I, X554R, X654L, X660, X727P, X763I, X845, X847, X1100I, X1135, X1218, X1224N, X1333, X1335, and X1337 of the amino acid sequence provided in SEQ ID NO: 9, or a corresponding mutation in any of the amino acid sequences provided in SEQ ID NOs: 10-262, wherein X represents any amino acid.

16. The Cas9 domain of claim 15, wherein the amino acid sequence of the Cas9 domain comprises at least one mutation selected from the group consisting of D23N, Q394H, T474I, K554R, R654L, L727P, M763I, V1100I, and K1224N of the amino acid sequence provided in SEQ ID NO: 9, or a corresponding mutation in any of the amino acid sequences provided in SEQ ID NOs: 10-262.

17. The Cas9 domain of any one of claims 1-16, wherein the Cas9 domain comprises at least one mutation in an amino acid residue selected from the group consisting of amino acid residues 108, 175, 217, 230, 257, 262, 267, 294, 324, 409, 461, 466, 480, 543, 673, 694, 711, 777, 1063, 1207, 1219, 1256, 1264, and 1356 of the amino acid sequence provided by SEQ ID NO: 9, or in a corresponding amino acid residue in any of the amino acid sequences provided in SEQ ID NO: 10-262.

18. The Cas9 domain of claim 17, wherein the Cas9 domain comprises at least two, at least three, at least four, at least five, at least six, at least seven, at least eight, or at least nine mutations in an amino acid residue selected from the group consisting of amino acid residues 108, 175, 217, 230, 257, 262, 267, 294, 324, 409, 461, 466, 480, 543, 673, 694, 711, 777, 1063, 1207, 1219, 1256, 1264, and 1356 of the amino acid sequence provided by SEQ ID NO: 9, or in a corresponding amino acid residue in any of the amino acid sequences provided in SEQ ID NO: 10-262.

19. The Cas9 domain of claim 17 or 18, wherein the amino acid sequence of the Cas9 domain comprises at least one mutation selected from the group consisting of X108G, X175T, X217A, X230F, X257N, X262T, A267G, X294R, X324L, X409I, X461I, X466A, X480K, X543D, X673E, X694I, X711E, X777N, X1063V, X1207G, X1219V, X1256K, X1264Y, and X1356I of the amino acid sequence provided in SEQ ID NO: 9, or a corresponding mutation in any of the amino acid sequences provided in SEQ ID NOs: 10-262, wherein X represents any amino acid.

20. The Cas9 domain of claim 4, wherein the amino acid sequence of the Cas9 domain comprises at least two at least three, at least four, at least five, at least six, at least seven, at least eight, or at least nine mutations selected from the group consisting of X108G, X175T, X217A, X230F, X257N, X262T, A267G, X294R, X324L, X409I, X461I, X466A, X480K, X543D, X673E, X694I, X711E, X777N, X1063V, X1207G, X1219V, X1256K, X1264Y, and X1356I of the amino acid sequence provided in SEQ ID NO: 9, or a corresponding mutation in any of the amino acid sequences provided in SEQ ID NOs: 10-262, wherein X represents any amino acid.

21. The Cas9 domain of any one of claims 17-20, wherein the amino acid sequence of the Cas9 domain comprises at least one mutation selected from the group consisting of E108G, N175T, S217A, P230F, D257N, A262T, S267G, K294R, R324L, S409I, R461I, T466A, E480K, E543D, K673E, M694I, A711E, S777N, I1063V, E1207G, E1219V, Q1256K, H1264Y, and L1356I of the amino acid sequence provided in SEQ ID NO: 9, or a corresponding mutation in any of the amino acid sequences provided in SEQ ID NOs: 10-262.

22. The Cas9 domain of claim 21, wherein the amino acid sequence of the Cas9 domain comprises at least two at least three, at least four, at least five, at least six, at least seven, at least eight, or at least nine mutations selected from the group consisting of E108G, N175T, S217A, P230F, D257N, A262T, S267G, K294R, R324L, S409I, R461I, T466A, E480K, E543D, K673E, M694I, A711E, S777N, I1063V, E1207G, E1219V, Q1256K, H1264Y, and L1356I of the amino acid sequence provided in SEQ ID NO: 9, or a corresponding mutation in any of the amino acid sequences provided in SEQ ID NOs: 10-262.

23. The Cas9 domain of any one of claims 17-22, wherein the Cas9 domain comprises at least one, at least two, at least three, at least four, at least five, at least six, at least seven, at least eight, or at least nine mutations in an amino acid residue selected from the group consisting of amino acid residues 108, 217, 262, 409, 480, 543, 694, 1219, and 1356 of the amino acid sequence provided in SEQ ID NO: 9, or in a corresponding amino acid residue in any of the amino acid sequences provided in SEQ ID NOs: 10-262.

24. The Cas9 domain of claim 23, wherein the amino acid sequence of the Cas9 domain comprises at least one, at least two, at least three, at least four, at least five, at least six, at least seven, at least eight, or at least nine mutations selected from the group consisting of X108G, X217A, X262T, X409I, X480K, X543D, X694I, X1219V, and X1356I of the amino acid sequence provided in SEQ ID NO: 9, or a corresponding mutation in any of the amino acid sequences provided in SEQ ID NOs: 10-262, wherein X represents any amino acid at the position.

25. The Cas9 domain of claim 23 or 24, wherein the amino acid sequence of the Cas9 domain comprises at least one, at least two, at least three, at least four, at least five, at least six, at least seven, at least eight, or at least nine mutations selected from the group consisting of E108G, S217A, A262T, S409I, E480K, E543D, M694I, E1219V, and L1356I of the amino acid sequence provided in SEQ ID NO: 9, or a corresponding mutation in any of the amino acid sequences provided in SEQ ID NOs: 10-262.

26. The Cas9 domain of claim 25, wherein the Cas9 domain comprises a E108G, S217A, A262T, S409I, E480K, E543D, M694I, E1219V, and L1356I mutation of the amino acid sequence provided in SEQ ID NO: 9, or the corresponding mutations in any of the amino acid sequences provided in SEQ ID NOs: 10-262.

27. The Cas9 domain of any one of claims 17-22, wherein the Cas9 domain comprises at least one, at least two, at least three, at least four, at least five, at least six, or at least seven mutations in an amino acid residue selected from the group consisting of amino acid residues 262, 324, 409, 480, 543, 694, and 1219 of the amino acid sequence provided in SEQ ID NO: 9, or in a corresponding amino acid residue in any of the amino acid sequences provided in SEQ ID NOs: 10-262.

28. The Cas9 domain of claim 27, wherein the amino acid sequence of the Cas9 domain comprises at least one, at least two, at least three, at least four, at least five, at least six, or at least seven mutations selected from the group consisting of X262T, X324L, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid at the corresponding position.

29. The Cas9 domain of claim 27 or 28, wherein the amino acid sequence of the Cas9 domain comprises at least one, at least two, at least three, at least four, at least five, at least six, or at least seven mutations selected from the group consisting of A262T, R324L, S409I, E480K, E543D, M694I, and E1219V of the amino acid sequence provided in SEQ ID NO: 9, or a corresponding mutation in any of the amino acid sequences provided in SEQ ID NOs: 10-262.

30. The Cas9 domain of claim 29, wherein the Cas9 domain comprises a A262T, R324L, S409I, E480K, E543D, M694I, and E1219V mutation of the amino acid sequence provided in SEQ ID NO: 9, or the corresponding mutations in any of the amino acid sequences provided in SEQ ID NOs: 10-262.

31. The Cas9 domain of any one of claims 1-30, wherein the Cas9 domain further comprises a D10A and/or a H840A mutation of the amino acid sequence provided in SEQ ID NO: 9, or a corresponding mutation in any of the amino acid sequences provided in SEQ ID NOs: 10-262.

32. The Cas9 domain of claim 31, wherein the Cas9 domain comprises a D10X₁ and/or a H840X₂ mutation of the amino acid sequence provided in SEQ ID NO: 9, or a corresponding mutation in any of the amino acid sequences provided in SEQ ID NOs: 10-262, wherein X₁ is any amino acid except for D, and wherein X₂ is any amino acid except for H.

33. The Cas9 domain of claim 31 or 32, wherein the Cas9 domain comprises a D10A mutation of the amino acid sequence provided in SEQ ID NO: 9, or a corresponding mutation in any of the amino acid sequences provided in SEQ ID NOs: 10-262.

34. The Cas9 domain of claim 33, wherein the Cas9 domain comprises an H at amino acid residue 840 of the amino acid sequence provided in SEQ ID NO: 9, or a corresponding residue in any of the amino acid sequences provided in SEQ ID NOs: 10-262.

35. The Cas9 domain of claim 31 or 32, wherein the Cas9 domain comprises an H840A mutation of the amino acid sequence provided in SEQ ID NO: 9, or a corresponding mutation in any of the amino acid sequences provided in SEQ ID NOs: 10-262.

36. The Cas9 domain of claim 35, wherein the recombinant Cas9 protein comprises a D at amino acid residue 10 of the amino acid sequence provided in SEQ ID NO: 9, or a corresponding residue in any of the amino acid sequences provided in SEQ ID NOs: 10-262.

37. The Cas9 domain of any one of claims 1-36, wherein the Cas9 domain exhibits activity on a target sequence having a 3′ end that is not directly adjacent to the canonical 5′-NGG-3′ protospacer-adjacent motif (PAM) sequence, or a target sequence that does not comprise the canonical 5′-NGG-3′ PAM sequence, wherein N is A, C, G, or T.

38. The Cas9 domain of claim 37, wherein the Cas9 domain exhibits activity on a target sequence having a 3′ end that is not directly adjacent to the canonical PAM sequence (5′-NGG-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of the Streptococcus pyogenes Cas9 domain as provided by SEQ ID NO: 9 on the same target sequence, wherein N is A, C, G, or T.

39. The Cas9 domain of claim 37 or 38, wherein the 3′-end of the target sequence is directly adjacent to an NGT, NGA, NGC, or NNG PAM sequence, wherein N is an A, C, G, or T.

40. The Cas 9 domain of any one of claims 37-39, wherein the 3′ end of the target sequence is directly adjacent to a AAA, AAC, AAG, AAT, CAA, CAC, CAG, CAT, GAA, GAC, GAG, GAT, TAA, TAC, TAG, TAT, ACA, ACC, ACG, ACT, CCA, CCC, CCG, CCT, GCA, GCC, GCG, GCT, TCA, TCC, TCG, TCT, AGA, AGC, AGT, CGA, CGC, CGT, GGA, GGC, GGT, TGA, TGC, TGT, ATA, ATC, ATG, ATT, CTA, CTC, CTG, CTT, GTA, GTC, GTG, GTT, TTA, TTC, TTG, or TTT PAM sequence.

41. The Cas9 domain of any one of claims 37-40, wherein the 3′-end of the target sequence is directly adjacent to an CGG, AGT, TGG, AGT, CGT, GGG, CGT, TGT, GGT, AGC, CGC, TGC, GGC, AGA, CGA, TGA, GGA, GAA, GAT, or CAA sequence.

42. The Cas9 domain of any one of claims 37-41, wherein the activity is binding to the target sequence, or wherein the activity is increased binding activity at the target sequence.

43. The Cas9 domain of any one of claims 1-42, wherein the Cas9 domain exhibits lower off-target activity as compared to the off-target activity of the Streptococcus pyogenes Cas9 domain as provided by SEQ ID NO: 9.

44. The Cas9 domain of any one of claims 37-43, wherein the Cas9 domain activity is measured by a nuclease assay, a deamination assay, a transcriptional activation assay, or by PCR or sequencing.

45. The Cas9 domain of claim 44, wherein the transcriptional activation assay is a GFP activation assay.

46. The Cas9 domain of claim 44, wherein sequencing is used to measure indel formation.

47. A fusion protein comprising (i) the Cas9 domain of any one of claims 1-44, and (ii) an effector domain.

48. The fusion protein of claim 47, wherein the effector domain is a domain that comprises nuclease activity, nickase activity, recombinase activity, deaminase activity, methyltransferase activity, methylase activity, acetylase activity, acetyltransferase activity, transcriptional activation activity, or transcriptional repression activity.

49. The fusion protein of claim 47 or 48, wherein the effector domain is a nucleic acid editing domain.

50. The fusion protein of claim 49, wherein the nucleic acid editing domain comprises a deaminase domain.

51. The fusion protein of claim 50, wherein the deaminase domain is a cytidine deaminase domain.

52. The fusion protein of claim 51, wherein the cytidine deaminase domain is an apolipoprotein B mRNA-editing complex (APOBEC) family deaminase.

53. The fusion protein of claim 51 or 52, wherein the cytidine deaminase domain is at least 80%, at least 85%, at least 90%, at least 92%, at least 95%, at least 96%, at least 97%, at least 98%, at least 99%, or at least 99.5% identical to the cytidine deaminase domain of any one of SEQ ID NOs: 350-389.

54. The fusion protein of claim 51 or 52, wherein the cytidine deaminase domain comprises the amino acid sequence of any one of SEQ ID NOs: 350-389.

55. The fusion protein of any one of claims 47-54, wherein the fusion protein further comprises a uracil glycosylase inhibitor (UGI) domain.

56. The fusion protein of claim 55, wherein the UGI domain comprises the amino acid sequence of SEQ ID NO: 500.

57. The fusion protein of any one of claims 47-56, wherein the fusion protein comprises the amino acid sequence of SEQ ID NO: 534.

58. The fusion protein of any one of claims 47-56, wherein the fusion protein comprises the amino acid sequence of SEQ ID NO: 538.

59. The fusion protein of any one of claims 47-58, wherein the fusion protein further comprises a second UGI domain.

60. The fusion protein of claim 59, wherein the fusion protein comprises the amino acid sequence of SEQ ID NO: 540.

61. The fusion protein of claim 59, wherein the fusion protein comprises the amino acid sequence of SEQ ID NO: 541.

62. The fusion protein of claim 50, wherein the deaminase domain is an adenosine deaminase domain.

63. The fusion protein of claim 62 further comprising a second adenosine deaminase domain.

64. The fusion protein of claim 63, wherein the first adenosine deaminase domain and the second adenosine deaminase domain comprises an ecTadA domain, or variant thereof.

65. The fusion protein of claim 64, wherein the first adenosine deaminase domain and the second adenosine deaminase domain comprise the amino acid sequence of any one of SEQ ID NOs: 400-458.

66. The fusion protein of claim 65, wherein the first adenosine deaminase comprises the amino acid sequence of SEQ ID NO: 400.

67. The fusion protein of claim 65, wherein the second adenosine deaminase comprises the amino acid sequence of SEQ ID NO: 458.

68. The fusion protein of any one of claims 62-67, wherein the fusion protein comprises the amino acid sequence of SEQ ID NO: 535.

69. The fusion protein of any one of claims 62-67, wherein the fusion protein comprises the amino acid sequence of SEQ ID NO: 539.

70. A complex comprising the fusion protein of any one of claims 47-69, and a guide RNA bound to the recombinant Cas9 protein.

71. The complex of claim 70, wherein the guide RNA is about 15-100 nucleotides long and comprises a sequence of at least 10 contiguous nucleotides that is complementary to a target sequence.

72. The complex of claim 71, wherein the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long.

73. The complex of any one of claims 70-72, wherein the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

74. The complex of any one of claims 71-73, wherein the target sequence is a DNA sequence.

75. The complex of claim 74, wherein the target sequence is a sequence in the genome of a mammal.

76. The complex of claim 75, wherein the target sequence is a sequence in the genome of a human.

77. The complex of any one of claims 71-76, wherein the target sequence comprises a sequence associated with a disease or disorder.

78. The complex of claim 77, wherein the target sequence comprises a point mutation associated with a disease or disorder.

79. The complex of claim 78, wherein the complex edits a point mutation in the target sequence.

80. The complex of claim 79, wherein the point mutation is located between about 10 to about 20 nucleotides upstream of the PAM in the target sequence.

81. The complex of claim 79 or 80, wherein the target sequence comprises a T→C point mutation.

82. The complex of claim 87, wherein the complex deaminates the target C point mutation, and wherein the deamination results in a sequence that is not associated with a disease or disorder.

83. The complex of claim 82, wherein the target C point mutation is present in the DNA strand that is not complementary to the guide RNA.

84. The complex of claim 79 or 80, wherein the target sequence comprises a G→A point mutation.

85. The complex of claim 84, wherein the complex deaminates the target A point mutation, and wherein the deamination results in a sequence that is not associated with a disease or disorder.

86. The complex of claim 85, wherein the target A point mutation is present in the DNA strand that is not complementary to the guide RNA.

87. The complex of any one of claims 70-86, wherein the complex exhibits increased deamination efficiency of a point mutation in a target sequence that does not comprise the canonical PAM (5′-NGG-3′) at its 3′ end as compared to the deamination efficiency of a complex comprising Streptococcus pyogenes Cas9 domain as provided by SEQ ID NO: 9.

88. The complex of claim 87, wherein the complex exhibits increased deamination efficiency of a point mutation in a target sequence having a 3′ end that is not directly adjacent to the canonical PAM sequence (5′-NGG-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the deamination efficiency of complex comprising the Streptococcus pyogenes Cas9 domain as provided by SEQ ID NO: 9 on the same target sequence.

89. The complex of claim 87 or 88, wherein the 3′ end of the target sequence is directly adjacent to a sequence selected from the group consisting of NGT, NGA, NGC, and NNG, wherein N is an A, G, T, or C.

90. The complex of any one of claims 87-89, wherein the 3′ end of the target sequence is directly adjacent to a sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA.

91. The complex of any one of claims 69-72, wherein deamination activity is measured using a deamination assay, PCR, or sequencing.

92. The complex of any one of claims 70-91, wherein the complex produces fewer indels in a target sequence that does not comprise the canonical PAM (5′-NGG-3′) at its 3′ end as compared to the amount of indels produced by a complex comprising Streptococcus pyogenes Cas9 domain as provided by SEQ ID NO: 9.

93. The complex of claim 92, wherein the complex produces fewer indels in a target sequence having a 3′ end that is not directly adjacent to the canonical PAM sequence (5′-NGG-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold lower as compared to the amount of indels produced by a complex comprising Streptococcus pyogenes Cas9 domain as provided by SEQ ID NO: 9 on the same target sequence.

94. The complex of claim 92 or 93, wherein the 3′ end of the target sequence is directly adjacent to a sequence selected from the group consisting of NGT, NGA, NGC, and NNG, wherein N is an A, G, T, or C.

95. The complex of any one of claims 92-94, wherein the 3′ end of the target sequence is directly adjacent to a sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA.

96. The complex of any one of claims 92-95, wherein indels are measured using high-throughput sequencing.

97. The complex of any one of claims 70-96, wherein the complex exhibits a decreased off-target activity as compared to the off-target activity of a complex comprising Streptococcus pyogenes Cas9 domain as provided by SEQ ID NO: 9.

98. The complex of claim 97, wherein the off-target activity of the complex is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold decreased as compared to the off-target activity of a complex comprising Streptococcus pyogenes Cas9 domain as provided by SEQ ID NO: 9.

99. The complex of any one of claims 71-98, wherein the target sequence is in the genome of an organism.

100. The complex of claim 99, wherein the organism is a prokaryote.

101. The complex of claim 100, wherein the prokaryote is a bacterium.

102. The complex of claim 99, wherein the organism is a eukaryote.

103. The complex of claim 99, wherein the organism is a plant or fungus.

104. The complex of claim 99, wherein the organism is a vertebrate.

105. The complex of claim 104, wherein the vertebrate is a mammal.

106. The complex of claim 105, wherein the mammal is a human.

107. The complex of claim 99, wherein the organism is a cell.

108. The complex of claim 107, wherein the cell is a human cell.

109. A method comprising contacting a nucleic acid with the fusion protein of any one of claims 47-69, and with a guide RNA, wherein the guide RNA is about 15-100 nucleotides long and comprises a sequence of at least 10 contiguous nucleotides that is complementary to a target sequence.

110. A method comprising contacting a cell with the fusion protein of any one of claims 47-69, and with a guide RNA, wherein the guide RNA is about 15-100 nucleotides long and comprises a sequence of at least 10 contiguous nucleotides that is complementary to a target sequence.

111. A method comprising contacting a nucleic acid with the complex of any one of claims 70-108.

112. A method comprising contacting a cell with the complex of any one of claims 70-108.

113. The method of any one of claims 109-112, wherein the contacting is performed in vitro.

114. The method of any one of claims 109-112, wherein the contacting is performed in vivo.

115. A method comprising administering to a subject the fusion protein of any one of claims 47-69, and a guide RNA, wherein the guide RNA is about 15-100 nucleotides long and comprises a sequence of at least 10 contiguous nucleotides that is complementary to a target sequence.

116. A method comprising administering to a subject the complex of any one of claims 70-108.

117. The method of any one of claims 109-116, wherein the target sequence of the nucleic acid is a DNA sequence.

118. The method of any one of claims 109-117, wherein the 3′ end of the target sequence is not immediately adjacent to the canonical PAM sequence (5′-NGG-3′).

119. The method of claim 118, wherein the 3′ end of the target sequence is directly adjacent to a sequence selected from the group consisting of NGT, NGA, NGC, and NNG, wherein N is an A, G, T, or C.

120. The complex of claim 118 or 119, wherein the 3′ end of the target sequence is directly adjacent to a sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA.

121. The method of any one of claims 109-120, wherein the target sequence comprises a sequence associated with a disease or disorder.

122. The method of claim 121, wherein the target DNA sequence comprises a point mutation associated with a disease or disorder.

123. The method of any one of claims 109-116, wherein the activity of the fusion protein, or the activity of the complex, results in a correction of the point mutation.

124. The method of any one of claims 121-123, wherein the target DNA sequence comprises a T→C point mutation associated with a disease or disorder, and wherein the deamination of the mutant C base results in a sequence that is not associated with a disease or disorder.

125. The method of claim 124, wherein the target DNA sequence encodes a protein and wherein the point mutation is in a codon and results in a change in the amino acid encoded by the mutant codon as compared to the wild-type codon.

126. The method of claim 125, wherein the deamination of the mutant C results in a change of the amino acid encoded by the mutant codon.

127. The method of claim 126, wherein the deamination of the mutant C results in the codon encoding the wild-type amino acid.

128. The method of any one of claims 121-123, wherein the target DNA sequence comprises a G to A point mutation associated with a disease or disorder, and wherein the deamination of the mutant A base results in a sequence that is not associated with a disease or disorder.

129. The method of claim 128, wherein the target DNA sequence encodes a protein and wherein the point mutation is in a codon and results in a change in the amino acid encoded by the mutant codon as compared to the wild-type codon.

130. The method of claim 129, wherein the deamination of the mutant A results in a change of the amino acid encoded by the mutant codon.

131. The method of claim 130, wherein the deamination of the mutant A results in the codon encoding the wild-type amino acid.

132. The method of any one of claims 109-131, wherein the contacting is in vivo in a subject.

133. The method of claim 132, wherein the subject has or has been diagnosed with a disease or disorder.

134. The method of claim 132 or 133, wherein the disease or disorder is a proliferative disease, a genetic disease, a neoplastic disease, a metabolic disease, or a lysosomal storage disease.

135. The method of claim 134, wherein the disease or disorder is associated with a T to C or A to G mutation in a gene selected from the genes disclosed in Table 5 and 18, respectively.

136. The method of claim 134, wherein the disease or disorder is associated with a G to A or C to T mutation in a gene.

137. A kit comprising a nucleic acid construct, comprising:
(a) a nucleic acid sequence encoding the Cas9 domain of any one of claims 1-43, or the fusion protein of any one of claims 47-69; and
(b) a heterologous promoter that drives expression of the sequence of (a).

138. A kit comprising a nucleic acid construct, comprising:
(a) a nucleic acid sequence encoding the complex of any one of claims 70-108; and
(b) a heterologous promoter that drives expression of the sequence of (a).

139. The kit of claim 137 further comprising an expression construct encoding a guide RNA backbone, wherein the construct comprises a cloning site positioned to allow the cloning of a nucleic acid sequence identical or complementary to a target sequence into the guide RNA backbone.

140. A polynucleotide encoding the Cas9 domain of any one of claims 1-43, the fusion protein of any one of claims 47-69, or the complex of any one of claims 70-108.

141. A vector comprising a polynucleotide of claim 140.

142. The vector of claim 141, wherein the vector comprises a heterologous promoter driving expression of the polynucleotide encoding the fusion protein or the polynucleotide encoding the complex.

143. A method comprising contacting a cell with the vector of claim 141 or 142.

144. The method of claim 143, wherein the cell vector is transfected into the cell.

145. The method of claim 144, wherein the vector is transfected into the cell using electroporation, heat shock, or a composition comprising a cationic lipid.

146. A cell comprising the Cas9 domain of any one of claims 1-43, the fusion protein of any one of claims 47-69, a nucleic acid molecule encoding the Cas9 domain of any one of claims 1-43, or a nucleic acid molecule encoding the fusion protein of any one of claims 47-69.

147. A cell comprising the complex of any one of claims 70-108, or a nucleic acid molecule encoding the complex of any one of claims 70-108.

148. A cell comprising the vector of claim 141 or 142.

149. A pharmaceutical composition comprising the Cas9 domain of any one of claims 1-43.

150. A pharmaceutical composition comprising the fusion protein of any one of claims 47-69.

151. A pharmaceutical composition comprising the complex of any one of claims 70-108.

152. The pharmaceutical composition of any one of claims 149-151, further comprising a pharmaceutically acceptable excipient.
